(19) United States
(12) Patent Application Publication
(10) Pub. No.: US 2022/0313813 A1
(43) Pub. Date: Oct. 6, 2022

(54) ROTAVIRUS MRNA VACCINE

(71) Applicant: CureVac AG, Tübingen (DE)

(72) Inventors: Susanne RAUCH, Tübingen (DE); Sandro ROIER, Tübingen (DE); Benjamin PETSCH, Tübingen (DE)

(73) Assignee: CureVac AG, Tübingen (DE)

(21) Appl. No.: 17/620,047

(22) PCT Filed: Jun. 18, 2020
(86) PCT No.: PCT/EP2020/067036

§ 371 Date: Dec. 16, 2021

(30) Foreign Application Priority Data
Jun. 18, 2019 (EP) PCT/EP2019/066107

(51) Int. Cl. A61K 39/15; C12N 7/00; C07K 14/005; A61K 39/145; A61P 31/14

(52) U.S. Cl. CPC A61K 39/15; C12N 7/00; C07K 14/005; A61K 39/145; A61P 31/14; C12N 2720/12334; A61K 2039/53

(57) ABSTRACT

The invention is directed to a coding RNA for a Rotavirus vaccine. The coding RNA comprises at least one coding region encoding at least one antigenic peptide or protein of a Rotavirus, in particular VPS* of a Rotavirus, or immunogenic fragment or immunogenic variant thereof. The present invention is also directed to compositions and vaccines comprising said coding RNA in association with a polymeric carrier, a polycationic protein or peptide, or a lipid nanoparticle (LNP). Further, the invention concerns a kit, particularly a kit of parts comprising the coding RNA, or the composition, or the vaccine. The invention is also directed to a kit or kit of parts, medical treatments, and the first and second medical uses.

### INTRODUCTION

The present invention is directed to a coding RNA for a Rotavirus vaccine. The coding RNA comprises at least one heterologous untranslated region (UTR), preferably a 3′-UTR and/or a 5′-UTR, and a coding region (cds) encoding at least one antigenic peptide or protein of a Rotavirus, in particular at least one antigenic protein derived from VP8* of a Rotavirus. The present invention is also directed to compositions and vaccines comprising at least one of said coding RNAs in association with a polymeric carrier, a polycationic protein or peptide, or a lipid nanoparticle (LNP). Further, the invention concerns a kit, particularly a kit of parts comprising the coding RNA, or the composition, or the vaccine. The invention is also directed to first and second medical uses of the coding RNA, the composition, the vaccine, and the kit, and to methods of treating or preventing a Rotavirus infection.

Rotavirus infections are the globally leading cause of severe diarrhoea in children younger than five years of age and account for 50% of hospitalisations for childhood diarrhoea. Worldwide more than 450,000 children under five years die from rotavirus infection each year. While Rotavirus is endemic worldwide with almost every child having been infected by the age of five, Rotavirus infection is most problematic in the developing world: the majority of deaths occur in Africa and Asia, of which India is the country most heavily affected.

At present, there are two licensed oral vaccines available, which are both based on live-attenuated forms of the virus. RotaTeq® (Merck) is based on a bovine Rotavirus strain engineered to express outer layer proteins from human strains. Rotarix® (GlaxoSmithKline) is based on a live-attenuated human Rotavirus strain. Both vaccines are given orally and exhibit high efficacy in the developed world. However, the efficacy of oral Rotavirus vaccination is significantly reduced in developing countries. This is likely to be caused by several factors. Firstly, the virus-based vaccine can be inactivated by pre-existing antibodies, e.g. transferred to babies by breastfeeding. Secondly, malnutrition can have a negative impact on the efficacy of oral vaccinations. Furthermore, unrelated infections of the gastrointestinal tract which are more prevalent in developing countries compared to developed countries, might be a major factor in reducing vaccine efficacy. In addition, an increased risk of intussuseption has been described for live-attenuated oral rotavirus vaccines in the past.

PATH (an international nonprofit organization) is currently investigating non-replicating rotavirus vaccine (NRRV) candidates that may eliminate the risk of intussusception completely. NRRVs that would be administered via non-oral routes may overcome factors that can lessen an oral vaccine's impact including co-infections in the digestive system or the presence of maternally-derived antibodies (Wen, Xiaobo, et at. “Inclusion of a universal tetanus toxoid CD4+ T cell epitope P2 significantly enhanced the immunogenicity of recombinant rotavirus ΔVP8* subunit parenteral vaccines.” Vaccine 32.35 (2014): 4420-4427). The employed antigen design P2-VP8* (P2 is a T cell epitope derived from the tetanus toxoid) has successfully been tested as a protein subunit vaccine in guinea pigs and gnotobiotic pigs and is currently been tested in clinical trials (Groome, Michelle J., et al. “Safety and immunogenicity of a parenteral P2-VP8-P [8] subunit rotavirus vaccine in toddlers and infants in South Africa: a randomised, double-blind, placebo-controlled trial.” The Lancet Infectious Diseases 17.8 (2017): 843-853).

Potential disadvantages of subunit vaccines may be that they generally require strong adjuvants (e.g. aluminium hydroxide) and these adjuvants often induce tissue reactions, the duration of immunity is generally shorter than live vaccines and that they often need to be linked to carriers to enhance their immunogenicity. Furthermore a pathogen can escape immune responses to a single epitope versus multiple epitope vaccines.

Although live oral rotavirus vaccines have been shown to provide protection against rotavirus gastroenteritis caused by rotavirus strains with and without G and P genotypes shared with the vaccine strain, this crossprotection might not occur with subunit vaccines, and a multivalent vaccine with P[4], P[6], and P[8] antigens might be required to provide protection against the common circulating rotavirus strains. Therefore, Groome et al are undertaking a multicentre study to investigate the safety and immunogenicity of a trivalent P2-VP8-P[4/6/8] vaccine (Groome, Michelle J., et al. “Safety and immunogenicity of a parenteral P2-VP8-P [8] subunit rotavirus vaccine in toddlers and infants in South Africa: a randomised, double-blind, placebo-controlled trial.” The Lancet Infectious Diseases 17.8 (2017): 843-853).

Accordingly, there is an urgent need for providing new and improved vaccines, which would be of particular importance for developing countries. Preferably, the new and improved vaccine should allow parenteral, e.g. intramuscular delivery, and thus avoid efficacy reduction induced via oral vaccine delivery. Moreover, the new vaccine should allow cost-effective production. Furthermore, especially for the use in developing countries, there is a need for a temperature stabile Rotavirus vaccine which is not dependent on cooling for storage and distribution.

The objects underlying the present invention are inter alia solved by providing a coding RNA for a Rotavirus vaccine or an RNA based composition/vaccine as further defined in the claims and the description. Further, it would be desirable that such a coding RNA, or a composition/vaccine comprising said coding RNA has at least some of the following advantageous features:
Improved translation of coding RNA constructs at the site of injection (e.g. muscle);
Very efficient induction of antigen-specific immune responses against the encoded Rotavirus protein at a very low dosages and dosing regimen;
Suitability for vaccination of infants and/or newborns;
Suitability for intramuscular administration;
Induction of specific and functional humoral immune response against Rotavirus;
Induction of broad, functional cellular T-cell responses against Rotavirus;
Induction of specific B-cell memory against Rotavirus;
Induction of specific immunoglobulin A (IgA) antibodies;
Fast onset of immune protection against Rotavirus;
Longevity of the induced immune responses against Rotavirus;
Induction of high levels of virus neutralizing antibodies to prevent a Rotavirus infection,
Induction of high levels of virus neutralizing antibodies against homologous and heterologous Rotavirus strains;
No excessive induction of systemic cytokine or chemokine response after application of the vaccine; which could lead to an undesired high reactogenicity upon vaccination
Well tolerability, no side-effects, non-toxicity of the vaccine;
Advantageous stability characteristics of the RNA-based vaccine;
Speed, adaptability, simplicity and scalability of Rotavirus vaccine production;
Advantageous vaccination regimen that only requires one or two vaccination for sufficient protection.

### Definitions

For the sake of clarity and readability the following definitions are provided. Any technical feature mentioned for these definitions may be read on each and every embodiment of the invention. Additional definitions and explanations may be specifically provided in the context of these embodiments.

Percentages in the context of numbers should be understood as relative to the total number of the respective items. In other cases, and unless the context dictates otherwise, percentages should be understood as percentages by weight (wt.-%).

About: The term “about” is used when determinants or values do not need to be identical, i.e. 100% the same. Accordingly, “about” means, that a determinant or values may diverge by 0.1% to 20%, preferably by 0.1% to 10%; in particular, by 0.5%, 1%, 2%, 3%, 4%, 5%, 6%, 7%, 8%, 9%, 10%, 11%, 12%, 13%, 14%, 15%, 16%, 17%, 18%, 19%, 20%. The skilled person will know that e.g. certain parameters or determinants may slightly vary based on the method how the parameter was determined. For example, if a certain determinants or value is defined herein to have e.g. a length of “about 1000 nucleotides”, the length may diverge by 0.1% to 20%, preferably by 0.1% to 10%; in particular, by 0.5%, 1%, 2%, 3%, 4%, 5%, 6%, 7%, 8%, 9%, 10%, 11%, 12%, 13%, 14%, 15%, 16%, 17%, 18%, 19%, 20%. Accordingly, the skilled person will know that in that specific example, the length may diverge by 1 to 200 nucleotides, preferably by 1 to 200 nucleotides; in particular, by 5, 10, 20, 30, 40, 50, 60, 70, 80, 90, 100, 110, 120, 130, 140, 150, 160, 170, 180, 190, 200 nucleotides.

Adaptive immune response: The term “adaptive immune response” as used herein will be recognized and understood by the person of ordinary skill in the art, and is e.g. intended to refer to an antigen-specific response of the immune system (the adaptive immune system). Antigen specificity allows for the generation of responses that are tailored to specific pathogens or pathogen-infected cells. The ability to mount these tailored responses is usually maintained in the body by “memory cells” (B-cells). In the context of the invention, the antigen is provided by the coding RNA encoding at least one antigenic peptide or protein derived from Rotavirus.

Antigen: The term “antigen” as used herein will be recognized and understood by the person of ordinary skill in the art, and is e.g. intended to refer to a substance which may be recognized by the immune system, preferably by the adaptive immune system, and is capable of triggering an antigen-specific immune response, e.g. by formation of antibodies and/or antigen-specific T cells as part of an adaptive immune response. Typically, an antigen may be or may comprise a peptide or protein which may be presented by the MHC to T-cells. Also fragments, variants and derivatives of peptides or proteins derived from e.g. VP8* comprising at least one epitope are understood as antigens in the context of the invention. In the context of the present invention, an antigen may be the product of translation of a provided mRNA as specified herein.

Antigenic peptide or protein: The term “antigenic peptide or protein” or “immunogenic peptide or protein” will be recognized and understood by the person of ordinary skill in the art, and is e.g. intended to refer to a peptide, protein derived from a (antigenic or immunogenic) protein which stimulates the body's adaptive immune system to provide an adaptive immune response. Therefore an antigenic/immunogenic peptide or protein comprises at least one epitope (as defined herein) or antigen (as defined herein) of the protein it is derived from (e.g., VP8* of a Rotavirus).

Cationic: Unless a different meaning is clear from the specific context, the term “cationic” means that the respective structure bears a positive charge, either permanently or not permanently, but in response to certain conditions such as pH. Thus, the term “cationic” covers both “permanently cationic” and “cationisable”.

Cationisable: The term “cationisable” as used herein means that a compound, or group or atom, is positively charged at a lower pH and uncharged at a higher pH of its environment. Also in non-aqueous environments where no pH value can be determined, a cationisable compound, group or atom is positively charged at a high hydrogen ion concentration and uncharged at a low concentration or activity of hydrogen ions. It depends on the individual properties of the cationisable or polycationisable compound, in particular the pKa of the respective cationisable group or atom, at which pH or hydrogen ion concentration it is charged or uncharged. In diluted aqueous environments, the fraction of cationisable compounds, groups or atoms bearing a positive charge may be estimated using the so-called Henderson-Hasselbalch equation which is well-known to a person skilled in the art. E.g., in some embodiments, if a compound or moiety is cationisable, it is preferred that it is positively charged at a pH value of about 1 to 9, preferably 4 to 9, 5 to 8 or even 6 to 8, more preferably of a pH value of or below 9, of or below 8, of or below 7, most preferably at physiological pH values, e.g. about 7.3 to 7.4, i.e. under physiological conditions, particularly under physiological salt conditions of the cell in vivo. In other embodiments, it is preferred that the cationisable compound or moiety is predominantly neutral at physiological pH values, e.g. about 7.0-7.4, but becomes positively charged at lower pH values. In some embodiments, the preferred range of pKa for the cationisable compound or moiety is about 5 to about 7.

Coding sequence/coding region: The terms “coding sequence” or “coding region” and the corresponding abbreviation “cds” as used herein will be recognized and understood by the person of ordinary skill in the art, and are e.g. intended to refer to a sequence of several nucleotide triplets, which may be translated into a peptide or protein. A coding sequence in the context of the present invention is preferably an RNA sequence, consisting of a number of nucleotides that may be divided by three, which starts with a start codon and which preferably terminates with a stop codon. A cds is preferably a part of the coding RNA.

Derived from: The term “derived from” as used throughout the present specification in the context of a nucleic acid, i.e. for a nucleic acid “derived from” (another) nucleic acid, means that the nucleic acid, which is derived from (another) nucleic acid, shares e.g. at least 60%, 70%, 80%, 81%, 82%, 83%, 84%, 85%, 86%, 87%, 88%, 89%, 90%, 91%, 92%, 93%, 94%, 95%, 96%, 97%, 98%, or 99% sequence identity with the nucleic acid from which it is derived. The skilled person is aware that sequence identity is typically calculated for the same types of nucleic acids, i.e. for DNA sequences or for RNA sequences. Thus, it is understood, if a DNA is “derived from” an RNA or if an RNA is “derived from” a DNA, in a first step the RNA sequence is converted into the corresponding DNA sequence (in particular by replacing the uracils (U) by thymidines (T) throughout the sequence) or, vice versa, the DNA sequence is converted into the corresponding RNA sequence (in particular by replacing the T by U throughout the sequence). Thereafter, the sequence identity of the DNA sequences or the sequence identity of the RNA sequences is determined. Preferably, a nucleic acid “derived from” a nucleic acid also refers to nucleic acid, which is modified in comparison to the nucleic acid from which it is derived, e.g. in order to increase RNA stability even further and/or to prolong and/or increase protein production. In the context of amino acid sequences (e.g. antigenic peptides or proteins) the term “derived from” means that the amino acid sequence, which is derived from (another) amino acid sequence, shares e.g. at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence from which it is derived.

Epitope: The term “epitope” (also called “antigen determinant” in the art) as used herein will be recognized and understood by the person of ordinary skill in the art, and is e.g. intended to refer to T cell epitopes and B cell epitopes. T cell epitopes or parts of the antigenic peptides or proteins may comprise fragments preferably having a length of about 6 to about 20 or even more amino acids, e.g. fragments as processed and presented by MHC class I molecules, preferably having a length of about 8 to about 10 amino acids, e.g. 8, 9, or 10, (or even 11, or 12 amino acids), or fragments as processed and presented by MHC class II molecules, preferably having a length of about 13 to about 20 or even more amino acids. These fragments are typically recognized by T cells in form of a complex consisting of the peptide fragment and an MHC molecule, i.e. the fragments are typically not recognized in their native form. B cell epitopes are typically fragments located on the outer surface of (native) protein or peptide antigens, preferably having 5 to 15 amino acids, more preferably having 5 to 12 amino acids, even more preferably having 6 to 9 amino acids, which may be recognized by antibodies, i.e. in their native form. Such epitopes of proteins or peptides may furthermore be selected from any of the herein mentioned variants of such proteins or peptides. In this context epitopes can be conformational or discontinuous epitopes which are composed of segments of the proteins or peptides as defined herein that are discontinuous in the amino acid sequence of the proteins or peptides as defined herein but are brought together in the three-dimensional structure or continuous or linear epitopes which are composed of a single polypeptide chain.

Fragment: The term “fragment” as used throughout the present specification in the context of a nucleic acid sequence (e.g. RNA sequence) or an amino acid sequence may typically be a shorter portion of a full-length sequence of e.g. a nucleic acid sequence or an amino acid sequence. Accordingly, a fragment, typically, consists of a sequence that is identical to the corresponding stretch within the full-length sequence. A preferred fragment of a sequence in the context of the present invention, consists of a continuous stretch of entities, such as nucleotides or amino acids corresponding to a continuous stretch of entities in the molecule the fragment is derived from, which represents at least 40%, 50%, 60%, 70%, 80%, 90%, 95% of the total (i.e. full-length) molecule from which the fragment is derived (e.g. VP8* of a Rotavirus). The term “fragment” as used throughout the present specification in the context of proteins or peptides may, typically, comprise a sequence of a protein or peptide as defined herein, which is, with regard to its amino acid sequence, N-terminally and/or C-terminally truncated compared to the amino acid sequence of the original protein. Such truncation may thus occur either on the amino acid level or correspondingly on the nucleic acid level. A sequence identity with respect to such a fragment as defined herein may therefore preferably refer to the entire protein or peptide as defined herein or to the entire (coding) nucleic acid molecule of such a protein or peptide. Fragments of proteins or peptides may comprise at least one epitope of those proteins or peptides.

Heterologous: The terms “heterologous” or “heterologous sequence” as used throughout the present specification in the context of a nucleic acid sequence or an amino acid sequence refers to a sequence (e.g. RNA, amino acid) will be recognized and understood by the person of ordinary skill in the art, and is intended to refer to a sequence that is derived from another gene, from another allele, from another species. Two sequences are typically understood to be “heterologous” if they are not derivable from the same gene or in the same allele. I.e., although heterologous sequences may be derivable from the same organism, they naturally (in nature) do not occur in the same RNA molecule or protein.

Humoral immune response: The terms “humoral immunity” or “humoral immune response” will be recognized and understood by the person of ordinary skill in the art, and are e.g. intended to refer to B-cell mediated antibody production and optionally to accessory processes accompanying antibody production. A humoral immune response may be typically characterized, e.g. by Th2 activation and cytokine production, germinal center formation and isotype switching, affinity maturation and memory cell generation. Humoral immunity may also refer to the effector functions of antibodies, which include pathogen and toxin neutralization, classical complement activation, and opsonin promotion of phagocytosis and pathogen elimination.

Identity (of a sequence): The term “identity” as used throughout the present specification in the context of a nucleic acid sequence or an amino acid sequence will be recognized and understood by the person of ordinary skill in the art, and is e.g. intended to refer to the percentage to which two sequences are identical. To determine the percentage to which two sequences are identical, e.g. nucleic acid sequences or amino acid (aa) sequences as defined herein, preferably the aa sequences encoded by the nucleic acid sequence as defined herein or the aa sequences themselves, the sequences can be aligned in order to be subsequently compared to one another. Therefore, e.g. a position of a first sequence may be compared with the corresponding position of the second sequence. If a position in the first sequence is occupied by the same residue as is the case at a position in the second sequence, the two sequences are identical at this position. If this is not the case, the sequences differ at this position. If insertions occur in the second sequence in comparison to the first sequence, gaps can be inserted into the first sequence to allow a further alignment. If deletions occur in the second sequence in comparison to the first sequence, gaps can be inserted into the second sequence to allow a further alignment. The percentage to which two sequences are identical is then a function of the number of identical positions divided by the total number of positions including those positions which are only occupied in one sequence. The percentage to which two sequences are identical can be determined using an algorithm, e.g. an algorithm integrated in the BLAST program.

Immunogen, immunogenic: The terms “immunogen” or “immunogenic” will be recognized and understood by the person of ordinary skill in the art, and are e.g. intended to refer to a compound that is able to stimulate/induce an immune response. Preferably, an immunogen is a peptide, polypeptide, or protein. An immunogen in the sense of the present invention is the product of translation of a provided mRNA, comprising at least one coding sequence encoding at least one antigenic peptide, protein derived from VP8* as defined herein. Typically, an immunogen elicits an adaptive immune response.

Immune response: The term “immune response” will be recognized and understood by the person of ordinary skill in the art, and is e.g. intended to refer to a specific reaction of the adaptive immune system to a particular antigen (so called specific or adaptive immune response) or an unspecific reaction of the innate immune system (so called unspecific or innate immune response), or a combination thereof.

Immune system: The term “immune system” will be recognized and understood by the person of ordinary skill in the art, and is e.g. intended to refer to a system of the organism that may protect the organisms from infection. If a pathogen succeeds in passing a physical barrier of an organism and enters this organism, the innate immune system provides an immediate, but non-specific response. If pathogens evade this innate response, vertebrates possess a second layer of protection, the adaptive immune system. Here, the immune system adapts its response during an infection to improve its recognition of the pathogen. This improved response is then retained after the pathogen has been eliminated, in the form of an immunological memory, and allows the adaptive immune system to mount faster and stronger attacks each time this pathogen is encountered. According to this, the immune system comprises the innate and the adaptive immune system. Each of these two parts typically contains so called humoral and cellular components.

Innate immune system: The term “innate immune system” (also known as non-specific or unspecific immune system) will be recognized and understood by the person of ordinary skill in the art, and is e.g. intended to refer to a system typically comprising the cells and mechanisms that defend the host from infection by other organisms in a non-specific manner. This means that the cells of the innate system may recognize and respond to pathogens in a generic way, but unlike the adaptive immune system, it does not confer long-lasting or protective immunity to the host. The innate immune system may be activated by ligands of pattern recognition receptor e.g. Toll-like receptors, NOD-like receptors, or RIG-1 like receptors etc.

Lipidoid compound: A lipidoid compound, also simply referred to as lipidoid, is a lipid-like compound, i.e. an amphiphilic compound with lipid-like physical properties. In the context of the present invention the term lipid is considered to encompass lipidoid compounds.

Monovalent vaccine, monovalent composition: The terms “monovalent vaccine”, “monovalent composition” “univalent vaccine” or “univalent composition” will be recognized and understood by the person of ordinary skill in the art, and are e.g. intended to refer to a composition or a vaccine comprising only one antigen or antigen construct from a pathogen. Accordingly, said vaccine or composition comprises only one coding RNA species encoding a single antigen or antigen construct of a single organism. The term “monovalent vaccine” includes the immunization against a single valence. In the context of the invention, a monovalent Rotavirus vaccine or composition would comprise an coding RNA encoding one single antigenic peptide or protein derived from VP8* of one specific Rotavirus.

Nucleic acid: The terms “nucleic acid” or “nucleic acid molecule” will be recognized and understood by the person of ordinary skill in the art. The term nucleic acid molecule preferably refers to DNA or RNA molecules. It is preferably used synonymous with the term polynucleotide. Preferably, a nucleic acid or a nucleic acid molecule is a polymer comprising or consisting of nucleotide monomers, which are covalently linked to each other by phosphodiester-bonds of a sugar/phosphate-backbone. The term “nucleic acid molecule” also encompasses modified nucleic acid molecules, such as base-modified, sugar-modified or backbone-modified DNA or RNA molecules as defined herein.

Nucleic acid sequence/RNA sequence/amino acid sequence: The terms “nucleic acid sequence”, “RNA sequence” or “amino acid sequence” will be recognized and understood by the person of ordinary skill in the art, and e.g. refer to a particular and individual order of the succession of its nucleotides or amino acids.

Permanently cationic: The term “permanently cationic” as used herein will be recognized and understood by the person of ordinary skill in the art, and means, e.g., that the respective compound, or group or atom, is positively charged at any pH value or hydrogen ion activity of its environment. Typically, the positive charge results from the presence of a quaternary nitrogen atom. Where a compound carries a plurality of such positive charges, it may be referred to as permanently polycationic, which is a subcategory of permanently cationic.

Polyvalent/multivalent vaccine, polyvalent/multivalent composition: The terms “polyvalent vaccine”, “polyvalent composition” “multivalent vaccine” or “multivalent composition” will be recognized and understood by the person of ordinary skill in the art, and are e.g. intended to refer to a composition or a vaccine comprising antigens from more than one Rotavirus, or comprising different antigens or antigen constructs of the same Rotavirus, or any combination thereof. The terms describe that said vaccine or composition has more than one valence. In the context of the invention, a polyvalent Rotavirus vaccine would comprise coding RNA encoding antigenic peptides or proteins derived from several different Rotaviruses or comprising coding RNA encoding different antigens or antigen constructs from the same Rotavirus, or a combination thereof. In preferred embodiment, a polyvalent Rotavirus vaccine or composition comprises more than one, preferably 2, 3, 4, 5 or even more different coding RNA species each encoding at least one peptide or protein of Rotavirus (e.g. different VP8* constructs). In particularly preferred embodiments, a polyvalent Rotavirus vaccine or composition is a trivalent Rotavirus vaccine or composition comprising 3 different coding RNA species each encoding VP8* (or a fragment of VP8*) derived from a different serotype (e.g. Serotype [P4], [P6], [P8])).

Stabilized RNA: The term “stabilized RNA” refer to an RNA that is modified such, that it is more stable to disintegration or degradation, e.g., by environmental factors or enzymatic digest, such as by exo- or endonuclease degradation, compared to an RNA without such modification. Preferably, a stabilized RNA in the context of the present invention is stabilized in a cell, such as a prokaryotic or eukaryotic cell, preferably in a mammalian cell, such as a human cell. The stabilization effect may also be exerted outside of cells, e.g. in a buffer solution etc., e.g., for storage of a composition comprising the stabilized RNA.

T-cell responses: The terms “cellular immunity” or “cellular immune response” or “cellular T-cell responses” as used herein will be recognized and understood by the person of ordinary skill in the art, and are for example intended to refer to the activation of macrophages, natural killer cells (NK), antigen-specific cytotoxic T-lymphocytes, and the release of various cytokines in response to an antigen. In more general terms, cellular immunity is not based on antibodies, but on the activation of cells of the immune system. Typically, a cellular immune response may be characterized e.g. by activating antigen-specific cytotoxic T-lymphocytes that are able to induce apoptosis in cells, e.g. specific immune cells like dendritic cells or other cells, displaying epitopes of foreign antigens on their surface (e.g. VP8*).

Variant (of a sequence): The term “variant” as used throughout the present specification in the context of a nucleic acid sequence will be recognized and understood by the person of ordinary skill in the art, and is e.g. intended to refer to a variant of a nucleic acid sequence derived from another nucleic acid sequence. E.g., a variant of a nucleic acid sequence may exhibit one or more nucleotide deletions, insertions, additions and/or substitutions compared to the nucleic acid sequence from which the variant is derived. A variant of a nucleic acid sequence may at least 50%, 60%, 70%, 80%, 90%, or 95% identical to the nucleic acid sequence the variant is derived from. The variant is a functional variant in the sense that the variant has retained at least 50%, 60%, 70%, 80%, 90%, or 95% or more of the function of the sequence where it is derived from. A “variant” of a nucleic acid sequence may have at least 70%, 75%, 80%, 85%, 90%, 95%, 98% or 99% nucleotide identity over a stretch of at least 10, 20, 30, 50, 75 or 100 nucleotide of such nucleic acid sequence.

The term “variant” as used throughout the present specification in the context of proteins or peptides is e.g. intended to refer to a proteins or peptide variant having an amino acid sequence which differs from the original sequence in one or more mutation(s)/substitution(s), such as one or more substituted, inserted and/or deleted amino acid(s). Preferably, these fragments and/or variants have the same, or a comparable specific antigenic property (immunogenic variants, antigenic variants). Insertions and substitutions are possible, in particular, at those sequence positions which cause no modification to the three-dimensional structure or do not affect the binding region. Modifications to a three-dimensional structure by insertion(s) or deletion(s) can easily be determined e.g. using CD spectra (circular dichroism spectra). A “variant” of a protein or peptide may have at least 70%, 75%, 80%, 85%, 90%, 95%, 98% or 99% amino acid identity over a stretch of at least 10, 20, 30, 50, 75 or 100 amino acids of such protein or peptide. Preferably, a variant of a protein comprises a functional variant of the protein, which means, in the context of the invention, that the variant exerts essentially the same, or at least 40%, 50%, 60%, 70%, 80%, 90% of the immunogenicity as the protein it is derived from.

### SHORT DESCRIPTION OF THE INVENTION

The present invention is based on the inventor's surprising finding that at least one peptide or protein derived from Rotavirus, provided by the coding RNA of the first aspect, can efficiently be expressed in human cells and induces strong and efficient immune responses (see e.g. Example 2 and 3). Through different optimizations in Rotavirus VP8* antigen design by including e.g. heterologous elements the immune responses and the expression could be further improved (e.g. Example 2, Example 9). Even more unexpected, the inventors showed that the coding RNA of the invention induces cross-reactive responses against other P-serotypes (Example 3, Example 9). Through different optimizations in mRNA design encoding the Rotavirus antigenic protein VP8* (e.g. through cap1, a poly(A)-sequence located exactly at the 3′ terminus and/or inventive UTR-combination) expression and immune responses could be further remarkably improved, particularly regarding neutralizing titers (VNTs) and T-cell responses in comparison to adjuvanted recombinant Rotavirus protein (Example 4, 5, 6), indicating that the coding RNA or the compostions/vaccine of the invention is therefore suitable for use as a vaccine, e.g. as a vaccine in human subjects.

In a first aspect, the present invention provides a coding RNA, preferably a coding RNA for a Rotavirus vaccine, comprising at least one 5′ untranslated region (UTR) and/or at least one 3′ untranslated region (UTR), and at least one coding sequence operably linked to said 3′-UTR and/or 5′-UTR encoding at least one antigenic peptide or protein of a Rotavirus, preferably a Rotavirus VP8*, or an immunogenic fragment or immunogenic variant thereof.

In a second aspect, the present invention provides a composition, preferably an immunogenic composition comprising at least one coding RNA of the first aspect. Suitably, the composition may comprise at least one coding RNA complexed with, encapsulated in, or associated with one or more lipids, thereby forming lipid nanoparticles.

In a third aspect, the present invention provides a Rotavirus vaccine wherein the vaccine comprises at least one coding RNA of the first aspect or the composition of the second aspect.

In a fourth aspect, the present invention provides a kit or kit of parts comprising at least one coding RNA of the first aspect, and/or at least one composition of the second aspect, and/or at least one vaccine of the third aspect.

The invention further concerns a method of treating or preventing Rotavirus infection in a subject, and first and second medical uses of the coding RNA, compositions, and vaccines. Also provided are methods of manufacturing the coding RNA, the composition, or the vaccine.

### DETAILED DESCRIPTION OF THE INVENTION

The present application is filed together with a sequence listing in electronic format, which is part of the description of the present application (WIPO standard ST.25). The information contained in the sequence listing is incorporated herein by reference in its entirety. Where reference is made herein to a “SEQ ID NO”, the corresponding nucleic acid sequence or amino acid (aa) sequence in the sequence listing having the respective identifier is referred to. For many sequences, the sequence listing also provides additional detailed information, e.g. regarding certain structural features, sequence optimizations, GenBank or NCBI identifiers, or additional detailed information regarding its coding capacity. In particular, such information is provided under numeric identifier <223> in the WIPO standard ST.25 sequence listing. Accordingly, information provided under said numeric identifier <223> is explicitly included herein in its entirety and has to be understood as integral part of the description of the underlying invention.

Coding RNA for a Rotavirus Vaccine:

In a first aspect, the invention relates to a coding RNA, preferably a coding RNA suitable for a Rotavirus vaccine, comprising at least one coding sequence encoding a Rotavirus antigenic peptide or protein.

In embodiments of the invention the coding RNA for a Rotavirus vaccine comprises
a) at least one heterologous 5′ untranslated region (5′-UTR) and/or at least one heterologous 3′ untranslated region (3′-UTR); and
b) at least one coding sequence operably linked to said 3′-UTR and/or 5′-UTR encoding at least one antigenic peptide or protein of a Rotavirus, or an immunogenic fragment or immunogenic variant thereof.

The coding RNA of the first aspect may form the basis for an RNA based vaccine. The vaccine based on the inventive coding RNA allows parenteral delivery that is not affected by possible efficacy reductions which may occur via the oral route. Generally, protein-based vaccines, or live attenuated vaccines known in the art are suboptimal in developing countries due to their high production costs. In contrast, the coding RNA or the RNA-based vaccines according to the present invention allow very cost-effective production. Therefore, in comparison with known vaccines the vaccine based on the inventive coding RNA can be produced significantly cheaper, which is very advantageous particularly for use in developing countries. One further advantage of a vaccine based on the inventive coding RNA may be its temperature-stable nature in comparison with the life oral rotavirus vaccines available or with other protein or peptide-based vaccine compositions.

The term “coding RNA” as used herein will be recognized and understood by the person of ordinary skill in the art, and are e.g. intended to refer to an RNA comprising a coding sequence (“cds”) comprising several nucleotide triplets, wherein said cds may be translated into a peptide or protein (e.g. upon administration to a cell or an organism).

The term “coding RNA for a vaccine” as used herein has to be understood as a coding RNA having certain advantageous features that makes the RNA suitable for in vivo administration to a cell or subject, e.g. a human. Moreover, a “coding RNA for a vaccine” is preferably expressed, that is translated into protein, when administered to a subject, e.g. a human. In addition, the “coding RNA for a vaccine” preferably induces a specific immune response against the encoded protein after administration to a subject, e.g. a human.

Preferably, intramuscular or intradermal administration of said “coding RNA for a vaccine” results in expression of the encoded Rotavirus antigen in a subject.

The term “immunogenic fragment” or “immunogenic variant” has to be understood as a fragment/variant of the corresponding antigen (e.g. Rotavirus VP8*) that is capable of raising an immune response in a subject.

In general, the RNA of the invention may be composed of a protein-coding region (also referred to as coding sequence “cds”, or “ORF”), and 5′ and/or 3′ untranslated regions (UTRs). The 3′-UTR is variable in sequence and size; it typically spans between the stop codon and the poly(A) tail. Importantly, the 3′-UTR sequence harbors several regulatory motifs that determine RNA turnover, stability and localization, and thus governs many aspects of post-transcriptional regulation. In medical application of RNA (e.g. immunotherapy applications, vaccination) the regulation of RNA translation into protein is of paramount importance to therapeutic safety and efficacy. The present inventors surprisingly discovered that certain RNA constructs enable the rapid and transient expression of high amounts of Rotavirus antigenic peptides or proteins. Further, said RNA molecules induce, when administered to a subject, a balanced immune response, comprising both cellular and humoral immunity. Accordingly, the coding RNA provided herein is particularly useful and suitable for various applications in vivo, including the vaccination against Rotavirus, and may, accordingly, be a suitable component of a vaccine for treating and/or preventing Rotavirus infections.

Rotavirus possesses a double stranded, segmented RNA genome that encodes for six structural and six non-structural proteins and forms non-enveloped particles with three-layered icosahedral capsids. The six structural proteins (VPs—viral proteins) form the virus particle (virion) and are called VP1, VP2, VP3, VP4, VP6 and VP7. The six non-structural proteins (NSPs) are called NSP1, NSP2, NSP3, NSP4, NSP5 and NSP6 and are important for viral mRNA translation, for genome replication, genome encapsidation and capsid assembly. In addition, non-structural proteins are involved in antagonizing the antiviral host response and in subverting important cellular processes to enable successful virus replication.

The term “peptide or protein of a Rotavirus” relates to any Rotavirus proteins, but also to fragments, variants or derivatives thereof, preferably to immunogenic fragments or immunogenic variants thereof.

In embodiments, the at least one antigenic peptide or protein of a Rotavirus may be selected from a structural protein selected from VP1, VP2, VP3, VP4, VP6 and VP7. Suitable amino acid sequences may be selected from SEQ ID NOs: 1-26 of published PCT application WO2017/081110A1, or fragments and variants thereof, SEQ ID NOs: 1-26 and the disclosure provided in WO2017/081110A1 relating thereto herewith incorporated by reference.

In embodiments, the at least one antigenic peptide or protein of a Rotavirus may be selected from a non-structural protein selected from NSP1, NSP2, NSP3, NSP4, NSP5 and NSP6. Suitable amino acid sequences may be selected from SEQ ID NOs: 27-39 of published PCT application WO2017/081110A1, or fragments and variants thereof, SEQ ID NOs: 27-39 and the disclosure provided in WO2017/081110A1 relating thereto herewith incorporated by reference.

In other embodiments, the at least one antigenic peptide or protein of a Rotavirus may be selected from VP4 or VP7 or, preferably, a cleavage product of VP4. These proteins are particularly preferred because they are components of the outermost protein layer of Rotavirus which may be especially relevant for an immune response. In preferred embodiments the protein is identical or is derived from a cleavage product of VP4, preferably VP5* (e.g. according to SEQ ID NO: 40 of published PCT application WO2017/081110A1) or VP8* (e.g. according to SEQ ID NO: 41, SEQ ID NO: 45, SEQ ID NO: 47 or SEQ ID NO: 49 of published PCT application WO2017/081110A1), wherein VP8* is particularly preferred. Although VP5* and VP8* are (in vivo) cleavage products of the protein VP4 they are nevertheless referred to as proteins. In that context, SEQ ID NOs: 40 to 49 of WO2017/081110A1, or fragments and variants thereof, and the disclosure provided in WO2017/081110A1 relating thereto are herewith incorporated by reference.

In various other embodiments, the at least one antigenic peptide or protein of a Rotavirus may be selected from any of the amino acid sequences according to SEQ ID NOs: 1-827 of WO2017/081110A1 or fragments and variants thereof. Accordingly, SEQ ID NOs: 1-827 of WO2017/081110A1, or fragments and variants thereof, and the disclosure provided in WO2017/081110A1 relating thereto herewith incorporated by reference.

In a preferred embodiment, the coding RNA for a Rotavirus vaccine comprises
a) at least one heterologous 5′ untranslated region (5′-UTR) and/or at least one heterologous 3′ untranslated region (3′-UTR); and
b) at least one coding sequence operably linked to said 3′-UTR and/or 5′-UTR encoding at least one antigenic protein of a Rotavirus, wherein said antigenic protein is or is derived from VP8* or an immunogenic fragment or immunogenic variant thereof.

As described above, VP8* is a protein (or a protein cleavage product) that is generated upon a naturally occurring proteolytic cleavage of the viral cell surface protein VP4 to VP5* and VP8*.

As used herein, the term Rotavirus relates to any Rotavirus strains, (serological) species (e.g. Rotavirus A), serotype (e.g. Serotype [P8] of Rotavirus A) etc. capable of causing a Rotavirus infection in a subject.

Accordingly, the at least one antigenic peptide or protein of a Rotavirus, preferably VP8*, may be derived from any Rotavirus as defined herein.

Rotaviruses belong to the family of Reoviridae and have been subdivided into eight species, namely five serological species (Rotavirus A to E) and two additional tentative species (Rotavirus F and G). These species are commonly referred to as “RV groups”. Three species thereof (A, B and C) can infect humans and animals. The other species D, E, F and G have been identified in animals, mostly in birds. Rotavirus A is responsible for more than 90% of all human infections and is most important for human infection and disease. It is transmitted by the faecal-oral route and targets enterocytes in the villi of the small intestine, leading to cell damage and gastroenteritis.

Within the species Rotavirus A, there are different strains (serotypes or genotypes), which are classified by a dual system based on the structural proteins VP7 and VP4. VP7 and VP4 are components of the outermost protein layer (outer capsid), and both carry neutralizing epitopes. VP7 is a glycoprotein (G) that forms the outer layer or surface of the virion. VP7 determines the G-type of the strain. 27 different G-serotypes (G1-G27) have been described. VP4 is a surface protein that protrudes as a spike. VP4 is essential for virus-cell interaction and determines host range and virulence of the virus. VP4 is protease sensitive (P) and determines the P-type of the virus. There are 35 P-serotypes (P[1]-P[35]). This dual classification system may be applied to any Rotavirus (e.g. Rotavirus A to E).

Preferably in the context of the invention, the Rotavirus from which the Rotavirus antigenic peptide or protein is derived from is a Rotavirus species selected form the species A, B, C, D, E, F, G or H, wherein it is particularly preferred that the rotavirus is selected from species A, B or C. Species A, B and C are known to infect humans and various animals. Rotavirus is a Rotavirus A (RVA) is of particular relevance for human infections.

In preferred embodiments, the Rotavirus is selected from species A, B or C. In particularly preferred embodiments, the Rotavirus is Rotavirus A (RVA). Accordingly, the at least one antigenic peptide or protein of a Rotavirus, preferably VP8*, may be derived form a Rotavirus A (RVA).

The Rotavirus, preferably the Rotavirus A, may be selected from any one of the following G-serotypes and P-serotypes: G1, G2, G3, G4, G5, G6, G7, G8, G9, G10, G11, G12, G13, G14, G15, G16, G17, G18, G19, G20, G21, G22, G23, G24, G25, G26, G27, P[1], P[2], P[3], P[4], P[5], P[6], P[7], P[8], P[9], P[10], P[11], P[12], P[13], P[14], P[15], P[16], P[17], P[18], P[19], P[20], P[21], P[22], P[23], P[24], P[25], P[26], P[27], P[28], P[29], P[30], P[31], P[32], P[33], P[34], or P[35].

In humans, around 90% of infections are caused by G1, G2, G3 or G4 and also G9 and G12. With respect to the P-types P[4], P[6] and P[8] are the most prevalent. Importantly, infection with one serotype does not induce cross-protection against infection by a different serotype.

Accordingly, in preferred embodiments of the first aspect, the Rotavirus is selected from the G-serotypes or P-serotypes G1, G2, G3, G4, G9, G12, P[4], P[6] or P[8]. Accordingly, the at least one antigenic peptide or protein of a Rotavirus, preferably VP8*, may be derived form a Rotavirus A (RVA) selected from the G-serotypes or P-serotypes G1, G2, G3, G4, G9, G12, P[4], P[6] or P[8].

In particularly preferred embodiments of the first aspect, the Rotavirus is selected from P-serotypes P[4], P[6] or P[8].

Accordingly, the at least one antigenic peptide or protein of a Rotavirus, preferably VP8*, may suitably be derived form a Rotavirus A (RVA), preferably selected from the G-serotypes or P-serotypes G1, G2, G3, G4, G9, G12, P[4], P[6] or P[8], more preferably from P-serotypes P[4], P[6] or P[8].

Accordingly, the at least one antigenic peptide or protein of a Rotavirus may be derived from Rotavirus strains with the following NCBI Taxonomy ID (NCBI: txid or taxID): Rotavirus: 10912; Rotavirus A: 28875, 10941, 10950, 10970, 35336, 42567, 72132, 73034, 73036, 75918, 79064, 79065, 141265, 215680, 263595, 290547, 370529, 380390, 401074, 408598, 408599, 416557, 416558, 478084, 573015, 573016, 573017, 574984, 574985, 574986, 641314, 641316, 641323, 641324, 641328, 641330, 641331, 641332, 641333, 641336, 641338, 641339, 641342, 641343, 641348, 641349, 641351, 641354, 641356, 641358, 641359, 641360, 641361, 641363, 641364, 748549, 748550, 749226, 757016, 757017, 757018, 757019, 757020, 757021, 757022, 757025, 758889, 758890, 758891, 758894, 758896, 758899, 758900, 758901, 758903, 758905, 758906, 758907, 758910, 1004793, 1004798, 1004799, 1004800, 1004804, 1004811, 1004812, 1004815, 1004816, 1004822, 1004823, 1004825, 1004826, 1004827, 1004829, 1004832, 1004833, 1004834, 1004835, 1004838, 1004841, 1004842, 1004845, 1004847, 1004850, 1004854, 1004857, 1004858, 1004860, 1004861, 1004862, 1004863, 1004865, 1004866, 1004867, 1004869, 1004870, 1004871, 1004873, 1004877, 1004878, 1004879, 1004886, 1004888, 1004941, 1004943, 1004946, 1046565, 1133023, 1133024, 1133025, 1133026, 1133027, 1146934, 1146935, 1146936, 1148771, 1193386, 1193388, 1193396, 1193398, 1193399, 1307162, 1307163, 1307165, 1349389, 1454883, 1454885, 1454886, 1454887, 1454888, 1454889, 1454890, 1454891, 1454892, 1882333, 1906931, 1954144, 1954145, 1973200, 1973201, 1973204, 1973205, 1973207, 2040590, 2051936; Rotavirus B: 28876; Rotavirus C: 36427; Rotavirus D: 335100; Rotavirus F: 183405; Rotavirus G: 183407; Rotavirus H: 1348384; Rotavirus I: 1637496; unclassified Rotavirus: 101358. Preferably, the at least one antigenic peptide or protein of a Rotavirus may be derived from a Rotavirus A strain and/or from a Rotavirus that can infect humans.

Preferably, the at least one antigenic peptide or protein of a Rotavirus may be derived from the following Rotavirus A strains, preferably from Rotaviruses that can infect humans, selected from RVA/Human-wt/BEL/BE1058/2008/G2P[4], Hu/BEL/F01322/2009/G3P[6], RVA/Human-wt/BEL/BE1128/2009/G1P[8], Wa variant VirWa, SEROTYPE 2/STRAIN DS1, RVA/Human-tc/USA/DS-1/1976/G2P1B[4], DS-1, 1076, Wa, L26, RVA/Human-wt/BEL/BE1141/2009/G1P[8], RV3, ST3, RVA/Human-tc/GBR/ST3/1975/G4P2A[6], Wa variant TC-ParWa, Wa variant Wag7/8re, Wa variant Wag5re, Human-tc/USA/Wa/1974/G1P[8], RVA/Vaccine/USA/Rotarix-A41CB052A/1988/G1P1A[8], RVA/Vaccine/USA/RotaTeq-W179-4/1992/G6P1A[8], RVA/Human-wt/BEL/BE1175/2009/G1P[8], RVA/Human-tc/NGA/HMG035/1999/G8P[1], RVA/Vaccine/USA/RotaTeq-W179-9/1992/G1P7[5], RVA/Vaccine/USA/RotaTeq-SC2-9/1992/G2P7[5], RVA/Vaccine/USA/RotaTeq-W178-8/1992/G3P7[5], RVA/Vaccine/USA/RotaTeq-BrB-9/1996/G4P7[5], RVA/Human-TC/USA/Rotarix/2009/G1P[8], RVA/Vaccine/USA/RotaTeq-W179-4/1992/G6P1A[8], human-wt/ITA/ME848/12/2012/G12P9, RVA/Human-tc/IDN/57M/1980/G4P[10], RVA/Human-wt/BEL/B4106/2000/G3P[14], RVA/Human_wt/VNM/30378/2009/G26P[19], RVA/Human-wt/BGD/Dhaka6/2001/G11P[25], Ecu534.

In preferred embodiments, the Rotavirus is selected from Human rotavirus A BE1058 (RVA/Human-wt/BEL/BE1058/2008/G2P[4], G2P[4], JN849123.1, GI:371455744, AEX30665.1, acronym: RVA/BE1058/P[4]), Human rotavirus A F01322 (Hu/BEL/F01322/2009/G3P[6], G3P[6], JF460826.1, GI:37531451, AFA51886.1, acronym: RVA/F01322/P[6]), Human rotavirus A BE1128 (RVA/Human-wt/BEL/BE1128/2009/G1P[8], G1P[8], JN849135.1, GI:371455756, AEX30671.1, acronym: RVA/BE1128/P[8]), Human rotavirus A WA-VirWa (Wa variant VirWa, G1P[8], ACR22783.1, GI: 237846292, FJ423116.1, acronym: RVA/Wa-VirWa/P[8]), Human rotavirus A DS-1 (SEROTYPE 2/STRAIN DS1, G2P[4], CAD62680.1, GI: 28268530, AJ540227.1; RVA/Human-tc/USA/DS-1/1976/G2P1B[4], G2P[4], ABV53252.1, GI: 157389072, EF672577.1, acronym: RVA/DS-1/P[4]), Human rotavirus A 1076 (G2P[6], AAA47337.1, GI: 333858, M88480.1, acronym: RVA/1076/P[6]), Human rotavirus A WA (G1P[8], AAA47290.1, GI: 333780, M96825.1; AAA66953.1, GI: 507317, L34161.1, acronym: RVA/Wa/P[8]).

In particularly preferred embodiments, the Rotavirus is selected from Human rotavirus A BE1058 (RVA/Human-wt/BEL/BE1058/2008/G2P[4], G2P[4], JN849123.1, GI: 371455744, AEX30665.1, acronym: RVA/BE1058/P[4]), Human rotavirus A F01322 (Hu/BEL/F01322/2009/G3P[6], G3P[6], JF460826.1. GI: 37531451, AFA51886.1, acronym: RVA/F01322/P[6]), Human rotavirus A BE1128 (RVA/Human-wt/BEL/BE1128/2009/G1P[8], G1P[8], JN849135.1. GI: 371455756, AEX30671, acronym: RVA/BE1128/P[8]), or Human rotavirus A WA-VirWa (Wa variant VirWa, G1P[8], ACR22783.1, GI: 237846292, FJ423116, acronym: RVA/Wa-VirWa/P[8]).

In embodiments, the coding RNA of the invention encodes at least one antigenic protein that is or is derived from VP8*, wherein the VP8* is a full length VP8* protein having an amino acid sequence comprising or consisting of amino acid 1 to amino acid 240 (some Rotavirus A strains exhibit a VP8* with a full length of 241 or 243 amino acids). In other embodiments, the coding RNA of the invention encodes at least one antigenic protein that is or is derived from VP8*, wherein the VP8* is a fragment of a VP8* protein.

It has to be noted that where reference is made to amino acid (aa) residues and their position in VP8*, any numbering used herein—unless stated otherwise—relates to the position of the respective amino acid residue in a corresponding VP8* of BE1128 according to SEQ ID NO: 24. Respective amino acid positions are, throughout the disclosure, exemplarily indicated for VP8* of BE1128 (RVA/BE1128/P[8], RVA/Human-wt/BEL/BE1128/2009/G1P[8], G1P[8], JN849135.1, GI:371455756, AEX30671.1, abbreviated herein as “BE1128”). The person skilled in the art will of course be able to adapt the teaching relating to VP8* of BE1128 to each and every VP8* as provided herein, in particular to each and every full length VP8* and to each and every VP8* fragment as provided below and in the sequence listing (e.g., SEQ ID NOs: 1-585, 586-1737, 1862-1882, 1885-1898, 1899-1906, or 1907-1930).

Each of the amino acid sequences for VP8* full length being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 19-27, or an immunogenic fragment or immunogenic variant of any of these sequences, may be the “at least one antigenic protein” of the invention. Particularly preferred are amino acid sequences for VP8* full length being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 19, 22, 24 or 25. Additional information regarding each of these suitable amino acid sequences encoding proteins derived from Rotavirus may also be derived from the sequence listing, in particular from the details provided therein under identifier <223> as explained in the following.

In embodiments, the coding RNA of the invention encodes at least one antigenic protein that is or is derived from VP8*, wherein the VP8* is a fragment of a VP8* protein.

A “fragment of a VP8* protein” has to be understood as an N-terminal and/or a C-terminal truncated version of a (full length) VP8* protein that typically comprises 240 amino acids (amino acid 1 to amino acid 240) (according to reference VP8* of BE1128, SEQ ID NO: 24). In the context of the invention, the N- and/or C-terminal truncation has to be selected by the skilled person in a way that no important T-cell and/or B-cell epitopes are removed. Suitably, a “fragment of a VP8* protein” is large enough to elicit an adaptive immune response in a subject (wherein, in the context of the invention, the fragment of a VP8* protein is provided by the coding RNA). Therefore, a “fragment of a VP8* protein” comprises or consists of an amino acid sequence that has a length of at least about 95%, 90%, 85%, 80%, 75%, 70%, 65%, 60%, 55%, or 50% of the full length VP8* protein amino acid sequence (comprising typically 240 amino acids). A “fragment of a VP8* protein” may comprise an amino acid sequence that has a length of at least about 230, 225, 220, 215, 210, 205, 200, 195, 190, 185, 180, 175, 170, 165, 160, 155, 150, 145, 140, 135, 130, 125, 120 amino acids of a corresponding full length VP8* protein (comprising e.g. 240 amino acids) (according to reference VP8* of BE1128, SEQ ID NO: 24).

In preferred embodiments, the at least one antigenic protein derived from Rotavirus VP8* comprise an amino acid sequence stretch derived from VP8*, wherein said stretch corresponds to at least 50% full length VP8*, 55% full length VP8*, 60% full length VP8*, 65% full length VP8*, 70% full length VP8*, 75% full length VP8*, 80% full length VP8*, 85% full length VP8*, 90% full length VP8*, 95% full length VP8*, 96% full length VP8*, 97% full length VP8*, 98% full length VP8*, 99% full length VP8*, or 97% full length VP8*, wherein the amino acid stretch is preferably derived from VP8*of RVA/BE1058/P[4], RVA/F01322/P[6], RVA/BE1128/P[8] or RVA/Wa-VirWa/P[8] according to reference VP8* of BE1128, SEQ ID NO: 24), wherein full length VP8* (that is 100% full length) has a length of 240 amino acids.

“Corresponds to” in that context has to be understood as an amino acid sequence being identical, or at least 80%, 85%, 90%, 95%, 96%, 97%, 98%, 99% identical to the an amino acid sequence of VP8*, in particular to the an amino acid sequence of VP8* that is or is derived from strains RVA/BE1058/P[4], RVA/F01322/P[6], RVA/BE1128/P[8] or RVA/Wa-VirWa/P[8] according to SEQ ID NOs: 19, 22, 24 or 25.

In embodiments, the fragment of a VP8* protein is N-terminally truncated, lacking the N-terminal amino acids 1 to up to 100 of the full length VP8*. Such a fragment of a VP8* protein may have the following amino acids (aa) of a corresponding full length VP8*: 2-240, 3-240, 4-240, 5-240, 6-240, 7-240, 8-240, 9-240, 10-240, 11-240, 12-240, 13-240, 14-240, 15-240, 16-240, 17-240, 18-240, 19-240, 20-240, 21-240, 22-240, 23-240, 24-240, 25-240, 26-240, 27-240, 28-240, 29-240, 30-240, 31-240, 32-240, 33-240, 34-240, 35-240, 36-240, 37-240, 38-240, 39-240, 40-240, 41-240, 42-240, 43-240, 44-240, 45-240, 46-240, 47-240, 48-240, 49-240, 50-240, 51-240, 52-240, 53-240, 54-240, 55-240, 56-240, 57-240, 58-240, 59-240, 60-240, 61-240, 62-240, 63-240, 64-240, 65-240, 66-240, 67-240, 68-240, 69-240, 70-240, 71-240, 72-240, 73-240, 75-240, 75-240, 76-240, 77-240, 78-240, 79-240, 80-240, 81-240, 82-240, 83-240, 84-240, 85-240, 86-240, 87-240, 88-240, 89-240, 90-240, 91-240, 92-240, 93-240, 94-240, 95-240, 96-240, 97-240, 98-240, 99-240, 100-240 (according to reference VP8* of BE1128, SEQ ID NO: 24).

In embodiments, the fragment of a VP8* protein is C-terminally truncated, lacking the C-terminal amino acids 1 to up to 100 of the full length VP8*. Such a fragment of a VP8* protein may have the following amino acids (aa) of a corresponding full length VP8*: 1-239, 1-238, 1-237, 1-236, 1-235, 1-234, 1-233, 1-232, 1-231, 1-230, 1-229, 1-228, 1-227, 1-226, 1-225, 1-224, 1-223, 1-222, 1-221, 1-220, 1-219, 1-218, 1-217, 1-216, 1-215, 1-214, 1-213, 1-212, 1-211, 1-210, 1-209, 1-208, 1-207, 1-206, 1-205, 1-204, 1-203, 1-202, 1-201, 1-200, 1-199, 1-198, 1-197, 1-196, 1-195, 1-194, 1-193, 1-192, 1-191, 1-190, 1-189, 1-188, 1-187, 1-186, 1-185, 1-184, 1-183, 1-182, 1-181, 1-180, 1-179, 1-178, 1-177, 1-176, 1-175, 1-174, 1-173, 1-172, 1-171, 1-170, 1-169, 1-168, 1-167, 1-166, 1-165, 1-164, 1-163, 1-162, 1-161, 1-160, 1-159, 1-158, 1-157, 1-156, 1-155, 1-154, 1-153, 1-152, 1-151, 1-150, 1-149, 1-148, 1-147, 1-146, 1-145, 1-144, 1-143, 1-142, 1-141, 1-140 (according to reference VP8* of BE1128, SEQ ID NO: 24).

In embodiments, the fragment of a VP8* protein is N-terminally truncated as defined above and additionally C-terminally truncated as defined above. Any combination of N-terminal and C-terminal truncation of VP8* is envisaged herein and may be used as suitable “fragment of a VP8* protein” in the context of the invention (according to reference VP8* of BE1128, SEQ ID NO: 24).

In preferred embodiments, the fragment of a VP8* protein lacks the N-terminal alpha-helix domain (usually aa 1-26). In preferred embodiments, the fragment of a VP8* protein lacks the N-terminal part (including alpha-helix domain) and comprises a lectin domain (starting with about aa 41). Suitably, the VP8* fragment comprises the lectin domain of VP8* protein (aa65 to aa223) (amino acids positions according to reference VP8* of BE1128, SEQ ID NO: 24, further detailed information regarding VP8* domains and amino acid positions can be found in “Xue, Miaoge, et al. “Characterization and protective efficacy in an animal model of a novel truncated rotavirus VP8 subunit parenteral vaccine candidate.” Vaccine 33.22 (2015): 2606-2613.

In particularly preferred embodiments, the fragment of VP8* comprises the lectin domain of VP8* and lacks the N-terminal alpha helix-domain.

In specific embodiments, the fragment of a VP8* protein has an amino acid sequence comprising or consisting of amino acid 1 to amino acid 223, amino acid 41 to amino acid 223, amino acid 65 to amino acid 223, amino acid 41 to amino acid 230, or amino acid 65 to amino acid 230 (of a corresponding full length VP8* according to reference VP8* of BE1128, SEQ ID NO: 24).

In preferred embodiments, the fragment of a VP8* protein has an amino acid sequence comprising or consisting of amino acid 41 to amino acid 223, or amino acid 65 to amino acid 223 (of a corresponding full length VP8*). Accordingly, in a preferred embodiment, the at least one antigenic peptide or protein of a Rotavirus, preferably VP8*, may suitably be derived form a Rotavirus A (RVA), preferably selected from the G-serotypes or P-serotypes G1, G2, G3, G4, G9, G12, P[4], P[6] or P[8], more preferably from P-serotypes P[4], P[6] or P[8], wherein VP8* is a fragment of a VP8* protein, wherein the fragment of a VP8* protein has an amino acid sequence comprising or consisting of amino acid 41 to amino acid 223, or amino acid 65 to amino acid 223 (of a corresponding full length VP8* of BE1128, SEQ ID NO: 24).

Each of the amino acid sequences for VP8* fragments being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 28-45, or an immunogenic fragment or immunogenic variant of any of these sequences may be the “at least one antigenic protein” of the invention. Additional information regarding each of these suitable amino acid sequences encoding proteins from Rotavirus may also be derived from the sequence listing, in particular from the details provided therein under identifier <223> as explained in the following.

Further VP8* fragments may be derived from Table 1 of published PCT application WO2017/081110A1. Accordingly, any of the provided amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs of Table 1 of published PCT application WO2017/081110A1, or an immunogenic fragment or immunogenic variant of any of these sequences, may suitably be used in the context of the invention.

In various embodiments, the amino acid sequences of the at least one antigenic protein derived from Rotavirus, in particular Rotavirus VP8*, is mutated/substituted to delete at least one predicted or potential glycosylation site.

It may suitable in the context of the invention that glycosylation sites in the encoded amino acid sequence are mutated/substituted which means that encoded amino acids which may be glycosylated, e.g. after translation of the coding RNA upon in vivo administration, are exchanged to a different amino acid. Accordingly, on nucleic acid level, codons encoding asparagine which are predicted to be glycosylated (N glycosylation sites) are substituted with codons encoding glutamine.

In embodiments, the coding region encoding at least one rotavirus protein, or a fragment, variant or derivative thereof, is mutated in a way to delete at least one predicted or potential glycosylation site. Glycosylation is an important post-translational or co-translational modification of proteins. The majority of proteins synthesized in the rough endoplasmatic reticulum (ER) undergo glycosylation. There are mainly two types of glycosylation: a) In N-glycosylation, the addition of sugar chains takes place at the amide nitrogen on the side-chain of the asparagine or arginine. b) In O-glycosylation, the addition of sugar chains takes place on the hydroxyl oxygen on the side-chain of hydroxylysine, hydroxyproline, serine, tyrosine or threonine. Moreover, phospho-glycans linked through the phosphate of a phospho-serine and C-linked glycans, a rare form of glycosylation where a sugar is added to a carbon on a tryptophan side-chain, are known. Since the encoded Rotavirus protein, e.g. VP8*, is not glycosylated in the viral life cycle, entry in the ER might lead to modifications by glycosylation that could lead to epitope shielding and therefore prevent an efficient immune response. Therefore, it is particularly advantageous to delete the potential glycosylation sites of the encoded Rotavirus protein, in particular VP8*. By mutation/substitution of the relevant amino acids, the glycosylation may be prevented. In this context at least one codon coding for an asparagine, arginine, serine, threonine, tyrosine, lysine, proline or tryptophan is modified in such a way that a different amino acid is encoded thereby deleting at least one predicted or potential glycosylation site. The predicted glycosylation sites may be predicted by using artificial neural networks that examine the sequence for common glycosylation sites, e.g. N-glycosylation sites may be predicted by using the NetNGlyc 1.0 Server.

In preferred embodiments, the at least one antigenic protein from Rotavirus, preferably of VP8* of a Rotavirus, is mutated to delete at least one predicted or potential glycosylation site, e.g. asparagine (N) is substituted by a glutamine (Q). Accordingly, on nucleic acid level, the nucleic acid sequence is modified to encode for Q instead of N at predicted N-glycosylation sites, for example at predicted N-glycosylation sites of the encoded VP8* protein, or a fragment, variant or derivative thereof. In this context the term “mutated VP8* means that at least one (predicted) glycosylation site is mutated.

In various embodiments, the amino acid sequences of the at least one antigenic protein from Rotavirus, in particular Rotavirus VP8*, is mutated to delete all predicted or potential glycosylation sites.

Accordingly, it may be particularly preferred that all predicted glycosylation sites of the amino acid sequences of the at least one antigenic protein, in particular Rotavirus VP8* are mutated to completely prevent glycosylation of the resulting protein or peptide. This aspect of the invention may apply for e.g. all N-glycosylation sites or for all O-glycosylation sites or for all glycosylation sites irrespective of their biochemical nature.

A suitable amino acid sequence for mutated VP8* of P-serotype P[4] is provided in SEQ ID NO: 125 of published PCT application WO2017/081110A1, wherein N-glycosylation modifications are N67Q; N91Q; N132Q; N148Q; N230Q. A further suitable amino acid sequence for mutated VP8* of P-serotype P[6] is provided in SEQ ID NO: 126 of published PCT application WO2017/081110A1, wherein N-glycosylation modifications are N67Q; N91Q; N132Q; N146Q. A further suitable amino acid sequence for mutated VP8* of P-serotype P[6] is provided in SEQ ID NO: 3210 of published PCT application WO2017/081110A1, wherein N-glycosylation modifications are N67Q; N91Q; N132Q; N146Q. A further suitable amino acid sequence for mutated VP8* of P-serotype P[8] is provided in SEQ ID NOs: 127 and 128 of published PCT application WO2017/081110A1, wherein N-glycosylation modifications were done at N67Q; N91Q; N132Q. Accordingly, SEQ ID NOs: 125-128 and 3210 of published PCT application WO2017/081110A1 are herewith incorporated by reference.

In various embodiments, the coding RNA of the first aspect comprises at least one coding sequence encoding at least one antigenic protein, preferably VP8*, comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 10-45, or an immunogenic fragment or immunogenic variant of any of these sequences. Additional information regarding each of these suitable amino acid sequences encoding VP8* antigen constructs may also be derived from the sequence listing, in particular from the details provided therein under identifier <223> as explained in the following.

According to various preferred embodiments, the coding RNA of the invention encodes at least one antigenic peptide or protein from Rotavirus, preferably VP8* or a VP8* fragment as defined herein, and additionally at least one heterologous peptide or protein element.

Suitably, the at least one heterologous peptide or protein element may promote secretion of the encoded antigenic peptide or protein of the invention (e.g. via secretory signal sequences), promote anchoring of the encoded antigenic peptide or protein of the invention in the plasma membrane (e.g. via transmembrane elements), promote formation of antigen complexes (e.g. via multimerization domains or antigen clustering domains), promote virus-like particle formation (VLP forming sequence). In addition, the coding RNA may additionally encode peptide linker elements, self-cleaving peptides, immunologic adjuvant sequences or dendritic cell targeting sequences. Trimerization and tetramerization elements may be selected from e.g. engineered leucine zippers (engineered α-helical coiled coil peptide that adopt a parallel trimeric state), fibritin foldon domain from enterobacteria phage T4, GCN4pII, GCN4-pLI, and p53. Suitable VLP forming sequences may be selected from the list of amino acid sequences according to SEQ ID NOs: 1168-1227 of the patent application WO2017/081082, or fragments or variants of these sequences. Suitable peptide linkers may be selected from the list of amino acid sequences according to SEQ ID NOs: 1509-1565 of the patent application WO2017/081082, or fragments or variants of these sequences. Suitable self-cleaving peptides may be selected from the list of amino acid sequences according to SEQ ID NOs: 1434-1508 of the patent application WO2017/081082, or fragments or variants of these sequences. Suitable immunologic adjuvant sequences may be selected from the list of amino acid sequences according to SEQ ID NOs: 1360-1421 of the patent application WO2017/081082, or fragments or variants of these sequences. Suitable dendritic cell (DCs) targeting sequences may be selected from the list of amino acid sequences according to SEQ ID NOs: 1344-1359 of the patent application WO2017/081082, or fragments or variants of these sequences.

Suitably, the at least one coding sequence additionally encodes one or more heterologous peptide or protein elements selected from a signal peptide, a linker peptide, a helper epitope, an antigen clustering domain, or a transmembrane domain.

In preferred embodiments, the coding RNA of the invention encoding at least one antigenic protein derived from VP8* of a Rotavirus, and additionally encodes at least one heterologous secretory signal peptide.

Suitably, the secretory signal peptide is or is derived from tissue plasminogen activator (TPA or HsPLAT), human serum albumin (HSA or HsALB), or immunoglobulin IgE (IgE).

In preferred embodiments, the secretory signal peptide is or is derived from HsPLAT, HsALB, or IgE, wherein the amino acid sequences of said heterologous signal peptides is identical or at least 70%, 80%, 85%, 86%, 87%, 88%, 89%, 90%, 91%, 92%, 93%, 94%, 95%, 96%, 97%, 98%, or 99% identical to any one of amino acid sequences SEQ ID NOs: 1738-1740, or fragment or variant of any of these.

In particularly preferred embodiments, the secretory signal peptide is or is derived from IgE, wherein the amino acid sequences of said heterologous signal peptide is identical or at least 70%, 80%, 85%, 86%, 87%, 88%, 89%, 90%, 91%, 92%, 93%, 94%, 95%, 96%, 97%, 98%, or 99% identical to any one of amino acid sequence SEQ ID NO: 1738, or fragment or variant of any of these.

In embodiments where the coding RNA of the invention additionally encodes heterologous secretory signal peptides, it is particularly preferred and suitable to generate a fusion protein comprising a heterologous N-terminal secretory signal peptide and a C-terminal peptide or protein derived from VP8*, wherein said C-terminal peptide or protein derived from VP8* is preferably lacking an endogenous N-terminal secretory signal peptide.

Constructs comprising an N-terminal secretory signal peptide may ideally improve the secretion of the Rotavirus protein, preferably the VP8* protein (that is encoded by the coding RNA of the first aspect). Accordingly, improved secretion of the Rotavirus protein, preferably the VP8* protein, upon administration of the coding RNA of the first aspect, may be advantageous for the induction of humoral immune responses against the encoded Rotavirus antigenic protein.

Further suitable secretory signal peptides may be selected from the list of amino acid sequences according to SEQ ID NOs: 1-1115 and SEQ ID NO: 1728 of published PCT patent application WO2017/081082, or fragments or variants of these sequences, wherein said secretory signal peptides are N-terminally fused to a Rotavirus protein (or fragment), e.g. to VP8*, lacking the endogenous secretory signal sequence.

In some embodiments, the signal peptide is selected from: SEQ ID NOs: 423-427 of patent application WO2017/070624A1 or a fragment or variant of any of these sequences. In this context SEQ ID NOs: 423-427, of patent application WO2017/070624A1, and the disclosure related thereto, are herewith incorporated by reference.

Suitable examples of Rotavirus VP8* constructs comprising an N-terminal heterologous secretory signal sequence are SP-IgE_P2_VP8*(65-223), SP-IgE_P2_VP8*(41-223), SP-IgE_P2_VP8*(41-223)_Ferritin, SP-IgE_P2_VP8*(41-223)_TM domain-HA, HsALB_VP8*(2-230), HsALB_VP8*(11-223), HsALB_VP8*(41-223), HsALB_P2_VP8*(41-223), HsPLAT_VP8*(41-223), HsPLAT_P2_VP8*(41-223) or HsPLAT_VP8*(2-230), wherein SP-IgE_P2_VP8*(65-223) and SP-IgE_P2_VP8*(41-223) are particularly preferred. The corresponding amino acid sequences for each of the above listed constructs can be found in Table 1.

In preferred embodiments, the coding RNA of the invention encodes at least one antigenic protein of a Rotavirus as defined herein and additionally at least one heterologous helper epitope. A helper epitope may enhance the immune response of the RNA encoding the Rotavirus antigen, preferably VP8*.

In particularly preferred embodiments, the heterologous helper epitope is derived from P2 helper peptide (P2) according to SEQ ID NO: 1750. In further preferred embodiments, the helper epitope is derived from PADRE helper epitope (pan HLA DR-binding epitope, PADRE) according to SEQ ID NO: 1754.

In preferred embodiments, the helper epitope is or is derived from P2, wherein the amino acid sequences of said helper epitopes is identical or at least 70%, 80%, 85%, 86%, 87%, 88%, 89%, 90%, 91%, 92%, 93%, 94%, 95%, 96%, 97%, 98%, or 99% identical to any one of amino acid sequences SEQ ID NOs: 1750 or 1754, or fragment or variant of any of these.

In embodiments where the coding RNA of the invention additionally encodes heterologous helper epitope, it is particularly preferred and suitable to generate a fusion protein comprising a heterologous N-terminal helper epitope, optionally a linker element, and a C-terminal peptide or protein derived from VP8*. Constructs comprising an N-terminal terminal helper epitope may enhance the immune response of the RNA encoding the Rotavirus antigen, preferably VP8*. Additionally, such constructs may additionally comprise an N-terminal secretory signal sequence (as defined above). Alternatively, the helper epitope may be at the C-terminus, and the protein derived from VP8* may be at the N-terminus.

Preferably, the amino acid sequence of P2 helper epitope of tetanus toxin according to SEQ ID NO: 1750 (GenBank X04436.1 or NC_004565.1 derived from Kovacs-Nolan et al.; PMID 16978788; P2: aa 830-844) may serve as a basis for advantageous designs of the inventive coding RNA. The inclusion of P2 in antigens has been demonstrated to strongly influence the antibody responses to poorly immunogenic B cell epitopes.

Preferably, the helper epitope P2 is derived from Tetanus toxin, or a fragment, variant or derivative thereof according to SEQ ID NO: 1750 (GenBank X04436.1 or NC_004565.1 derived from Kovacs-Nolan et al.; PMID 16978788; P2: aa 830-844). The inclusion of P2 in antigens has been demonstrated to strongly influence the antibody responses to poorly immunogenic B cell epitopes. In the context of a protein-based approach it has already been shown by Wen et al. (Vaccine. 2014 Jul. 31; 32(35):4420-4427) that the N-terminal P2 helper peptide derived from tetanus toxin was able to increase immune responses against Rotavirus VP8*. Now, the inventors were able to show that the addition of a sequence encoding a helper epitope may be particularly effective in enhancing the immune response in an mRNA-based vaccine approach.

Preferably, the helper epitope is pan HLA DR-binding epitope (PADRE) or a fragment, variant or derivative thereof according to SEQ ID NO: 1754. PADRE is an immunodominant helper CD4 T-cell epitope. CD4+ T-cells play an important role in the generation of CD8+T effector and memory T-cell immune responses. The CD4+ T cell immune response, and thus the corresponding antigen-specific CD8+ T cell response, can be enhanced by encoding at least one antigenic protein of a Rotavirus as defined herein and additionally at least the heterologous helper epitope pan HLA DR-binding epitope (PADRE).

Suitable Examples of Rotavirus VP8* constructs comprising an heterologous helper epitope are P2_VP8*(65-223), P2_VP8*(41-223), P2_VP8*(65-223)_Ferritin, P2_VP8*(41-223)_Ferritin, LumSynt_P2_VP8*(65-223), LumSynt_P2_VP8*(41-223), SP-IgE_P2_VP8*(65-223), SP-IgE_P2_VP8*(41-223), SP-IgE_P2_VP8*(41-223)_Ferritin, SP-IgE_P2_VP8*(41-223)_TM domain-HA, HsALB_P2_VP8*(41-223), HsPLAT_P2_VP8*(41-223) wherein P2_VP8*(65-223), P2_VP8*(41-223), P2_VP8*(65-223)_Ferritin, P2_VP8*(41-223)_Ferritin, LumSynt_P2_VP8*(65-223), LumSynt_P2_VP8*(41-223), SP-IgE_P2_VP8*(65-223) and SP-IgE_P2_VP8*(41-223) are particularly preferred. The corresponding amino acid sequences for each of the above listed constructs can be found in Table 1.

In preferred embodiments, the coding RNA of the invention encodes at least one antigenic protein of a Rotavirus or an immunogenic fragment or immunogenic variant as defined herein, and additionally encodes at least one antigen clustering domain or multimerization domain.

Suitably, the antigen clustering domain (multimerization domain) is or is derived from ferritin, lumazine-synthase (LS) or encapsulin.

In preferred embodiments, the antigen clustering domain (multimerization domain) is or is derived from ferritin or lumazine-synthase, wherein the amino acid sequences of said antigen clustering domain is identical or at least 70%, 80%, 85%, 86%, 87%, 88%, 89%, 90%, 91%, 92%, 93%, 94%, 95%, 96%, 97%, 98%, or 99% identical to any one of amino acid sequences (SEQ ID NOs: 1764 or 1759), or a fragment or variant of any of these.

In embodiments where the coding RNA of the invention additionally encodes heterologous antigen clustering domain, it is particularly preferred and suitable to generate a fusion protein comprising a heterologous antigen clustering domain, optionally a linker element, and a peptide or protein derived from VP8*. Constructs comprising an antigen clustering domain may enhance the antigen clustering and may therefore promote immune responses e.g. by multiple binding events that occur simultaneously between the clustered antigens and the host cell receptors (see further details in López-Sagaseta, Jacinto, et al. “Self-assembling protein nanoparticles in the design of vaccines”. Computational and structural biotechnology journal 14 (2016):58-68) of the RNA encoding the Rotavirus antigen, preferably VP8*. Additionally, such constructs may additionally comprise an N-terminal secretory signal sequence (as defined above).

Lumazine synthase (LS, LumSynth) is an enzyme with particle-forming properties, present in a broad variety of organisms and involved in riboflavin biosynthesis. Jardine et al reported their attempts to enhance the immunoreactivity of recombinant gp120 against HIV infection through the inclusion of Lumazine synthase (LS) for the optimization of vaccine candidates (Jardine, Joseph, et al. “Rational HIV immunogen design to target specific germline B cell receptors”. Science 340.6133 (2013):711-716).

The addition of lumazine synthase allows VP8* secretion aimed to increase VP8* accessibility to immune cells. Furthermore, it allows the formation of 60-mer nanoparticles aimed to optimize B-cell activation (which could mimic the rotavirus, presenting likewise 60 VP8* spikes on its surface).

In particularly preferred embodiments, lumazine-synthase is used to promote the antigen clustering and may therefore promote immune responses of the RNA encoding the Rotavirus antigen, preferably VP8*.

In particularly preferred embodiments, the antigen clustering domain (multimerization domain) is or is derived from lumazine-synthase (LS), wherein the amino acid sequences of said antigen clustering domain is preferably identical or at least 70%, 80%, 85%, 86%, 87%, 88%, 89%, 90%, 91%, 92%, 93%, 94%, 95%, 96%, 97%, 98%, or 99% identical to any one of amino acid sequences (SEQ ID NO: 1759), a fragment or variant of any of these.

Ferritin is a protein whose main function is intracellular iron storage. Almost all living organisms produce ferritin which is made of 24 subunits, each composed of a four-alpha-helix bundle, that self-assemble in a quaternary structure with octahedral symmetry. Its properties to self-assemble into nanoparticles are well-suited to carry and expose antigens.

In particularly preferred embodiments, ferritin is used to promote the antigen clustering and may therefore promote immune responses of the RNA encoding the Rotavirus antigen, preferably VP8*.

In particularly preferred embodiments, the antigen clustering domain (multimerization domain) is or is derived from ferritin wherein the amino acid sequences of said antigen clustering domain is preferably identical or at least 70%, 80%, 85%, 86%, 87%, 88%, 89%, 90%, 91%, 92%, 93%, 94%, 95%, 96%, 97%, 98%, or 99% identical to any one of amino acid sequences (SEQ ID NO: 1764), a fragment or variant of any of these.

Encapsulin, a novel protein cage nanoparticle isolated from thermophile Thermotoga maritima, may also be used as a platform to present antigens on the surface of self-assembling nanoparticles. Encapsulin is assembled from 60 copies of identical 31 kDa monomers.

Suitable examples of Rotavirus VP8* constructs comprising a heterologous antigen clustering domain are P2_VP8*(65-223)_Ferritin, P2_VP8*(41-223)_Ferritin, LumSynt_P2_VP8*(65-223), LumSynt_P2_VP8*(41-223), or SP-IgE_P2_VP8*(41-223)_Ferritin, wherein P2_VP8*(65-223) Ferritin, and LumSynt_P2_VP8*(41-223) are particularly preferred. The corresponding amino acid sequences for each of the above listed constructs can be found in Table 1.

Further suitable multimerization domains/antigen clustering domains may be selected from the list of amino acid sequences according to SEQ ID NOs: 1116-1167 of the patent application WO2017/081082, or fragments or variants of these sequences.

In preferred embodiments, the coding RNA of the invention encodes at least one antigenic protein of a Rotavirus as defined herein, and additionally encodes at least one heterologous transmembrane domain. A heterologous transmembrane domain promote membrane anchoring of the encoded Rotavirus antigen, preferably VP8*, and may thereby enhance the immune response (in particular cellular immune responses).

Suitably, the transmembrane domain is or is derived from an influenza HA transmembrane domain, preferably derived from an influenza A HAH1N1, more preferably from H1N1/A/Netherlands/602/2009, TM domain_HA, aa521-566, NCBI Acc. No.: ACQ45338.1, CY039527.1).

Further suitable transmembrane domains are derived from Human immunodeficiency virus 1, TM domain_Env, TM domain, aa19-35, BAF32550.1, AB253679.1; Human immunodeficiency virus 1, TM domain_Env, TM domain, aa515-536, BAF32550.1, AB253679.1; Human immunodeficiency virus 1, TM domain_Env, TM domain, aa680-702, BAF32550.1, AB253679.1; Equine infectious anemia virus, TM domain_Env, TM domain, aa450-472, AAC03762.1, AF016316.1; Equine infectious anemia virus, TM domain_Env, TM domain, aa614-636, AAC03762.1, AF016316.1; Equine infectious anemia virus, TM domain_Env, TM domain, aa798-819, AAC03762.1, AF016316.1; Murine leukemia virus, TM domain_Env, TM domain, AAA46526.1, M93052.1; Mouse mammary tumor virus, TM domain_Env, TM domain, BAA03768.1, D16249.1; Mouse mammary tumor virus, TM domain_Env, TM domain, NP_056883.1, NC_001503.1; Vesicular stomatitis virus, TM domain_G, TM domain, CAA24525.1, V01214.1; Rabies virus, TM domain_G, TM domain, AEV43288.1, JN234423.1.

In embodiments where the coding RNA of the invention additionally encodes heterologous transmembrane domain, it is particularly preferred and suitable to generate a fusion protein comprising a C-terminal heterologous transmembrane domain, optionally a linker element, and an N-terminal peptide or protein derived from VP8*. Constructs comprising heterologous transmembrane domain may promote membrane anchoring of the antigen and may therefore promote immune responses, in particular cellular immune responses, of the RNA encoding the Rotavirus antigen, preferably VP8*. Additionally, such constructs may additionally comprise an N-terminal secretory signal sequence (as defined above). Alternatively, the transmembrane domain may be in the N-terminus.

Further transmembrane elements/domains may be selected from the list of amino acid sequences according to SEQ ID NOs: 1228-1343 of the patent application WO2017/081082, or fragments or variants of these sequences.

A suitable examples of a Rotavirus VP8* construct comprising a heterologous transmembrane elements/domains is SP-IgE_P2_VP8*(41-223)_TM domain-HA. The corresponding amino acid sequence for the construct can be found in Table 1.

In preferred embodiments, the coding RNA of the invention additionally encodes at least one heterologous peptide linker element.

In preferred embodiments, the coding RNA of the invention may comprise at least one Rotavirus protein or fragment as defined above, and, at least one peptide linker element, wherein the peptide linker may be selected from the list of amino acid sequences according to SEQ ID NOs: 1509-1565 of the patent application WO2017/081082, or fragments or variants of these sequences.

In particularly preferred embodiments, the heterologous peptide linker element is selected from SEQ ID NOs: 1769, 1770 or 1771.

Suitable Examples of Rotavirus VP8* constructs comprising at least one peptide linker element are P2_VP8*(65-223), P2_VP8*(41-223), P2_VP8*(65-223)_Ferritin, P2_VP8*(41-223)_Ferritin, LumSynt_P2_VP8*(65-223), LumSynt_P2_VP8*(41-223), SP-IgE_P2_VP8*(65-223), SP-IgE_P2_VP8*(41-223). The corresponding amino acid sequences for each of the above listed constructs can be found in Table 1.

In preferred embodiments, the coding RNA for a Rotavirus vaccine comprises at least one coding sequence encoding the following protein elements, preferably in N-terminal to C-terminal direction:
a) helper epitope, VP8*protein or VP8*fragment; or
b) helper epitope, VP8*protein or VP8*fragment; antigen clustering domain; or
c) signal peptide, helper epitope, VP8*protein or fragment thereof; or
d) signal peptide, helper epitope, VP8*protein or VP8*fragment, antigen clustering domain; or
e) signal peptide, helper epitope, VP8*protein or VP8*fragment, transmembrane domain; or
f) antigen clustering domain, helper epitope; VP8*protein or VP8*fragment.

In particularly preferred embodiments, the coding RNA for a Rotavirus vaccine comprises at least one coding sequence encoding the following elements in N-terminal to C-terminal direction:
a) P2 helper epitope, VP8*fragment; or
b) P2 helper epitope, VP8*fragment, antigen clustering domain (e.g. ferritin); or
c) IgE signal peptide, P2 helper epitope, VP8*fragment; or
d) IgE signal peptide, P2 helper epitope, VP8*fragment, antigen clustering domain (e.g. ferritin); or
e) IgE signal peptide, P2 helper epitope, VP8*fragment, HA transmembrane domain; or
f) antigen clustering domain (e.g. lumazine synthase), P2 helper epitope, VP8*fragment.

In particularly preferred embodiments, the coding RNA for a Rotavirus vaccine comprises at least one coding sequence encoding the following elements in N-terminal to C-terminal direction:
a) P2 helper epitope, VP8*fragment; or
b) P2 helper epitope, VP8*fragment, antigen clustering domain ferritin; or
c) IgE signal peptide, P2 helper epitope, VP8*fragment; or
d) IgE signal peptide, P2 helper epitope, VP8*fragment, HA transmembrane domain; or
e) antigen clustering domain lumazine synthase, P2 helper epitope, VP8*fragment.

In embodiments, the elements mentioned above may be connected via one or more (peptide) linker elements as defined above. For example: IgE signal peptide-linker-P2 helper epitope-linker-VP8*fragment or IgE signal peptide-P2 helper epitope-linker-VP8*fragment or IgE signal peptide-linker-P2 helper epitope-VP8*fragment.

In particularly preferred embodiments, the coding RNA for a Rotavirus vaccine comprises at least one coding sequence encoding the following elements in N-terminal to C-terminal direction:
a) P2 helper epitope, peptide linker, VP8*fragment; or
b) P2 helper epitope, peptide linker, VP8*fragment, peptide linker, antigen clustering domain ferritin; or
c) IgE signal peptide, P2 helper epitope, peptide linker, VP8*fragment; or
d) IgE signal peptide, P2 helper epitope, peptide linker, VP8*fragment, peptide linker, HA transmembrane domain; or
e) antigen clustering domain lumazine synthase, peptide linker, P2 helper epitope, peptide linker, VP8*fragment.

A detailed description of particularly preferred and suitable Rotavirus antigen constructs is provided in Table 1.

In Table 1 all references made to amino acid (aa) residues and their position in an VP8* protein relates to the position of the respective aa residue in a corresponding VP8* full length protein according to reference VP8* of BE1128, SEQ ID NO: 24). Moreover, the abbreviations used to describe suitable VP8* antigen designs/constructs of Table 1 are also used throughout the description of the invention (as described above) as well as in the ST.25 sequence listing.

Column A of Table 1 provides a short description of VP4 and of suitable VP8* antigen constructs (Table X1 describes the therefore used heterologous fragments). Column B of Table 1 provides a description of the Rotavirus of which the respective VP8* is derived from. Column C of Table 1 indicates the amino acid stretch or stretches for each of the respective antigen designs corresponding to the full length VP8* reference (SEQ ID NO: 24). Column D of Table 1 provides protein SEQ ID NOs of respective VP8* antigen constructs. Preferred coding sequences (cds) encoding the constructs of Table 1 are provided in Table 3. Preferred coding RNA/mRNA sequences are provided in Table 4.

TABLE 1
Preferred Rotavirus VP8* antigen protein constructs

| A | B | C | D |
|---|---|---|---|
| VP4 | RVA/BE1058/P[4] | aa1-775 | 10 |
| VP4 | RVA/DS-1/P[4] | aa1-775 | 11 |
| VP4 | RVA/DS-1/P[4] | aa1-775 | 12 |
| VP4 | RVA/F01322/P[6] | aa1-775 | 13 |
| VP4 | RVA/1076/P[6] | aa1-775 | 14 |
| VP4 | RVA/BE1128/P[8] | aa1-775 | 15 |
| VP4 | RVA/Wa-VirWa/P[8] | aa1-775 | 16 |
| VP4 | RVA/Wa/P[8] | aa1-775 | 17 |
| VP4 | RVA/Wa/P[8] | aa1-775 | 18 |
| VP8* | RVA/BE1058/P[4] | aa1-240 | 9, 19 |
| VP8* | RVA/DS-1/P[4] | aa1-240 | 20 |
| VP8* | RVA/DS-1/P[4] | aa1-240 | 21 |
| VP8* | RVA/F01322/P[6] | aa1-240 | 8, 22 |
| VP8* | RVA/1076/P[6] | aa1-240 | 23 |
| VP8* | RVA/BE1128/P[8] | aa1-240 | 7, 24 |
| VP8* | RVA/Wa-VirWa/P[8] | aa1-240 | 25 |
| VP8* | RVA/Wa/P[8] | aa1-240 | 26 |
| VP8* | RVA/Wa/P[8] | aa1-240 | 27 |
| VP8*(65-223) | RVA/BE1058/P[4] | aa65-223 | 28 |
| VP8*(65-223) | RVA/DS-1/P[4] | aa65-223 | 29 |
| VP8*(65-223) | RVA/DS-1/P[4] | aa65-223 | 30 |
| VP8*(65-223) | RVA/F01322/P[6] | aa65-223 | 31 |
| VP8*(65-223) | RVA/1076/P[6] | aa65-223 | 32 |
| VP8*(65-223) | RVA/BE1128/P[8] | aa65-223 | 33 |
| VP8*(65-223) | RVA/Wa-VirWa/P[8] | aa65-223 | 34 |
| VP8*(65-223) | RVA/Wa/P[8] | aa65-223 | 35 |
| VP8*(65-223) | RVA/wa/P[8] | aa65-223 | 36 |
| VP8*(41-223) | RVA/BE1058/P[4] | aa41-223 | 37 |
| VP8*(41-223) | RVA/DS-1/P[4] | aa41-223 | 38 |
| VP8*(41-223) | RVA/DS-1/P[4] | aa41-223 | 39 |
| VP8*(41-223) | RVA/F01322/P[6] | aa41-223 | 40 |
| VP8*(41-223) | RVA/1076/P[6] | aa41-223 | 41 |
| VP8*(41-223) | RVA/BE1128/P[8] | aa41-223 | 42 |
| VP8*(41-223) | RVA/Wa-VirWa/P[8] | aa41-223 | 43 |
| VP8*(41-223) | RVA/Wa/P[8] | aa41-223 | 44 |
| VP8*(41-223) | RVA/Wa/P[8] | aa41-223 | 45 |
| P2_VP8*(65-223) | RVA/BE1058/P[4] | aa65-223 | 6, 46 |
| P2_VP8*(65-223) | RVA/DS-1/P[4] | aa65-223 | 47 |
| P2_VP8*(65-223) | RVA/DS-1/P[4] | aa65-223 | 48 |
| P2_VP8*(65-223) | RVA/F01322/P[6] | aa65-223 | 5, 49 |
| P2_VP8*(65-223) | RVA/1076/P[6] | aa65-223 | 50 |
| P2_VP8*(65-223) | RVA/BE1128/P[8] | aa65-223 | 4, 51 |
| P2_VP8*(65-223) | RVA/Wa-VirWa/P[8] | aa65-223 | 52 |
| P2_VP8*(65-223) | RVA/Wa/P[8] | aa65-223 | 53 |
| P2_VP8*(65-223) | RVA/Wa/P[8] | aa65-223 | 54 |
| P2_VP8*(41-223) | RVA/BE1058/P[4] | aa41-223 | 55 |
| P2_VP8*(41-223) | RVA/DS-1/P[4] | aa41-223 | 56 |
| P2_VP8*(41-223) | RVA/DS-1/P[4] | aa41-223 | 57 |
| P2_VP8*(41-223) | RVA/F01322/P[6] | aa41-223 | 58 |
| P2_VP8*(41-223) | RVA/1076/P[6] | aa41-223 | 59 |
| P2_VP8*(41-223) | RVA/BE1128/P[8] | aa41-223 | 60 |
| P2_VP8*(41-223) | RVA/Wa-VirWa/P[8] | aa41-223 | 61 |
| P2_VP8*(41-223) | RVA/Wa/P[8] | aa41-223 | 62 |
| P2_VP8*(41-223) | RVA/Wa/P[8] | aa41-223 | 63 |
| P2_VP8*(65-223)_Ferritin | RVA/BE1058/P[4] | aa65-223 | 64 |
| P2_VP8*(65-223)_Ferritin | RVA/DS-1/P[4] | aa65-223 | 65 |
| P2_VP8*(65-223)_Ferritin | RVA/DS-1/P[4] | aa65-223 | 66 |
| P2_VP8*(65-223)_Ferritin | RVA/F01322/P[6] | aa65-223 | 67 |
| P2_VP8*(65-223)_Ferritin | RVA/1076/P[6] | aa65-223 | 68 |
| P2_VP8*(65-223)_Ferritin | RVA/BE1128/P[8] | aa65-223 | 69 |
| P2_VP8*(65-223)_Ferritin | RVA/Wa-VirWa/P[8] | aa65-223 | 70 |
| P2_VP8*(65-223)_Ferritin | RVA/Wa/P[8] | aa65-223 | 71 |
| P2_VP8*(65-223)_Ferritin | RVA/Wa/P[8] | aa65-223 | 72 |
| P2_VP8*(41-223)_Ferritin | RVA/BE1058/P[4] | aa41-223 | 73 |
| P2_VP8*(41-223)_Ferritin | RVA/DS-1/P[4] | aa41-223 | 74 |
| P2_VP8*(41-223)_Ferritin | RVA/DS-1/P[4] | aa41-223 | 75 |
| P2_VP8*(41-223)_Ferritin | RVA/F01322/P[6] | aa41-223 | 76 |
| P2_VP8*(41-223)_Ferritin | RVA/1076/P[6] | aa41-223 | 77 |
| P2_VP8*(41-223)_Ferritin | RVA/BE1128/P[8] | aa41-223 | 78 |
| P2_VP8*(41-223)_Ferritin | RVA/Wa-VirWa/P[8] | aa41-223 | 79 |
| P2_VP8*(41-223)_Ferritin | RVA/Wa/P[8] | aa41-223 | 80 |
| P2_VP8*(41-223)_Ferritin | RVA/Wa/P[8] | aa41-223 | 81 |
| LumSynt_P2_VP8*(65-223) | RVA/BE1058/P[4] | aa65-223 | 82 |
| LumSynt_P2_VP8*(65-223) | RVA/DS-1/P[4] | aa65-223 | 83 |
| LumSynt_P2_VP8*(65-223) | RVA/DS-1/P[4] | aa65-223 | 84 |
| LumSynt_P2_VP8*(65-223) | RVA/F01322/P[6] | aa65-223 | 85 |
| LumSynt_P2_VP8*(65-223) | RVA/1076/P[6] | aa65-223 | 86 |
| LumSynt_P2_VP8*(65-223) | RVA/BE1128/P[8] | aa65-223 | 87 |
| LumSynt_P2_VP8*(65-223) | RVA/Wa-VirWa/P[8] | aa65-223 | 88 |
| LumSynt_P2_VP8*(65-223) | RVA/Wa/P[8] | aa65-223 | 89 |
| LumSynt_P2_VP8*(65-223) | RVA/Wa/P[8] | aa65-223 | 90 |
| LumSynt_P2_VP8*(41-223) | RVA/BE1058/P[4] | aa41-223 | 3, 91 |
| LumSynt_P2_VP8*(41-223) | RVA/DS-1/P[4] | aa41-223 | 92 |
| LumSynt_P2_VP8*(41-223) | RVA/DS-1/P[4] | aa41-223 | 93 |
| LumSynt_P2_VP8*(41-223) | RVA/F01322/P[6] | aa41-223 | 2, 94 |
| LumSynt_P2_VP8*(41-223) | RVA/1076/P[6] | aa41-223 | 95 |
| LumSynt_P2_VP8*(41-223) | RVA/BE1128/P[8] | aa41-223 | 1, 96 |
| LumSynt_P2_VP8*(41-223) | RVA/Wa-VirWa/P[8] | aa41-223 | 97 |
| LumSynt_P2_VP8*(41-223) | RVA/Wa/P[8] | aa41-223 | 98 |
| LumSynt_P2_VP8*(41-223) | RVA/Wa/P[8] | aa41-223 | 99 |
| SP-IgE_P2_VP8*(65-223) | RVA/BE1058/P[4] | aa65-223 | 100 |
| SP-IgE_P2_VP8*(65-223) | RVA/DS-1/P[4] | aa65-223 | 101 |
| SP-IgE_P2_VP8*(65-223) | RVA/DS-1/P[4] | aa65-223 | 102 |
| SP-IgE_P2_VP8*(65-223) | RVA/F01322/P[6] | aa65-223 | 103 |
| SP-IgE_P2_VP8*(65-223) | RVA/1076/P[6] | aa65-223 | 104 |
| SP-IgE_P2_VP8*(65-223) | RVA/BE1128/P[8] | aa65-223 | 105 |
| SP-IgE_P2_VP8*(65-223) | RVA/Wa-VirWa/P[8] | aa65-223 | 106 |
| SP-IgE_P2_VP8*(65-223) | RVA/Wa/P[8] | aa65-223 | 107 |
| SP-IgE_P2_VP8*(65-223) | RVA/Wa/P[8] | aa65-223 | 108 |
| SP-IgE_P2_VP8*(41-223) | RVA/BE1058/P[4] | aa41-223 | 109 |
| SP-IgE_P2_VP8*(41-223) | RVA/DS-1/P[4] | aa41-223 | 110 |
| SP-IgE_P2_VP8*(41-223) | RVA/DS-1/P[4] | aa41-223 | 111 |
| SP-IgE_P2_VP8*(41-223) | RVA/F01322/P[6] | aa41-223 | 112 |
| SP-IgE_P2_VP8*(41-223) | RVA/1076/P[6] | aa41-223 | 113 |
| SP-IgE_P2_VP8*(41-223) | RVA/BE1128/P[8] | aa41-223 | 114 |
| SP-IgE_P2_VP8*(41-223) | RVA/Wa-VirWa/P[8] | aa41-223 | 115 |
| SP-IgE_P2_VP8*(41-223) | RVA/Wa/P[8] | aa41-223 | 116 |
| SP-IgE_P2_VP8*(41-223) | RVA/Wa/P[8] | aa41-223 | 117 |
| P2_VP8*(64-223) | RVA/BE1058/P[4] | aa64-223 | 1899 |
| P2_VP8*(64-223) | RVA/F01322/P[6] | aa64-223 | 1900 |

Table X1 provides a short description of suitable heterologous elements for VP8* antigen constructs, information regarding the origin and preferred protein and coding sequences (opt1, opt4, opt5).

TABLE X1
Suitable heterologous elements for VP8* antigen constructs

| | | Amino | NCBI Accession | | CDS | CDS | CDS |
|---|---|---|---|---|---|---|---|
| Organism | Heterologous Element | Acids | No. Protein | PRT | opt1 | opt4 | opt5 |
| Homo | signal peptide_IgE (SP-IgE) | aa1-18 | AAB59424.1 | 1738 | 1741 | 1744 | 1747 |
| sapiens | | | | | | | |
| Homo | signal peptide_HsPLAT | aa1-22 | AAA61213.1 | 1739 | 1742 | 1745 | 1748 |
| sapiens | | | | | | | |
| Homo | signal peptide_HsALB | aa1-18 | NP_000468.1 | 1740 | 1743 | 1746 | 1749 |
| sapiens | | | | | | | |
| Clostridium | P2 helper peptide (P2) | aa830-844 | WP_023439719.1 | 1750 | 1751 | 1752 | 1753 |
| tetani/E88 | | | | | | | |
| Artificial | PADRE helper epitope | aa1-13 | | 1754 | 1755, | 1757 | 1758 |
| | | | | | 1756 | | |
| Aquifex | lumazine synthase | aa1-154 | | 1759 | 1760, | 1762 | 1763 |
| aeolicus/VF5 | (LumSynt) (LS) | | | | 1761 | | |
| Helicobacter | Ferritin | aa5-167 | | 1764 | 1765, | 1767 | 1768 |
| pylori/J99 | | | | | 1766 | | |
| Artificial | peptide linker 3 | aa1-5 | | 1769 | 1772 | 1775 | 1778 |
| Artificial | peptide linker 6 | aa1-15 | | 1770 | 1773 | 1776 | 1779 |
| Artificial | peptide linker 9 | aa1-3 | | 1771 | 1774 | 1777 | 1780 |

In various embodiments, the coding RNA of the first aspect comprises at least one coding sequence encoding at least one antigenic peptide or protein of Rotavirus comprising or consisting of at least one amino acid sequence being identical or at least 70%, 80%, 85%, 86%, 87%, 88%, 89%, 90%0, 91%, 92%, 93%, 94%, 95%, 96%, 97%, 98%, or 99% identical to any one of SEQ ID NOs: 1-117, 1899, 1900, or an immunogenic fragment or immunogenic variant of any of these sequences. Additional information regarding each of these suitable amino acid sequences encoding Rotavirus antigens may also be derived from the sequence listing, in particular from the details provided therein under identifier <223> as explained in the following.

In various embodiments, the coding RNA of the first aspect comprises at least one coding sequence encoding at least one antigenic peptide or protein of Rotavirus and at least one coding sequence encoding a P2 helper epitope comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 1-6, 46-117, 1899, 1900, or an immunogenic fragment or immunogenic variant of any of these sequences. Additional information regarding each of these suitable amino acid sequences encoding Rotavirus antigens may also be derived from the sequence listing, in particular from the details provided therein under identifier <223> as explained in the following.

In various embodiments, the coding RNA of the first aspect comprises at least one coding sequence encoding at least one antigenic peptide or protein of Rotavirus and at least one coding sequence encoding the antigen clustering domain ferritin comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 64-81, or an immunogenic fragment or immunogenic variant of any of these sequences. Additional information regarding each of these suitable amino acid sequences encoding Rotavirus antigens may also be derived from the sequence listing, in particular from the details provided therein under identifier <223> as explained in the following.

In various embodiments, the coding RNA of the first aspect comprises at least one coding sequence encoding at least one antigenic peptide or protein of Rotavirus and at least one coding sequence encoding the antigen clustering domain lumazine synthase epitope comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 1-3, 82-99, or an immunogenic fragment or immunogenic variant of any of these sequences. Additional information regarding each of these suitable amino acid sequences encoding Rotavirus antigens may also be derived from the sequence listing, in particular from the details provided therein under identifier <223> as explained in the following.

In various embodiments, the coding RNA of the first aspect comprises at least one coding sequence encoding at least one antigenic peptide or protein of Rotavirus and at least one coding sequence encoding a secretory signal peptide comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 100-117, or an immunogenic fragment or immunogenic variant of any of these sequences. Additional information regarding each of these suitable amino acid sequences encoding Rotavirus antigens may also be derived from the sequence listing, in particular from the details provided therein under identifier <223> as explained in the following.

In various embodiments, the coding RNA of the first aspect comprises at least one coding sequence encoding at least one secreted antigenic peptide or protein of Rotavirus and at least one coding sequence encoding a secretory signal peptide or the antigen clustering domain lumazine synthase epitope comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 1-3, 82-99, 100-117, or an immunogenic fragment or immunogenic variant of any of these sequences. Additional information regarding each of these suitable amino acid sequences encoding Rotavirus antigens may also be derived from the sequence listing, in particular from the details provided therein under identifier <223> as explained in the following.

In various embodiments, the coding RNA of the first aspect comprises at least one coding sequence encoding at least one cytoplasmic (not secreted) antigenic peptide or protein of Rotavirus comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 28-81, or an immunogenic fragment or immunogenic variant of any of these sequences. Additional information regarding each of these suitable amino acid sequences encoding Rotavirus antigens may also be derived from the sequence listing, in particular from the details provided therein under identifier <223> as explained in the following.

In preferred embodiments, the coding RNA of the first aspect comprises at least one coding sequence encoding at least one antigenic peptide or protein of Rotavirus comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 1-3, 4-6, 46-54, 64-72, 91-99 or 109-117, or an immunogenic fragment or immunogenic variant of any of these sequences. Additional information regarding each of these suitable amino acid sequences encoding Rotavirus antigens may also be derived from the sequence listing, in particular from the details provided therein under identifier <223> as explained in the following.

In other embodiments, the coding RNA of the first aspect comprises at least one coding sequence encoding at least one antigenic peptide or protein comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 1-827 of WO2017/081110A1 or a fragment or variant of any of these sequences. In this context SEQ ID NOs: 1-827 of WO2017/081110A1 and the disclosure related thereto herewith incorporated by reference.

Suitable Coding Sequences:

According to preferred embodiments, the coding RNA comprises at least one coding sequence encoding at least one antigenic protein as defined herein, preferably VP8*, or fragments and variants thereof. In that context, any coding sequence encoding at least one antigenic protein as defined herein, preferably VP8*, or fragments and variants thereof may be understood as suitable coding sequence and may therefore be comprised in the coding RNA of the invention.

In preferred embodiments, the coding RNA of the first aspect may comprise or consist of at least one coding sequence encoding at least one antigenic peptide or protein from VP8* as defined herein, preferably encoding any one of SEQ ID NOs: 1-117, 1899, 1900 or fragments of variants thereof. It has to be understood that, on nucleic acid level, any RNA sequence which encodes an amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 1-117, 1899, 1900 or fragments or variants thereof, may be selected and may accordingly be understood as suitable coding sequence and may therefore be comprised in the coding RNA of the first aspect.

In preferred embodiments, the coding RNA of the first aspect may comprise or consist of at least one coding sequence encoding any one of SEQ ID NOs: 1-6, 46-54, 64-72, 91-99 or 109-117 or fragments of variants thereof. It has to be understood that, on nucleic acid level, any RNA sequence which encodes an amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 1-6, 46-54, 64-72, 91-99 or 109-117 or fragments or variants thereof, may be selected and may accordingly be understood as suitable coding sequence and may therefore be comprised in the coding RNA of the first aspect.

In other embodiments, the coding RNA of the first aspect may comprise or consist of at least one coding sequence encoding any one of SEQ ID NOs: 1-827 of WO2017/081110A1 or fragments of variants thereof. It has to be understood that, on nucleic acid level, any RNA sequence which encodes an amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 1-827 of WO2017/081110A1 or fragments or variants thereof, may be selected and may accordingly be understood as suitable coding sequence and may therefore be comprised in the coding RNA of the invention.

In preferred embodiments, the coding RNA of the first aspect comprises a coding sequence that comprises at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 118-585, 1901-1906, or a fragment or a fragment or variant of any of these sequences. Additional information regarding each of these suitable nucleic acid sequences encoding may also be derived from the sequence listing, in particular from the details provided therein under identifier <223>.

In other embodiments, the coding RNA of the first aspect comprises a coding sequence that comprises at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to any one of the nucleic acid sequences provided in Tables 2, 4, 6, 8, 10-13, 15, 17, and 19 of WO2017/081110A1 or a fragment or a fragment or variant of any of these sequences. The respective nucleic acid sequences provided in Tables 2, 4, 6, 8, 10-13, 15, 17, and 19 of WO2017/081110A1, and the disclosure relating thereto, herewith incorporated by reference.

In preferred embodiments, the coding RNA of the first aspect is an artificial RNA.

The term “artificial RNA” as used herein is intended to refer to an RNA that does not occur naturally. In other words, an artificial RNA may be understood as a non-natural RNA molecule. Such RNA molecules may be non-natural due to its individual sequence (e.g. G/C content modified coding sequence, UTRs) and/or due to other modifications, e.g. structural modifications of nucleotides. Typically, artificial RNA may be designed and/or generated by genetic engineering to correspond to a desired artificial sequence of nucleotides. In this context an artificial RNA is a sequence that may not occur naturally, i.e. a sequence that differs from the wild type sequence/the naturally occurring sequence by at least one nucleotide. The term “artificial RNA” is not restricted to mean “one single molecule” but is understood to comprise an ensemble of essentially identical RNA molecules. Accordingly, it may relate to a plurality of essentially identical RNA molecules.

In preferred embodiments, the coding RNA of the first aspect is a modified and/or stabilized RNA, preferably a modified and/or stabilized artificial RNA

According to preferred embodiments, the RNA of the present invention may thus be provided as a “stabilized artificial RNA” or “stabilized coding RNA” that is to say an RNA showing improved resistance to in vivo degradation and/or an RNA showing improved stability in vivo, and/or an RNA showing improved translatability in vivo. In the following, specific suitable modifications/adaptations in this context are described which are suitably to “stabilize” the RNA.

Such stabilization may be effected by providing a “dried RNA” and/or a “purified RNA” as specified herein. Alternatively or in addition to that, such stabilization can be effected, for example, by a modified phosphate backbone of the coding RNA of the present invention. A backbone modification in connection with the present invention is a modification in which phosphates of the backbone of the nucleotides contained in the RNA are chemically modified. Nucleotides that may be preferably used in this connection contain e.g. a phosphorothioate-modified phosphate backbone, preferably at least one of the phosphate oxygens contained in the phosphate backbone being replaced by a sulfur atom. Stabilized RNAs may further include, for example: non-ionic phosphate analogues, such as, for example, alkyl and aryl phosphonates, in which the charged phosphonate oxygen is replaced by an alkyl or aryl group, or phosphodiesters and alkylphosphotriesters, in which the charged oxygen residue is present in alkylated form. Such backbone modifications typically include, without implying any limitation, modifications from the group consisting of methylphosphonates, phosphoramidates and phosphorothioates (e.g. cytidine-5′-O-(1-thiophosphate)).

In the following, suitable modifications are described that are capable of “stabilizing” the RNA of the invention.

According to embodiments, the coding RNA is a modified RNA, wherein the modification refers to chemical modifications comprising backbone modifications as well as sugar modifications or base modifications.

A modified RNA may comprise nucleotide analogues/modifications, e.g. backbone modifications, sugar modifications or base modifications. A backbone modification in the context of the invention is a modification, in which phosphates of the backbone of the nucleotides of the RNA are chemically modified. A sugar modification in the context of the invention is a chemical modification of the sugar of the nucleotides of the RNA. Furthermore, a base modification in the context of the invention is a chemical modification of the base moiety of the nucleotides of the RNA. In this context, nucleotide analogues or modifications are preferably selected from nucleotide analogues which are applicable for transcription and/or translation.

In particularly preferred embodiments, the nucleotide analogues/modifications which may be incorporated into a modified RNA as described herein are preferably selected from 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-0-Methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-Iodo-2′-deoxycytidine-5′-triphosphate, 5-iodouridine-5′-triphosphate, 5-Iodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, pseudouridine-5′-triphosphate, or puromycin-5′-triphosphate, xanthosine-5-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, 5′-O-(1-thiophosphate)-adenosine, 5′-O-(1-thiophosphate)-cytidine, 5′-O-(1-thiophosphate)-guanosine, 5′-O-(1-thiophosphate)-uridine, 5′-O-(1-thiophosphate)-pseudouridine, 6-aza-cytidine, 2-thio-cytidine, alpha-thio-cytidine, Pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, Pyrrolo-cytidine, inosine, alpha-thio-guanosine, 6-methyl-guanosine, 5-methyl-cytidine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, alpha-thio-adenosine, 8-azido-adenosine, 7-deaza-adenosine.

In some embodiments, the at least one modified nucleotide is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

In some embodiments, 100% of the uracil in the coding sequence have a chemical modification, preferably a chemical modification is in the 5-position of the uracil.

Particularly preferred in the context of the invention are pseudouridine (ψ), N1-methylpseudouridine (m1ψ), 5-methylcytosine, and 5-methoxyuridine.

Incorporating modified nucleotides such as pseudouridine (ψ), N1-methylpseudouridine (m1ψ), 5-methylcytosine, and/or 5-methoxyuridine into the coding sequence of the RNA of the first aspect may be advantageous as unwanted innate immune responses (upon administration of the coding RNA or the Rotavirus vaccine) may be adjusted or reduced.

In embodiments, the coding RNA comprises at least one coding sequence encoding a Rotavoirus antigenic protein as defined herein, preferably VP8* as defined herein, wherein said coding sequence comprises at least one modified nucleotide selected from pseudouridine (ψ) and N1-methylpseudouridine (m1ψ).

In preferred embodiments, the coding RNA comprises at least one coding sequence, wherein said coding sequence comprises at least one pseudouridine (ψ) nucleotide.

In a embodiments, the coding RNA comprises at least one coding sequence encoding a Rotavoirus antigenic protein as defined herein, preferably VP8*, wherein said coding sequence comprises at least one modified nucleotide selected from pseudouridine (ψ) and/or N1-methylpseudouridine (m1ψ), wherein all uracil nucleotides are replaced by pseudouridine (ψ) nucleotides and/or N1-methylpseudouridine (m1ψ) nucleotides.

In preferred embodiments, the coding RNA comprises at least one coding sequence, wherein said coding sequence comprises pseudouridine (ψ) nucleotides, wherein all uracil nucleotides are replaced by pseudouridine (ψ) nucleotides.

In preferred embodiments, the coding RNA comprises at least one codon modified coding sequence.

In preferred embodiments, the at least one coding sequence (of the coding RNA) is a codon modified coding sequence, wherein the amino acid sequence encoded by the at least one codon modified coding sequence is preferably not being modified compared to the amino acid sequence encoded by the corresponding wild type coding sequence.

The term “codon modified coding sequence” relates to coding sequences that differ in at least one codon (triplets of nucleotides coding for one amino acid) compared to the corresponding wild type coding sequence. Suitably, a codon modified coding sequence in the context of the invention may show improved resistance to in vivo degradation and/or improved stability in vivo, and/or improved translatability in vivo. Codon modifications in the broadest sense make use of the degeneracy of the genetic code wherein multiple codons may encode the same amino acid and may be used interchangeably (cf. Table 2) to optimize/modify the coding sequence for in vivo applications as outlined above.

In particularly preferred embodiments, the at least one coding sequence of the coding RNA is a codon modified coding sequence, wherein the codon modified coding sequence is selected from C maximized coding sequence, CAI maximized coding sequence, human codon usage adapted coding sequence, G/C content modified coding sequence, and G/C optimized coding sequence, or any combination thereof, or any combination thereof.

In embodiments, the coding RNA may be modified, wherein the C content of the at least one coding sequence may be increased, preferably maximized, compared to the C content of the corresponding wild type coding sequence (herein referred to as “C maximized coding sequence”). The amino acid sequence encoded by the C maximized coding sequence of the RNA is preferably not modified compared to the amino acid sequence encoded by the respective wild type coding sequence. The generation of a C maximized nucleic acid sequences may suitably be carried out using a modification method according to WO2015/062738. In this context, the disclosure of WO2015/062738 is included herewith by reference.

In embodiments, the coding RNA may be modified, wherein the G/C content of the at least one coding sequence may be modified compared to the G/C content of the corresponding wild type coding sequence (herein referred to as “G/C content modified coding sequence”). In this context, the terms “G/C optimization” or “G/C content modification” relate to an RNA that comprises a modified, preferably an increased number of guanosine and/or cytosine nucleotides as compared to the corresponding wild type coding sequence. Such an increased number may be generated by substitution of codons containing adenosine or thymidine nucleotides by codons containing guanosine or cytosine nucleotides. Advantageously, RNA sequences having an increased G/C content are more stable or show a better expression than sequences having an increased A/U. The amino acid sequence encoded by the G/C content modified coding sequence of the RNA is preferably not modified as compared to the amino acid sequence encoded by the respective wild type sequence. Preferably, the G/C content of the coding sequence of the RNA is increased by at least 10%, 20%, 30%, preferably by at least 40% compared to the G/C content of the coding sequence of the corresponding wild type RNA sequence.

In preferred embodiments, the coding RNA may be modified, wherein the G/C content of the at least one coding sequence may be optimized compared to the G/C content of the corresponding wild type coding sequence (herein referred to as “G/C content optimized coding sequence”). “Optimized” in that context refers to a coding sequence wherein the G/C content is preferably increased to the essentially highest possible G/C content. The amino acid sequence encoded by the G/C content optimized coding sequence of the RNA is preferably not modified as compared to the amino acid sequence encoded by the respective wild type coding sequence. The generation of a G/C content optimized RNA sequence may be carried out using a method according to WO2002/098443. In this context, the disclosure of WO2002/098443 is included in its full scope in the present invention. Throughout the description, including the <223> identifier of the sequence listing, G/C optimized coding sequences are indicated by the abbreviations “opt1” or “opt5”.

In embodiments, the coding RNA may be modified, wherein the codons in the at least one coding sequence may be adapted to human codon usage (herein referred to as “human codon usage adapted coding sequence”). Codons encoding the same amino acid occur at different frequencies in humans. Accordingly, the coding sequence of the RNA is preferably modified such that the frequency of the codons encoding the same amino acid corresponds to the naturally occurring frequency of that codon according to the human codon usage. For example, in the case of the amino acid Ala, the wild type coding sequence is preferably adapted in a way that the codon “GCC” is used with a frequency of 0.40, the codon “GCT” is used with a frequency of 0.28, the codon “GCA” is used with a frequency of 0.22 and the codon “GCG” is used with a frequency of 0.10 etc. (see Table 2). Accordingly, such a procedure (as exemplified for Ala) is applied for each amino acid encoded by the coding sequence of the RNA to obtain sequences adapted to human codon usage. Throughout the description, including the <223> identifier of the sequence listing, human codon usage adapted coding sequences are indicated by the abbreviation “opt3”.

TABLE 2
Human codon usage table with frequencies
indicated for each amino acid

| Amino | | | Amino | | |
|---|---|---|---|---|---|
| acid | codon | frequency | acid | codon | frequency |
| Ala | GCG | 0.10 | Pro | CCG | 0.11 |
| Ala | GCA | 0.22 | Pro | CCA | 0.27 |
| Ala | GCT | 0.28 | Pro | CCT | 0.29 |
| Ala | GCC* | 0.40 | Pro | CCC* | 0.33 |
| Cys | TGT | 0.42 | Gln | CAG* | 0.73 |
| Cys | TGC* | 0.58 | Gln | CAA | 0.27 |
| Asp | GAT | 0.44 | Arg | AGG | 0.22 |
| Asp | GAC* | 0.56 | Arg | AGA* | 0.21 |
| Glu | GAG* | 0.59 | Arg | CGG | 0.19 |
| Glu | GAA | 0.41 | Arg | CGA | 0.10 |
| Phe | TTT | 0.43 | Arg | CGT | 0.09 |
| Phe | TTC* | 0.57 | Arg | CGC | 0.19 |
| Gly | GGG | 0.23 | Ser | AGT | 0.14 |
| Gly | GGA | 0.26 | Ser | AGC* | 0.25 |
| Gly | GGT | 0.18 | Ser | TCG | 0.06 |
| Gly | GGC* | 0.33 | Ser | TCA | 0.15 |
| His | CAT | 0.41 | Ser | TCT | 0.18 |
| His | CAC* | 0.59 | Ser | TCC | 0.23 |
| Ile | ATA | 0.14 | Thr | ACG | 0.12 |
| Ile | ATT | 0.35 | Thr | ACA | 0.27 |
| Ile | ATC* | 0.52 | Thr | ACT | 0.23 |
| Lys | AAG* | 0.60 | Thr | ACC* | 0.38 |
| Lys | AAA | 0.40 | Val | GTG* | 0.48 |
| Leu | TTG | 0.12 | Val | GTA | 0.10 |
| Leu | TTA | 0.06 | Val | GTT | 0.17 |
| Leu | CTG* | 0.43 | Val | GTC | 0.25 |
| Leu | CTA | 0.07 | Trp | TGG* | 1 |
| Leu | CTT | 0.12 | Tyr | TAT | 0.42 |
| Leu | CTC | 0.20 | Tyr | TAC* | 0.58 |
| Met | ATG* | 1 | Stop | TGA* | 0.61 |
| Asn | AAT | 0.44 | Stop | TAG | 0.17 |
| Asn | AAC* | 0.56 | Stop | TAA | 0.22 |

*most frequent human codon

In preferred embodiments, the coding RNA may be modified, wherein the codon adaptation index (CAI) may be increased or preferably maximised in the at least one coding sequence (herein referred to as “CAI maximized coding sequence”). It is preferred that all codons of the wild type nucleic acid sequence that are relatively rare in e.g. a human are exchanged for a respective codon that is frequent in the e.g. a human, wherein the frequent codon encodes the same amino acid as the relatively rare codon. Suitably, the most frequent codons are used for each amino acid of the encoded protein (see Table 2, most frequent human codons are marked with asterisks). Suitably, the coding RNA comprises at least one coding sequence, wherein the codon adaptation index (CAI) of the at least one coding sequence is at least 0.5, at least 0.8, at least 0.9 or at least 0.95. Most preferably, the codon adaptation index (CAI) of the at least one coding sequence is 1 (CAI=1). For example, in the case of the amino acid Ala, the wild type coding sequence may be adapted in a way that the most frequent human codon “GCC” is always used for said amino acid. Accordingly, such a procedure (as exemplified for Ala) may be applied for each amino acid encoded by the coding sequence of the RNA to obtain CAI maximized coding sequences. Throughout the description, including the <223> identifier of the sequence listing, CAI maximized coding sequences are indicated by the abbreviation “opt4”.

Suitable VP8* proteins/constructs as defined herein and their particular coding sequences are disclosed in Table 3. Therein, each row corresponds to suitable VP8* constructs (compare with Table 1, columns A and B).

Column A of Table 3 provides a short description of suitable antigen constructs (see Table X1 for the description of heterologous fragments). Column B of Table 3 provides a description of the Rotavirus of which the respective VP8* is derived from. Column C of Table 3 provides protein SEQ ID NOs of respective VP8* antigen constructs. Column D of Table 3 provides SEQ ID NO of the corresponding wild type RNA coding sequences. Column E of Table 3 provides SEQ ID NO of the corresponding G/C optimized RNA coding sequences (opt1). Column F of Table 3 provides SEQ ID NO of the corresponding G/C optimized RNA coding sequences (opt5). Column G of Table 3 provides SEQ ID NO of the corresponding CAI maximized coding sequence (opt4).

Notably, the description of the invention explicitly includes the information provided under <223> identifier of the ST25 sequence listing of the present application. Preferred mRNA sequences comprising the coding sequences of Table 3 are provided in Table 4.

TABLE 3
Preferred coding sequences encoding Rotavirus VP8* antigen constructs:

| A | B | C | D | E | F | G |
|---|---|---|---|---|---|---|
| VP4 | RVA/BE1058/P[4] | 10 | 118 | 154, 262 | 370 | 478 |
| VP4 | RVA/DS-1/P[4] | 11 | 119 | 155, 263 | 371 | 479 |
| VP4 | RVA/DS-1/P[4] | 12 | 120 | 156, 264 | 372 | 480 |
| VP4 | RVA/F01322/P[6] | 13 | 121 | 157, 265 | 373 | 481 |
| VP4 | RVA/1076/P[6] | 14 | 122 | 158, 266 | 374 | 482 |
| VP4 | RVA/BE1128/P[8] | 15 | 123 | 159, 267 | 375 | 483 |
| VP4 | RVA/Wa-VirWa/P[8] | 16 | 124 | 160, 268 | 376 | 484 |
| VP4 | RVA/Wa/P[8] | 17 | 125 | 161, 269 | 377 | 485 |
| VP4 | RVA/Wa/P[8] | 18 | 126 | 162, 270 | 378 | 486 |
| VP8* | RVA/BE1058/P[4] | 9, 19 | 127 | 163, 271 | 379 | 487 |
| VP8* | RVA/DS-1/P[4] | 20 | 128 | 164, 272 | 380 | 488 |
| VP8* | RVA/DS-1/P[4] | 21 | 129 | 165, 273 | 381 | 489 |
| VP8* | RVA/F01322/P[6] | 8, 22 | 130 | 166, 274 | 382 | 490 |
| VP8* | RVA/1076/P[6] | 23 | 131 | 167, 275 | 383 | 491 |
| VP8* | RVA/BE1128/P[8] | 7, 24 | 132 | 168, 276 | 384 | 492 |
| VP8* | RVA/Wa-VirWa/P[8] | 25 | 133 | 169, 277 | 385 | 493 |
| VP8* | RVA/Wa/P[8] | 26 | 134 | 170, 278 | 386 | 494 |
| VP8* | RVA/Wa/P[8] | 27 | 135 | 171, 279 | 387 | 495 |
| VP8*(65-223) | RVA/BE1058/P[4] | 28 | 136 | 172, 280 | 388 | 496 |
| VP8*(65-223) | RVA/DS-1/P[4] | 29 | 137 | 173, 281 | 389 | 497 |
| VP8*(65-223) | RVA/DS-1/P[4] | 30 | 138 | 174, 282 | 390 | 498 |
| VP8*(65-223) | RVA/F01322/P[6] | 31 | 139 | 175, 283 | 391 | 499 |
| VP8*(65-223) | RVA/1076/P[6] | 32 | 140 | 176, 284 | 392 | 500 |
| VP8*(65-223) | RVA/BE1128/P[8] | 33 | 141 | 177, 285 | 393 | 501 |
| VP8*(65-223) | RVA/Wa-VirWa/P[8] | 34 | 142 | 178, 286 | 394 | 502 |
| VP8*(65-223) | RVA/Wa/P[8] | 35 | 143 | 179, 287 | 395 | 503 |
| VP8*(65-223) | RVA/Wa/P[8] | 36 | 144 | 180, 288 | 396 | 504 |
| VP8*(41-223) | RVA/BE1058/P[4] | 37 | 145 | 181, 289 | 397 | 505 |
| VP8*(41-223) | RVA/DS-1/P[4] | 38 | 146 | 182, 290 | 398 | 506 |
| VP8*(41-223) | RVA/DS-1/P[4] | 39 | 147 | 183, 291 | 399 | 507 |
| VP8*(41-223) | RVA/F01322/P[6] | 40 | 148 | 184, 292 | 400 | 508 |
| VP8*(41-223) | RVA/1076/P[6] | 41 | 149 | 185, 293 | 401 | 509 |
| VP8*(41-223) | RVA/BE1128/P[8] | 42 | 150 | 186, 294 | 402 | 510 |
| VP8*(41-223) | RVA/Wa-VirWa/P[8] | 43 | 151 | 187, 295 | 403 | 511 |
| VP8*(41-223) | RVA/Wa/P[8] | 44 | 152 | 188, 296 | 404 | 512 |
| VP8*(41-223) | RVA/Wa/P[8] | 45 | 153 | 189, 297 | 405 | 513 |
| P2_VP8*(65-223) | RVA/BE1058/P[4] | 6, 46 | | 190, 298, 1903 | 406 | 514 |
| P2_VP8*(65-223) | RVA/DS-1/P[4] | 47 | | 191, 299 | 407 | 515 |
| P2_VP8*(65-223) | RVA/DS-1/P[4] | 48 | | 192, 300 | 408 | 516 |
| P2_VP8*(65-223) | RVA/F01322/P[6] | 5, 49 | | 193, 301, 1904 | 409 | 517 |
| P2_VP8*(65-223) | RVA/1076/P[6] | 50 | | 194, 302 | 410 | 518 |
| P2_VP8*(65-223) | RVA/BE1128/P[8] | 4, 51 | | 195, 303 | 411 | 519 |
| P2_VP8*(65-223) | RVA/Wa-VirWa/P[8] | 52 | | 196, 304 | 412 | 520 |
| P2_VP8*(65-223) | RVA/Wa/P[8] | 53 | | 197, 305 | 413 | 521 |
| P2_VP8*(65-223) | RVA/Wa/P[8] | 54 | | 198, 306 | 414 | 522 |
| P2_VP8*(41-223) | RVA/BE1058/P[4] | 55 | | 199, 307 | 415 | 523 |
| P2_VP8*(41-223) | RVA/DS-1/P[4] | 56 | | 200, 308 | 416 | 524 |
| P2_VP8*(41-223) | RVA/DS-1/P[4] | 57 | | 201, 309 | 417 | 525 |
| P2_VP8*(41-223) | RVA/F01322/P[6] | 58 | | 202, 310 | 418 | 526 |
| P2_VP8*(41-223) | RVA/1076/P[6] | 59 | | 203, 311 | 419 | 527 |
| P2_VP8*(41-223) | RVA/BE1128/P[8] | 60 | | 204, 312 | 420 | 528 |
| P2_VP8*(41-223) | RVA/Wa-VirWa/P[8] | 61 | | 205, 313 | 421 | 529 |
| P2_VP8*(41-223) | RVA/Wa/P[8] | 62 | | 206, 314 | 422 | 530 |
| P2_VP8*(41-223) | RVA/Wa/P[8] | 63 | | 207, 315 | 423 | 531 |
| P2_VP8*(65-223)_Ferritin | RVA/BE1058/P[4] | 64 | | 208, 316 | 424 | 532 |
| P2_VP8*(65-223)_Ferritin | RVA/DS-1/P[4] | 65 | | 209, 317 | 425 | 533 |
| P2_VP8*(65-223)_Ferritin | RVA/DS-1/P[4] | 66 | | 210, 318 | 426 | 534 |
| P2_VP8*(65-223)_Ferritin | RVA/F01322/P[6] | 67 | | 211, 319 | 427 | 535 |
| P2_VP8*(65-223)_Ferritin | RVA/1076/P[6] | 68 | | 212, 320 | 428 | 536 |
| P2_VP8*(65-223)_Ferritin | RVA/BE1128/P[8] | 69 | | 213, 321 | 429 | 537 |
| P2_VP8*(65-223)_Ferritin | RVA/Wa-VirWa/P[8] | 70 | | 214, 322 | 430 | 538 |
| P2_VP8*(65-223)_Ferritin | RVAA/Wa/P[8] | 71 | | 215, 323 | 431 | 539 |
| P2_VP8*(65-223)_Ferritin | RVA/Wa/P[8] | 72 | | 216, 324 | 432 | 540 |
| P2_VP8*(41-223)_Ferritin | RVA/BE1058/P[4] | 73 | | 217, 325 | 433 | 541 |
| P2_VP8*(41-223)_Ferritin | RVA/DS-1/P[4] | 74 | | 218, 326 | 434 | 542 |
| P2_VP8*(41-223)_Ferritin | RVA/DS-1/P[4] | 75 | | 219, 327 | 435 | 543 |
| P2_VP8*(41-223)_Ferritin | RVA/F01322/P[6] | 76 | | 220, 328 | 436 | 544 |
| P2_VP8*(41-223)_Ferritin | RVA/1076/P[6] | 77 | | 221, 329 | 437 | 545 |
| P2_VP8*(41-223)_Ferritin | RVA/BE1128/P[8] | 78 | | 222, 330 | 438 | 546 |
| P2_VP8*(41-223)_Ferritin | RVA/Wa-VirWa/P[8] | 79 | | 223, 331 | 439 | 547 |
| P2_VP8*(41-223)_Ferritin | RVA/Wa/P[8] | 80 | | 224, 332 | 440 | 548 |
| P2_VP8*(41-223)_Ferritin | RVA/Wa/P[8] | 81 | | 225, 333 | 441 | 549 |
| LumSynt_P2_VP8*(65-223) | RVA/BE1058/P[4] | 82 | | 226, 334 | 442 | 550 |
| LumSynt_P2_VP8*(65-223) | RVA/DS-1/P[4] | 83 | | 227, 335 | 443 | 551 |
| LumSynt_P2_VP8*(65-223) | RVA/DS-1/P[4] | 84 | | 228, 336 | 444 | 552 |
| LumSynt_P2_VP8*(65-223) | RVA/F01322/P[6] | 85 | | 229, 337 | 445 | 553 |
| LumSynt_P2_VP8*(65-223) | RVA/1076/P[6] | 86 | | 230, 338 | 446 | 554 |
| LumSynt_P2_VP8*(65-223) | RVA/BE1128/P[8] | 87 | | 231, 339 | 447 | 555 |
| LumSynt_P2_VP8*(65-223) | RVA/Wa-VirWa/P[8] | 88 | | 232, 340 | 448 | 556 |
| LumSynt_P2_VP8*(65-223) | RVA/Wa/P[8] | 89 | | 233, 341 | 449 | 557 |
| LumSynt_P2_VP8*(65-223) | RVA/Wa/P[8] | 90 | | 234, 342 | 450 | 558 |
| LumSynt_P2_VP8*(41-223) | RVA/BE1058/P[4] | 3, 91 | | 235, 343, 1905 | 451 | 559 |
| LumSynt_P2_VP8*(41-223) | RVA/DS-1/P[4] | 92 | | 236, 344 | 452 | 560 |
| LumSynt_P2_VP8*(41-223) | RVA/DS-1/P[4] | 93 | | 237, 345 | 453 | 561 |
| LumSynt_P2_VP8*(41-223) | RVA/F01322/P[6l | 2, 94 | | 238, 346, 1906 | 454 | 562 |
| LumSynt_P2_VP8*(41-223) | RVA/1076/P[6] | 95 | | 239, 347 | 455 | 563 |
| LumSynt_P2_VP8*(41-223) | RVA/BE1128/P[8] | 1, 96 | | 240, 348 | 456 | 564 |
| LumSynt_P2_VP8*(41-223) | RVA/Wa-VirWa/P[8] | 97 | | 241, 349 | 457 | 565 |
| LumSynt_P2_VP8*(41-223) | RVA/Wa/P[8] | 98 | | 242, 350 | 458 | 566 |
| LumSynt_P2_VP8*(41-223) | RVA/Wa/P{8] | 99 | | 243, 351 | 459 | 567 |
| SP-IgE_P2_VP8*(65-223) | RVA/BE1058/P[4] | 100 | | 244, 352 | 460 | 568 |
| SP-IgE_P2_VP8*(65-223) | RVA/DS-1/P[4] | 101 | | 245, 353 | 461 | 569 |
| SP-IgE_P2_VP8*(65-223) | RVA/DS-1/P[41 | 102 | | 246, 354 | 462 | 570 |
| SP-IgE_P2_VP8*(65-223) | RVA/F01322/P[6] | 103 | | 247, 355 | 463 | 571 |
| SP-IgE_P2_VP8*(65-223) | RVA/1076/P[6] | 104 | | 248, 356 | 464 | 572 |
| SP-IgE_P2_VP8*(65-223) | RVA/BE1128/P[8] | 105 | | 249, 357 | 465 | 573 |
| SP-IgE_P2_VP8*(65-223) | RVA/Wa-VirWa/P[8] | 106 | | 250, 358 | 466 | 574 |
| SP-IgE_P2_VP8*(65-223) | RVA/Wa/P[8] | 107 | | 251, 359 | 467 | 575 |
| SP-IgE_P2_VP8*(65-223) | RVA/Wa/P[8] | 108 | | 252, 360 | 468 | 576 |
| SP-IgE_P2_VP8*(41-223) | RVA/BE1058/P[4] | 109 | | 253, 361 | 469 | 577 |
| SP-IgE_P2_VP8*(41-223) | RVA/DS-1/P[4] | 110 | | 254, 362 | 470 | 578 |
| SP-IgE_P2_VP8*(41-223) | RVA/DS-1/P[4] | 111 | | 255, 363 | 471 | 579 |
| SP-IgE_P2_VP8*(41-223) | RVA/F01322/P[6] | 112 | | 256, 364 | 472 | 580 |
| SP-IgE_P2_VP8*(41-223) | RVA/1076/P[6] | 113 | | 257, 365 | 473 | 581 |
| SP-IgE_P2_VP8*(41-223) | RVA/BE1128/P[8] | 114 | | 258, 366 | 474 | 582 |
| SP-IgE_P2_VP8*(41-223) | RVA/Wa-VirWa/P[8] | 115 | | 259, 367 | 475 | 583 |
| SP-IgE_P2_VP8*(41-223) | RVA/Wa/P[8] | 116 | | 260, 368 | 476 | 584 |
| SP-IgE_P2_VP8*(41-223) | RVA/Wa/P[8] | 117 | | 261, 369 | 477 | 585 |
| P2_VP8*(64-223) | RVA/BE1058/P[4] | 1899 | | 1901 | | |
| P2_VP8*(64-223) | RVA/F01322/P[6] | 1900 | | 1902 | | |

In preferred embodiments, the coding RNA of the first aspect comprises at least one coding sequence comprising or consisting a codon modified nucleic acid sequence which is identical or at least 70%, 80%, 85%, 86%, 87%, 88%, 89%, 90%, 91%, 92%, 93%, 94%, 95%, 96%, 97%, 98%, or 99% identical to a codon modified nucleic acid sequence selected from the group consisting of SEQ ID NOs: 154-585, 1901-1906 or a fragment or variant of any of these sequences. Additional information regarding each of these suitable nucleic acid sequences encoding may also be derived from the sequence listing, in particular from the details provided therein under identifier <223>.

In particularly preferred embodiments, the coding RNA of the first aspect comprises at least one coding sequence comprising or consisting a codon modified nucleic acid sequence which is identical or at least 70%, 80%, 85%, 86%, 87%, 88%, 89%, 90%, 91%, 92%, 93%, 94%, 95%, 96%, 97%, 98%, or 99% identical to a codon modified nucleic acid sequence selected from the group consisting of SEQ ID NOs: 154-369, 1901-1906 or a fragment or variant of any of these sequences. Additional information regarding each of these suitable nucleic acid sequences encoding may also be derived from the sequence listing, in particular from the details provided therein under identifier <223>.

RNA Elements, mRNA Elements:

In embodiments, the coding RNA of the first aspect may be monocistronic, bicistronic, or multicistronic.

The term “monocistronic” will be recognized and understood by the person of ordinary skill in the art, and is e.g. intended to refer to an RNA that comprises only one coding sequences. The terms “bicistronic”, or “multicistronic” as used herein will be recognized and understood by the person of ordinary skill in the art, and are e.g. intended to refer to an RNA that may comprise two (bicistronic) or more (multicistronic) coding sequences.

In preferred embodiments, the coding RNA of the first aspect is monocistronic.

In embodiments, the coding RNA is monocistronic and the coding sequence of said RNA encodes at least two different antigenic peptides or proteins derived from a Rotavirus (e.g. VP8*). Accordingly, said coding sequence may encode at least two, three, four, five, six, seven, eight and more antigenic peptides or proteins derived from a Rotavirus (e.g. VP8*), linked with or without an amino acid linker sequence, wherein said linker sequence can comprise rigid linkers, flexible linkers, cleavable linkers, or a combination thereof. Such constructs are herein referred to as “multi-antigen-constructs”.

In embodiments, the coding RNA may be bicistronic or multicistronic and comprises at least two coding sequences, wherein the at least two coding sequences encode two or more different antigenic peptides or proteins derived from Rotavirus (e.g. VP8*). Accordingly, the coding sequences in a bicistronic or multicistronic RNA suitably encodes distinct antigenic proteins or peptides as defined herein or immunogenic fragments or immunogenic variants thereof. Preferably, the coding sequences in said bicistronic or multicistronic constructs may be separated by at least one IRES (internal ribosomal entry site) sequence. Thus, the term “encoding two or more antigenic peptides or proteins” may mean, without being limited thereto, that the bicistronic or multicistronic RNA encodes e.g. at least two, three, four, five, six or more (preferably different) antigenic peptides or proteins of different Rotaviruses. Alternatively, the bicistronic or multicistronic RNA may encode e.g. at least two, three, four, five, six or more (preferably different) antigenic peptides or proteins derived from the same Rotavirus. In that context, suitable IRES sequences may be selected from the list of nucleic acid sequences according to SEQ ID NOs: 1566-1662 of the patent application WO2017/081082, or fragments or variants of these sequences. In this context, the disclosure of WO2017/081082 relating to IRES sequences is herewith incorporated by reference.

It has to be understood that, in the context of the invention, certain combinations of coding sequences may be generated by any combination of monocistronic, bicistronic and multicistronic RNA constructs and/or multi-antigen-constructs to obtain a composition encoding multiple antigenic peptides or proteins as defined herein.

Preferably, the coding RNA of the first aspect typically comprises about 50 to about 20000 nucleotides, or about 500 to about 10000 nucleotides, or about 1000 to about 10000 nucleotides, or preferably about 1000 to about 5000 nucleotides, or even more preferably about 1000 to about 2500 nucleotides.

According to preferred embodiments, the coding RNA of the first aspect is an mRNA, a self-replicating RNA, a circular RNA, a viral RNA, or a replicon RNA.

In embodiments, the coding RNA of the first aspect is a circular RNA. As used herein, “circular RNA” or “circRNAs” have to be understood as a circular polynucleotide constructs that encode at least one antigenic peptide or protein as defined herein. Accordingly, in preferred embodiments, said circRNA comprises at least one coding sequence encoding at least one antigenic protein from a Rotavirus (e.g., VP8*), or an immunogenic fragment or an immunogenic variant thereof.

In embodiments, the coding RNA is a replicon RNA. The term “replicon RNA” will be recognized and understood by the person of ordinary skill in the art, and is e.g. intended to be an optimized self-replicating RNA. Such constructs may include replicase elements derived from e.g. alphaviruses (e.g. SFV, SIN, VEE, or RRV) and the substitution of the structural virus proteins with the nucleic acid of interest (that is, the coding sequence encoding a Rotavirus protein (e.g., VP8*)). Alternatively, the replicase may be provided on an independent coding RNA construct. Downstream of the replicase may be a sub-genomic promoter that controls replication of the replicon RNA.

In preferred embodiments, the coding RNA of the first aspect is an mRNA.

The terms “RNA” and “mRNA” will be recognized and understood by the person of ordinary skill in the art, and are e.g. intended to be a ribonucleic acid molecule, i.e. a polymer consisting of nucleotides. These nucleotides are usually adenosine-monophosphate, uridine-monophosphate, guanosine-monophosphate and cytidine-monophosphate monomers which are connected to each other along a so-called backbone. The backbone is formed by phosphodiester bonds between the sugar, i.e. ribose, of a first and a phosphate moiety of a second, adjacent monomer. The specific succession of the monomers is called the RNA-sequence. The mRNA (messenger RNA) provides the nucleotide sequence that may be translated into an amino-acid sequence of a particular peptide or protein.

In the context of the invention, the coding RNA, preferably the mRNA, may provide at least one coding sequence encoding an antigenic protein from a Rotavirus (e.g. VP8*) that is translated into a functional antigen after administration (e.g. after administration to a subject, e.g. a human subject).

Accordingly, the coding RNA, preferably the mRNA, is suitable for a vaccine, preferably a Rotavirus vaccine.

Suitably, the RNA may be modified by the addition of a 5′-cap structure, which preferably stabilizes the coding RNA and/or enhances expression of the encoded antigen and/or reduces the stimulation of the innate immune system (after administration to a subject). A 5′-cap structure is of particular importance in embodiments where the coding RNA is a linear, e.g. a linear mRNA or a linear coding replicon RNA.

Accordingly, in preferred embodiments, the coding RNA, in particular the mRNA of the first aspect comprises a 5′-cap structure, preferably cap0, cap1, cap2, a modified cap0, or a modified cap1 structure.

The term “5′-cap structure” as used herein will be recognized and understood by the person of ordinary skill in the art, and is e.g. intended to refer to a 5′ modified nucleotide, particularly a guanine nucleotide, positioned at the 5′-end of an RNA molecule, e.g. an mRNA molecule. Preferably, the 5′-cap structure is connected via a 5′-5′-triphosphate linkage to the RNA.

5′-cap structures which may be suitable in the context of the present invention are cap0 (methylation of the first nucleobase, e.g. m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse cap analogue), modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

A 5′-cap (cap0 or cap1) structure may be formed in chemical RNA synthesis or in RNA in vitro transcription (co-transcriptional capping) using cap analogues.

The term “cap analogue” as used herein will be recognized and understood by the person of ordinary skill in the art, and is e.g. intended to refer to a non-polymerizable di-nucleotide or tri-nucleotide that has cap functionality in that it facilitates translation or localization, and/or prevents degradation of a nucleic acid molecule, particularly of an RNA molecule, when incorporated at the 5′-end of the nucleic acid molecule. Non-polymerizable means that the cap analogue will be incorporated only at the 5′-terminus because it does not have a 5′ triphosphate and therefore cannot be extended in the 3′-direction by a template-dependent polymerase, particularly, by template-dependent RNA polymerase. Examples of cap analogues include, but are not limited to, a chemical structure selected from the group consisting of m7GpppG, m7GpppA, m7GpppC; unmethylated cap analogues (e.g. GpppG); dimethylated cap analogue (e.g. m2,7GpppG), trimethylated cap analogue (e.g. m2,2,7GpppG), dimethylated symmetrical cap analogues (e.g. m7Gpppm7G), or anti reverse cap analogues (e.g. ARCA; m7,2′OmeGpppG, m7,2′dGpppG, m7,3′OmeGpppG, m7,3′dGpppG and their tetraphosphate derivatives). Further cap analogues have been described previously (WO2008/016473, WO2008/157688, WO2009/149253, WO2011/015347, and WO2013/059475). Further suitable cap analogues in that context are described in WO2017/066793, WO2017/066781, WO2017/066791, WO2017/066789, WO2017/053297, WO2017/066782, WO2018/075827 and WO2017/066797 wherein the disclosures referring to cap analogues are incorporated herewith by reference.

In embodiments, a modified cap1 structure is generated using tri-nucleotide cap analogue as disclosed in WO2017/053297, WO2017/066793, WO2017/066781, WO2017/066791, WO2017/066789, WO2017/066782, WO2018/075827 and WO2017/066797. In particular, any cap structures derivable from the structure disclosed in claim 1-5 of WO2017/053297 may be suitably used to co-transcriptionally generate a modified cap1 structure. Further, any cap structures derivable from the structure defined in claim 1 or claim 21 of WO2018/075827 may be suitably used to co-transcriptionally generate a modified cap1 structure.

In particularly preferred embodiments, the coding RNA, in particular the mRNA of the first aspect comprises a cap1 structure. As shown in the Example section, the presence of a cap1 structure is of particular importance as the induction of a specific immune response against Rotavirus VP8* could be increased (see Examples 4, 5, and 6).

In preferred embodiments, the 5′-cap structure may suitably be added co-transcriptionally using tri-nucleotide cap analogue as defined herein in an RNA in vitro transcription reaction as defined herein. Accordingly, as supported by the example section, it is surprisingly advantageous that the coding RNA comprises a cap1 structure, wherein said cap1 structure is obtainable by co-transcriptional capping. As shown in the Example section, the presence of a Cap1 structure obtainable by co-transcriptional capping is advantageous for the induction of a specific immune response against Rotavirus VP8* (see Example 6).

In preferred embodiments, the cap1 structure of the coding RNA of the invention is formed using co-transcriptional capping using tri-nucleotide cap analogues m7G(5′)ppp(5′)(2′OMeA)pG or m7G(5′)ppp(5′)(2′OMeG)pG. A preferred cap1 analogus in that context is m7G(5′)ppp(5′)(2′OMeA)pG.

An exemplary protocol of a co-transcriptional capping procedure is provided in the Examples section (see Example 1). As shown in the Example section, using a coding RNA comprising a cap1 structure obtainable by co-transcriptional capping induced stronger immune responses than a coding RNA comprising a cap1 structure obtainable by enzymatic capping. Without being bound to theory, that surprising effect may be explained by an improved capping efficiency using co-transcriptional capping compared to enzymatic capping, and/or that enzymatic capping can generate intermediate cap1 structures (e.g. partial methylation of the 5′ cap and/or partial of the ribose following the 5′ cap). Both factors (reduced capping efficiency and presence of cap intermediates) may reduce the efficiency/potency of the coding RNA when used as e.g. a vaccine.

In other embodiments, the 5′-cap structure is formed via enzymatic capping using capping enzymes (e.g. vaccinia virus capping enzymes and/or cap-dependent 2′-0 methyltransferases) to generate cap0 or cap1 or cap2 structures. The 5′-cap structure (cap0 or cap1) may be added using immobilized capping enzymes and/or cap-dependent 2′-0 methyltransferases using methods and means disclosed in WO2016/193226.

In preferred embodiments, about 70%, 75%, 80%, 85%, 90%, 95% of the coding RNA (species) comprises a cap1 structure as determined using a capping assay. In preferred embodiments, less than about 20%, 15%, 10%, 5%, 4%, 3%, 2%, 1% of the coding RNA (species) does not comprises a cap1 structure as determined using a capping assay. In preferred embodiments, less than about 20%, 15%, 10%, 5%, 4%, 3%, 2%, 1% of the coding RNA (species) comprises a cap0 structure as determined using a capping assay. In preferred embodiments, less than about 20%, 15%, 10%, 5%, 4%, 3%, 2%, 1% of the coding RNA (species) comprises a cap1 intermediate structure as determined using a capping assay.

The term “coding RNA species” is not restricted to mean “one single molecule” but is understood to comprise an ensemble of essentially identical RNA molecules. Accordingly, it may relate to a plurality of essentially identical coding RNA molecules.

For determining the capping degree or the presence of cap1 intermediates, a capping assays as described in published PCT application WO2015/101416, in particular, as described in Claims 27 to 46 of published PCT application WO2015/101416 can be used. Other capping assays that may be used to determine the capping degree of the coding RNA are described in PCT/EP2018/08667, or published PCT applications WO2014/152673 and WO2014/152659.

In preferred embodiments, the coding RNA comprises an m7G(5′)ppp(5′)(2′OMeA) cap structure. In such embodiments, the coding RNA comprises a 5-terminal m7G cap, and an additional methylation of the ribose of the adjacent nucleotide of m7GpppN, in that case, a 2′O methylated Adenosine. Preferably, about 70%, 75%, 80%, 85%, 90%, 95% of the coding RNA (species) comprises such a cap1 structure as determined using a capping assay.

In other preferred embodiments, the coding RNA of the first aspect comprises an m7G(5′)ppp(5′)(2′OMeG) cap structure. In such embodiments, the coding RNA comprises a 5-terminal m7G cap, and an additional methylation of the ribose of the adjacent nucleotide, in that case, a 2′O methylated guanosine. Preferably, about 70%, 75%, 80%, 85%, 90%, 95% of the coding RNA (species) comprises such a cap1 structure as determined using a capping assay.

Accordingly, whenever reference is made to suitable RNA or mRNA sequences in the context of the invention, the first nucleotide of said RNA or mRNA sequence, that is, the nucleotide downstream of the m7G(5′)ppp structure, may be a 2′O methylated guanosine or a 2′O methylated adenosine.

In embodiments, the A/U content in the environment of the ribosome binding site of the coding RNA may be increased compared to the A/U content in the environment of the ribosome binding site of its respective wild type RNA. This modification (an increased A/U content around the ribosome binding site) increases the efficiency of ribosome binding to the coding RNA. An effective binding of the ribosomes to the ribosome binding site in turn has the effect of an efficient translation of the coding RNA.

Accordingly, in a particularly preferred embodiment, the coding RNA comprises a ribosome binding site, also referred to as “Kozak sequence” identical to or at least 80%, 85%, 90%, 95% identical to any one of the sequences SEQ ID NOs: 1821 or 1822, or fragments or variants thereof.

In preferred embodiments, the RNA of the invention comprises at least one poly(N) sequence, e.g. at least one poly(A) sequence, at least one poly(U) sequence, at least one poly(C) sequence, or combinations thereof.

In preferred embodiments, the RNA of the invention comprises at least one poly(A) sequence.

The terms “poly(A) sequence”, “poly(A) tail” or “3′-poly(A) tail” as used herein will be recognized and understood by the person of ordinary skill in the art, and are e.g. intended to be a sequence of adenosine nucleotides, typically located at the 3′-end of an RNA, of up to about 1000 adenosine nucleotides. Preferably, said poly(A) sequence is essentially homopolymeric, e.g. a poly(A) sequence of e.g. 100 adenosine nucleotides has essentially the length of 100 nucleotides. In other embodiments, the poly(A) sequence may be interrupted by at least one nucleotide different from an adenosine nucleotide, e.g. a poly(A) sequence of e.g. 100 adenosine nucleotides may have a length of more than 100 nucleotides (comprising 100 adenosine nucleotides and in addition said at least one nucleotide different from an adenosine nucleotide).

The poly(A) sequence may comprise about 10 to about 500 adenosine nucleotides, about 10 to about 200 adenosine nucleotides, about 40 to about 200 adenosine nucleotides, or about 40 to about 150 adenosine nucleotides. Suitably, the length of the poly(A) sequence may be at least about or even more than about 10, 50, 64, 75, 100, 200, 300, 400, or 500 adenosine nucleotides.

In preferred embodiments, the coding RNA comprises at least one poly(A) sequence comprising about 30 to about 200 adenosine nucleotides. In preferred embodiments, the poly(A) sequence comprises about 64 adenosine nucleotides (A64). In particularly preferred embodiments, the poly(A) sequence comprises about 100 adenosine nucleotides (A100). In particularly preferred embodiments, the poly(A) sequence comprises about 150 adenosine nucleotides.

The poly(A) sequence as defined herein is suitably located at the 3′ terminus of the coding RNA. Accordingly it is preferred that the 3-terminal nucleotide of the coding RNA (that is the last 3′-terminal nucleotide in the polynucleotide chain) is the 3′-terminal A nucleotide of the at least one poly(A) sequence. The term “located at the 3′ terminus” has to be understood as being located exactly at the 3′ terminus—in other words, the 3′ terminus of the coding RNA consists of a poly(A) sequence terminating with an A nucleotide. Examples of sequences having a 3′ terminus consisting of a poly(A) sequence are e.g. SEQ ID NOs: 586-594, 604-612, 631-639, 649-666, 676-684, 703-711, 721-738, 748-756, 775-783, 793-810, 820-828, 847-855, 865-882, 892-900, 919-927, 937-954, 964-972, 991-999, 1009-1026, 1036-1044, 1063-1071, 1081-1098, 1108-1116, 1135-1143, 1153-1170, 1180-1188, 1207-1215, 1225-1242, 1252-1260, 1279-1287, 1297-1314, 1324-1332, 1351-1359, 1369-1386, 1396-1404, 1423-1431, 1441-1458, 1468-1476, 1495-1503, 1513-1530, 1540-1548, 1567-1575, 1585-1602, 1612-1620, 1639-1647, 1657-1674, 1684-1692, 1711-1719, 1729-1737, 1862, 1863, 1866, 1867, 1872, 1873, 1875, 1876, 1898, 1907-1930. For further examples of sequences having a poly(A) sequence located (exactly) at the 3′ terminus see also Table 4 (columns with A100 or hSL-A100). The presence of a poly(A) sequence exactly at the 3′ terminus of the coding RNA encoding a Rotavirus antigenic protein (e.g. VP8*) is surprisingly advantageous and of particular importance in the context of the invention as the induction of a specific immune response against Rotavirus VP8* could be dramatically increased (see Examples 5 and 6)

Preferably, the poly(A) sequence of the RNA is obtained from a DNA template during RNA in vitro transcription. In other embodiments, the poly(A) sequence is obtained in vitro by common methods of chemical synthesis without being necessarily transcribed from a DNA template. In other embodiments, poly(A) sequences are generated by enzymatic polyadenylation of the RNA (after RNA in vitro transcription) using commercially available polyadenylation kits and corresponding protocols known in the art, or alternatively, by using immobilized poly(A)polymerases e.g. using a methods and means as described in WO2016/174271.

The coding RNA may comprise a poly(A) sequence obtained by enzymatic polyadenylation, wherein the majority of RNA molecules comprise about 100 (+/−20) to about 500 (+/−50), preferably about 250 (+/−20) adenosine nucleotides.

In embodiments, the RNA may comprise a poly(A) sequence derived from a template DNA and may additionally comprise at least one additional poly(A) sequence generated by enzymatic polyadenylation, e.g. as described in WO2016/091391.

In embodiments, the RNA may comprise at least one poly(C) sequence.

The term “poly(C) sequence” as used herein will be recognized and understood by the person of ordinary skill in the art, and are for example intended to be a sequence of cytosine nucleotides of up to about 200 cytosine nucleotides. In preferred embodiments, the poly(C) sequence comprises about 10 to about 200 cytosine nucleotides, about 10 to about 100 cytosine nucleotides, about 20 to about 70 cytosine nucleotides, about 20 to about 60 cytosine nucleotides, or about 10 to about 40 cytosine nucleotides. In a particularly preferred embodiment, the poly(C) sequence comprises about 30 cytosine nucleotides.

In particularly preferred embodiments, the coding RNA of the invention does comprise a poly(A) sequence as defined herein, preferably A100 located (exactly) at the 3′ terminus, and does not comprise a poly(C) sequence.

In a particularly preferred embodiment, the coding RNA of the invention comprises a cap1 structure as defined herein and at least one poly(A) sequence as defined in herein. Preferably, said cap1 structure is obtainable by co-transcriptional capping as defined herein, and said poly(A) sequence is preferably (exactly) at the 3′ terminus (e.g., A100, hSL-A100).

Examples of sequences having a poly(A) sequence (exactly) at the 3′ terminus (see Table 4, columns D, E, F, G) and a cap1 structure are e.g. SEQ ID NOs: 586-594, 604-612, 631-639, 649-666, 676-684, 703-711, 721-738, 748-756, 775-783, 793-810, 820-828, 847-855, 865-882, 892-900, 919-927, 937-954, 964-972, 991-999, 1009-1026, 1036-1044, 1063-1071, 1081-1098, 1108-1116, 1135-1143, 1153-1170, 1180-1188, 1207-1215, 1225-1242, 1252-1260, 1279-1287, 1297-1314, 1324-1332, 1351-1359, 1369-1386, 1396-1404, 1423-1431, 1441-1458, 1468-1476, 1495-1503, 1513-1530, 1540-1548, 1567-1575, 1585-1602, 1612-1620, 1639-1647, 1657-1674, 1684-1692, 1711-1719, 1729-1737, 1862, 1863, 1866, 1867, 1872, 1873, 1875, 1876, 1898, 1907-1930. The presence of cap1 structure and poly(A) sequence exactly at the 3′ terminus of the coding RNA encoding a Rotavirus antigenic protein (e.g. VP8*) is surprisingly advantageous and of particular importance in the context of the invention as the induction of a specific immune response against Rotavirus VP8* could be dramatically increased (see Example 4, 5, 6)

In preferred embodiments, the RNA of the first aspect comprises at least one histone stem-loop (hSL).

The term “histone stem-loop” (abbreviated as “hSL” in e.g. the sequence listing) as used herein will be recognized and understood by the person of ordinary skill in the art, and are for example intended to refer to nucleic acid sequences that are predominantly found in histone mRNAs.

Histone stem-loop sequences/structures may suitably be selected from histone stem-loop sequences as disclosed in WO2012/019780, the disclosure relating to histone stem-loop sequences/histone stem-loop structures incorporated herewith by reference. A histone stem-loop sequence that may be used within the present invention may preferably be derived from formulae (I) or (II) of WO2012/019780. According to a further preferred embodiment the coding RNA may comprise at least one histone stem-loop sequence derived from at least one of the specific formulae (Ia) or (IIa) of the patent application WO2012/019780.

In preferred embodiments, the coding RNA of the invention comprises at least one histone stem-loop sequence, wherein said histone stem-loop sequence (hSL) comprises or consists a nucleic acid sequence identical or at least 70%, 80%, 90%, 91%, 92%, 93%, 94%, 95%, 96%, 97%, 98%, or 99% identical to SEQ ID NOs: 1819 or 1820, or fragments or variants thereof.

In embodiments, the coding RNA of the invention comprises a 3′-terminal sequence element. Said 3′-terminal sequence element comprises a poly(A) sequence and a histone-stem-loop sequence, wherein said sequence element is located at the 3′ terminus (exactly at the 3′ terminus) of the RNA of the invention.

Accordingly, the RNA of the invention comprises at least one 3′-terminal sequence element comprising or consisting of a nucleic acid sequence being identical or at least 70%, 80%, 90%, 91%, 92%, 93%, 94%, 95%, 96%, 97%, 98%, or 99% identical to SEQ ID NOs: 1825-1856, or a fragment or variant thereof.

A preferred 3′-terminal sequence element is hSL-A100 according to SEQ ID NOs: 1827, 1836 or 1837. A further preferred 3′-terminal sequence element is A100 according to SEQ ID NOs: 1826, 1834 or 1835.

In various embodiments, the RNA may comprise a 5′-terminal sequence element according to SEQ ID NOs: 1823 or 1824, or a fragment or variant thereof. Such a 5′-terminal sequence element comprises e.g. a binding site for T7 RNA polymerase. Further, the first nucleotide of said 5′-terminal start sequence may preferably comprise a 2′O methylation, e.g. 2′O methylated guanosine or a 2′O methylated adenosine.

In embodiments, the RNA may comprise a sequence element which represents a cleavage site for a catalytic nucleic acid molecule, wherein the catalytic nucleic acid molecule may be a Ribozyme or a DNAzyme. Said elements may, e.g., allow for the analysis of capping efficiency/quality of the RNA as described in WO2015/101416, or allow for the analysis of poly(N) sequences length/quality of the RNA as described in WO2017/001058. A cleavage site for a catalytic nucleic acid molecule may be located in proximity to the 5′ terminus of the RNA (that is, about 1, 2, 3, 4, 5, 6, 7, 8, 9, 10, 1-30, 1-20, 5-15 nucleotides from the 5′-terminal cap structure). Alternatively, or in addition, a cleavage site for a catalytic nucleic acid molecule as described above may also be positioned in proximity to the 3′ terminus of the RNA (that is, about 50-300, 50-200, 50-150 nucleotides from the 3′ terminus). Said elements may, e.g., allow for the analysis of poly(N) sequences length/quality of the RNA as described in WO2017/001058.

UTRs:

The RNA of the invention may comprise a protein-coding region (“coding sequence” or “cds”), and 5′-UTR and/or 3′-UTR. Notably, UTRs may harbor regulatory sequence elements that determine RNA turnover, stability, and localization. Moreover, UTRs may harbor sequence elements that enhance translation. In medical application of RNA, translation of the RNA into at least one peptide or protein is of paramount importance to therapeutic efficacy. Certain combinations of 3′-UTRs and/or 5′-UTRs may enhance the expression of operably linked coding sequences encoding peptides or proteins of the invention. RNA molecules harboring said UTR combinations advantageously enable rapid and transient expression of antigenic peptides or proteins after administration to a subject, preferably after intramuscular administration. Accordingly, the coding RNA comprising certain combinations of 3′-UTRs and/or 5′-UTRs as provided herein is particularly suitable for administration as a vaccine, in particular, suitable for administration into the muscle, the dermis, or the epidermis of a subject.

Suitably, the coding RNA may comprise at least one heterologous 5′-UTR and/or at least one heterologous 3′-UTR. Said heterologous 5′-UTRs or 3′-UTRs may be derived from naturally occurring genes or may be synthetically engineered. In preferred embodiments, the RNA of the first aspect comprises at least one coding sequence operably linked to at least one (heterologous) 3′-UTR and/or at least one (heterologous) 5′-UTR.

In preferred embodiments, the coding RNA comprises at least one heterologous 3′-UTR.

The term “3′-untranslated region” or “3′-UTR” or “3′-UTR element” will be recognized and understood by the person of ordinary skill in the art, and are e.g. intended to refer to a part of a nucleic acid molecule located 3′ (i.e. downstream) of a coding sequence and which is not translated into protein. A 3′-UTR may be part of an RNA, e.g. an mRNA, located between a cds and a terminal poly(A) sequence. A 3′-UTR may comprise elements for controlling gene expression, also called regulatory elements. Such regulatory elements may be, e.g., ribosomal binding sites, miRNA binding sites etc.

Preferably the coding RNA comprises a 3′-UTR, which may be derivable from a gene that relates to an RNA with enhanced half-life (i.e. that provides a stable RNA).

In some embodiments, a 3′-UTR comprises one or more of a polyadenylation signal, a binding site for proteins that affect an RNA stability of location in a cell, or one or more miRNA or binding sites for miRNAs.

MicroRNAs (or miRNA) are 19-25 nucleotide long noncoding RNAs that bind to the 3′-UTR of nucleic acid molecules and down-regulate gene expression either by reducing nucleic acid molecule stability or by inhibiting translation. E.g., microRNAs are known to regulate RNA, and thereby protein expression, e.g. in liver (miR-122), heart (miR-Id, miR-149), endothelial cells (miR-17-92, miR-126), adipose tissue (let-7, miR-30c), kidney (miR-192, miR-194, miR-204), myeloid cells (miR-142-3p, miR-142-5p, miR-16, miR-21, miR-223, miR-24, miR-27), muscle (miR-133, miR-206, miR-208), and lung epithelial cells (let-7, miR-133, miR-126). The RNA may comprise one or more microRNA target sequences, microRNA sequences, or microRNA seeds. Such sequences may e.g. correspond to any known microRNA such as those taught in US2005/0261218 and US2005/0059005.

Accordingly, miRNA, or binding sites miRNAs as defined above may be removed from the 3′-UTR or introduced into the 3′-UTR in order to tailor the expression of the RNA to desired cell types or tissues (e.g. muscle cells).

In preferred embodiments of the first aspect, the coding RNA comprises at least one heterologous 3′-UTR, wherein the at least one heterologous 3′-UTR comprises a nucleic acid sequence derived from a 3′-UTR of a gene selected from PSMB3, ALB7, alpha-globin (referred to as “muag”), CASP1, COX6B1, GNAS, NDUFA1 and RPS9, or from a homolog, a fragment or variant of any one of these genes according to SEQ ID NOs: 1803-1818. Particularly preferred nucleic acid sequences in that context can be derived from published PCT application WO2019/077001A1, in particular, claim 9 of WO2019/077001A1. The corresponding 3′-UTR sequences of claim 9 of WO2019/077001A1 are herewith incorporated by reference (e.g., SEQ ID NOs: 23-34 of WO2019/077001A1, or fragments or variants thereof).

In embodiments, the RNA may comprise a 3′-UTR derived from an alpha-globin gene. Said 3′-UTR derived from a alpha-globin gene (“muag”) may comprise or consist of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 1817 or 1818 or a fragment or a variant thereof.

In preferred embodiments, the RNA may comprise a 3′-UTR derived from a PSMB3 gene. Said 3′-UTR derived from a PSMB3 gene may comprise or consist of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 1803 or 1804 or a fragment or a variant thereof.

In other embodiments, the coding RNA may comprise a 3′-UTR as described in WO2016/107877, the disclosure of WO2016/107877 relating to 3′-UTR sequences herewith incorporated by reference. Suitable 3-UTRs are SEQ ID NOs: 1-24 and SEQ ID NOs: 49-318 of WO2016/107877, or fragments or variants of these sequences. In other embodiments, the coding RNA comprises a 3′-UTR as described in WO2017/036580, the disclosure of WO2017/036580 relating to 3′-UTR sequences herewith incorporated by reference. Suitable 3′-UTRs are SEQ ID NOs: 152-204 of WO2017/036580, or fragments or variants of these sequences. In other embodiments, the coding RNA comprises a 3′-UTR as described in WO2016/022914, the disclosure of WO2016/022914 relating to 3′-UTR sequences herewith incorporated by reference. Particularly preferred 3′-UTRs are nucleic acid sequences according to SEQ ID NOs: 20-36 of WO2016/022914, or fragments or variants of these sequences.

In preferred embodiments, the coding RNA comprises at least one heterologous 5′-UTR.

The terms “5′-untranslated region” or “5′-UTR” or “5′-UTR element” will be recognized and understood by the person of ordinary skill in the art, and are e.g. intended to refer to a part of a nucleic acid molecule located 5′ (i.e. “upstream”) of a coding sequence and which is not translated into protein. A 5′-UTR may be part of an RNA located 5′ of the coding sequence. Typically, a 5′-UTR starts with the transcriptional start site and ends before the start codon of the coding sequence. A 5′-UTR may comprise elements for controlling gene expression, also called regulatory elements. Such regulatory elements may be, e.g., ribosomal binding sites, miRNA binding sites etc. The 5′-UTR may be post-transcriptionally modified, e.g. by enzymatic or post-transcriptional addition of a 5′-cap structure (as defined above).

Preferably the coding RNA comprises a 5′-UTR, which may be derivable from a gene that relates to an RNA with enhanced half-life (i.e. that provides a stable RNA).

In some embodiments, a 5′-UTR comprises one or more of a binding site for proteins that affect an RNA stability of location in a cell, or one or more miRNA or binding sites for miRNAs (as defined above).

Accordingly, miRNA or binding sites miRNAs as defined above may be removed from the 5′-UTR or introduced into the 5′-UTR in order to tailor the expression of the RNA to desired cell types or tissues.

In preferred embodiments, the coding RNA comprises at least one heterologous 5′-UTR, wherein the at least one heterologous 5′-UTR comprises a nucleic acid sequence derived from a 5′-UTR of gene selected from HSD17B4, RPL32, ASAH1, ATP5A1, MP68, NDUFA4, NOSIP, RPL31, SLC7A3, TUBB4B, and UBQLN2, or from a homolog, a fragment or variant of any one of these genes according to SEQ ID NOs: 1781-1802. Particularly preferred nucleic acid sequences in that context can be selected from published PCT application WO2019/077001A1, in particular, claim 9 of WO2019/077001A1. The corresponding 5′-UTR sequences of claim 9 of WO2019/077001A1 are herewith incorporated by reference (e.g., SEQ ID NOs: 1-20 of WO2019/077001A1, or fragments or variants thereof).

In preferred embodiments, the RNA may comprise a 5′-UTR derived from a HSD17B4 gene, wherein said 5′-UTR derived from a HSD1764 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 1781 or 1782 or a fragment or a variant thereof.

In other embodiments, the coding RNA comprises a 5′-UTR as described in WO2013/143700, the disclosure of WO2013/143700 relating to 5′-UTR sequences herewith incorporated by reference. Particularly preferred 5′-UTRs are nucleic acid sequences derived from SEQ ID NOs: 1-1363, SEQ ID NO: 1395, SEQ ID NO: 1421 and SEQ ID NO: 1422 of WO2013/143700, or fragments or variants of these sequences. In other embodiments, the coding RNA comprises a 5′-UTR as described in WO2016/107877, the disclosure of WO2016/107877 relating to 5′-UTR sequences herewith incorporated by reference. Particularly preferred 5′-UTRs are nucleic acid sequences according to SEQ ID NOs: 25-30 and SEQ ID NOs: 319-382 of WO2016/107877, or fragments or variants of these sequences. In other embodiments, the coding RNA comprises a 5′-UTR as described in WO2017/036580, the disclosure of WO2017/036580 relating to 5′-UTR sequences herewith incorporated by reference. Particularly preferred 5′-UTRs are nucleic acid sequences according to SEQ ID NOs: 1-151 of WO2017/036580, or fragments or variants of these sequences. In other embodiments, the coding RNA comprises a 5′-UTR as described in WO2016/022914, the disclosure of WO2016/022914 relating to 5′-UTR sequences herewith incorporated by reference. Particularly preferred 5′-UTRs are nucleic acid sequences according to SEQ ID NOs: 3-19 of WO2016/022914, or fragments or variants of these sequences.

In preferred embodiments of the coding RNA comprises at least one coding sequence as specified herein encoding at least one antigenic protein as defined herein, preferably VP8*, wherein said coding sequence is operably linked to a 5′-UTR selected from HSD17B4, RPL32, ASAH1, ATP5A1, MP68, NDUFA4, NOSIP, RPL31, SLC7A3, TUBB4B, and UBQLN2, or from a homolog, a fragment or variant of any one of these genes according to SEQ ID NOs: 1781-1802 and a 3′-UTR selected from PSMB3, ALB7, alpha-globin (referred to as “muag”), CASP1, COX6B1, GNAS, NDUFA1 and RPS9, or from a homolog, a fragment or variant of any one of these genes according to SEQ ID NOs: 1803-1818.

In preferred embodiments of the coding RNA comprises at least one coding sequence as specified herein encoding at least one antigenic protein as defined herein, preferably VP8*, wherein said coding sequence is operably linked to a 5′-UTR and a 3′-UTR derived from published PCT application WO2019/077001A1, in particular, claim 9 of WO2019/077001A1. The corresponding 3′-UTR sequences of claim 9 of WO2019/077001A1 are herewith incorporated by reference (e.g., SEQ ID NOs: 23-34 of WO2019/077001A1, or fragments or variants thereof) and the corresponding 5′-UTR sequences of claim 9 of WO2019/077001A1 are herewith incorporated by reference (e.g., SEQ ID NOs: 1-20 of WO2019/077001A1, or fragments or variants thereof).

In particularly preferred embodiments of the coding RNA comprises at least one coding sequence as specified herein encoding at least one antigenic protein as defined herein, preferably VP8*, wherein said coding sequence is operably linked to a 5′-UTR selected from HSD17B4 and a 3′-UTR selected from PSMB3 (HSD17B4/PSMB3).

Accordingly, the RNA of the first aspect comprises at least one coding sequence encoding at least one peptide or protein as defined herein, wherein said coding sequence as defined herein is operably linked to at least one heterologous 5′-UTR and/or to at least one heterologous 3′-UTR, wherein suitably
the at least one heterologous 5′-UTR is derived from a 5′-UTR of a HSD17B4 gene, or from a corresponding RNA sequence, homolog, fragment or variant thereof and the at least one 3′-UTR is derived from a 3′-UTR of a PSMB3 gene, or from a corresponding RNA sequence, homolog, fragment or variant thereof, wherein, preferably, said 5′-UTR comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 1781 or 1782 or a fragment or a variant thereof, and said 3′-UTR comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 1803 or 1804 or a fragment or a variant thereof
the at least one heterologous 3′-UTR is derived from a 3′-UTR of a alpha-globin gene gene (muag), or from a corresponding RNA sequence, homolog, fragment or variant thereof wherein, preferably, said 3′-UTR comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 1817 or 1818 or a fragment or a variant thereof.

Suitable Coding RNA for a Rotavirus Vaccine:

In various embodiments the coding RNA comprises, preferably in 5′- to 3′-direction, the following elements:
A) 5′-cap structure, preferably as specified herein;
B) 5-terminal start element, preferably as specified herein;
C) optionally, a cleavage site for a catalytic nucleic acid molecule, preferably as specified herein;
D) optionally, a 5′-UTR, preferably as specified herein;
E) a ribosome binding site, preferably as specified herein;
F) at least one coding sequence, preferably as specified herein;
G) 3′-UTR, preferably as specified herein;
H) optionally, poly(A) sequence, preferably as specified herein;
I) optionally, poly(C) sequence, preferably as specified herein;
J) optionally, histone stem-loop preferably as specified herein;
K) optionally, 3-terminal sequence element, preferably as specified herein.

In preferred embodiments the coding RNA, preferably the mRNA, comprises the following elements preferably in 5′- to 3′-direction:
A) 5′-cap structure selected from m7G(5′), m7G(5′)ppp(5′)(2′OMeA), or m7G(5′)ppp(5′)(2′OMeG);
B) 5-terminal start element selected from SEQ ID NOs: 1823, 1824 or fragments or variants thereof;
C) optionally, a cleavage site for a catalytic nucleic acid molecule, preferably as specified herein;
D) optionally, a 5′-UTR derived from a HSD17B4 gene;
E) a ribosome binding site selected from SEQ ID NOs: 1821, 1822 or fragments or variants thereof;
F) at least one coding sequence selected from SEQ ID NOs: 118-585, 1901-1906, or fragments or variants thereof;
G) 3′-UTR derived from a 3′-UTR of a PSMB3 gene or an alpha-globin gene (muag);
H) optionally, poly(A) sequence comprising about 30 to about 500 adenosines;
I) optionally, poly(C) sequence comprising about 10 to about 100 cytosines;
J) optionally, histone stem-loop selected from SEQ ID NOs: 1819 or 1820;
K) optionally, 3′-terminal sequence element SEQ ID NOs: 1825-1856.

In particularly preferred embodiments the mRNA comprises the following elements in 5′- to 3′-direction:
A) cap1 structure, preferably obtainable by co-transcriptional capping as defined herein;
B) 5′-UTR derived from a HSD1784 gene as defined herein, preferably according to SEQ ID NO: 1781 or 1782;
C) coding sequence selected from SEQ ID NOs: 118-585, 1901-1906 or fragments or variants thereof;
D) 3′-UTR derived from a 3′-UTR of a PSMB3 gene as defined herein, preferably according to SEQ ID NO: 1803 or 1804;
E) optionally, histone stem-loop selected from SEQ ID NOs: 1819 or 1820;
F) poly(A) sequence comprising about 100 A nucleotides, representing the 3′ terminus.

Preferred amino acid sequences and coding RNA/mRNA sequences of the invention are provided in Table 4. Therein, each row represents a specific suitable Rotavirus VP8* construct of the invention (compare with Table 1 and Table 3, columns A and B as reference), wherein the description of the Rotavirus VP8* construct is indicated in column A, column B of Table 4 provides a description of the Rotavirus of which the respective VP8* is derived from, the SEQ ID NOs of the amino acid sequence of the respective Rotavirus VP8* construct is provided in column C.

The corresponding SEQ ID NOs of the coding sequences encoding the respective Rotavirus VP8* constructs are provided in in Table 3 (wild type cds) and D (opt1, opt4, opt5). Further information is provided under <223> identifier of the respective SEQ ID NOs in the sequence listing.

The corresponding coding RNA sequences, in particular mRNA sequences comprising preferred coding sequences are provided in columns D, E, F, and G, wherein column D provides RNA sequences with an UTR combination “HSD17B4/PSMB3” as defined herein and a poly(A) sequence exactly at the 3′ terminus (A100), wherein column E provides RNA sequences with an “alpha-globin” UTR as defined herein and a poly(A) sequence exactly at the 3′ terminus (A100), wherein column F provides RNA sequences with an UTR combination “HSD17B4/PSMB3” as defined herein and a poly(A) sequence exactly at the 3′ terminus (hSL-A100) and wherein column G provides RNA sequences with an “alpha-globin” UTR as defined herein and a poly(A) sequence exactly at the 3′ terminus (hSL-A100).

TABLE 4
Preferred coding RNA, e.g. mRNA, encoding Rotavirus VP8* antigen constructs

| A | B | C | D | E | F | G |
|---|---|---|---|---|---|---|
| P2_VP8*(65-223) | RVA/BE1058/P[4] | 6, 46 | 586, 658, | 874, 946, | 1162, 1234, | 1450, 1522, |
| | | | 730, 802, | 1018, 1090, | 1306, 1378, | 1594, 1666, |
| | | | 1909 | 1915 | 1921 | 1927 |
| P2_VP8*(65-223) | RVA/DS-1/P[4] | 47 | 587, 659, | 875, 947, | 1163, 1235, | 1451, 1523, |
| | | | 731, 803 | 1019, 1091 | 1307, 1379 | 1595, 1667 |
| P2_VP8*(65-223) | RVA/DS-1/P[4] | 48 | 588, 660, | 876, 948, | 1164, 1236, | 1452, 1524, |
| | | | 732, 804 | 1020, 1092 | 1308, 1380 | 1596, 1668 |
| P2_VP8*(65-223) | RVA/F01322/P[6] | 5, 49 | 589, 661, | 877, 949, | 1165, 1237, | 1453, 1525, |
| | | | 733, 805, | 1021, 1093, | 1309, 1381, | 1597, 1669, |
| | | | 1910 | 1916 | 1922 | 1928 |
| P2_VP8*(65-223) | RVA/1076/P[6] | 50 | 590, 662, | 878, 950, | 1166, 1238, | 1454, 1526, |
| | | | 734, 806 | 1022, 1094 | 1310, 1382 | 1598, 1670 |
| P2_VP8*(65-223) | RVA/BE1128/P[8] | 4, 51 | 591, 663, | 879, 951, | 1167, 1239, | 1455, 1527, |
| | | | 735, 807 | 1023, 1095 | 1311, 1383 | 1599, 1671 |
| P2_VP8*(65-223) | RVA/Wa- | 52 | 592, 664, | 880, 952, | 1168, 1240, | 1456, 1528, |
| | VirWa/P[8] | | 736, 808 | 1024, 1096 | 1312, 1384 | 1600, 1672 |
| P2_VP8*(65-223) | RVA/Wa/P[8] | 53 | 593, 665, | 881, 953, | 1169, 1241, | 1457, 1529, |
| | | | 737, 809 | 1025, 1097 | 1313, 1385 | 1601, 1673 |
| P2_VP8*(65-223) | RVA/Wa/P[8] | 54 | 594, 666, | 882, 954, | 1170, 1242, | 1458, 1530, |
| | | | 738, 810 | 1026, 1098 | 1314, 1386 | 1602, 1674 |
| P2_VP8*(41-223) | RVA/BE1058/P[4] | 55 | 595, 667, | 883, 955, | 1171, 1243, | 1459, 1531, |
| | | | 739, 811 | 1027, 1099 | 1315, 1387 | 1603, 1675 |
| P2_VP8*(41-223) | RVA/DS-1/P[4] | 56 | 596, 668, | 884, 956, | 1172, 1244, | 1460, 1532, |
| | | | 740, 812 | 1028, 1100 | 1316, 1388 | 1604, 1676 |
| P2_VP8*(41-223) | RVA/DS-1/P[4] | 57 | 597, 669, | 885, 957, | 1173, 1245, | 1461, 1533, |
| | | | 741, 813 | 1029, 1101 | 1317, 1389 | 1605, 1677 |
| P2_VP8*(41-223) | RVA/F01322/P[6] | 58 | 598, 670, | 886, 958, | 1174, 1246, | 1462, 1534, |
| | | | 742, 814 | 1030, 1102 | 1318, 1390 | 1606, 1678 |
| P2_VP8*(41-223) | RVA/1076/P[6] | 59 | 599, 671, | 887, 959, | 1175, 1247, | 1463, 1535, |
| | | | 743, 815 | 1031, 1103 | 1319, 1391 | 1607, 1679 |
| P2_VP8*(41-223) | RVA/BE1128/P[8] | 60 | 600, 672, | 888, 960, | 1176, 1248, | 1464, 1536, |
| | | | 744, 816 | 1032, 1104 | 1320, 1392 | 1608, 1680 |
| P2_VP8*(41-223) | RVA/Wa- | 61 | 601, 673, | 889, 961, | 1177, 1249, | 1465, 1537, |
| | VirWa/P[8] | | 745, 817 | 1033, 1105 | 1321, 1393 | 1609, 1681 |
| P2_VP8*(41-223) | RVA/Wa/P[8] | 62 | 602, 674, | 890, 962, | 1178, 1250, | 1466, 1538, |
| | | | 746, 818 | 1034, 1106 | 1322, 1394 | 1610, 1682 |
| P2_VP8*(41-223) | RVA/Wa/P[8] | 63 | 603, 675, | 891, 963, | 1179, 1251, | 1467, 1539, |
| | | | 747, 819 | 1035, 1107 | 1323, 1395 | 1611, 1683 |
| P2_VP8*(65-223)_Ferritin | RVA/BE1058/P[4] | 64 | 604, 676, | 892, 964, | 1180, 1252, | 1468, 1540, |
| | | | 748, 820 | 1036, 1108 | 1324, 1396 | 1612, 1684 |
| P2_VP8*(65-223)_Ferritin | RVA/DS-1/P[4] | 65 | 605, 677, | 893, 965, | 1181, 1253, | 1469, 1541, |
| | | | 749, 821 | 1037, 1109 | 1325, 1397 | 1613, 1685 |
| P2_VP8*(65-223)_Ferritin | RVA/DS-1/P[4] | 66 | 606, 678, | 894, 966, | 1182, 1254, | 1470, 1542, |
| | | | 750, 822 | 1038, 1110 | 1326, 1398 | 1614, 1686 |
| P2_VP8*(65-223)_Ferritin | RVA/F01322/P[6] | 67 | 607, 679, | 895, 967, | 1183, 1255, | 1471, 1543, |
| | | | 751, 823 | 1039, 1111 | 1327, 1399 | 1615, 1687 |
| P2_VP8*(65-223)_Ferritin | RVA/1076/P[6] | 68 | 608, 680, | 896, 968, | 1184, 1256, | 1472, 1544, |
| | | | 752, 824 | 1040, 1112 | 1328, 1400 | 1616, 1688 |
| P2_VP8*(65-223)_Ferritin | RVA/BE1128/P[8] | 69 | 609, 681, | 897, 969, | 1185, 1257, | 1473, 1545, |
| | | | 753, 825 | 1041, 1113 | 1329, 1401 | 1617, 1689 |
| P2_VP8*(65-223)_Ferritin | RVA/Wa- | 70 | 610, 682, | 898, 970, | 1186, 1258, | 1474, 1546, |
| | VirWa/P[8] | | 754, 826 | 1042, 1114 | 1330, 1402 | 1618, 1690 |
| P2_VP8*(65-223)_Ferritin | RVA/Wa/P[8] | 71 | 611, 683, | 899, 971, | 1187, 1259, | 1475, 1547, |
| | | | 755, 827 | 1043, 1115 | 1331, 1403 | 1619, 1691 |
| P2_VP8*(65-223)_Ferritin | RVA/Wa/P[8] | 72 | 612, 684, | 900, 972, | 1188, 1260, | 1476, 1548, |
| | | | 756, 828 | 1044, 1116 | 1332, 1404 | 1620, 1692 |
| P2_VP8*(41-223)_Ferritin | RVA/BE1058/P[4] | 73 | 613, 685, | 901, 973, | 1189, 1261, | 1477, 1549, |
| | | | 757, 829 | 1045, 1117 | 1333, 1405 | 1621, 1693 |
| P2_VP8*(41-223)_Ferritin | RVA/DS-1/P[4] | 74 | 614, 686, | 902, 974, | 1190, 1262, | 1478, 1550, |
| | | | 758, 830 | 1046, 1118 | 1334, 1406 | 1622, 1694 |
| P2_VP8*(41-223)_Ferritin | RVA/DS-1/P[4] | 75 | 615, 687, | 903, 975, | 1191, 1263, | 1479, 1551, |
| | | | 759, 831 | 1047, 1119 | 1335, 1407 | 1623, 1695 |
| P2_VP8*(41-223)_Ferritin | RVA/F01322/P[6] | 76 | 616, 688, | 904, 976, | 1192, 1264, | 1480, 1552, |
| | | | 760, 832 | 1048, 1120 | 1336, 1408 | 1624, 1696 |
| P2_VP8*(41-223)_Ferritin | RVA/1076/P[6] | 77 | 617, 689, | 905, 977, | 1193, 1265, ' | 1481, 1553, |
| | | | 761, 833 | 1049, 1121 | 1337, 1409 | 1625, 1697 |
| P2_VP8*(41-223)_Ferritin | RVA/BE1128/P[8] | 78 | 618, 690, | 906, 978, | 1194, 1266, | 1482, 1554, |
| | | | 762, 834 | 1050, 1122 | 1338, 1410 | 1626, 1698 |
| P2_VP8*(41-223)_Ferritin | RVA/Wa- | 79 | 619, 691, | 907, 979, | 1195, 1267, | 1483, 1555, |
| | VirWa/P[8] | | 763, 835 | 1051, 1123 | 1339, 1411 | 1627, 1699 |
| P2_VP8*(41-223)_Ferritin | RVA/Wa/P[8] | 80 | 620, 692, | 908, 980, | 1196, 1268, | 1484, 1556, |
| | | | 764, 836 | 1052, 1124 | 1340, 1412 | 1628, 1700 |
| P2_VP8*(41-223)_Ferritin | RVA/Wa/P[8] | 81 | 621, 693, | 909, 981, | 1197, 1269, | 1485, 1557, |
| | | | 765, 837 | 1053, 1125 | 1341, 1413 | 1629, 1701 |
| LumSynt_P2_VP8*(65- | RVA/BE1058/P[4] | 82 | 622, 694, | 910, 982, | 1198, 1270, | 1486, 1558, |
| 223) | | | 766, 838 | 1054, 1126 | 1342, 1414 | 1630, 1702 |
| LumSynt_P2_VP8*(65- | RVA/DS-1/P[4] | 83 | 623, 695, | 911, 983, | 1199, 1271, | 1487, 1559, |
| 223) | | | 767, 839 | 1055, 1127 | 1343, 1415 | 1631, 1703 |
| LumSynt_P2_VP8*(65- | RVA/DS-1/P[4] | 84 | 624, 696, | 912, 984, | 1200, 1272, | 1488, 1560, |
| 223) | | | 768, 840 | 1056, 1128 | 1344, 1416 | 1632, 1704 |
| LumSynt_P2_VP8*(65- | RVA/F01322/P[6] | 85 | 625, 697, | 913, 985, | 1201, 1273, | 1489, 1561, |
| 223) | | | 769, 841 | 1057, 1129 | 1345, 1417 | 1633, 1705 |
| LumSynt_P2_VP8*(65- | RVA/1076/P[6] | 86 | 626, 698, | 914, 986, | 1202, 1274, | 1490, 1562, |
| 223) | | | 770, 842 | 1058, 1130 | 1346, 1418 | 1634, 1706 |
| LumSynt_P2_VP8*(65- | RVA/BE1128/P[8] | 87 | 627, 699, | 915, 987, | 1203, 1275, | 1491, 1563, |
| 223) | | | 771, 843 | 1059, 1131 | 1347, 1419 | 1635, 1707 |
| LumSynt_P2_VP8*(65- | RVA/Wa- | 88 | 628, 700, | 916, 988, | 1204, 1276, | 1492, 1564, |
| 223) | VirWa/P[8] | | 772, 844 | 1060, 1132 | 1348, 1420 | 1636, 1708 |
| LumSynt_P2_VP8*(65- | RVA/Wa/P[8] | 89 | 629, 701, | 917, 989, | 1205, 1277, | 1493, 1565, |
| 223) | | | 773, 845 | 1061, 1133 | 1349, 1421 | 1637, 1709 |
| LumSynt_P2_VP8*(65- | RVA/Wa/P[8] | 90 | 630, 702, | 918, 990, | 1206, 1278, | 1494, 1566, |
| 223) | | | 774, 846 | 1062, 1134 | 1350, 1422 | 1638, 1710 |
| LumSynt_P2_VP8*(41- | RVA/BE1058/P[4] | 3, 91 | 631, 703, | 919, 991, | 1207, 1279, | 1495, 1567, |
| 223) | | | 775, 847, | 1063, 1135, | 1351, 1423, | 1639, 1711, |
| | | | 1911 | 1917 | 1923 | 1929 |
| LumSynt_P2_VP8*(41- | RVA/DS-1/P[4] | 92 | 632, 704, | 920, 992, | 1208, 1280, | 1496, 1568, |
| 223) | | | 776, 848 | 1064, 1136 | 1352, 1424 | 1640, 1712 |
| LumSynt_P2_VP8*(41- | RVA/DS-1/P[4] | 93 | 633, 705, | 921, 993, | 1209, 1281, | 1497, 1569, |
| 223) | | | 777, 849 | 1065, 1137 | 1353, 1425 | 1641, 1713 |
| LumSynt_P2_VP8*(41- | RVA/F01322/P[6] | 2, 94 | 634, 706, | 922, 994, | 1210, 1282, | 1498, 1570, |
| 223) | | | 778, 850, | 1066, 1138, | 1354, 1426, | 1642, 1714, |
| | | | 1912 | 1918 | 1924 | 1930 |
| LumSynt_P2_VP8*(41- | RVA/1076/P[6] | 95 | 635, 707, | 923, 995, | 1211, 1283, | 1499, 1571, |
| 223) | | | 779, 851 | 1067, 1139 | 1355, 1427 | 1643, 1715 |
| LumSynt_P2_VP8*(41- | RVA/BE1128/P[8] | 1, 96 | 636, 708, | 924, 996, | 1212, 1284, | 1500, 1572, |
| 223) | | | 780, 852 | 1068, 1140 | 1356, 1428 | 1644, 1716 |
| LumSynt_P2_VP8*(41- | RVA/Wa- | 97 | 637, 709, | 925, 997, | 1213, 1285, | 1501, 1573, |
| 223) | VirWa/P[8] | | 781, 853 | 1069, 1141 | 1357, 1429 | 1645, 1717 |
| LumSynt_P2_VP8*(41- | RVA/Wa/P[8] | 98 | 638, 710, | 926, 998, | 1214, 1286, | 1502, 1574, |
| 223) | | | 782, 854 | 1070, 1142 | 1358, 1430 | 1646, 1718 |
| LumSynt_P2_VP8*(41- | RVA/Wa/P[8] | 99 | 639, 711, | 927, 999, | 1215, 1287, | 1503, 1575, |
| 223) | | | 783, 855 | 1071, 1143 | 1359, 1431 | 1647, 1719 |
| SP-IgE_P2_VP8*(65-223) | RVA/BE1058/P[4] | 100 | 640, 712, | 928, 1000, | 1216, 1288, | 1504, 1576, |
| | | | 784, 856 | 1072, 1144 | 1360, 1432 | 1648, 1720 |
| SP-IgE_P2_VP8*(65-223) | RVA/DS-1/P[4] | 101 | 641, 713, | 929, 1001, | 1217, 1289, | 1505, 1577, |
| | | | 785, 857 | 1073, 1145 | 1361, 1433 | 1649, 1721 |
| SP-IgE_P2_VP8*(65-223) | RVA/DS-1/P[4] | 102 | 642, 714, | 930, 1002, | 1218, 1290, | 1506, 1578, |
| | | | 786, 858 | 1074, 1146 | 1362, 1434 | 1650, 1722 |
| SP-IgE_P2_VP8*(65-223) | RVA/F01322/P[6] | 103 | 643, 715, | 931, 1003, | 1219, 1291, | 1507, 1579, |
| | | | 787, 859 | 1075, 1147 | 1363, 1435 | 1651, 1723 |
| SP-IgE_P2_VP8*(65-223) | RVA/1076/P[6] | 104 | 644, 716, | 932, 1004, | 1220, 1292, | 1508, 1580, |
| | | | 788, 860 | 1076, 1148 | 1364, 1436 | 1652, 1724 |
| SP-IgE_P2_VP8*(65-223) | RVA/BE1128/P[8] | 105 | 645, 717, | 933, 1005, | 1221, 1293, | 1509, 1581, |
| | | | 789, 861 | 1077, 1149 | 1365, 1437 | 1653, 1725 |
| SP-IgE_P2_VP8*(65-223) | RVA/Wa- | 106 | 646, 718, | 934, 1006, | 1222, 1294, | 1510, 1582, |
| | VirWa/P[8] | | 790, 862 | 1078, 1150 | 1366, 1438 | 1654, 1726 |
| SP-IgE_P2_VP8*(65-223) | RVA/Wa/P[8] | 107 | 647, 719, | 935, 1007, | 1223, 1295, | 1511, 1583, |
| | | | 791, 863 | 1079, 1151 | 1367, 1439 | 1655, 1727 |
| SP-IgE_P2_VP8*(65-223) | RVA/Wa/P[8] | 108 | 648, 720, | 936, 1008, | 1224, 1296, | 1512, 1584, |
| | | | 792, 864 | 1080, 1152 | 1368, 1440 | 1656, 1728 |
| SP-IgE_P2_VP8*(41-223) | RVA/BE1058/P[4] | 109 | 649, 721, | 937, 1009, | 1225, 1297, | 1513, 1585, |
| | | | 793, 865 | 1081, 1153 | 1369, 1441 | 1657, 1729 |
| SP-IgE_P2_VP8*(41-223) | RVA/DS-1/P[41 | 110 | 650, 722, | 938, 1010, | 1226, 1298, | 1514, 1586, |
| | | | 794, 866 | 1082, 1154 | 1370, 1442 | 1658, 1730 |
| SP-IgE_P2_VP8*(41-223) | RVA/DS-1/P[4] | 111 | 651, 723, | 939, 1011, | 1227, 1299, | 1515, 1587, |
| | | | 795, 867 | 1083, 1155 | 1371, 1443 | 1659, 1731 |
| SP-IgE_P2_VP8*(41-223) | RVA/F01322/P[6] | 112 | 652, 724, | 940, 1012, | 1228, 1300, | 1516, 1588, |
| | | | 796, 868 | 1084, 1156 | 1372, 1444 | 1660, 1732 |
| SP-IgE_P2_VP8*(41-223) | RVA/1076/P[6] | 113 | 653, 725, | 941, 1013, | 1229, 1301, | 1517, 1589, |
| | | | 797, 869 | 1085, 1157 | 1373, 1445 | 1661, 1733 |
| SP-IgE_P2_VP8*(41-223) | RVA/BE1128/P[8] | 114 | 654, 726, | 942, 1014, | 1230, 1302, | 1518, 1590, |
| | | | 798, 870 | 1086, 1158 | 1374, 1446 | 1662, 1734 |
| SP-IgE_P2_VP8*(41-223) | RVA/Wa- | 115 | 655, 727, | 943, 1015, | 1231, 1303, | 1519, 1591, |
| | VirWa/P[8] | | 799, 871 | 1087, 1159 | 1375, 1447 | 1663, 1735 |
| SP-IgE_P2_VP8*(41-223) | RVA/Wa/P[8] | 116 | 656, 728, | 944, 1016, | 1232, 1304, | 1520, 1592, |
| | | | 800, 872 | 1088, 1160 | 1376, 1448 | 1664, 1736 |
| SP-IgE_P2_VP8*(41-223) | RVA/Wa/P[8] | 117 | 657, 729, | 945, 1017, | 1233, 1305, | 1521, 1593, |
| | | | 801, 873 | 1089, 1161 | 1377, 1449 | 1665, 1737 |
| P2_VP8*(64-223) | RVA/BE1058/P[4] | 1899 | 1907 | 1913 | 1919 | 1925 |
| P2_VP8*(64-223) | RVA/F01322/P[6] | 1900 | 1908 | 1914 | 1920 | 1926 |

Further suitable amino acid sequences and coding RNA/mRNA sequences of the invention are provided in Table X4. Therein, each row represents a specific suitable Rotavirus VP8* construct of the invention (compare with Table 1 and Table 3, columns A and B as reference), wherein the description of the Rotavirus VP8* construct is indicated in column A, column B of Table X4 provides a description of the Rotavirus of which the respective VP8A is derived from, the SEQ ID NOs of the amino acid sequence of the respective Rotavirus VP8 construct is provided in column F. The corresponding coding RNA sequences, in particular mRNA sequences comprising UTR combinations (column C) and defined 3-ends (column D), are provided in column E. The following descriptions in column D correspond to a poly(A) sequence located (exactly) at the 3′ terminus of the coding RNA: A100, hSL-A100, + enzymatic poly(A). A64-N5-C30-hSL-N5 describes a poly(A) sequence which is not located (exactly at the 3′ terminus of the coding RNA.

TABLE X4
Coding RNA, e.g. mRNA, encoding Rotavirus VPS* antigen constructs and others

| | | | C | | | |
|---|---|---|---|---|---|---|
| | | | 5′-UTR/ | | E | F |
| RNA | A | B | 3′-UTR; | D | SEQ ID | SEQ ID |
| ID | Construct | Organism | UTR Design | 3′-end | NO: RNA | NO: PRT |
| R8131 | P2_VP8*(65-223) (opt1) | RVA/BE1128/ | HSD17B4/ | A100 | 1862, 591 | 4, 51 |
| | | P[8] | PSMB3; a-1 | | | |
| R8580 | P2_VP8*(65-223) (opt1) | RVA/BE1128/ | HSD17B4/ | A100 | 1862, 591 | 4, 51 |
| | | P[8] | PSMB3; a-1 | | | |
| R8581 | P2_VP8*(65-223) (opt1) | RVA/BE1128/ | HSD17B4/ | A100 | 1862, 591 | 4, 51 |
| | | P[8] | PSMB3; a-1 | | | |
| R8575 | P2_VP8*(65-223) (opt1) | RVA/BE1128/ | HSD17B4/ | hSL-A100 | 1863, 1167 | 4, 51 |
| | | P[8] | PSMB3; a-1 | | | |
| R8576 | P2_VP8*(65-223) (opt1) | RVA/BE1128/ | HSD17B4/ | hSL-A100 | 1863, 1167 | 4, 51 |
| | | P[8] | PSMB3; a-1 | | | |
| R8628 | P2_VP8*(65-223) (opt1) | RVA/BE1128/ | HSD17B4/ | hSL-A100 | 1863, 1167 | 4, 51 |
| | | P[8] | PSMB3; a-1 | | | |
| R8044 | P2_VP8*(65-223) (opt1) | RVA/BE1128/ | HSD17B4/ | A64-N5-C30-hSL-N5 | 1864 | 4, 51 |
| | | P[8] | PSMB3; a-1 | | | |
| R8134 | P2_VP8*(65-223) (opt1) | RVA/BE1128/ | HSD17B4/ | A64-N5-C30-hSL-N5 + | 1864 | 4, 51 |
| | | P[8] | PSMB3; a-1 | enzymatic poly(A) | | |
| R5470 | P2_VP8*(65-223) (opt1) | RVA/BE1128/ | RPL32/ | A64-N5-C30-hSL-N5 | 1865 | 4, 51 |
| | | P[8] | ALB7; i-2 | | | |
| R5471 | P2_VP8*(65-223) (opt1) | RVA/BE1128/ | RPL32/ | A64-N5-C30-hSL-N5 | 1865 | 4, 51 |
| | | P[8] | ALB7; i-2 | | | |
| R7877 | P2_VP8*(65-223) (opt1) | RVA/BE1128/ | RPL32/ | A64-N5-C30-hSL-N5 | 1865 | 4, 51 |
| | | P[8] | ALB7; i-2 | | | |
| R7967 | P2_VP8*(65-223) (opt1) | RVA/BE1128/ | RPL32/ | A64-N5-C30-hSL-N5 | 1865 | 4, 51 |
| | | P[8] | ALB7; i-2 | | | |
| R8045 | P2_VP8*(65-223) (opt1) | RVA/BE1128/ | RPL32/ | A64-N5-C30-hSL-N5 | 1865 | 4, 51 |
| | | P[8] | ALB7; i-2 | | | |
| R8135 | P2_VP8*(65-223) (opt1) | RVA/BE1128/ | RPL32/ | A64-N5-C30-hSL-N5 | 1865 | 4, 51 |
| | | P[8] | ALB7; i-2 | | | |
| R8046 | P2_VP8*(65-223) (opt1) | RVA/BE1128/ | RPL32/ | A64-N5-C30-hSL-N5 + | 1865 | 4, 51 |
| | | P[8] | ALB7; i-2 | enzymatic poly(A) | | |
| R8133 | P2_VP8*(65-223) (opt3) | RVA/BE1128/ | HSD17B4/ | A100 | 1866 | 4, 51 |
| | | P[8] | PSMB3; a-1 | | | |
| R8629 | P2_VP8*(65-223) (opt3) | RVA/BE1128/ | HSD17B4/ | KSL-A100 | 1867 | 4, 51 |
| | | P[8] | PSMB3; a-1 | | | |
| R8049 | P2_VP8*(65-223) (opt3) | RVA/BE1128/ | HSD17B4/ | A64-N5-C30-hSL-N5 | 1868 | 4, 51 |
| | | P[8] | PSMB3; a-1 | | | |
| R8136 | P2_VP8*(65-223) (opt3) | RVA/BE1128/ | HSD17B4/ | A64-N5-C30-hSL-N5 + | 1868 | 4, 51 |
| | | P[8] | PSMB3; a-1 | enzymatic poly(A) | | |
| R7043 | P2_VP8*(65-223) (opt3) | RVA/BE1128/ | 3′-UTR | A64-N5-C30-hSL-N5 | 1869 | 4, 51 |
| | | P[8] | muag; i-3 | | | |
| R7411 | P2_VP8*(65-223) (opt3) | RVA/BE1128/ | 3-UTR | A64-N5-C30-hSL-N5 + | 1869 | 4, 51 |
| | | P[8] | muag; i-3 | enzymatic poly(A) | | |
| R8137 | P2_VP8*(65-223) (opt3) | RVA/BE1128/ | 3′-UTR | A64-N5-C30-hSL-N5 | 1869 | 4, 51 |
| | | P[8] | muag; i-3 | | | |
| R8047 | P2_VP8*(65-223) (opt3) | RVA/BE1128/ | RPL32/ | A64-N5-C30-hSL-N5 | 1870 | 4, 51 |
| | | P[8] | ALB7; i-2 | | | |
| R5472 | P2_VP8*(41-223) (opt1) | RVA/BE1128/ | RPL32/ | A64-N5-C30-hSL-N5 | 1871 | 60 |
| | | P[8] | ALB7; i-2 | | | |
| R5473 | P2_VP8*(41-223) (opt1) | RVA/BE1128/ | RPL32/ | A64-N5-C30-hSL-N5 | 1871 | 60 |
| | | P[8] | ALB7; i-2 | | | |
| R8582 | P2_VP8*(65- | RVA/BE1128/ | HSD17B4/ | A100 | 1872, 609 | 69 |
| | 223)_Ferritin (opt1) | P[8] | PSMB3; a-1 | | | |
| R8577 | P2_VP8*(65- | RVA/BE1128/ | HSD17B4/ | hSL-A100 | 1873, 1185 | 69 |
| | 223)_Ferritin (opt1) | P[8] | PSMB3; a-1 | | | |
| R6326 | P2_VP8*(65- | RVA/BE1128/ | RPL32/ | A64-N5-C30-hSL-N5 | 1874 | 69 |
| | 223)_Ferritin (opt1) | P[8] | ALB7; i-2 | | | |
| R6327 | P2_VP8*(65- | RVA/BE1128/ | RPL32/ | A64-N5-C30-hSL-N5 | 1874 | 69 |
| | 223)_Ferritin (opt1) | P[8] | ALB7; i-2 | | | |
| R8583 | LumSynt_P2_VP8*(41- | RVA/BE1128/ | HSD17B4/ | A100 | 1875, 636 | 1, 96 |
| | 223) (opt1) | P[8] | PSMB3; a-1 | | | |
| R8578 | LumSynt_P2_VP8*(41- | RVA/BE1128/ | HSD17B4/ | hSL-A100 | 1876, 1212 | 1, 96 |
| | 223) (opt1) | P[8] | PSMB3; a-1 | | | |
| R6328 | LumSynt_P2_VP8*(41- | RVA/BE1128/ | RPL32/ | A64-N5-C30-hSL-N5 | 1877 | 1, 96 |
| | 223) (opt1) | P[8] | ALB7; i-2 | | | |
| R6329 | LumSynt_P2_VP8*(41- | RVA/BE1128/ | RPL32/ | A64-N5-C30-hSL-N5 | 1877 | 1, 96 |
| | 223) (opt1) | P[8] | ALB7; i-2 | | | |
| R8584 | SP-IgE_P2_VP8*(41- | RVA/BE1128/ | HSD17B4/ | A100 | 1878, 654 | 114 |
| | 223) (opt1) | P[8] | PSMB3; a-1 | | | |
| R8579 | SP-IgE_P2_VP8*(41- | RVA/BE1128/ | HSD17B4/ | hSL-A100 | 1879, 1230 | 114 |
| | 223) (opt1) | P[8] | PSMB3; a-1 | | | |
| R5488 | SP-IgE_P2_VP8*(41- | RVA/BE1128/ | RPL32/ | A64-N5-C30-hSL-N5 | 1880 | 114 |
| | 223) (opt1) | P[8] | ALB7; i-2 | | | |
| R5489 | SP-IgE_P2_VP8*(41- | RVA/BE1128/ | RPL32/ | A64-N5-C30-hSL-N5 | 1880 | 114 |
| | 223) (opt1) | P[8] | ALB7; i-2 | | | |
| R6322 | SP-IgE_P2_VP8*(41- | RVA/BE1128/ | RPL32/ | A64-N5-C30-hSL-N5 | 1881 | |
| | 223)_Ferritin (opt1) | P[8] | ALB7; i-2 | | | |
| R6323 | SP-IgE_P2_VP8*(41- | RVA/BE1128/ | RPL32/ | A64-N5-C30-hSL-N5 | 1881 | |
| | 223)_Ferritin (opt1) | P[8] | ALB7; i-2 | | | |
| R6324 | SP-IgE_P2_VP8*(41- | RVA/BE1128/ | RPL32/ | A64-N5-C30-KSL-N5 | 1882 | |
| | 223)_TM domain-HA | P[8] | ALB7; i-2 | | | |
| | (opt1) | | | | | |
| R6325 | SP-IgE_P2_VP8*(41- | RVA/BE1128/ | RPL32/ | A64-N5-C30-KSL-N5 | 1882 | |
| | 223)_TM domain-HA | P[8] | ALB7; i-2 | | | |
| | (opt1) | | | | | |
| R2569 | luciferase_PpLuc (opt1) | Photinus | 3′-UTR | A64-N5-C30-hSL-N5 | 1883 | |
| | | pyralis | muag; i-3 | | | |
| R4865 | capsid_protein (opt1) | NOV/Hu/GII.4/ | RPL32/ | A64-N5-C30-hSL-N5 | 1884 | |
| | | 031693/USA/2003 | ALB7; i-2 | | | |
| R4509 | P2_VP8*(65-223) (opt1) | RVA/Wa- | RPL32/ | A64-N5-C30-hSL-N5 | 1885 | 52 |
| | | VirWa/P[8] | ALB7; i-2 | | | |
| R4510 | P2_VP8*(65-223) (opt1) | RVA/Wa- | RPL32/ | A64-N5-C30-hSL-N5 | 1885 | 52 |
| | | VirWa/P[8] | ALB7; i-2 | | | |
| R3705 | VP8* (opt1) | RVA/BE1058/ | RPL32/ | A64-N5-C30-hSL-N5 | 1886 | 9, 19 |
| | | P[4] | ALB7; i-2 | | | |
| R3706 | VP8* (opt1) | RVA/BE1058/ | RPL32/ | A64-N5-C30-hSL-N5 | 1886 | 9, 19 |
| | | P[4] | ALB7; i-2 | | | |
| R3715 | VP8* (opt1) | RVA/F01322/ | RPL32/ | A64-N5-C30-hSL-N5 | 1887 | 8, 22 |
| | | P[6] | ALB7; i-2 | | | |
| R3718 | VP8* (opt1) | RVA/F01322/ | RPL32/ | A64-N5-C30-hSL-N5 | 1887 | 8, 22 |
| | | P[6] | ALB7; i-2 | | | |
| R3685 | VP8* (opt1) | RVA/BE1128/ | RPL32/ | A64-N5-C30-hSL-N5 | 1888 | 7, 24 |
| | | P[8] | ALB7; i-2 | | | |
| R3686 | VP8* (opt1) | RVA/BE1128/ | RPL32/ | A64-N5-C30-hSL-N5 | 1888 | 7, 24 |
| | | P[8] | ALB7; i-2 | | | |
| R6937 | VP4 (opt1) | RVA/Wa- | 3-UTR | A64-N5-C30-hSL-N5 | 1889 | 16 |
| | | VirWa/P[8] | muag; i-3 | | | |
| R6938 | VP4 (opt1) | RVA/Wa- | 3-UTR | A64-N5-C30-hSL-N5 | 1889 | 16 |
| | | VirWa/P[8] | muag; i-3 | | | |
| R5436 | HsPLAT_VP8*(21- | RVA/F01322/ | RPL32/ | A64-N5-C30-hSL-N5 | 1890 | |
| | 240, N32Q, N56Q, N97Q, | P[6] | ALB7; i-2 | | | |
| | N111Q, N114Q, N132Q, | | | | | |
| | N171Q, N182Q) (opt1) | | | | | |
| R5597 | HsPLAT_VP8*(21- | RVA/F01322/ | RPL32/ | A64-N5-C30-hSL-N5 | 1890 | |
| | 240, N32Q, N56Q, N97Q, | P[6] | ALB7; i-2 | | | |
| | N111Q, N114Q, N132Q, | | | | | |
| | N171Q, N182Q) (opt1) | | | | | |
| R5480 | HsALB_VP8*(2-230) | RVA/BE1128/ | RPL32/ | A64-N5-C30-hSL-N5 | 1891 | |
| | (opt1) | P[8] | ALB7; i-2 | | | |
| R5481 | HsALB_VP8*(2-230) | RVA/BE1128/ | RPL32/ | A64-N5-C30-hSL-N5 | 1891 | |
| | (opt1) | P[8] | ALB7; i-2 | | | |
| R5482 | HsALB_VP8*(11-223) | RVA/BE1128/ | RPL32/ | A64-N5-C30-hSL-N5 | 1892 | |
| | (opt1) | P[8] | ALB7; i-2 | | | |
| R5483 | HsALB_VP8*(11-223) | RVA/BE1128/ | RPL32/ | A64-N5-C30-hSL-N5 | 1892 | |
| | (opt1) | P[8] | ALB7; i-2 | | | |
| R5484 | HsALB_VP8*(41-223) | RVA/BE1128/ | RPL32/ | A64-N5-C30-hSL-N5 | 1893 | |
| | (opt1) | P[8] | ALB7; i-2 | | | |
| R5485 | HsALB_VP8*(41-223) | RVA/BE1128/ | RPL32/ | A64-N5-C30-hSL-N5 | 1893 | |
| | (opt1) | P[8] | ALB7; i-2 | | | |
| R5486 | HsALB_P2_VP8*(41- | RVA/BE1128/ | RPL32/ | A64-N5-C30-hSL-N5 | 1894 | |
| | 223) (opt1) | P[8] | ALB7; i-2 | | | |
| R5487 | HsALB_P2_VP8*(41- | RVA/BE1128/ | RPL32/ | A64-N5-C30-hSL-N5 | 1894 | |
| | 223) (opt1) | P[8] | ALB7; i-2 | | | |
| R5433 | HsPLAT_VP8*(41-223) | RVA/F01322/ | RPL32/ | A64-N5-C30-hSL-N5 | 1895 | |
| | (opt1) | P[6] | ALB7; i-2 | | | |
| R5594 | HsPLAT_VP8*(41-223) | RVA/F01322/ | RPL32/ | A64-N5-C30-hSL-N5 | 1895 | |
| | (opt1) | P[6] | ALB7; i-2 | | | |
| R5434 | HsPLAT_P2_VP8*(41- | RVA/F01322/ | RPL32/ | A64-N5-C30-hSL-N5 | 1896 | |
| | 223) (opt1) | P[6] | ALB7; i-2 | | | |
| R5595 | HsPLAT_P2_VP8*(41- | RVA/F01322/ | RPL32/ | A64-N5-C30-hSL-N5 | 1896 | |
| | 223) (opt1) | P[6] | ALB7; i-2 | | | |
| R5435 | HsPLAT_VP8*(2-230) | RVA/F01322/ | RPL32/ | A64-N5-C30-hSL-N5 | 1897 | |
| | (opt1) | P[6] | ALB7; i-2 | | | |
| R5596 | HsPLAT_VP8*(2-230) | RVA/F01322/ | RPL32/ | A64-N5-C30-hSL-N5 | 1897 | |
| | (opt1) | P[6] | ALB7; i-2 | | | |
| R8138 | P2_VP8*(65-223) (opt1) | RVA/BE1128/ | RPL32/ | A100 | 1898 | 4, 51 |
| | | P[8] | ALB7; i-2 | | | |
| R9247 | P2_VP8*(64-223) (opt1) | RVA/BE1058/ | HSD17B4/ | hSL-A100 | 1919 | 1899 |
| | | P[4] | PSMB3; a-1 | | | |
| R9246 | P2_VP8*(64-223) (opt1) | RVA/F01322/ | HSD17B4/ | hSL-A100 | 1920 | 1900 |
| | | P[6] | PSMB3; a-1 | | | |
| R9078 | P2_VP8*(65-223) (opt1) | RVA/BE1058/ | HSD17B4/ | hSL-A100 | 1921 | 6, 46 |
| | | P[4] | PSMB3; a-1 | | | |
| R9077 | P2_VP8*(65-223) (opt1) | RVA/F01322/ | HSD17B4/ | hSL-A100 | 1922 | 5, 49 |
| | | P[6] | PSMB3; a-1 | | | |
| R9092 | LumSynt_P2_VP8*(41- | RVA/BE1058/ | HSD17B4/ | hSL-A100 | 1923 | 3, 91 |
| | 223) (opt1) | P[4] | PSMB3; a-1 | | | |
| R9091 | LumSynt_P2_VP8*(41- | RVA/F01322/ | HSD17B4/ | hSL-A100 | 1924 | 2, 94 |
| | 223) (opt1) | P[6] | PSMB3; a-1 | | | |

In preferred embodiments, the coding RNA, preferably the mRNA, comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 586-1737, 1862-1882, 1885-1898, 1907-1930 or a fragment or variant of any of these sequences. Further information is provided under <223> identifier of the respective SEQ ID NO in the sequence listing.

In particularly preferred embodiments, the coding RNA, preferably the mRNA, comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 586-594, 604-612, 631-639, 649-666, 676-684, 703-711, 721-738, 748-756, 775-783, 793-810, 820-828, 847-855, 865-882, 892-900, 919-927, 937-954, 964-972, 991-999, 1009-1026, 1036-1044, 1063-1071, 1081-1098, 1108-1116, 1135-1143, 1153-1170, 1180-1188, 1207-1215, 1225-1242, 1252-1260, 1279-1287, 1297-1314, 1324-1332, 1351-1359, 1369-1386, 1396-1404, 1423-1431, 1441-1458, 1468-1476, 1495-1503, 1513-1530, 1540-1548, 1567-1575, 1585-1602, 1612-1620, 1639-1647, 1657-1674, 1684-1692, 1711-1719, 1729-1737, 1862-1870, 1872-1877, 1885, 1898, 1907-1930 or a fragment or variant of any of these sequences. Further information is provided under <223> identifier of the respective SEQ ID NO in the sequence listing.

It is further preferred that the coding RNA, preferably the mRNA, comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 586-594, 604-612, 631-639, 649-666, 676-684, 703-711, 721-738, 748-756, 775-783, 793-810, 820-828, 847-855, 865-882, 892-900, 919-927, 937-954, 964-972, 991-999, 1009-1026, 1036-1044, 1063-1071, 1081-1098, 1108-1116, 1135-1143, 1153-1170, 1180-1188, 1207-1215, 1225-1242, 1252-1260, 1279-1287, 1297-1314, 1324-1332, 1351-1359, 1369-1386, 1396-1404, 1423-1431, 1441-1458, 1468-1476, 1495-1503, 1513-1530, 1540-1548, 1567-1575, 1585-1602, 1612-1620, 1639-1647, 1657-1674, 1684-1692, 1711-1719, 1729-1737, 1862-1870, 1872-1877, 1885, 1898, 1907-1930, wherein said RNA sequences comprise a cap1 structure.

It is particularly preferred that the coding RNA, preferably the mRNA, comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 586-594, 604-612, 631-639, 649-666, 676-684, 703-711, 721-738, 748-756, 775-783, 793-810, 820-828, 847-855, 865-882, 892-900, 919-927, 937-954, 964-972, 991-999, 1009-1026, 1036-1044, 1063-1071, 1081-1098, 1108-1116, 1135-1143, 1153-1170, 1180-1188, 1207-1215, 1225-1242, 1252-1260, 1279-1287, 1297-1314, 1324-1332, 1351-1359, 1369-1386, 1396-1404, 1423-1431, 1441-1458, 1468-1476, 1495-1503, 1513-1530, 1540-1548, 1567-1575, 1585-1602, 1612-1620, 1639-1647, 1657-1674, 1684-1692, 1711-1719, 1729-1737, 1862, 1863, 1866, 1867, 1872, 1873, 1875, 1876, 1898, 1907-1930, wherein said RNA sequences comprise a cap1 structure and wherein said RNA sequences comprise a 3′-terminal poly(A) sequence obtained from a DNA template during RNA in vitro transcription.

It is further preferred that the coding RNA, preferably the mRNA, comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 586-594, 604-612, 631-639, 649-666, 676-684, 703-711, 721-738, 748-756, 775-783, 793-810, 820-828, 847-855, 865-882, 892-900, 919-927, 937-954, 964-972, 991-999, 1009-1026, 1036-1044, 1063-1071, 1081-1098, 1108-1116, 1135-1143, 1153-1170, 1180-1188, 1207-1215, 1225-1242, 1252-1260, 1279-1287, 1297-1314, 1324-1332, 1351-1359, 1369-1386, 1396-1404, 1423-1431, 1441-1458, 1468-1476, 1495-1503, 1513-1530, 1540-1548, 1567-1575, 1585-1602, 1612-1620, 1639-1647, 1657-1674, 1684-1692, 1711-1719, 1729-1737, 1862, 1863, 1866, 1867, 1872, 1873, 1875, 1876, 1898, 1907-1930, wherein said RNA sequences comprise a cap1 structure, wherein said RNA sequences comprise a 3-terminal poly(A) sequence obtained by enzymatic polyadenylation, wherein the majority of RNA molecules comprise about 100 (+/−20) to about 500 (+/−50), preferably about 250 (+/−20) adenosine nucleotides.

It is further preferred that the coding RNA, preferably the mRNA, comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 586-594, 604-612, 631-639, 649-666, 676-684, 703-711, 721-738, 748-756, 775-783, 793-810, 820-828, 847-855, 865-882, 892-900, 919-927, 937-954, 964-972, 991-999, 1009-1026, 1036-1044, 1063-1071, 1081-1098, 1108-1116, 1135-1143, 1153-1170, 1180-1188, 1207-1215, 1225-1242, 1252-1260, 1279-1287, 1297-1314, 1324-1332, 1351-1359, 1369-1386, 1396-1404, 1423-1431, 1441-1458, 1468-1476, 1495-1503, 1513-1530, 1540-1548, 1567-1575, 1585-1602, 1612-1620, 1639-1647, 1657-1674, 1684-1692, 1711-1719, 1729-1737, 1862-1870, 1872-1877, 1885, 1898, 1907-1930, wherein said RNA sequences comprise a cap1 structure, and, wherein at least one, preferably all uracil nucleotides in said RNA sequences are replaced by pseudouridine (ψ) nucleotides and/or N1-methylpseudouridine (m1ψ) nucleotides.

It is further preferred that the coding RNA, preferably the mRNA, comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 586-594, 604-612, 631-639, 649-666, 676-684, 703-711, 721-738, 748-756, 775-783, 793-810, 820-828, 847-855, 865-882, 892-900, 919-927, 937-954, 964-972, 991-999, 1009-1026, 1036-1044, 1063-1071, 1081-1098, 1108-1116, 1135-1143, 1153-1170, 1180-1188, 1207-1215, 1225-1242, 1252-1260, 1279-1287, 1297-1314, 1324-1332, 1351-1359, 1369-1386, 1396-1404, 1423-1431, 1441-1458, 1468-1476, 1495-1503, 1513-1530, 1540-1548, 1567-1575, 1585-1602, 1612-1620, 1639-1647, 1657-1674, 1684-1692, 1711-1719, 1729-1737, 1862-1870, 1872-1877, 1885, 1898, 1907-1930, wherein said RNA sequences comprise a Cap1 structure, and wherein at least one, preferably all uracil nucleotides in said RNA sequences are replaced by pseudouridine (ψ) nucleotides and/or N1-methylpseudouridine (m1ψ) nucleotides, and, wherein said RNA sequences comprise a 3-terminal poly(A) sequence obtained from a DNA template during RNA in vitro transcription.

It is further preferred that the coding RNA, preferably the mRNA, comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 586-594, 604-612, 631-639, 649-666, 676-684, 703-711, 721-738, 748-756, 775-783, 793-810, 820-828, 847-855, 865-882, 892-900, 919-927, 937-954, 964-972, 991-999, 1009-1026, 1036-1044, 1063-1071, 1081-1098, 1108-1116, 1135-1143, 1153-1170, 1180-1188, 1207-1215, 1225-1242, 1252-1260, 1279-1287, 1297-1314, 1324-1332, 1351-1359, 1369-1386, 1396-1404, 1423-1431, 1441-1458, 1468-1476, 1495-1503, 1513-1530, 1540-1548, 1567-1575, 1585-1602, 1612-1620, 1639-1647, 1657-1674, 1684-1692, 1711-1719, 1729-1737, 1862, 1863, 1866, 1867, 1872, 1873, 1875, 1876, 1898, 1907-1930, wherein said RNA sequences comprise a cap1 structure, and wherein at least one, preferably all uracil nucleotides in said RNA sequences are replaced by pseudouridine (ψ) nucleotides and/or N1-methylpseudouridine (m1ψ) nucleotides, and, wherein said RNA sequences comprise a 3-terminal poly(A) sequence obtained by enzymatic polyadenylation, wherein the majority of RNA molecules comprise about 100 (+/−20) to about 500 (+/−50), preferably about 250 (+/−20) adenosine nucleotides.

As outlined throughout the specification, additional information regarding suitable amino acid sequences or nucleic acid sequences (coding sequences, mRNA sequences) may also be derived from the sequence listing, in particular from the details provided therein under identifier <223> as explained in the following.

It has to be noted that throughout the sequence listing, information provided under numeric identifier <223> follows the same structure: “<SEQUENCE_DESCRIPTOR> from <CONSTRUCT_IDENTIFIER>”. The <SEQUENCE_DESCRIPTOR> relates to the type of sequence (e.g., “derived and/or modified protein sequence”, “derived and/or modified CDS sequence” “mRNA product design a-1 comprising derived and/or modified sequence” or “mRNA product Design i-3 comprising derived and/or modified sequence”, etc.) and whether the sequence comprises or consists of a wild type sequence (“wt”) or whether the sequence comprises or consists of a sequence-optimized sequence (e.g. “opt1”, “opt4”, “opt5”; sequence optimizations are described in further detail below). The <CONSTRUCT_IDENTIFIER> provided under numeric identifier <223> has the following structures: (“organism_construct name”, or “organism_accession number_construct name”) and is intended to help the person skilled in the art to explicitly derive suitable nucleic acid sequences (e.g., RNA, mRNA) encoding the same VP8* protein construct according to the invention.

RNA Manufacturing Methods:

The coding RNA, preferably the mRNA of the invention may be prepared using any method known in the art, including chemical synthesis such as e.g. solid phase RNA synthesis, as well as in vitro methods, such as RNA in vitro transcription reactions.

In a preferred embodiment, the coding RNA, preferably the mRNA is obtained by RNA in vitro transcription.

Accordingly, the coding RNA of the invention is preferably an in vitro transcribed RNA.

The terms “RNA in vitro transcription” or “in vitro transcription” relate to a process wherein RNA is synthesized in a cell-free system (in vitro). RNA may be obtained by DNA-dependent in vitro transcription of an appropriate DNA template, which according to the present invention is a linearized plasmid DNA template or a PCR-amplified DNA template. The promoter for controlling RNA in vitro transcription can be any promoter for any DNA-dependent RNA polymerase. Particular examples of DNA-dependent RNA polymerases are the T7, T3, SP6, or Syn5 RNA polymerases. In a preferred embodiment of the present invention the DNA template is linearized with a suitable restriction enzyme, before it is subjected to RNA in vitro transcription.

Reagents used in RNA in vitro transcription typically include: a DNA template (linearized plasmid DNA or PCR product) with a promoter sequence that has a high binding affinity for its respective RNA polymerase such as bacteriophage-encoded RNA polymerases (T7, T3, SP6, or Syn5); ribonucleotide triphosphates (NTPs) for the four bases (adenine, cytosine, guanine and uracil); optionally, a cap analogue as defined herein; optionally, further modified nucleotides as defined herein; a DNA-dependent RNA polymerase capable of binding to the promoter sequence within the DNA template (e.g. T7, T3, SP6, or Syn5 RNA polymerase); optionally, a ribonuclease (RNase) inhibitor to inactivate any potentially contaminating RNase; optionally, a pyrophosphatase to degrade pyrophosphate, which may inhibit RNA in vitro transcription; MgCl2, which supplies Mg2+ ions as a co-factor for the polymerase; a buffer (TRIS or HEPES) to maintain a suitable pH value, which can also contain antioxidants (e.g. DTT), and/or polyamines such as spermidine at optimal concentrations, e.g. a buffer system comprising TRIS-Citrate as disclosed in WO2017/109161.

In preferred embodiments, the cap1 structure of the coding RNA of the invention is formed using co-transcriptional capping using tri-nucleotide cap analogues m7G(5′)ppp(5′)(2′OMeA)pG or m7G(5′)ppp(5′)(2′OMeG)pG. A preferred cap1 analogue that may suitably be used in manufacturing the coding RNA of the invention is m7G(5′)ppp(5′)(2′OMeA)pG.

In embodiments, the nucleotide mixture used in RNA in vitro transcription may additionally comprises modified nucleotides as defined herein. In that context, preferred modified nucleotides may be selected from pseudouridine (ψ), N1-methylpseudouridine (m1ψ), 5-methylcytosine, and 5-methoxyuridine. In particular embodiments, uracil nucleotides in the nucleotide mixture are replaced (either partially or completely) by pseudouridine (ψ) and/or N1-methylpseudouridine (m1ψ) to obtain a modified coding RNA.

In preferred embodiments, the nucleotide mixture (i.e. the fraction of each nucleotide in the mixture) used for RNA in vitro transcription reactions may be optimized for the given RNA sequence, preferably as described WO2015/188933.

In embodiment where more than one different coding RNA as defined herein have to be produced, e.g. where 2, 3, 4, 5, 6, 7, 8, 9, 10 or even more different coding RNAs have to be produced (e.g. encoding different VP8* antigen constructs derived from different Rotavirus A serotypes; see second aspect), procedures as described in WO2017/109134 may be suitably be used.

In the context of RNA vaccine production, it may be required to provide GMP-grade RNA. GMP-grade RNA may be produced using a manufacturing process approved by regulatory authorities. Accordingly, in a particularly preferred embodiment, RNA production is performed under current good manufacturing practice (GMP), implementing various quality control steps on DNA and RNA level, preferably according to WO2016/180430. In preferred embodiments, the RNA of the invention is a GMP-grade RNA, particularly a GMP-grade mRNA. Accordingly, a coding RNA for a vaccine is a GMP grade RNA.

The obtained RNA products are preferably purified using PureMessenger® (CureVac, Tübingen, Germany; RP-HPLC according to WO2008/077592) and/or tangential flow filtration (as described in WO2016/193206) and/or oligo d(T) purification.

In a further preferred embodiment, the coding RNA, particularly the purified coding RNA, is lyophilized (e.g. according to WO2016/165831 or WO2011/069586) to yield a temperature stable dried coding RNA (powder) as defined herein. The RNA of the invention, particularly the purified RNA may also be dried using spray-drying or spray-freeze drying (e.g. according to WO2016/184575 or WO2016/184576) to yield a temperature stable RNA (powder) as defined herein. Accordingly, in the context of manufacturing and purifying RNA, the disclosures of WO2017/109161, WO2015/188933, WO2016/180430, WO2008/077592, WO2016/193206, WO2016/165831, WO2011/069586, WO2016/184575, and WO2016/184576 are incorporated herewith by reference.

Accordingly, in preferred embodiments, the coding RNA is a dried RNA, particularly a dried mRNA.

The term “dried RNA” as used herein has to be understood as RNA that has been lyophilized, or spray-dried, or spray-freeze dried as defined above to obtain a temperature stable dried RNA (powder).

In preferred embodiments, the coding RNA of the invention is a purified RNA, particularly purified mRNA.

The term “purified RNA” or “purified mRNA” as used herein has to be understood as RNA which has a higher purity after certain purification steps (e.g. HPLC, TFF, Oligo d(T) purification, precipitation steps) than the starting material (e.g. in vitro transcribed RNA). Typical impurities that are essentially not present in purified RNA comprise peptides or proteins (e.g. enzymes derived from DNA dependent RNA in vitro transcription, e.g. RNA polymerases, RNases, pyrophosphatase, restriction endonuclease, DNase), spermidine, BSA, abortive RNA sequences, RNA fragments (short double stranded RNA fragments, abortive sequences etc.), free nucleotides (modified nucleotides, conventional NTPs, cap analogue), template DNA fragments, buffer components (HEPES, TRIS, MgCl2) etc. Other potential impurities that may be derived from e.g. fermentation procedures comprise bacterial impurities (bioburden, bacterial DNA) or impurities derived from purification procedures (organic solvents etc.). Accordingly, it is desirable in this regard for the “degree of RNA purity” to be as close as possible to 100%. It is also desirable for the degree of RNA purity that the amount of full-length RNA transcripts is as close as possible to 100%. Accordingly “purified RNA” as used herein has a degree of purity of more than 75%, 80%, 85%, very particularly 90%, 91%, 92%, 93%, 94%, 95%, 96%, 97%, 98% and most favorably 99% or more. The degree of purity may for example be determined by an analytical HPLC, wherein the percentages provided above correspond to the ratio between the area of the peak for the target RNA and the total area of all peaks representing the by-products. Alternatively, the degree of purity may for example be determined by an analytical agarose gel electrophoresis or capillary gel electrophoresis.

It has to be understood that “dried RNA” as defined herein and “purified RNA” as defined herein or “GMP-grade RNA” as defined herein may have superior stability characteristics (in vitro, in vivo) and improved efficiency (e.g. better translatability of the mRNA in vivo) and are therefore particularly suitable for a medical purpose, e.g. a vaccine.

Following co-transcriptional capping as defined herein, and following purification as defined herein, the capping degree of the obtained coding RNA may be determined using capping assays as described in published PCT application WO2015/101416, in particular, as described in Claims 27 to 46 of published PCT application WO20151101416 can be used. Alternatively, a capping assays described in PCT/EP2018/08667 may be used.

Composition, Pharmaceutical Composition:

A second aspect relates to a composition comprising at least one coding RNA of the first aspect.

Notably, embodiments relating to the composition of the second aspect may likewise be read on and be understood as suitable embodiments of the vaccine of the third aspect. Also, embodiments relating to the vaccine of the third aspect may likewise be read on and be understood as suitable embodiments of the composition of the second aspect (comprising the RNA of the first aspect).

In preferred embodiments, said composition comprises at least one coding RNA encoding a Rotavirus antigen, preferably VP8* according to the first aspect, or an immunogenic fragment or immunogenic variant thereof, wherein said composition is to be, preferably, administered intramuscularly or intradermal.

Preferably, intramuscular or intradermal administration of said composition results in expression of the encoded VP8* antigen construct in a subject. Preferably, the composition of the second aspect is suitable for a vaccine, in particular, suitable for a Rotavirus vaccine.

In the context of the invention, a “composition” refers to any type of composition in which the specified ingredients (e.g. RNA encoding VP8* e.g. in association with a polymeric carrier or LNP), may be incorporated, optionally along with any further constituents, usually with at least one pharmaceutically acceptable carrier or excipient. The composition may be a dry composition such as a powder or granules, or a solid unit such as a lyophilized form. Alternatively, the composition may be in liquid form, and each constituent may be independently incorporated in dissolved or dispersed (e.g. suspended or emulsified) form.

In a preferred embodiment of the second aspect, the composition comprises at least one coding RNA of the first aspect and, optionally, at least one pharmaceutically acceptable carrier or excipient.

In particularly preferred embodiments of the second aspect, the composition comprises at least one coding RNA, wherein the coding RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 586-594, 604-612, 631-639, 649-666, 676-684, 703-711, 721-738, 748-756, 775-783, 793-810, 820-828, 847-855, 865-882, 892-900, 919-927, 937-954, 964-972, 991-999, 1009-1026, 1036-1044, 1063-1071, 1081-1098, 1108-1116, 1135-1143, 1153-1170, 1180-1188, 1207-1215, 1225-1242, 1252-1260, 1279-1287, 1297-1314, 1324-1332, 1351-1359, 1369-1386, 1396-1404, 1423-1431, 1441-1458, 1468-1476, 1495-1503, 1513-1530, 1540-1548, 1567-1575, 1585-1602, 1612-1620, 1639-1647, 1657-1674, 1684-1692, 1711-1719, 1729-1737, 1862-1870, 1872-1877, 1885, 1898, 1907-1930, and, optionally, at least one pharmaceutically acceptable carrier or excipient.

In other embodiments, the composition comprises at least one coding RNA, wherein the coding RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 828-3146 or 3306-3593 of WO2017/081110A1, and, optionally, at least one pharmaceutically acceptable carrier or excipient.

The term “pharmaceutically acceptable carrier” or “pharmaceutically acceptable excipient” as used herein preferably includes the liquid or non-liquid basis of the composition for administration. If the composition is provided in liquid form, the carrier may be water, e.g. pyrogen-free water; isotonic saline or buffered (aqueous) solutions, e.g. phosphate, citrate etc. buffered solutions. Water or preferably a buffer, more preferably an aqueous buffer, may be used, containing a sodium salt, preferably at least 50 mM of a sodium salt, a calcium salt, preferably at least 0.01 mM of a calcium salt, and optionally a potassium salt, preferably at least 3 mM of a potassium salt. According to preferred embodiments, the sodium, calcium and, optionally, potassium salts may occur in the form of their halogenides, e.g. chlorides, iodides, or bromides, in the form of their hydroxides, carbonates, hydrogen carbonates, or sulfates, etc. Examples of sodium salts include NaCl, NaI, NaBr, Na₂CO₃, NaHCO₃, Na₂SO₄, examples of the optional potassium salts include KCl, KI, KBr, K₂CO₃, KHCO₃, K₂SO₄, and examples of calcium salts include CaCl₂, CaI₂, CaBr₂, CaCO₃, CaSO₄, Ca(OH)₂.

Furthermore, organic anions of the aforementioned cations may be in the buffer. Accordingly, in embodiments, the RNA composition of the invention may comprise pharmaceutically acceptable carriers or excipients using one or more pharmaceutically acceptable carriers or excipients to e.g. increase stability, increase cell transfection, permit the sustained or delayed, increase the translation of encoded VP8* protein construct in vivo, and/or alter the release profile of encoded VP8* protein in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present invention can include, without limitation, lipidoids, liposomes, lipid nanoparticles, polymers, lipoplexes, core-shell nanoparticles, peptides, proteins, cells transfected with polynucleotides, hyaluronidase, nanoparticle mimics and combinations thereof. In embodiments, one or more compatible solid or liquid fillers or diluents or encapsulating compounds may be used as well, which are suitable for administration to a subject. The term “compatible” as used herein means that the constituents of the composition are capable of being mixed with the at least one RNA and, optionally, a plurality of RNAs of the composition, in such a manner that no interaction occurs, which would substantially reduce the biological activity or the pharmaceutical effectiveness of the composition under typical use conditions (e.g., intramuscular or intradermal administration). Pharmaceutically acceptable carriers or excipients must have sufficiently high purity and sufficiently low toxicity to make them suitable for administration to a subject to be treated. Compounds which may be used as pharmaceutically acceptable carriers or excipients may be sugars, such as, for example, lactose, glucose, trehalose, mannose, and sucrose; starches, such as, for example, corn starch or potato starch; dextrose; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate; vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; alginic acid.

The at least one pharmaceutically acceptable carrier or excipient of the composition may preferably be selected to be suitable for intramuscular or intradermal delivery/administration of said composition. Accordingly, the composition is preferably a pharmaceutical composition, suitably a composition for intramuscular administration.

Subjects to which administration of the compositions, preferably the pharmaceutical composition, is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Pharmaceutical compositions of the present invention may suitably be sterile and/or pyrogen-free.

In embodiments, the composition as defined herein may comprise a plurality or at least more than one of the coding RNA species as defined in the context of the first aspect of the invention. Preferably, the composition as defined herein may comprise 2, 3, 4, 5, 6, 7, 8, 9, or 10 different coding RNAs each defined in the context of the first aspect.

In embodiment, the composition may comprise at least 2, 3, 4, 5, 6, 7, 8, 9, 10 or even more different coding RNA species as defined in the context of the first aspect, each encoding at least one antigenic peptide or protein derived from the same Rotavirus, or a fragment or variant thereof. Particularly, said (genetically) same Rotavirus expresses (essentially) the same repertoire of proteins or peptides, wherein all proteins or peptides have (essentially) the same amino acid sequence. Particularly, said (genetically) same Rotavirus expresses essentially the same proteins, peptides or polyproteins, wherein these protein, peptide or polyproteins preferably do not differ in their amino acid sequence(s).

In embodiments, the composition comprises at least 2, 3, 4, 5, 6, 7, 8, 9, 10 or even more different coding RNA species as defined in the context of the first aspect, each encoding at least one peptide or protein derived from a genetically different Rotavirus (e.g. a different Rotavirus A serotype), or a fragment or variant thereof. The terms “different” or “different Rotavirus” as used throughout the present specification have to be understood as the difference between at least two respective Rotaviruses (e.g. a different Rotavirus A serotype), wherein the difference is manifested on the genome of the respective different Rotaviruses. Particularly, said (genetically) different Rotaviruses may express at least one different protein, peptide or polyprotein, wherein the at least one different protein, peptide or polyprotein differs in at least one amino acid.

In embodiments, the composition comprises at least 2, 3, 4, 5, 6, 7, 8, 9, 10 or even more coding RNA species each encoding a different Rotavirus antigen (constructs), wherein each of the different Rotavirus antigen (constructs) may be selected from VP1, VP2, VP3, VP4, VP5*, VP6, VP7, VP8*, NSP1, NSP2, NSP3, NSP4, NSP5 and NSP6, or combinations, or immunogenic fragments, or immunogenic variants of any of these.

In preferred embodiments, the composition comprises at least 2, 3, 4, 5, 6, 7, 8, 9, 10 or even more coding RNA construct species each encoding a different VP8* Rotavirus antigen (constructs) as defined in the first aspect, preferably wherein each of the coding RNA constructs are selected from SEQ ID NOs: 586-1737, 1862-1882, 1885-1898, 1907-1930.

In particularly preferred embodiments, the composition comprises at least 2, 3, 4, 5, 6, 7, 8, 9, 10 or even more coding RNA construct species each encoding the same VP8* Rotavirus antigen (constructs) derived from a genetically different Rotavirus (e.g. a different Rotavirus A serotype), or a fragment or variant thereof as defined in the first aspect, preferably wherein each of the coding RNA constructs are selected from SEQ ID NOs: 586-1737, 1862-1882, 1885-1898, 1907-1930.

In preferred embodiments, the composition of the second aspect comprises
(i) at least one coding RNA encoding at least one antigenic protein that is or is derived from VP8* of a Rotavirus A from a P[4] serotype as specified herein; and
(ii) at least one coding RNA encoding at least one antigenic protein that is or is derived from VP8* of a Rotavirus A from a P[6] serotype as specified herein; and
(iii) at least one coding RNA encoding at least one antigenic protein that is or is derived from VP8* of a Rotavirus A from a P[8] serotype as specified herein.

In particularly preferred embodiments, the composition of the second aspect comprises
(i) at least one coding RNA encoding at least one antigenic protein that is or is derived from VP8* of a Rotavirus A from a P[4] serotype as specified herein, preferably according to SEQ ID NOs: 586-588, 595-597, 604-606, 613-615, 622-624, 631-633, 640-642, 649-651, 658-660, 667-669, 676-678, 685-687, 694-696, 703-705, 712-714, 721-723, 730-732, 739-741, 748-750, 757-759, 766-768, 775-777, 784-786, 793-795, 802-804, 811-813, 820-822, 829-831, 838-840, 847-849, 856-858, 865-867, 874-876, 883-885, 892-894, 901-903, 910-912, 919-921, 928-930, 937-939, 946-948, 955-957, 964-966, 973-975, 982-984, 991-993, 1000-1002, 1009-1011, 1018-1020, 1027-1029, 1036-1038, 1045-1047, 1054-1056, 1063-1065, 1072-1074, 1081-1083, 1090-1092, 1099-1101, 1108-1110, 1117-1119, 1126-1128, 1135-1137, 1144-1146, 1153-1155, 1162-1164, 1171-1173, 1180-1182, 1189-1191, 1198-1200, 1207-1209, 1216-1218, 1225-1227, 1234-1236, 1243-1245, 1252-1254, 1261-1263, 1270-1272, 1279-1281, 1288-1290, 1297-1299, 1306-1308, 1315-1317, 1324-1326, 1333-1335, 1342-1344, 1351-1353, 1360-1362, 1369-1371, 1378-1380, 1387-1389, 1396-1398, 1405-1407, 1414-1416, 1423-1425, 1432-1434, 1441-1443, 1450-1452, 1459-1461, 1468-1470, 1477-1479, 1486-1488, 1495-1497, 1504-1506, 1513-1515, 1522-1524, 1531-1533, 1540-1542, 1549-1551, 1558-1560, 1567-1569, 1576-1578, 1585-1587, 1594-1596, 1603-1605, 1612-1614, 1621-1623, 1630-1632, 1639-1641, 1648-1650, 1657-1659, 1666-1668, 1675-1677, 1684-1686, 1693-1695, 1702-1704, 1711-1713, 1720-1722, 1729-1731, 1886, 1907, 1909, 1911, 1913, 1915, 1917, 1919, 1921, 1923, 1925, 1927, 1929 or fragments or variants thereof;
(ii) at least one coding RNA encoding at least one antigenic protein that is or is derived from VP8* of a Rotavirus A from a P[6] serotype as specified herein, preferably according to SEQ ID NOs: 589, 590, 598, 599, 607, 608, 616, 617, 625, 626, 634, 635, 643, 644, 652, 653, 661, 662, 670, 671, 679, 680, 688, 689, 697, 698, 706, 707, 715, 716, 724, 725, 733, 734, 742, 743, 751, 752, 760, 761, 769, 770, 778, 779, 787, 788, 796, 797, 805, 806, 814, 815, 823, 824, 832, 833, 841, 842, 850, 851, 859, 860, 868, 869, 877, 878, 886, 887, 895, 896, 904, 905, 913, 914, 922, 923, 931, 932, 940, 941, 949, 950, 958, 959, 967, 968, 976, 977, 985, 986, 994, 995, 1003, 1004, 1012, 1013, 1021, 1022, 1030, 1031, 1039, 1040, 1048, 1049, 1057, 1058, 1066, 1067, 1075, 1076, 1084, 1085, 1093, 1094, 1102, 1103, 1111, 1112, 1120, 1121, 1129, 1130, 1138, 1139, 1147, 1148, 1156, 1157, 1165, 1166, 1174, 1175, 1183, 1184, 1192, 1193, 1201, 1202, 1210, 1211, 1219, 1220, 1228, 1229, 1237, 1238, 1246, 1247, 1255, 1256, 1264, 1265, 1273, 1274, 1282, 1283, 1291, 1292, 1300, 1301, 1309, 1310, 1318, 1319, 1327, 1328, 1336, 1337, 1345, 1346, 1354, 1355, 1363, 1364, 1372, 1373, 1381, 1382, 1390, 1391, 1399, 1400, 1408, 1409, 1417, 1418, 1426, 1427, 1435, 1436, 1444, 1445, 1453, 1454, 1462, 1463, 1471, 1472, 1480, 1481, 1489, 1490, 1498, 1499, 1507, 1508, 1516, 1517, 1525, 1526, 1534, 1535, 1543, 1544, 1552, 1553, 1561, 1562, 1570, 1571, 1579, 1580, 1588, 1589, 1597, 1598, 1606, 1607, 1615, 1616, 1624, 1625, 1633, 1634, 1642, 1643, 1651, 1652, 1660, 1661, 1669, 1670, 1678, 1679, 1687, 1688, 1696, 1697, 1705, 1706, 1714, 1715, 1723, 1724, 1732, 1733, 1887, 1890, 1895-1897, 1908, 1910, 1912, 1914, 1916, 1918, 1920, 1922, 1924, 1926, 1928, 1930 or fragments or variants thereof; and
(iii) at least one coding RNA encoding at least one antigenic protein that is or is derived from VP8* of a Rotavirus A from a P[8] serotype as specified herein, according to SEQ ID NOs: 591-594, 600-603, 609-612, 618-621, 627-630, 636-639, 645-648, 654-657, 663-666, 672-675, 681-684, 690-693, 699-702, 708-711, 717-720, 726-729, 735-738, 744-747, 753-756, 762-765, 771-774, 780-783, 789-792, 798-801, 807-810, 816-819, 825-828, 834-837, 843-846, 852-855, 861-864, 870-873, 879-882, 888-891, 897-900, 906-909, 915-918, 924-927, 933-936, 942-945, 951-954, 960-963, 969-972, 978-981, 987-990, 996-999, 1005-1008, 1014-1017, 1023-1026, 1032-1035, 1041-1044, 1050-1053, 1059-1062, 1068-1071, 1077-1080, 1086-1089, 1095-1098, 1104-1107, 1113-1116, 1122-1125, 1131-1134, 1140-1143, 1149-1152, 1158-1161, 1167-1170, 1176-1179, 1185-1188, 1194-1197, 1203-1206, 1212-1215, 1221-1224, 1230-1233, 1239-1242, 1248-1251, 1257-1260, 1266-1269, 1275-1278, 1284-1287, 1293-1296, 1302-1305, 1311-1314, 1320-1323, 1329-1332, 1338-1341, 1347-1350, 1356-1359, 1365-1368, 1374-1377, 1383-1386, 1392-1395, 1401-1404, 1410-1413, 1419-1422, 1428-1431, 1437-1440, 1446-1449, 1455-1458, 1464-1467, 1473-1476, 1482-1485, 1491-1494, 1500-1503, 1509-1512, 1518-1521, 1527-1530, 1536-1539, 1545-1548, 1554-1557, 1563-1566, 1572-1575, 1581-1584, 1590-1593, 1599-1602, 1608-1611, 1617-1620, 1626-1629, 1635-1638, 1644-1647, 1653-1656, 1662-1665, 1671-1674, 1680-1683, 1689-1692, 1698-1701, 1707-1710, 1716-1719, 1725-1728, 1734-1737, 1862-1882, 1885, 1888, 1889, 1891-1894, 1898, or fragments or variants thereof.

In particularly preferred embodiments, the composition of the second aspect comprises
(i) one coding RNA encoding at least one antigenic protein that is or is derived from VP8* of a Rotavirus A from a P[4] serotype as specified herein selected from SEQ ID NOs: 586-588, 595-597, 604-606, 613-615, 622-624, 631-633, 640-642, 649-651, 658-660, 667-669, 676-678, 685-687, 694-696, 703-705, 712-714, 721-723, 730-732, 739-741, 748-750, 757-759, 766-768, 775-777, 784-786, 793-795, 802-804, 811-813, 820-822, 829-831, 838-840, 847-849, 856-858, 865-867, 874-876, 883-885, 892-894, 901-903, 910-912, 919-921, 928-930, 937-939, 946-948, 955-957, 964-966, 973-975, 982-984, 991-993, 1000-1002, 1009-1011, 1018-1020, 1027-1029, 1036-1038, 1045-1047, 1054-1056, 1063-1065, 1072-1074, 1081-1083, 1090-1092, 1099-1101, 1108-1110, 1117-1119, 1126-1128, 1135-1137, 1144-1146, 1153-1155, 1162-1164, 1171-1173, 1180-1182, 1189-1191, 1198-1200, 1207-1209, 1216-1218, 1225-1227, 1234-1236, 1243-1245, 1252-1254, 1261-1263, 1270-1272, 1279-1281, 1288-1290, 1297-1299, 1306-1308, 1315-1317, 1324-1326, 1333-1335, 1342-1344, 1351-1353, 1360-1362, 1369-1371, 1378-1380, 1387-1389, 1396-1398, 1405-1407, 1414-1416, 1423-1425, 1432-1434, 1441-1443, 1450-1452, 1459-1461, 1468-1470, 1477-1479, 1486-1488, 1495-1497, 1504-1506, 1513-1515, 1522-1524, 1531-1533, 1540-1542, 1549-1551, 1558-1560, 1567-1569, 1576-1578, 1585-1587, 1594-1596, 1603-1605, 1612-1614, 1621-1623, 1630-1632, 1639-1641, 1648-1650, 1657-1659, 1666-1668, 1675-1677, 1684-1686, 1693-1695, 1702-1704, 1711-1713, 1720-1722, 1729-1731, 1886, 1907, 1909, 1911, 1913, 1915, 1917, 1919, 1921, 1923, 1925, 1927, 1929 or fragments or variants thereof;
(ii) one coding RNA encoding at least one antigenic protein that is or is derived from VP8* of a Rotavirus A from a P[6] serotype as specified herein selected from SEQ ID NOs: 589, 590, 598, 599, 607, 608, 616, 617, 625, 626, 634, 635, 643, 644, 652, 653, 661, 662, 670, 671, 679, 680, 688, 689, 697, 698, 706, 707, 715, 716, 724, 725, 733, 734, 742, 743, 751, 752, 760, 761, 769, 770, 778, 779, 787, 788, 796, 797, 805, 806, 814, 815, 823, 824, 832, 833, 841, 842, 850, 851, 859, 860, 868, 869, 877, 878, 886, 887, 895, 896, 904, 905, 913, 914, 922, 923, 931, 932, 940, 941, 949, 950, 958, 959, 967, 968, 976, 977, 985, 986, 994, 995, 1003, 1004, 1012, 1013, 1021, 1022, 1030, 1031, 1039, 1040, 1048, 1049, 1057, 1058, 1066, 1067, 1075, 1076, 1084, 1085, 1093, 1094, 1102, 1103, 1111, 1112, 1120, 1121, 1129, 1130, 1138, 1139, 1147, 1148, 1156, 1157, 1165, 1166, 1174, 1175, 1183, 1184, 1192, 1193, 1201, 1202, 1210, 1211, 1219, 1220, 1228, 1229, 1237, 1238, 1246, 1247, 1255, 1256, 1264, 1265, 1273, 1274, 1282, 1283, 1291, 1292, 1300, 1301, 1309, 1310, 1318, 1319, 1327, 1328, 1336, 1337, 1345, 1346, 1354, 1355, 1363, 1364, 1372, 1373, 1381, 1382, 1390, 1391, 1399, 1400, 1408, 1409, 1417, 1418, 1426, 1427, 1435, 1436, 1444, 1445, 1453, 1454, 1462, 1463, 1471, 1472, 1480, 1481, 1489, 1490, 1498, 1499, 1507, 1508, 1516, 1517, 1525, 1526, 1534, 1535, 1543, 1544, 1552, 1553, 1561, 1562, 1570, 1571, 1579, 1580, 1588, 1589, 1597, 1598, 1606, 1607, 1615, 1616, 1624, 1625, 1633, 1634, 1642, 1643, 1651, 1652, 1660, 1661, 1669, 1670, 1678, 1679, 1687, 1688, 1696, 1697, 1705, 1706, 1714, 1715, 1723, 1724, 1732, 1733, 1887, 1890, 1895-1897, 1908, 1910, 1912, 1914, 1916, 1918, 1920, 1922, 1924, 1926, 1928, 1930, or fragments or variants thereof; and
(iii) one coding RNA encoding at least one antigenic protein that is or is derived from VP8* of a Rotavirus A from a P[8] serotype as specified herein selected from SEQ ID NOs: 591-594, 600-603, 609-612, 618-621, 627-630, 636-639, 645-648, 654-657, 663-666, 672-675, 681-684, 690-693, 699-702, 708-711, 717-720, 726-729, 735-738, 744-747, 753-756, 762-765, 771-774, 780-783, 789-792, 798-801, 807-810, 816-819, 825-828, 834-837, 843-846, 852-855, 861-864, 870-873, 879-882, 888-891, 897-900, 906-909, 915-918, 924-927, 933-936, 942-945, 951-954, 960-963, 969-972, 978-981, 987-990, 996-999, 1005-1008, 1014-1017, 1023-1026, 1032-1035, 1041-1044, 1050-1053, 1059-1062, 1068-1071, 1077-1080, 1086-1089, 1095-1098, 1104-1107, 1113-1116, 1122-1125, 1131-1134, 1140-1143, 1149-1152, 1158-1161, 1167-1170, 1176-1179, 1185-1188, 1194-1197, 1203-1206, 1212-1215, 1221-1224, 1230-1233, 1239-1242, 1248-1251, 1257-1260, 1266-1269, 1275-1278, 1284-1287, 1293-1296, 1302-1305, 1311-1314, 1320-1323, 1329-1332, 1338-1341, 1347-1350, 1356-1359, 1365-1368, 1374-1377, 1383-1386, 1392-1395, 1401-1404, 1410-1413, 1419-1422, 1428-1431, 1437-1440, 1446-1449, 1455-1458, 1464-1467, 1473-1476, 1482-1485, 1491-1494, 1500-1503, 1509-1512, 1518-1521, 1527-1530, 1536-1539, 1545-1548, 1554-1557, 1563-1566, 1572-1575, 1581-1584, 1590-1593, 1599-1602, 1608-1611, 1617-1620, 1626-1629, 1635-1638, 1644-1647, 1653-1656, 1662-1665, 1671-1674, 1680-1683, 1689-1692, 1698-1701, 1707-1710, 1716-1719, 1725-1728, 1734-1737, 1862-1882, 1885, 1888, 1889, 1891-1894, 1898, or fragments or variants thereof.

In that context, the term “one coding RNA” has to be understood as an ensemble of essentially identical RNA molecule species. The term “one coding RNA” should not be understood as one individual RNA molecule. In preferred embodiments, the one coding RNA of (i), (ii) and (iii) encode the same antigen constructs.

In embodiments, the composition comprises
(i) at least one coding RNA encoding at least one antigenic protein that is or is derived from VP8* of a Rotavirus A as specified herein, wherein the antigen construct encodes a secreted protein selected from SEQ ID NOs: mRNAs 622-657, 694-729, 766-801, 838-873, 910-945, 982-1017, 1054-1089, 1126-1161, 1198-1233, 1270-1305, 1342-1377, 1414-1449, 1486-1521, 1558-1593, 1630-1665, 1702-1737, 1875-1882, 1890-1897, 1911-1912, 1917-1918, 1923-1924, 1929-1930, or fragments or variants thereof;
(ii) at least one coding RNA encoding at least one antigenic protein that is or is derived from VP8* of a Rotavirus A as specified herein, wherein the antigen construct encodes a cytosolic protein selected from SEQ ID NOs: 586-621, 658-693, 730-765, 802-837, 874-909, 946-981, 1018-1053, 1090-1125, 1162-1197, 1234-1269, 1306-1341, 1378-1413, 1450-1485, 1522-1557, 1594-1629, 1666-1701, 1862-1874, 1885, 1898, or fragments or variants thereof; Such a composition comprising at least one coding RNA encoding a secreted VP8* antigen and at least one coding RNA encoding a non-secreted antigen (cytosolic) may be advantageous as strong cellular and strong humoral immune responses, upon administration of the composition, may be induced.

In various particularly preferred embodiments, the composition of the second aspect comprises
(i) at least one coding RNA encoding at least one antigenic protein that is or is derived from VP8* (P2-VP8*) of a Rotavirus A from a P[4] serotype as specified herein, preferably according to SEQ ID NOs: 586-588, 595-597, 658-660, 667-669, 730-732, 739-741, 802-804, 811-813, 874-876, 883-885, 946-948, 955-957, 1018-1020, 1027-1029, 1090-1092, 1099-1101, 1162-1164, 1171-1173, 1234-1236, 1243-1245, 1306-1308, 1315-1317, 1378-1380, 1387-1389, 1450-1452, 1459-1461, 1522-1524, 1531-1533, 1594-1596, 1603-1605, 1666-1668, 1675-1677, 1911, 1917, 1923, 1929 or fragments or variants thereof;
(ii) at least one coding RNA encoding at least one antigenic protein that is or is derived from VP8* (P2-VP8*) of a Rotavirus A from a P[6] serotype as specified herein, preferably according to SEQ ID NOs: 589, 590, 598, 599, 661, 662, 670, 671, 733, 734, 742, 743, 805, 806, 814, 815, 877, 878, 886, 887, 949, 950, 958, 959, 1021, 1022, 1030, 1031, 1093, 1094, 1102, 1103, 1165, 1166, 1174, 1175, 1237, 1238, 1246, 1247, 1309, 1310, 1318, 1319, 1381, 1382, 1390, 1391, 1453, 1454, 1462, 1463, 1525, 1526, 1534, 1535, 1597, 1598, 1606, 1607, 1669, 1670, 1678, 1679, 1912, 1918, 1924, 1939, or fragments or variants thereof; and
(iii) at least one coding RNA encoding at least one antigenic protein that is or is derived from VP8* (P2-VP8*) of a Rotavirus A from a P[8] serotype as specified herein, according to SEQ ID NOs: 591-594, 600-603, 663-666, 672-675, 735-738, 744-747, 807-810, 816-819, 879-882, 888-891, 951-954, 960-963, 1023-1026, 1032-1035, 1095-1098, 1104-1107, 1167-1170, 1176-1179, 1239-1242, 1248-1251, 1311-1314, 1320-1323, 1383-1386, 1392-1395, 1455-1458, 1464-1467, 1527-1530, 1536-1539, 1599-1602, 1608-1611, 1671-1674, 1680-1683, 1862-1871, 1885, 1898, or fragments or variants thereof.

In various particularly preferred embodiments, the composition of the second aspect comprises
(i) at least one coding RNA encoding at least one antigenic protein that is or is derived from VP8* comprising the heterologous antigen clustering domain ferritin of a Rotavirus A from a P[4] serotype as specified herein (comprising the heterologous antigen clustering domain ferritin), preferably according to SEQ ID NOs: 604-606, 613-615, 676-678, 685-687, 748-750, 757-759, 820-822, 829-831, 892-894, 901-903, 964-966, 973-975, 1036-1038, 1045-1047, 1108-1110, 1117-1119, 1180-1182, 1189-1191, 1252-1254, 1261-1263, 1324-1326, 1333-1335, 1396-1398, 1405-1407, 1468-1470, 1477-1479, 1540-1542, 1549-1551, 1612-1614, 1621-1623, 1684-1686, 1693-1695, or fragments or variants thereof;
(ii) at least one coding RNA encoding at least one antigenic protein that is or is derived from VP8* of a Rotavirus A from a P[6] serotype as specified herein (comprising the heterologous antigen clustering domain ferritin), preferably according to SEQ ID NOs: 607, 608, 616, 617, 679, 680, 688, 689, 751, 752, 760, 761, 823, 824, 832, 833, 895, 896, 904, 905, 967, 968, 976, 977, 1039, 1040, 1048, 1049, 1111, 1112, 1120, 1121, 1183, 1184, 1192, 1193, 1255, 1256, 1264, 1265, 1327, 1328, 1336, 1337, 1399, 1400, 1408, 1409, 1471, 1472, 1480, 1481, 1543, 1544, 1552, 1553, 1615, 1616, 1624, 1625, 1687, 1688, 1696, 1697, or fragments or variants thereof; and
(iii) at least one coding RNA encoding at least one antigenic protein that is or is derived from VP8* of a Rotavirus A from a P[8] serotype as specified herein (comprising the heterologous antigen clustering domain ferritin), according to SEQ ID NOs: 609-612, 618-621, 681-684, 690-693, 753-756, 762-765, 825-828, 834-837, 897-900, 906-909, 969-972, 978-981, 1041-1044, 1050-1053, 1113-1116, 1122-1125, 1185-1188, 1194-1197, 1257-1260, 1266-1269, 1329-1332, 1338-1341, 1401-1404, 1410-1413, 1473-1476, 1482-1485, 1545-1548, 1554-1557, 1617-1620, 1626-1629, 1689-1692, 1698-1701, 1872-1874, or fragments or variants thereof.

In various particularly preferred embodiments, the composition of the second aspect comprises
(i) at least one coding RNA encoding at least one antigenic protein that is or is derived from VP8* of a Rotavirus A from a P[4] serotype as specified herein (comprising the heterologous antigen clustering domain lumazine synthase epitope), preferably according to SEQ ID NOs: 622-624, 631-633, 694-696, 703-705, 766-768, 775-777, 838-840, 847-849, 910-912, 919-921, 982-984, 991-993, 1054-1056, 1063-1065, 1126-1128, 1135-1137, 1198-1200, 1207-1209, 1270-1272, 1279-1281, 1342-1344, 1351-1353, 1414-1416, 1423-1425, 1486-1488, 1495-1497, 1558-1560, 1567-1569, 1630-1632, 1639-1641, 1702-1704, 1711-1713, 1911, 1917, 1923, 1929 or fragments or variants thereof;
(ii) at least one coding RNA encoding at least one antigenic protein that is or is derived from VP8* of a Rotavirus A from a P[6] serotype as specified herein (comprising the heterologous antigen clustering domain lumazine synthase epitope), preferably according to SEQ ID NOs: 625, 626, 634, 635, 697, 698, 706, 707, 769, 770, 778, 779, 841, 842, 850, 851, 913, 914, 922, 923, 985, 986, 994, 995, 1057, 1058, 1066, 1067, 1129, 1130, 1138, 1139, 1201, 1202, 1210, 1211, 1273, 1274, 1282, 1283, 1345, 1346, 1354, 1355, 1417, 1418, 1426, 1427, 1489, 1490, 1498, 1499, 1561, 1562, 1570, 1571, 1633, 1634, 1642, 1643, 1705, 1706, 1714, 1715, 1912, 1918, 1924, 1939 or fragments or variants thereof; and
(iii) at least one coding RNA encoding at least one antigenic protein that is or is derived from VP8* of a Rotavirus A from a P[8] serotype as specified herein (comprising the heterologous antigen clustering domain lumazine synthase epitope), according to SEQ ID NOs: 627-630, 636-639, 699-702, 708-711, 771-774, 780-783, 843-846, 862-855, 915-918, 924-927, 987-990, 996-999, 1059-1062, 1068-1071, 1131-1134, 1140-1143, 1203-1206, 1212-1215, 1275-1278, 1284-1287, 1347-1350, 1356-1359, 1419-1422, 1428-1431, 1491-1494, 1500-1503, 1563-1566, 1572-1575, 1635-1638, 1644-1647, 1707-1710, 1716-1719, 1875-1877, or fragments or variants thereof.

In various particularly preferred embodiments, the composition of the second aspect comprises
(i) at least one coding RNA encoding at least one antigenic protein that is or is derived from VP8* of a Rotavirus A from a P[4] serotype as specified herein (comprising the heterologous signal sequence IgE), preferably according to SEQ ID NOs: 640-642, 649-651, 712-714, 721-723, 784-786, 793-795, 856-858, 865-867, 928-930, 937-939, 1000-1002, 1009-1011, 1072-1074, 1081-1083, 1144-1146, 1153-1155, 1216-1218, 1225-1227, 1288-1290, 1297-1299, 1360-1362, 1369-1371, 1432-1434, 1441-1443, 1504-1506, 1513-1515, 1576-1578, 1585-1587, 1648-1650, 1657-1659, 1720-1722, 1729-1731, or fragments or variants thereof;
(ii) at least one coding RNA encoding at least one antigenic protein that is or is derived from VP8* of a Rotavirus A from a P[6] serotype as specified herein (comprising the heterologous signal sequence IgE), preferably according to SEQ ID NOs: 643, 644, 652, 653, 715, 716, 724, 725, 787, 788, 796, 797, 859, 860, 868, 869, 931, 932, 940, 941, 1003, 1004, 1012, 1013, 1075, 1076, 1084, 1085, 1147, 1148, 1156, 1157, 1219, 1220, 1228, 1229, 1291, 1292, 1300, 1301, 1363, 1364, 1372, 1373, 1435, 1436, 1444, 1445, 1507, 1508, 1516, 1517, 1579, 1580, 1588, 1589, 1651, 1652, 1660, 1661, 1723, 1724, 1732, 1733, or fragments or variants thereof; and
(iii) at least one coding RNA encoding at least one antigenic protein that is or is derived from VP8* of a Rotavirus A from a P[8] serotype as specified herein (comprising the heterologous signal sequence IgE), according to SEQ ID NOs: 645-648, 654-657, 717-720, 726-729, 789-792, 798-801, 861-864, 870-873, 933-936, 942-945, 1005-1008, 1014-1017, 1077-1080, 1086-1089, 1149-1152, 1158-1161, 1221-1224, 1230-1233, 1293-1296, 1302-1305, 1365-1368, 1374-1377, 1437-1440, 1446-1449, 1509-1512, 1518-1521, 1581-1584, 1590-1593, 1653-1656, 1662-1665, 1725-1728, 1734-1737, 1878-1880, or fragments or variants thereof.

In particularly preferred embodiments, the composition of the second aspect comprises
(i) at least one coding RNA encoding at least one antigenic protein that is or is derived from VP8* of a Rotavirus A from a P[4] serotype as specified herein, preferably according to SEQ ID NOs: 586-588, 595-597, 604-606, 613-615, 622-624, 631-633, 640-642, 649-651, 658-660, 667-669, 676-678, 685-687, 694-696, 703-705, 712-714, 721-723, 730-732, 739-741, 748-750, 757-759, 766-768, 775-777, 784-786, 793-795, 802-804, 811-813, 820-822, 829-831, 838-840, 847-849, 856-858, 865-867, 874-876, 883-885, 892-894, 901-903, 910-912, 919-921, 928-930, 937-939, 946-948, 955-957, 964-966, 973-975, 982-984, 991-993, 1000-1002, 1009-1011, 1018-1020, 1027-1029, 1036-1038, 1045-1047, 1054-1056, 1063-1065, 1072-1074, 1081-1083, 1090-1092, 1099-1101, 1108-1110, 1117-1119, 1126-1128, 1135-1137, 1144-1146, 1153-1155, 1162-1164, 1171-1173, 1180-1182, 1189-1191, 1198-1200, 1207-1209, 1216-1218, 1225-1227, 1234-1236, 1243-1245, 1252-1254, 1261-1263, 1270-1272, 1279-1281, 1288-1290, 1297-1299, 1306-1308, 1315-1317, 1324-1326, 1333-1335, 1342-1344, 1351-1353, 1360-1362, 1369-1371, 1378-1380, 1387-1389, 1396-1398, 1405-1407, 1414-1416, 1423-1425, 1432-1434, 1441-1443, 1450-1452, 1459-1461, 1468-1470, 1477-1479, 1486-1488, 1495-1497, 1504-1506, 1513-1515, 1522-1524, 1531-1533, 1540-1542, 1549-1551, 1558-1560, 1567-1569, 1576-1578, 1585-1587, 1594-1596, 1603-1605, 1612-1614, 1621-1623, 1630-1632, 1639-1641, 1648-1650, 1657-1659, 1666-1668, 1675-1677, 1684-1686, 1693-1695, 1702-1704, 1711-1713, 1720-1722, 1729-1731, 1886, 1911, 1917, 1923, 1929 or fragments or variants thereof;
(ii) at least one coding RNA encoding at least one antigenic protein that is or is derived from VP8* of a Rotavirus A from a P[6] serotype as specified herein, preferably according to SEQ ID NOs: 589, 590, 598, 599, 607, 608, 616, 617, 625, 626, 634, 635, 643, 644, 652, 653, 661, 662, 670, 671, 679, 680, 688, 689, 697, 698, 706, 707, 715, 716, 724, 725, 733, 734, 742, 743, 751, 752, 760, 761, 769, 770, 778, 779, 787, 788, 796, 797, 805, 806, 814, 815, 823, 824, 832, 833, 841, 842, 850, 851, 859, 860, 868, 869, 877, 878, 886, 887, 895, 896, 904, 905, 913, 914, 922, 923, 931, 932, 940, 941, 949, 950, 958, 959, 967, 968, 976, 977, 985, 986, 994, 995, 1003, 1004, 1012, 1013, 1021, 1022, 1030, 1031, 1039, 1040, 1048, 1049, 1057, 1058, 1066, 1067, 1075, 1076, 1084, 1085, 1093, 1094, 1102, 1103, 1111, 1112, 1120, 1121, 1129, 1130, 1138, 1139, 1147, 1148, 1156, 1157, 1165, 1166, 1174, 1175, 1183, 1184, 1192, 1193, 1201, 1202, 1210, 1211, 1219, 1220, 1228, 1229, 1237, 1238, 1246, 1247, 1255, 1256, 1264, 1265, 1273, 1274, 1282, 1283, 1291, 1292, 1300, 1301, 1309, 1310, 1318, 1319, 1327, 1328, 1336, 1337, 1345, 1346, 1354, 1355, 1363, 1364, 1372, 1373, 1381, 1382, 1390, 1391, 1399, 1400, 1408, 1409, 1417, 1418, 1426, 1427, 1435, 1436, 1444, 1445, 1453, 1454, 1462, 1463, 1471, 1472, 1480, 1481, 1489, 1490, 1498, 1499, 1507, 1508, 1516, 1517, 1525, 1526, 1534, 1535, 1543, 1544, 1552, 1553, 1561, 1562, 1570, 1571, 1579, 1580, 1588, 1589, 1597, 1598, 1606, 1607, 1615, 1616, 1624, 1625, 1633, 1634, 1642, 1643, 1651, 1652, 1660, 1661, 1669, 1670, 1678, 1679, 1687, 1688, 1696, 1697, 1705, 1706, 1714, 1715, 1723, 1724, 1732, 1733, 1887, 1890, 1895-1897, 1912, 1918, 1924, 1939 or fragments or variants thereof; and
(iii) at least one coding RNA encoding at least one antigenic protein that is or is derived from VP8* of a Rotavirus A from a P[8] serotype as specified herein, according to SEQ ID NOs: 591-594, 600-603, 609-612, 618-621, 627-630, 636-639, 645-648, 654-657, 663-666, 672-675, 681-684, 690-693, 699-702, 708-711, 717-720, 726-729, 735-738, 744-747, 753-756, 762-765, 771-774, 780-783, 789-792, 798-801, 807-810, 816-819, 825-828, 834-837, 843-846, 852-855, 861-864, 870-873, 879-882, 888-891, 897-900, 906-909, 915-918, 924-927, 933-936, 942-945, 951-954, 960-963, 969-972, 978-981, 987-990, 996-999, 1005-1008, 1014-1017, 1023-1026, 1032-1035, 1041-1044, 1050-1053, 1059-1062, 1068-1071, 1077-1080, 1086-1089, 1095-1098, 1104-1107, 1113-1116, 1122-1125, 1131-1134, 1140-1143, 1149-1152, 1158-1161, 1167-1170, 1176-1179, 1185-1188, 1194-1197, 1203-1206, 1212-1215, 1221-1224, 1230-1233, 1239-1242, 1248-1251, 1257-1260, 1266-1269, 1275-1278, 1284-1287, 1293-1296, 1302-1305, 1311-1314, 1320-1323, 1329-1332, 1338-1341, 1347-1350, 1356-1359, 1365-1368, 1374-1377, 1383-1386, 1392-1395, 1401-1404, 1410-1413, 1419-1422, 1428-1431, 1437-1440, 1446-1449, 1455-1458, 1464-1467, 1473-1476, 1482-1485, 1491-1494, 1500-1503, 1509-1512, 1518-1521, 1527-1530, 1536-1539, 1545-1548, 1554-1557, 1563-1566, 1572-1575, 1581-1584, 1590-1593, 1599-1602, 1608-1611, 1617-1620, 1626-1629, 1635-1638, 1644-1647, 1653-1656, 1662-1665, 1671-1674, 1680-1683, 1689-1692, 1698-1701, 1707-1710, 11716-1719, 1725-1728, 1734-1737, 1862-1882, 1885, 1888, 1889, 1891-1894, 1898, or fragments or variants thereof,

wherein the coding RNAs comprise a cap1 structure, preferably obtainable by co-transcriptional capping using a trinucleotide cap1 analog.

In particularly preferred embodiments, the composition of the second aspect comprises
(i) at least one coding RNA encoding at least one antigenic protein that is or is derived from VP8* of a Rotavirus A from a P[4] serotype as specified herein, preferably according to SEQ ID NOs: 586-588, 595-597, 604-606, 613-615, 622-624, 631-633, 640-642, 649-651, 658-660, 667-669, 676-678, 685-687, 694-696, 703-705, 712-714, 721-723, 730-732, 739-741, 748-750, 757-759, 766-768, 775-777, 784-786, 793-795, 802-804, 811-813, 820-822, 829-831, 838-840, 847-849, 856-858, 865-867, 874-876, 883-885, 892-894, 901-903, 910-912, 919-921, 928-930, 937-939, 946-948, 955-957, 964-966, 973-975, 982-984, 991-993, 1000-1002, 1009-1011, 1018-1020, 1027-1029, 1036-1038, 1045-1047, 1054-1056, 1063-1065, 1072-1074, 1081-1083, 1090-1092, 1099-1101, 1108-1110, 1117-1119, 1126-1128, 1135-1137, 1144-1146, 1153-1155, 1162-1164, 1171-1173, 1180-1182, 1189-1191, 1198-1200, 1207-1209, 1216-1218, 1225-1227, 1234-1236, 1243-1245, 1252-1254, 1261-1263, 1270-1272, 1279-1281, 1288-1290, 1297-1299, 1306-1308, 1315-1317, 1324-1326, 1333-1335, 1342-1344, 1351-1353, 1360-1362, 1369-1371, 1378-1380, 1387-1389, 1396-1398, 1405-1407, 1414-1416, 1423-1425, 1432-1434, 1441-1443, 1450-1452, 1459-1461, 1468-1470, 1477-1479, 1486-1488, 1495-1497, 1504-1506, 1513-1515, 1522-1524, 1531-1533, 1540-1542, 1549-1551, 1558-1560, 1567-1569, 1576-1578, 1585-1587, 1594-1596, 1603-1605, 1612-1614, 1621-1623, 1630-1632, 1639-1641, 1648-1650, 1657-1659, 1666-1668, 1675-1677, 1684-1686, 1693-1695, 1702-1704, 1711-1713, 1720-1722, 1729-1731, 1911, 1917, 1923, 1929 or fragments or variants thereof;
(ii) at least one coding RNA encoding at least one antigenic protein that is or is derived from VP8* of a Rotavirus A from a P[6] serotype as specified herein, preferably according to SEQ ID NOs: 589, 590, 598, 599, 607, 608, 616, 617, 625, 626, 634, 635, 643, 644, 652, 653, 661, 662, 670, 671, 679, 680, 688, 689, 697, 698, 706, 707, 715, 716, 724, 725, 733, 734, 742, 743, 751, 752, 760, 761, 769, 770, 778, 779, 787, 788, 796, 797, 805, 806, 814, 815, 823, 824, 832, 833, 841, 842, 850, 851, 859, 860, 868, 869, 877, 878, 886, 887, 895, 896, 904, 905, 913, 914, 922, 923, 931, 932, 940, 941, 949, 950, 958, 959, 967, 968, 976, 977, 985, 986, 994, 995, 1003, 1004, 1012, 1013, 1021, 1022, 1030, 1031, 1039, 1040, 1048, 1049, 1057, 1058, 1066, 1067, 1075, 1076, 1084, 1085, 1093, 1094, 1102, 1103, 1111, 1112, 1120, 1121, 1129, 1130, 1138, 1139, 1147, 1148, 1156, 1157, 1165, 1166, 1174, 1175, 1183, 1184, 1192, 1193, 1201, 1202, 1210, 1211, 1219, 1220, 1228, 1229, 1237, 1238, 1246, 1247, 1255, 1256, 1264, 1265, 1273, 1274, 1282, 1283, 1291, 1292, 1300, 1301, 1309, 1310, 1318, 1319, 1327, 1328, 1336, 1337, 1345, 1346, 1354, 1355, 1363, 1364, 1372, 1373, 1381, 1382, 1390, 1391, 1399, 1400, 1408, 1409, 1417, 1418, 1426, 1427, 1435, 1436, 1444, 1445, 1453, 1454, 1462, 1463, 1471, 1472, 1480, 1481, 1489, 1490, 1498, 1499, 1507, 1508, 1516, 1517, 1525, 1526, 1534, 1535, 1543, 1544, 1552, 1553, 1561, 1562, 1570, 1571, 1579, 1580, 1588, 1589, 1597, 1598, 1606, 1607, 1615, 1616, 1624, 1625, 1633, 1634, 1642, 1643, 1651, 1652, 1660, 1661, 1669, 1670, 1678, 1679, 1687, 1688, 1696, 1697, 1705, 1706, 1714, 1715, 1723, 1724, 1732, 1733, 1912, 1918, 1924, 1939, or fragments or variants thereof; and
(iii) at least one coding RNA encoding at least one antigenic protein that is or is derived from VP8* of a Rotavirus A from a P[8] serotype as specified herein, according to SEQ ID NOs: 591-594, 600-603, 609-612, 618-621, 627-630, 636-639, 645-648, 654-657, 663-666, 672-675, 681-684, 690-693, 699-702, 708-711, 717-720, 726-729, 735-738, 744-747, 753-756, 762-765, 771-774, 780-783, 789-792, 798-801, 807-810, 816-819, 825-828, 834-837, 843-846, 852-855, 861-864, 870-873, 879-882, 888-891, 897-900, 906-909, 915-918, 924-927, 933-936, 942-945, 951-954, 960-963, 969-972, 978-981, 987-990, 996-999, 1005-1008, 1014-1017, 1023-1026, 1032-1035, 1041-1044, 1050-1053, 1059-1062, 1068-1071, 1077-1080, 1086-1089, 1095-1098, 1104-1107, 1113-1116, 1122-1125, 1131-1134, 1140-1143, 1149-1152, 1158-1161, 1167-1170, 1176-1179, 1185-1188, 1194-1197, 1203-1206, 1212-1215, 1221-1224, 1230-1233, 1239-1242, 1248-1251, 1257-1260, 1266-1269, 1275-1278, 1284-1287, 1293-1296, 1302-1305, 1311-1314, 1320-1323, 1329-1332, 1338-1341, 1347-1350, 1356-1359, 1365-1368, 1374-1377, 1383-1386, 1392-1395, 1401-1404, 1410-1413, 1419-1422, 1428-1431, 1437-1440, 1446-1449, 1455-1458, 1464-1467, 1473-1476, 1482-1485, 1491-1494, 1500-1503, 1509-1512, 1518-1521, 1527-1530, 1536-1539, 1545-1548, 1554-1557, 1563-1566, 1572-1575, 1581-1584, 1590-1593, 1599-1602, 1608-1611, 1617-1620, 1626-1629, 1635-1638, 1644-1647, 1653-1656, 1662-1665, 1671-1674, 1680-1683, 1689-1692, 1698-1701, 1707-1710, 1716-1719, 1725-1728, 1734-1737, 1862, 1863, 1866, 1867, 1872, 1873, 1875, 1876, 1878, 1879, 1898, or fragments or variants thereof,

wherein the poly(A) sequence is suitably located at the 3′ terminus of the coding RNA and wherein preferably the coding RNAs comprise a cap1 structure, preferably obtainable by co-transcriptional capping using a trinucleotide cap1 analog.

In particularly preferred embodiments, the composition of the second aspect comprises
(i) at least one coding RNA encoding at least one antigenic protein that is or is derived from VP8* of a Rotavirus A from a P[4] serotype as specified herein, preferably according to SEQ ID NOs: 586-588, 595-597, 604-606, 613-615, 622-624, 631-633, 640-642, 649-651, 658-660, 667-669, 676-678, 685-687, 694-696, 703-705, 712-714, 721-723, 730-732, 739-741, 748-750, 757-759, 766-768, 775-777, 784-786, 793-795, 802-804, 811-813, 820-822, 829-831, 838-840, 847-849, 856-858, 865-867, 1162-1164, 1171-1173, 1180-1182, 1189-1191, 1198-1200, 1207-1209, 1216-1218, 1225-1227, 1234-1236, 1243-1245, 1252-1254, 1261-1263, 1270-1272, 1279-1281, 1288-1290, 1297-1299, 1306-1308, 1315-1317, 1324-1326, 1333-1335, 1342-1344, 1351-1353, 1360-1362, 1369-1371, 1378-1380, 1387-1389, 1396-1398, 1405-1407, 1414-1416, 1423-1425, 1432-1434, 1441-1443, 1911, 1917, 1923, 1929 or fragments or variants thereof;
(ii) at least one coding RNA encoding at least one antigenic protein that is or is derived from VP8* of a Rotavirus A from a P[6] serotype as specified herein, preferably according to SEQ ID NOs: 589, 590, 598, 599, 607, 608, 616, 617, 625, 626, 634, 635, 643, 644, 652, 653, 661, 662, 670, 671, 679, 680, 688, 689, 697, 698, 706, 707, 715, 716, 724, 725, 733, 734, 742, 743, 751, 752, 760, 761, 769, 770, 778, 779, 787, 788, 796, 797, 805, 806, 814, 815, 823, 824, 832, 833, 841, 842, 850, 851, 859, 860, 868, 869, 1165, 1166, 1174, 1175, 1183, 1184, 1192, 1193, 1201, 1202, 1210, 1211, 1219, 1220, 1228, 1229, 1237, 1238, 1246, 1247, 1255, 1256, 1264, 1265, 1273, 1274, 1282, 1283, 1291, 1292, 1300, 1301, 1309, 1310, 1318, 1319, 1327, 1328, 1336, 1337, 1345, 1346, 1354, 1355, 1363, 1364, 1372, 1373, 1381, 1382, 1390, 1391, 1399, 1400, 1408, 1409, 1417, 1418, 1426, 1427, 1435, 1436, 1444, 1445, 1912, 1918, 1924, 1939 or fragments or variants thereof; and
(iii) at least one coding RNA encoding at least one antigenic protein that is or is derived from VP8* of a Rotavirus A from a P[8] serotype as specified herein, according to SEQ ID NOs: 591-594, 600-603, 609-612, 618-621, 627-630, 636-639, 645-648, 654-657, 663-666, 672-675, 681-684, 690-693, 699-702, 708-711, 717-720, 726-729, 735-738, 744-747, 753-756, 762-765, 771-774, 780-783, 789-792, 798-801, 807-810, 816-819, 825-828, 834-837, 843-846, 852-855, 861-864, 870-873, 1167-1170, 1176-1179, 1185-1188, 1194-1197, 1203-1206, 1212-1215, 1221-1224, 1230-1233, 1239-1242, 1248-1251, 1257-1260, 1266-1269, 1275-1278, 1284-1287, 1293-1296, 1302-1305, 1311-1314, 1320-1323, 1329-1332, 1338-1341, 1347-1350, 1356-1359, 1365-1368, 1374-1377, 1383-1386, 1392-1395, 1401-1404, 1410-1413, 1419-1422, 1428-1431, 1437-1440, 1446-1449, 1862, 1863, 1866, 1867, 1872, 1873, 1875, 1876, 1878, 1879, or fragments or variants thereof,

wherein the coding RNA comprises at least one heterologous 5′-UTR derived from a 5′-UTR of a HSD17B4 gene and/or at least one heterologous 3′-UTR derived from a PSMB3 gene,

wherein the poly(A) sequence is suitably located at the 3′ terminus of the coding RNA and

wherein preferably the coding RNAs comprise a cap1 structure, preferably obtainable by co-transcriptional capping using a trinucleotide cap1 analogue.

Complexation:

In a preferred embodiment of the second aspect, the at least one coding RNA, or the plurality of coding RNAs (RNA species), is complexed or associated with to obtain a formulated composition. A formulation in that context may have the function of a transfection agent. A formulation in that context may also have the function of protecting the coding RNA from degradation.

In a preferred embodiment of the second aspect, the at least one coding RNA, or the plurality of coding RNAs (RNA species), is complexed or associated with or at least partially complexed or partially associated with one or more cationic or polycationic compound, preferably cationic or polycationic polymer, cationic or polycationic polysaccharide, cationic or polycationic lipid, cationic or polycationic protein, cationic or polycationic peptide, or any combinations thereof.

In embodiments where more than one or a plurality, e.g. 2, 3, 4, 5, 6, 7, 8, 9, 10 of the RNAs of the first aspect are comprised in the composition, said more than one or said plurality e.g. 2, 3, 4, 5, 6, 7, 8, 9, 10 of the RNAs may be complexed thereby forming complexes comprising more than one or a plurality, e.g. 2, 3, 4, 5, 6, 7, 8, 9, 10 of different RNAs (herein referred to as “co-formulation”)

Alternatively, in embodiments where more than one or a plurality, e.g. 2, 3, 4, 5, 6, 7, 8, 9, 10 of the RNAs of the first aspect are comprised in the composition, said more than one or said plurality e.g. 2, 3, 4, 5, 6, 7, 8, 9, 10 of the RNAs may be complexed as separate compositions e.g. as 2, 3, 4, 5, 6, 7, 8, 9, 10 separate compositions. Said separate compositions may be unified to form a composition comprising more than one or a plurality, e.g. 2, 3, 4, 5, 6, 7, 8, 9, 10 of the complexed RNA species.

The term “cationic or polycationic compound” as used herein will be recognized and understood by the person of ordinary skill in the art, and are for example intended to refer to a charged molecule, which is positively charged at a pH value ranging from about 1 to 9, at a pH value ranging from about 3 to 8, at a pH value ranging from about 4 to 8, at a pH value ranging from about 5 to 8, more preferably at a pH value ranging from about 6 to 8, even more preferably at a pH value ranging from about 7 to 8, most preferably at a physiological pH, e.g. ranging from about 7.2 to about 7.5. Accordingly, a cationic component, e.g. a cationic peptide, cationic protein, cationic polymer, cationic polysaccharide, cationic lipid may be any positively charged compound or polymer which is positively charged under physiological conditions. A “cationic or polycationic peptide or protein” may contain at least one positively charged amino acid, or more than one positively charged amino acid, e.g. selected from Arg, His, Lys or Orn. Accordingly, “polycationic” components are also within the scope exhibiting more than one positive charge under the given conditions.

Cationic or polycationic compounds, being particularly preferred in this context may be selected from the following list of cationic or polycationic peptides or proteins of fragments thereof: protamine, nucleoline, spermine or spermidine, or other cationic peptides or proteins, such as poly-L-lysine (PLL), poly-arginine, basic polypeptides, cell penetrating peptides (CPPs), including HIV-binding peptides, HIV-1 Tat (HIV), Tat-derived peptides, Penetratin, VP22 derived or analog peptides, HSV VP22 (Herpes simplex), MAP, KALA or protein transduction domains (PTDs), PpT620, prolin-rich peptides, arginine-rich peptides, lysine-rich peptides, MPG-peptide(s), Pep-1, L-oligomers, Calcitonin peptide(s), Antennapedia-derived peptides, pAntp, plsl, FGF, Lactoferrin, Transportan, Buforin-2, Bac715-24, SynB, SynB(1), pVEC, hCT-derived peptides, SAP, or histones. More preferably, the coding RNA, preferably the mRNA, is complexed with one or more polycations, preferably with protamine or oligofectamine, most preferably with protamine.

In preferred embodiment, the coding RNA, or the plurality of coding RNAs, is complexed with protamine.

Further preferred cationic or polycationic compounds, which can be used as transfection or complexation agent may include cationic polysaccharides, for example chitosan, polybrene etc.; cationic lipids, e.g. DOTMA, DMRIE, di-C14-amidine, DOTIM, SAINT, DC-Chol, BGTC, CTAP, DOPC, DODAP, DOPE: Dioleyl phosphatidylethanol-amine, DOSPA, DODAB, DOIC, DMEPC, DOGS, DIMRI, DOTAP, DC-6-14, CLIP1, CLIP6, CLIP9, oligofectamine; or cationic or polycationic polymers, e.g. modified polyaminoacids, such as beta-amino acid-polymers or reversed polyamides, etc., modified polyethylenes, such as PVP etc., modified acrylates, such as pDMAEMA etc., modified amidoamines such as pAMAM etc., modified polybetaaminoester (PBAE), such as diamine end modified 1,4 butanediol diacrylate-co-5-amino-1-pentanol polymers, etc., dendrimers, such as polypropylamine dendrimers or pAMAM based dendrimers, etc., polyimine(s), such as PEI, poly(propyleneimine), etc., polyallylamine, sugar backbone based polymers, such as cyclodextrin based polymers, dextran based polymers, etc., silan backbone based polymers, such as PMOXA-PDMS copolymers, etc., blockpolymers consisting of a combination of one or more cationic blocks (e.g. selected from a cationic polymer as mentioned above) and of one or more hydrophilic or hydrophobic blocks (e.g. polyethyleneglycole); etc.

In this context it is particularly preferred that the at least one coding RNA is complexed or at least partially complexed with a cationic or polycationic compound and/or a polymeric carrier, preferably cationic proteins or peptides. In this context, the disclosure of WO2010/037539 and WO2012/113513 is incorporated herewith by reference. Partially means that only a part of the coding RNA is complexed with a cationic compound and that the rest of the RNA is (comprised in the inventive (pharmaceutical) composition) in uncomplexed form (“free”).

In embodiments, the composition comprises at least one coding RNA complexed with one or more cationic or polycationic compounds, preferably protamine, and at least one free (non-complexed) coding RNA.

In this context it is particularly preferred that the at least one coding RNA is complexed, or at least partially complexed with protamine. Preferably, the molar ratio of the nucleic acid, particularly the RNA of the protamine-complexed RNA to the free RNA may be selected from a molar ratio of about 0.001:1 to about 1:0.001, including a ratio of about 1:1. Suitably, the complexed RNA is complexed with protamine by addition of protamine-trehalose solution to the RNA sample at a RNA:protamine weight to weight ratio (w/w) of 2:1.

Further preferred cationic or polycationic proteins or peptides that may be used for complexation can be derived from formula (Arg)l;(Lys)m;(His)n;(Orn)o;(Xaa)x of the patent application WO2009/030481 or WO2011/026641, the disclosure of WO2009/030481 or WO2011/026641 relating thereto incorporated herewith by reference.

In preferred embodiments, the at least one coding RNA is complexed, or at least partially complexed, with at least one cationic or polycationic proteins or peptides preferably selected from SEQ ID NOs: 1857-1861, or any combinations thereof.

According to various embodiments, the composition of the present invention comprises at least one coding RNA as defined in the context of the first aspect, and a polymeric carrier.

The term “polymeric carrier” as used herein will be recognized and understood by the person of ordinary skill in the art, and are e.g. intended to refer to a compound that facilitates transport and/or complexation of another compound (e.g. cargo RNA). A polymeric carrier is typically a carrier that is formed of a polymer. A polymeric carrier may be associated to its cargo (e.g. coding RNA) by covalent or non-covalent interaction. A polymer may be based on different subunits, such as a copolymer.

Suitable polymeric carriers in that context may include, for example, polyacrylates, polyalkycyanoacrylates, polylactide, polylactide-polyglycolide copolymers, polycaprolactones, dextran, albumin, gelatin, alginate, collagen, chitosan, cyclodextrins, protamine, PEGylated protamine, PEGylated PLL and polyethylenimine (PEI), dithiobis(succinimidylpropionate) (DSP), Dimethyl-3,3′-dithiobispropionimidate (DTBP), poly(ethylene imine) biscarbamate (PEIC), poly(L-lysine) (PLL), histidine modified PLL, poly(N-vinylpyrrolidone) (PVP), poly(propylenimine (PPI), poly(amidoamine) (PAMAM), poly(amido ethylenimine) (SS-PAEI), triehtylenetetramine (TETA), poly(β-aminoester), poly(4-hydroxy-L-proine ester) (PHP), poly(allylamine), poly(α-[4-aminobutyl]-L-glycolic acid (PAGA), Poly(D,L-lactic-co-glycolid acid (PLGA), Poly(N-ethyl-4-vinylpyridinium bromide), poly(phosphazene)s (PPZ), poly(phosphoester)s (PPE), poly(phosphoramidate)s (PPA), poly(N-2-hydroxypropylmethacrylamide) (pHPMA), poly(2-(dimethylamino)ethyl methacrylate) (pDMAEMA), poly(2-aminoethyl propylene phosphate) PPE_EA), galactosylated chitosan, N-dodecylated chitosan, histone, collagen and dextran-spermine. In one embodiment, the polymer may be an inert polymer such as, but not limited to, PEG. In one embodiment, the polymer may be a cationic polymer such as, but not limited to, PEI, PLL, TETA, poly(allylamine), Poly(N-ethyl-4-vinylpyridinium bromide), pHPMA and pDMAEMA. In one embodiment, the polymer may be a biodegradable PEI such as, but not limited to, DSP, DTBP and PEIC. In one embodiment, the polymer may be biodegradable such as, but not limited to, histine modified PLL, SS-PAEI, poly(β-aminoester), PHP, PAGA, PLGA, PPZ, PPE, PPA and PPE-EA.

A suitable polymeric carrier may be a polymeric carrier formed by disulfide-crosslinked cationic compounds. The disulfide-crosslinked cationic compounds may be the same or different from each other. The polymeric carrier can also contain further components. The polymeric carrier used according to the present invention may comprise mixtures of cationic peptides, proteins or polymers and optionally further components as defined herein, which are crosslinked by disulfide bonds (via —SH groups).

In this context, polymeric carriers according to formula {(Arg)l;(Lys)m;(His)n;(Orn)o;(Xaa′)x(Cys)y} and formula Cys,{(Arg)l;(Lys)m;(His)n;(Orn)o;(Xaa)x)Cys2 of the patent application WO2012/013326 are preferred, the disclosure of WO2012/013326 relating thereto incorporated herewith by reference.

In embodiments, the polymeric carrier used to complex the a least one coding RNA may be derived from a polymeric carrier molecule according formula (L-P¹—S—[S—P²—S]—S—P³-L) of the patent application WO2011/026641, the disclosure of WO2011/026641 relating thereto incorporated herewith by reference.

In embodiments, the polymeric carrier compound is formed by, or comprises or consists of the peptide elements CysArg12Cys (SEQ ID NO: 1857) or CysArg12 (SEQ ID NO: 1858) or TrpArg12Cys (SEQ ID NO: 1859). In particularly preferred embodiments, the polymeric carrier compound consists of a (R₁₂C)—(R₁₂C) dimer, a (WR₁₂C)—(WR₁₂C) dimer, or a (CR₁₂)—(CR₁₂C)—(CR₁₂) trimer, wherein the individual peptide elements in the dimer (e.g. (WR₁₂C)), or the trimer (e.g. (CR₁₂)), are connected via —SH groups.

In a preferred embodiment of the second aspect, the at least one coding RNA of the first aspect is complexed or associated with a polyethylene glycol/peptide polymer comprising HO-PEG5000-S-(S-CHHHHHHRRRRHHHHHHC-S-)7-S-PEG5000-OH (SEQ ID NO: 1860 as peptide monomer), HO-PEG5000-S-(S-CHHHHHHRRRRHHHHHHC-S-)4-S-PEG5000-OH (SEQ ID NO: 1860 as peptide monomer), HO-PEG5000-S-(S-CGHHHHHRRRRHHHHHGC-S-)7-S-PEG5000-OH (SEQ ID NO: 1861 as peptide monomer) and/or a polyethylene glycol/peptide polymer comprising HO-PEG5000-S-(S-CGHHHHHRRRRHHHHHGC-S-)4-S-PEG5000-OH (SEQ ID NO: 1861 of the peptide monomer).

In other embodiments, the composition comprises at least one coding RNA, wherein the at least one coding RNA is complexed or associated with polymeric carriers and, optionally, with at least one lipid component as described in WO2017/212008A1, WO2017/212006A1, WO2017/212007A1, and WO2017/212009A1. In this context, the disclosures of WO2017/212008A1, WO2017/212006A1, WO2017/212007A1, and WO2017/212009A1 are herewith incorporated by reference.

In a particularly preferred embodiment, the polymeric carrier (of the first and/or second component) is a peptide polymer, preferably a polyethylene glycol/peptide polymer as defined above, and a lipid component, preferably a lipidoid component.

A lipidoid (or lipidoit) is a lipid-like compound, i.e. an amphiphilic compound with lipid-like physical properties. The lipidoid is preferably a compound which comprises two or more cationic nitrogen atoms and at least two lipophilic tails. In contrast to many conventional cationic lipids, the lipidoid may be free of a hydrolysable linking group, in particular linking groups comprising hydrolysable ester, amide or carbamate groups. The cationic nitrogen atoms of the lipidoid may be cationisable or permanently cationic, or both types of cationic nitrogens may be present in the compound. In the context of the present invention the term lipid is considered to also encompass lipidoids.

In some embodiments of the inventions, the lipidoid may comprise a PEG moiety.

In preferred embodiments, the at least one coding RNA, preferably the mRNA, is complexed or associated with a polymeric carrier, preferably with a polyethylene glycol/peptide polymer as defined above, and a lipidoid component.

Suitably, the lipidoid is cationic, which means that it is cationisable or permanently cationic. In one embodiment, the lipidoid is cationisable, i.e. it comprises one or more cationisable nitrogen atoms, but no permanently cationic nitrogen atoms. In another embodiment, at least one of the cationic nitrogen atoms of the lipidoid is permanently cationic. Optionally, the lipidoid comprises two permanently cationic nitrogen atoms, three permanently cationic nitrogen atoms, or even four or more permanently cationic nitrogen atoms.

In a preferred embodiment, the lipidoid component may be any one selected from the lipidoids of the lipidoids provided in the table of page 50-54 of published PCT patent application WO2017/212009A1, the specific lipidoids provided in said table, and the specific disclosure relating thereto herewith incorporated by reference.

In preferred embodiments, the lipidoid component may be any one selected from 3-C12-OH, 3-C12-OH-cat, 3-C12-amide, 3-C12-amide monomethyl, 3-C12-amide dimethyl, RevPEG(10)-3-C12-OH, RevPEG(10)-DLin-pAbenzoic, 3C12amide-TMA cat., 3C12amide-DMA, 3C12amide-NH2, 3C12amide-OH, 3C12Ester-OH, 3C12 Ester-amin, 3C12Ester-DMA, 2C12Amid-DMA, 3C12-lin-amid-DMA, 2C12-sperm-amid-DMA, or 3C12-sperm-amid-DMA (see table of published PCT patent application WO2017/212009A1 (pages 50-54)). Particularly preferred are 3-C12-OH or 3-C12-OH-cat.

In preferred embodiments, the polyethylene glycol/peptide polymer comprising a lipidoid as specified above (e.g. 3-C12-OH or 3-C12-OH-cat), is used to complex the at least one coding RNA to form complexes having an N/P ratio from about 0.1 to about 20, or from about 0.2 to about 15, or from about 2 to about 15, or from about 2 to about 12, wherein the N/P ratio is defined as the mole ratio of the nitrogen atoms of the basic groups of the cationic peptide or polymer to the phosphate groups of the nucleic acid. In that context, the disclosure of published PCT patent application WO2017/212009A1, in particular claims 1 to 10 of WO2017/212009A1, and the specific disclosure relating thereto is herewith incorporated by reference.

Further suitable lipidoids may be derived from published PCT patent application WO2010/053572. In particular, lipidoids derivable from claims 1 to 297 of published PCT patent application WO2010/053572 may be used in the context of the invention, e.g. incorporated into the peptide polymer as described herein, or e.g. incorporated into the lipid nanoparticle (as described below). Accordingly, claims 1 to 297 of published PCT patent application WO2010/053572, and the specific disclosure relating thereto, is herewith incorporated by reference.

Encapsulation/Complexation in LNPs:

In preferred embodiments of the second aspect, the at least one coding RNA, preferably the plurality of coding RNAs, is complexed, encapsulated, partially encapsulated, or associated with one or more lipids (e.g. cationic lipids and/or neutral lipids), thereby forming liposomes, lipid nanoparticles (LNPs), lipoplexes, and/or nanoliposomes.

The liposomes, lipid nanoparticles (LNPs), lipoplexes, and/or nanoliposomes—incorporated RNA may be completely or partially located in the interior space of the liposomes, lipid nanoparticles (LNPs), lipoplexes, and/or nanoliposomes, within the lipid layer/membrane, or associated with the exterior surface of the lipid layer/membrane. The incorporation of a nucleic acid into liposomes/LNPs is also referred to herein as “encapsulation” wherein the RNA is entirely contained within the interior space of the liposomes, lipid nanoparticles (LNPs), lipoplexes, and/or nanoliposomes. The purpose of incorporating coding RNA into liposomes, lipid nanoparticles (LNPs), lipoplexes, and/or nanoliposomes is to protect the RNA from an environment which may contain enzymes or chemicals or conditions that degrade RNA and/or systems or receptors that cause the rapid excretion of the RNA. Moreover, incorporating coding RNA into liposomes, lipid nanoparticles (LNPs), lipoplexes, and/or nanoliposomes may promote the uptake of the RNA, and hence, may enhance the therapeutic effect of the RNA encoding antigenic Rotavirus proteins (e.g., VP8*). Accordingly, incorporating an coding RNA, or a plurality of coding RNA species into liposomes, lipid nanoparticles (LNPs), lipoplexes, and/or nanoliposomes may be particularly suitable for a Rotavirus vaccine, e.g. for intramuscular administration.

In this context, the terms “complexed” or “associated” refer to the essentially stable combination of coding RNA with one or more lipids into larger complexes or assemblies without covalent binding.

The term “lipid nanoparticle”, also referred to as “LNP”, is not restricted to any particular morphology, and include any morphology generated when a cationic lipid and optionally one or more further lipids are combined, e.g. in an aqueous environment and/or in the presence of RNA. For example, a liposome, a lipid complex, a lipoplex and the like are within the scope of a lipid nanoparticle (LNP).

Liposomes, lipid nanoparticles (LNPs), lipoplexes, and/or nanoliposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 nm and 500 nm in diameter.

LNPs of the invention are suitably characterized as microscopic vesicles having an interior aqua space sequestered from an outer medium by a membrane of one or more bilayers. Bilayer membranes of LNPs are typically formed by amphiphilic molecules, such as lipids of synthetic or natural origin that comprise spatially separated hydrophilic and hydrophobic domains. Bilayer membranes of the liposomes can also be formed by amphiphilic polymers and surfactants (e.g., polymerosomes, niosomes, etc.). In the context of the present invention, an LNP typically serves to transport the coding RNA, or the plurality of coding RNA species, to a target tissue.

Accordingly, in preferred embodiments of the second aspect, the at least one RNA, or the plurality of coding RNAs, is complexed with one or more lipids thereby forming lipid nanoparticles (LNP). Preferably, said LNP is particularly suitable for intramuscular and/or intradermal administration.

LNPs typically comprise a cationic lipid and one or more excipients selected from neutral lipids, charged lipids, steroids and polymer conjugated lipids (e.g. PEGylated lipid). The coding RNA may be encapsulated in the lipid portion of the LNP or an aqueous space enveloped by some or the entire lipid portion of the LNP. The coding RNA or a portion thereof may also be associated and complexed with the LNP. An LNP may comprise any lipid capable of forming a particle to which the nucleic acids are attached, or in which the one or more nucleic acids are encapsulated. Preferably, the LNP comprising nucleic acids comprises one or more cationic lipids, and one or more stabilizing lipids. Stabilizing lipids include neutral lipids and PEGylated lipids.

The cationic lipid of an LNP may be cationisable, i.e. it becomes protonated as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

Such lipids include, but are not limited to, DSDMA, N,N-dioleyl-N,N-dimethylammonium chloride (DODAC), N,N-distearyl-N,N-dimethylammonium bromide (DDAB), 1,2-dioleoyltrimethyl ammonium propane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), ckk-E12, ckk, 1,2-DiLinoleyloxy-N,N-dimethylaminopropane (DLinDMA), 1,2-Dilinolenyloxy-N,N-dimethylaminopropane (DLenDMA), 1,2-di-γ-linolenyloxy-N,N-dimethylaminopropane (γ-DLenDMA), 98N12-5, 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), ICE (Imidazol-based), HGT5000, HGT5001, DMDMA, CLinDMA, CpLinDMA, DMOBA, DOcarbDAP, DLincarbDAP, DLinCDAP, KLin-K-DMA, DLin-K-XTC2-DMA, XTC (2,2-Dilinoleyl-4-dimethylaminoethyl-[1,3]-dioxolane) HGT4003, 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DM A), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl-4-(dimethylamino)butanoate (MC3), ALNY-100 ((3aR,5s,6aS)—N,N-dimethyl-2,2-di((9Z,12Z)-octadeca-9,12-dienyl)tetrahydro-3aH-cyclopenta[d] [1,3]dioxol-5-amine)), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), NC98-5 (4,7, 13-tris(3-oxo-3-(undecylamino)propyl)-NI,N 16-diundecyl-4,7, 10,13-tetraazahexadecane-1,16-diamide), (6Z,9Z,28Z,31Z)-heptatriaconta-6,9,28,31-tetraen-19-yl 4-(dimethylamino) butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31Z)-heptatriaconta-6,9,28,31-tetraen-19-yloxy)-N,N-dimethylbutan-1-amine (MC4 Ether), LIPOFECTIN® (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.) or any combination of any of the foregoing. Further suitable cationic lipids for use in the compositions and methods of the invention include those described in international patent publications WO2010/053572 (and particularly, CI 2-200 described at paragraph [00225]) and WO2012/170930, both of which are incorporated herein by reference, HGT4003, HGT5000, HGTS001, HGT5001, HGT5002 (see US20150140070A1).

In embodiments, the cationic lipid may be an amino lipid.

Representative amino lipids include, but are not limited to, 1,2-dilinoleyoxy-3-(dimethylamino)acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA), 2,2-dilinoleyl-4-(2-dimethylaminoethyl)-[1,3]-dioxolane (DLin-KC2-DMA); dilinoleyl-methyl-4-dimethylaminobutyrate (DLin-MC3-DMA); MC3 (US20100324120).

In embodiments, the cationic lipid may be an aminoalcohol lipidoid.

Aminoalcohol lipidoids which may be used in the present invention may be prepared by the methods described in U.S. Pat. No. 8,450,298, herein incorporated by reference in its entirety. Suitable (ionizable) lipids can also be the compounds as disclosed in Tables 1, 2 and 3 and as defined in claims 1-24 of WO2017/075531A1, hereby incorporated by reference.

In another embodiment, suitable lipids can also be the compounds as disclosed in WO2015/074085A1 (i.e. ATX-001 to ATX-032 or the compounds as specified in claims 1-26), U.S. Appl. Nos. 61/905,724 and Ser. No. 15/614,499 or U.S. Pat. Nos. 9,593,077 and 9,567,296 hereby incorporated by reference in their entirety.

In other embodiments, suitable cationic lipids can also be the compounds as disclosed in WO2017/117530A1 (i.e. lipids 13, 14, 15, 16, 17, 18, 19, 20, or the compounds as specified in the claims), hereby incorporated by reference in its entirety.

In preferred embodiments, ionizable or cationic lipids may also be selected from the lipids disclosed in WO2018/078053A1 (i.e. lipids derived from formula I, II, and III of WO2018/078053A1, or lipids as specified in Claims 1 to 12 of WO2018/078053A1), the disclosure of WO2018/078053A1 hereby incorporated by reference in its entirety. In that context, lipids disclosed in Table 7 of WO2018/078053A1 (e.g. lipids derived from formula I-1 to I-41) and lipids disclosed in Table 8 of WO2018/078053A1 (e.g. lipids derived from formula II-1 to II-36) may be suitably used in the context of the invention. Accordingly, formula I-1 to formula I-41 and formula II-1 to formula II-36 of WO2018/078053A1, and the specific disclosure relating thereto, are herewith incorporated by reference.

In preferred embodiments, cationic lipids may be derived from formula III of published PCT patent application WO2018/078053A1. Accordingly, formula III of WO2018/078053A1, and the specific disclosure relating thereto, are herewith incorporated by reference.

In particularly preferred embodiments, the at least one coding RNA or the plurality of coding RNA species of the composition is complexed with one or more lipids thereby forming LNPs, wherein the cationic lipid of the LNP is selected from structures II-1 to III-36 of Table 9 of published PCT patent application WO2018/078053A1. Accordingly, formula III-1 to III-36 of WO2018/078053A1, and the specific disclosure relating thereto, are herewith incorporated by reference.

In particularly preferred embodiment of the second aspect, the coding RNA or the plurality of coding RNAs is complexed with one or more lipids thereby forming LNPs, wherein the LNPs comprises a cationic lipid according to formula III-3:

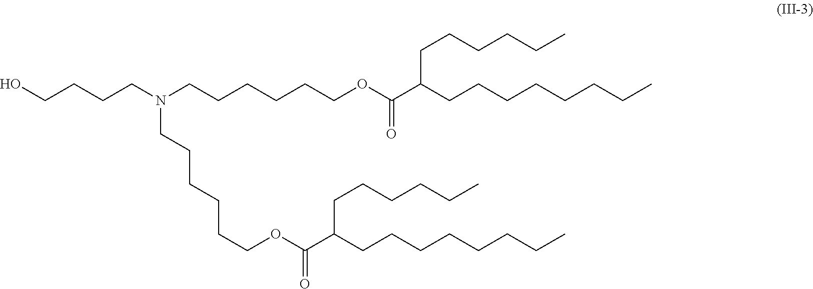 [SMILES: CCCCCCCCC(CCCCCC)C(=O)OCCCCCCN(CCCCO)CCCCCCOC(=O)C(CCCCCC)CCCCCCCC]

In certain embodiments, the cationic lipid as defined herein, more preferably cationic lipid compound III-3, is present in the LNP in an amount from about 30 to about 95 mole percent, relative to the total lipid content of the LNP. If more than one cationic lipid is incorporated within the LNP, such percentages apply to the combined cationic lipids.

In embodiments, the cationic lipid is present in the LNP in an amount from about 30 to about 70 mole percent. In one embodiment, the cationic lipid is present in the LNP in an amount from about 40 to about 60 mole percent, such as about 40, 41, 42, 43, 44, 45, 46, 47, 48, 49, 50, 51, 52, 53, 54, 55, 56, 57, 58, 59 or 60 mole percent, respectively. In embodiments, the cationic lipid is present in the LNP in an amount from about 47 to about 48 mole percent, such as about 47.0, 47.1, 47.2, 47.3, 47.4, 47.5, 47.6, 47.7, 47.8, 47.9, 50.0 mole percent, respectively, wherein 47.7 mole percent are particularly preferred.

In some embodiments, the cationic lipid is present in a ratio of from about 20 mol % to about 70 or 75 mol % or from about 45 to about 65 mol % or about 20, 25, 30, 35, 40, 45, 50, 55, 60, 65, or about 70 mol % of the total lipid present in the LNP. In further embodiments, the LNPs comprise from about 25% to about 75% on a molar basis of cationic lipid, e.g., from about 20 to about 70%, from about 35 to about 65%, from about 45 to about 65%, about 60%, about 57.5%, about 57.1%, about 50% or about 40% on a molar basis (based upon 100% total moles of lipid in the lipid nanoparticle). In some embodiments, the ratio of cationic lipid to coding RNA is from about 3 to about 15, such as from about 5 to about 13 or from about 7 to about 11.

Other suitable (cationic or ionizable) lipids are disclosed in WO2009/086558, WO2009/127060, WO2010/048536, WO2010/054406, WO2010/088537, WO2010/129709, WO2011/153493, WO 2013/063468, US2011/0256175, US2012/0128760, US2012/0027803, U.S. Pat. No. 8,158,601, WO2016/118724, WO2016/118725, WO2017/070613, WO2017/070620, WO2017/099823, WO2012/040184, WO2011/153120, WO2011/149733, WO2011/090965, WO2011/043913, WO2011/022460, WO2012/061259, WO2012/054365, WO2012/044638, WO2010/080724, WO2010/21865, WO2008/103276, WO2013/086373, WO2013/086354, U.S. Pat. Nos. 7,893,302, 7,404,969, 8,283,333, 8,466,122 and 8,569,256 and US Patent Publication No. US2010/0036115, US2012/0202871, US2013/0064894, US2013/0129785, US2013/0150625, US2013/0178541, US2013/0225836, US2014/0039032 and WO2017/112865. In that context, the disclosures of WO2009/086558, WO2009/127060, WO2010/048536, WO2010/054406, WO2010/088537, WO2010/129709, WO2011/153493, WO 2013/063468, US2011/0256175, US2012/0128760, US2012/0027803, U.S. Pat. No. 8,158,601, WO2016/118724, WO2016/118725, WO2017/070613, WO2017/070620, WO2017/099823, WO2012/040184, WO2011/153120, WO2011/149733, WO2011/090965, WO2011/043913, WO2011/022460, WO2012/061259, WO2012/054365, WO2012/044638, WO2010/080724, WO2010/21865, WO2008/103276, WO2013/086373, WO2013/086354, U.S. Pat. Nos. 7,893,302, 7,404,969, 8,283,333, 8,466,122 and 8,569,256 and US Patent Publication No. US2010/0036115, US2012/0202871, US2013/0064894, US2013/0129785, US2013/0150625, US2013/0178541, US2013/0225836 and US2014/0039032 and WO2017/112865 specifically relating to (cationic) lipids suitable for LNPs are incorporated herewith by reference.

In embodiments, amino or cationic lipids as defined herein have at least one protonatable or deprotonatable group, such that the lipid is positively charged at a pH at or below physiological pH (e.g. pH 7.4), and neutral at a second pH, preferably at or above physiological pH. It will, of course, be understood that the addition or removal of protons as a function of pH is an equilibrium process, and that the reference to a charged or a neutral lipid refers to the nature of the predominant species and does not require that all of lipids have to be present in the charged or neutral form. Lipids having more than one protonatable or deprotonatable group, or which are zwitterionic, are not excluded and may likewise suitable in the context of the present invention. In some embodiments, the protonatable lipids have a pKa of the protonatable group in the range of about 4 to about 11, e.g., a pKa of about 5 to about 7.

LNPs can comprise two or more (different) cationic lipids as defined herein. Cationic lipids may be selected to contribute to different advantageous properties. For example, cationic lipids that differ in properties such as amine pKa, chemical stability, half-life in circulation, half-life in tissue, net accumulation in tissue, or toxicity can be used in the LNP. In particular, the cationic lipids can be chosen so that the properties of the mixed-LNP are more desirable than the properties of a single-LNP of individual lipids.

The amount of the permanently cationic lipid or lipidoid may be selected taking the amount of the RNA cargo into account. In one embodiment, these amounts are selected such as to result in an N/P ratio of the nanoparticle(s) or of the composition in the range from about 0.1 to about 20. In this context, the N/P ratio is defined as the mole ratio of the nitrogen atoms (“N”) of the basic nitrogen-containing groups of the lipid or lipidoid to the phosphate groups (“P”) of the RNA which is used as cargo. The N/P ratio may be calculated on the basis that, for example, 1 ug RNA typically contains about 3 nmol phosphate residues, provided that the RNA exhibits a statistical distribution of bases. The “N”-value of the lipid or lipidoid may be calculated on the basis of its molecular weight and the relative content of permanently cationic and—if present—cationisable groups.

In vivo characteristics and behavior of LNPs can be modified by addition of a hydrophilic polymer coating, e.g. polyethylene glycol (PEG), to the LNP surface to confer steric stabilization. Furthermore, LNPs can be used for specific targeting by attaching ligands (e.g. antibodies, peptides, and carbohydrates) to its surface or to the terminal end of the attached PEG chains (e.g. via PEGylated lipids or PEGylated cholesterol).

In some embodiments, the LNPs comprise a polymer conjugated lipid. The term “polymer conjugated lipid” refers to a molecule comprising both a lipid portion and a polymer portion. An example of a polymer conjugated lipid is a PEGylated lipid. The term “PEGylated lipid” refers to a molecule comprising both a lipid portion and a polyethylene glycol portion. PEGylated lipids are known in the art and include 1-(monomethoxy-polyethyleneglycol)-2,3-dimyristoylglycerol (PEG-s-DMG) and the like.

In certain embodiments, the LNP comprises a stabilizing-lipid which is a polyethylene glycol-lipid (PEGylated lipid). Suitable polyethylene glycol-lipids include PEG-modified phosphatidylethanolamine, PEG-modified phosphatidic acid, PEG-modified ceramides (e.g. PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N-[(methoxy poly(ethylene glycol)2000)carbamyl]-1,2-dimyristyloxlpropyl-3-amine (PEG-c-DMA). In a preferred embodiment, the polyethylene glycol-lipid is PEG-2000-DMG. In one embodiment, the polyethylene glycol-lipid is PEG-c-DOMG). In other embodiments, the LNPs comprise a PEGylated diacylglycerol (PEG-DAG) such as 1-(monomethoxy-polyethyleneglycol)-2,3-dimyristoylglycerol (PEG-DMG), a PEGylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a PEGylated ceramide (PEG-cer), or a PEG dialkoxypropylcarbamate such as ω-methoxy(polyethoxy)ethyl-N-(2,3di(tetradecanoxy)propyl)carbamate or 2,3-di(tetradecanoxy)propyl-N-(ω-methoxy(polyethoxy)ethyl)carbamate.

In preferred embodiments, the PEGylated lipid is preferably derived from formula (IV) of published PCT patent application WO2018/078053A1. Accordingly, PEGylated lipids derived from formula (IV) of published PCT patent application WO2018/078053A1, and the respective disclosure relating thereto, are herewith incorporated by reference.

In a particularly preferred embodiments, the at least one coding RNA of the composition is complexed with one or more lipids thereby forming LNPs, wherein the LNP comprises a PEGylated lipid, wherein the PEG lipid is preferably derived from formula (IVa) of published PCT patent application WO2018/078053A1. Accordingly, PEGylated lipid derived from formula (IVa) of published PCT patent application WO2018/078053A1, and the respective disclosure relating thereto, is herewith incorporated by reference.

In a particularly preferred embodiment of the second aspect, the coding RNA or the plurality of coding RNA species is complexed with one or more lipids thereby forming lipid nanoparticles (LNP), wherein the LNP comprises a PEGylated lipid/PEG lipid. Preferably, said PEG lipid is of formula (IVa):

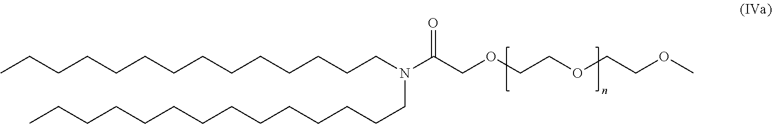

wherein n has a mean value ranging from 30 to 60, such as about 30±2, 32±2, 34±2, 36±2, 38±2, 40±2, 42±2, 44±2, 46±2, 48±2, 50±2, 52±2, 54±2, 56±2, 58±2, or 60±2. In a most preferred embodiment n is about 49.

Further examples of PEG-lipids suitable in that context are provided in US2015/0376115A1 and WO2015/199952, each of which is incorporated by reference in its entirety.

In some embodiments, LNPs include less than about 3, 2, or 1 mole percent of PEG or PEG-modified lipid, based on the total moles of lipid in the LNP. In further embodiments, LNPs comprise from about 0.1% to about 20% of the PEG-modified lipid on a molar basis, e.g., about 0.5 to about 10%, about 0.5 to about 5%, about 10%, about 5%, about 3.5%, about 3%, about 2.5%, about 2%, about 1.5%, about 1%, about 0.5%, or about 0.3% on a molar basis (based on 100% total moles of lipids in the LNP). In preferred embodiments, LNPs comprise from about 1.0% to about 2.0% of the PEG-modified lipid on a molar basis, e.g., about 1.2 to about 1.9%, about 1.2 to about 1.8%, about 1.3 to about 1.8%, about 1.4 to about 1.8%, about 1.5 to about 1.8%, about 1.6 to about 1.8%, in particular about 1.4%, about 1.5%, about 1.6%, about 1.7%, about 1.8%, about 1.9%, most preferably 1.7% (based on 100% total moles of lipids in the LNP). In various embodiments, the molar ratio of the cationic lipid to the PEGylated lipid ranges from about 100:1 to about 25:1.

In preferred embodiments, the LNP comprises one or more additional lipids which stabilize the formation of particles during their formation or during the manufacturing process (e.g. neutral lipid and/or one or more steroid or steroid analogue).

In preferred embodiments of the second aspect, the coding RNA or the plurality of coding RNAs is complexed with one or more lipids thereby forming lipid nanoparticles (LNP), wherein the LNP comprises one or more neutral lipid and/or one or more steroid or steroid analogue.

Suitable stabilizing lipids include neutral lipids and anionic lipids. The term “neutral lipid” refers to any one of a number of lipid species that exist in either an uncharged or neutral zwitterionic form at physiological pH. Representative neutral lipids include diacylphosphatidylcholines, diacylphosphatidylethanolamines, ceramides, sphingomyelins, dihydro sphingomyelins, cephalins, and cerebrosides.

In embodiments of the second aspect, the LNP comprises one or more neutral lipids, wherein the neutral lipid is selected from the group comprising distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE), or mixtures thereof.

In some embodiments, the LNPs comprise a neutral lipid selected from DSPC, DPPC, DMPC, DOPC, POPC, DOPE and SM. In various embodiments, the molar ratio of the cationic lipid to the neutral lipid ranges from about 2:1 to about 8:1.

In preferred embodiments, the neutral lipid is 1,2-distearoyl-sn-glycero-3-phosphocholine (DSPC). The molar ratio of the cationic lipid to DSPC may be in the range from about 2:1 to about 8:1.

In preferred embodiments, the steroid is cholesterol. The molar ratio of the cationic lipid to cholesterol may be in the range from about 2:1 to about 1:1. In some embodiments, the cholesterol may be PEGylated.

The sterol can be about 10 mol % to about 60 mol % or about 25 mol % to about 40 mol % of the lipid particle. In one embodiment, the sterol is about 10, 15, 20, 25, 30, 35, 40, 45, 50, 55, or about 60 mol % of the total lipid present in the lipid particle. In another embodiment, the LNPs include from about 5% to about 50% on a molar basis of the sterol, e.g., about 15% to about 45%, about 20% to about 40%, about 48%, about 40%, about 38.5%, about 35%, about 34.4%, about 31.5% or about 31% on a molar basis (based upon 100% total moles of lipid in the lipid nanoparticle).

Preferably, lipid nanoparticles (LNPs) comprise: (a) at least one coding RNA or a plurality of coding RNAs of the first aspect, (b) a cationic lipid, (c) an aggregation reducing agent (such as polyethylene glycol (PEG) lipid or PEG-modified lipid), (d) optionally a non-cationic lipid (such as a neutral lipid), and (e) optionally, a sterol.

In some embodiments, the cationic lipids (as defined above), non-cationic lipids (as defined above), cholesterol (as defined above), and/or PEG-modified lipids (as defined above) may be combined at various relative molar ratios. For example, the ratio of cationic lipid to non-cationic lipid to cholesterol-based lipid to PEGylated lipid may be between about 30-60:20-35:20-30:1-15, or at a ratio of about 40:30:25:5, 50:25:20:5, 50:27:20:3, 40:30:20:10, 40:32:20:8, 40:32:25:3 or 40:33:25:2, or at a ratio of about 50:25:20:5, 50:20:25:5, 50:27:20:3 40:30:20:10, 40:30:25:5 or 40:32:20:8, 40:32:25:3 or 40:33:25:2, respectively.

In some embodiments, the LNPs comprise a lipid of formula (III), at least one coding RNA or a plurality of coding RNAs as defined herein, a neutral lipid, a steroid and a PEGylated lipid. In preferred embodiments, the lipid of formula (III) is lipid compound III-3, the neutral lipid is DSPC, the steroid is cholesterol, and the PEGylated lipid is the compound of formula (IVa).

In a preferred embodiment of the second aspect, the LNP consists essentially of (i) at least one cationic lipid; (ii) a neutral lipid; (iii) a sterol, e.g., cholesterol; and (iv) a PEG-lipid, e.g. PEG-DMG or PEG-cDMA, in a molar ratio of about 20-60% cationic lipid:5-25% neutral lipid:25-55% sterol; 0.5-15% PEG-lipid.

In particularly preferred embodiments, the coding RNA, or the plurality of coding RNAs is complexed with one or more lipids thereby forming lipid nanoparticles (LNP), wherein the LNP comprises
(i) at least one cationic lipid as defined herein, preferably a lipid of formula (III), more preferably lipid III-3;
(ii) at least one neutral lipid as defined herein, preferably 1,2-distearoyl-sn-glycero-3-phosphocholine (DSPC);
(iii) at least one steroid or steroid analogue as defined herein, preferably cholesterol; and
(iv) at least one PEG-lipid as defined herein, e.g. PEG-DMG or PEG-cDMA, preferably a PEGylated lipid that is or is derived from formula (iVa).

In particularly preferred embodiments, the coding RNA, or the plurality of coding RNAs is complexed with one or more lipids thereby forming lipid nanoparticles (LNP), wherein the LNP comprises (i) to (iv) in a molar ratio of about 20-60% cationic lipid:5-25% neutral lipid:25-55% sterol; 0.5-15% PEG-lipid.

In one preferred embodiment, the lipid nanoparticle comprises: a cationic lipid with formula (III) and/or PEG lipid with formula (IV), optionally a neutral lipid, preferably 1,2-distearoyl-sn-glycero-3-phosphocholine (DSPC) and optionally a steroid, preferably cholesterol, wherein the molar ratio of the cationic lipid to DSPC is optionally in the range from about 2:1 to 8:1, wherein the molar ratio of the cationic lipid to cholesterol is optionally in the range from about 2:1 to 1:1.

In a particular preferred embodiment, the composition of the second aspect comprising the coding RNA or a plurality of coding RNA species, comprises lipid nanoparticles (LNPs), which have a molar ratio of approximately 50:10:38.5:1.5, preferably 47.5:10:40.8:1.7 or more preferably 47.4:10:40.9:1.7 (i.e. proportion (mol %) of cationic lipid (preferably lipid III-3), DSPC, cholesterol and PEG-lipid (preferably PEG-lipid of formula (IVa) with n=49); solubilized in ethanol).

The total amount of RNA in the lipid nanoparticles may vary and is defined depending on the e.g. RNA to total lipid w/w ratio. In one embodiment of the invention the RNA to total lipid ratio is less than 0.06 w/w, preferably between 0.03 w/w and 0.04 w/w.

In some embodiments, the composition comprises lipid nanoparticles (LNPs), which are composed of only three lipid components, namely imidazole cholesterol ester (ICE), 1,2-dioleoyl-sn-glycero-3-phosphoethanolamine (DOPE), and 1,2-dimyristoyl-sn-glycerol, methoxypolyethylene glycol (DMG-PEG-2K).

In one embodiment, the lipid nanoparticle of the composition comprises a cationic lipid, a steroid; a neutral lipid; and a polymer conjugated lipid, preferably a pegylated lipid. Preferably, the polymer conjugated lipid is a pegylated lipid or PEG-lipid. In a specific embodiment, lipid nanoparticles comprise a cationic lipid resembled by the cationic lipid COATSOME® SS-EC (former name: SS-33/4PE-15; NOF Corporation, Tokyo, Japan), in accordance with the following formula

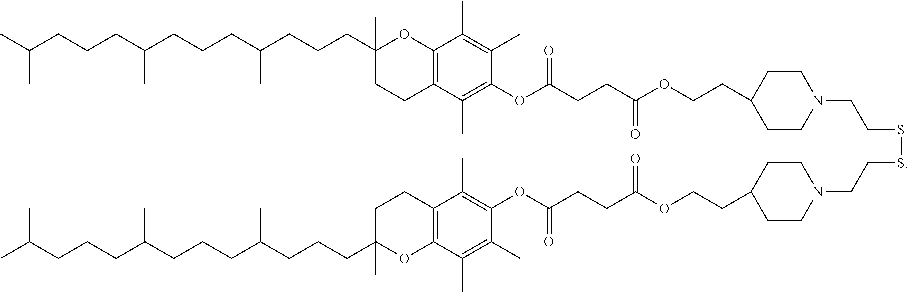 [SMILES: Cc1c(C)c2c(c(C)c1OC(=O)CCC(=O)OCCC1CCN(CCSSCCN3CCC(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2]

A described further below, those lipid nanoparticles are termed “GN01”.

Furthermore, in a specific embodiment, the GN01 lipid nanoparticles comprise a neutral lipid being resembled by the structure 1,2-diphytanoyl-sn-glycero-3-phosphoethanolamine (DPhyPE):

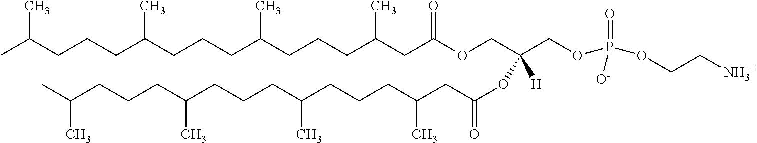 [SMILES: CC(C)CCCC(C)CCCC(C)CCCC(C)CC(=O)OC[C@H](COP(=O)([O-])OCC[NH3+])OC(=O)CC(C)CCCC(C)CCCC(C)CCCC(C)C]

Furthermore, in a specific embodiment, the GN01 lipid nanoparticles comprise a polymer conjugated lipid, preferably a pegylated lipid, being 1,2-dimyristoyl-rac-glycero-3-methoxypolyethylene glycol 2000 (DMG-PEG 2000) having the following structure:

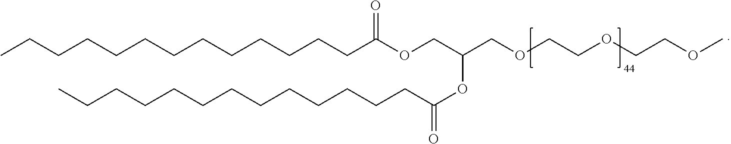

As used in the art, “DMG-PEG 2000” is considered a mixture of 1,2-DMG PEG2000 and 1,3-DMG PEG2000 in ˜97:3 ratio.

Accordingly, GN01 lipid nanoparticles (GN01-LNPs) according to one of the preferred embodiments comprise a SS-EC cationic lipid, neutral lipid DPhyPE, cholesterol, and the polymer conjugated lipid (pegylated lipid) 1,2-dimyristoyl-rac-glycero-3-methoxypolyethylene glycol (PEG-DMG).

In a preferred embodiment, the GN01 LNPs comprise:

(a) cationic lipid SS-EC (former name: SS-33/4PE-15; NOF Corporation, Tokyo, Japan) at an amount of 45-65 mol %;

(b) cholesterol at an amount of 25-45 mol %;

(c) DPhyPE at an amount of 8-12 mol %; and

(d) PEG-DMG 2000 at an amount of 1-3 mol %;

each amount being relative to the total molar amount of all lipidic excipients of the GN01 lipid nanoparticles.

In a further preferred embodiment, the GN01 lipid nanoparticles as described herein comprises 59 mol % cationic lipid, 10 mol % neutral lipid, 29.3 mol % steroid and 1.7 mol % polymer conjugated lipid, preferably pegylated lipid. In a most preferred embodiment, the GN01 lipid nanoparticles as described herein comprise 59 mol % cationic lipid SS-EC, 10 mol % DPhyPE, 29.3 mol % cholesterol and 1.7 mol % DMG-PEG 2000.

The amount of the cationic lipid relative to that of the nucleic acid in the GN01 lipid nanoparticle may also be expressed as a weight ratio (abbreviated f.e. “m/m”). For example, the GN01 lipid nanoparticles comprise the at least one nucleic acid, preferably the at least one RNA at an amount such as to achieve a lipid to RNA weight ratio in the range of about 20 to about 60, or about 10 to about 50. In other embodiments, the ratio of cationic lipid to nucleic acid or RNA is from about 3 to about 15, such as from about 5 to about 13, from about 4 to about 8 or from about 7 to about 11. In a very preferred embodiment of the present invention, the total lipid/RNA mass ratio is about 40 or 40, i.e. about 40 or 40 times mass excess to ensure RNA encapsulation. Another preferred RNA/lipid ratio is between about 1 and about 10, about 2 and about 5, about 2 and about 4, or preferably about 3.

Further, the amount of the cationic lipid may be selected taking the amount of the nucleic acid cargo such as the RNA compound into account. In one embodiment, the N/P ratio can be in the range of about 1 to about 50. In another embodiment, the range is about 1 to about 20, about 1 to about 10, about 1 to about 5. In one preferred embodiment, these amounts are selected such as to result in an N/P ratio of the GN01 lipid nanoparticles or of the composition in the range from about 10 to about 20. In a further very preferred embodiment, the N/P is 14 (i.e. 14 times mol excess of positive charge to ensure nucleic acid encapsulation).

In a preferred embodiment, GN01 lipid nanoparticles comprise 59 mol % cationic lipid COATSOME® SS-EC (former name: SS-33/4PE-15 as apparent from the examples section; NOF Corporation, Tokyo, Japan), 29.3 mol % cholesterol as steroid, 10 mol % DPhyPE as neutral lipid/phospholipid and 1.7 mol % DMG-PEG 2000 as polymer conjugated lipid. A further inventive advantage connected with the use of DPhyPE is the high capacity for fusogenicity due to its bulky tails, whereby it is able to fuse at a high level with endosomal lipids. For “GN01”, N/P (lipid to nucleic acid, e.g RNA mol ratio) preferably is 14 and total lipid/RNA mass ratio preferably is 40 (m/m).

In other embodiments, the at least one nucleic acid (e.g. DNA or RNA), preferably the at least one RNA is complexed with one or more lipids thereby forming lipid nanoparticles (LNP), wherein the LNP comprises
I at least one cationic lipid;
Ii at least one neutral lipid;
Iii at least one steroid or steroid analogue; and
Iiii at least one PEG-lipid as defined herein,

wherein the cationic lipid is DLin-KC2-DMA (50 mol %) or DLin-MC3-DMA (50 mol %), the neutral lipid is DSPC (10 mol %), the PEG lipid is PEG-DOMG (1.5 mol %) and the structural lipid is cholesterol (38.5 mol %). In other embodiments, the at least one nucleic acid (e.g. DNA or RNA), preferably the at least one RNA is complexed with one or more lipids thereby forming lipid nanoparticles (LNP), wherein the LNP comprises SS15/Chol/DOPE (or DOPC)/DSG-5000 at mol % 50/38.5/10/1.5.

In other embodiments, the nucleic acid of the invention may be formulated in liposomes, e.g. in liposomes as described in WO2019/222424, WO2019/226925, WO2019/232095, WO2019/232097, or WO2019/232208, the disclosure of WO2019/222424, WO2019/226925, WO2019/232095, WO2019/232097, or WO2019/232208 relating to liposomes or lipid-based carrier molecules herewith incorporated by reference.

In various embodiments, the LNP as defined herein have a mean diameter of from about 50 nm to about 200 nm, from about 60 nm to about 200 nm, from about 70 nm to about 200 nm, from about 80 nm to about 200 nm, from about 90 nm to about 200 nm, from about 90 nm to about 190 nm, from about 90 nm to about 180 nm, from about 90 nm to about 170 nm, from about 90 nm to about 160 nm, from about 90 nm to about 150 nm, from about 90 nm to about 140 nm, from about 90 nm to about 130 nm, from about 90 nm to about 120 nm, from about 90 nm to about 100 nm, from about 70 nm to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 160 nm, 170 nm, 180 nm, 190 nm, or 200 nm and are substantially non-toxic. As used herein, the mean diameter may be represented by the z-average as determined by dynamic light scattering as commonly known in the art.

The polydispersity index (PDI) of the nanoparticles is typically in the range of 0.1 to 0.5. In a particular embodiment, a PDI is below 0.2. Typically, the PDI is determined by dynamic light scattering.

In another preferred embodiment of the invention the lipid nanoparticles have a hydrodynamic diameter in the range from about 50 nm to about 300 nm, or from about 60 nm to about 250 nm, from about 60 nm to about 150 nm, or from about 60 nm to about 120 nm, respectively.

In another preferred embodiment of the invention the lipid nanoparticles have a hydrodynamic diameter in the range from about 50 nm to about 300 nm, or from about 60 nm to about 250 nm, from about 60 nm to about 150 nm, or from about 60 nm to about 120 nm, respectively.

In embodiments where more than one or a plurality, e.g. 2, 3, 4, 5, 6, 7, 8, 9, 10, 11, 12, 13, 14, 15 of coding RNA species are comprised in the composition, said more than one or said plurality e.g. 2, 3, 4, 5, 6, 7, 8, 9, 10, 11, 12, 13, 14, 15 of RNA species may be complexed within one or more lipids thereby forming LNPs comprising more than one or a plurality, e.g. 2, 3, 4, 5, 6, 7, 8, 9, 10, 11, 12, 13, 14, 15 of different coding RNA species.

In embodiments, the LNPs described herein may be lyophilized in order to improve storage stability of the formulation and/or the RNA. In embodiments, the LNPs described herein may be spray dried in order to improve storage stability of the formulation and/or RNA. Lyoprotectants for lyophilization and or spray drying may be selected from trehalose, sucrose, mannose, dextran and inulin. Preferred lyoprotectant is sucrose.

According to further embodiments, the composition of the second aspect may comprise at least one adjuvant. Suitably, the adjuvant is preferably added to enhance the immunostimulatory properties of the composition.

The term “adjuvant” as used herein will be recognized and understood by the person of ordinary skill in the art, and is for example intended to refer to a pharmacological and/or immunological agent that may modify, e.g. enhance, the effect of other agents (herein: the effect of the coding RNA) or that may be suitable to support administration and delivery of the composition. The term “adjuvant” refers to a broad spectrum of substances. Typically, these substances are able to increase the immunogenicity of antigens. For example, adjuvants may be recognized by the innate immune systems and, e.g., may elicit an innate immune response (that is, a non-specific immune response). “Adjuvants” typically do not elicit an adaptive immune response. In the context of the invention, adjuvants may enhance the effect of the antigenic peptide or protein provided by the coding RNA. In that context, the at least one adjuvant may be selected from any adjuvant known to a skilled person and suitable for the present case, i.e. supporting the induction of an immune response in a subject, e.g. in a human subject.

Accordingly, the composition of the second aspect may comprise at least one adjuvant, wherein the at least one adjuvant may be suitably selected from any adjuvant provided in WO2016/203025. Adjuvants disclosed in any of the claims 2 to 17 of WO2016/203025, preferably adjuvants disclosed in claim 17 of WO2016/203025 are particularly suitable, the specific content relating thereto herewith incorporated by reference.

The composition of the second aspect may comprise, besides the components specified herein, at least one further component which may be selected from the group consisting of further antigens (e.g. in the form of a peptide or protein) or further antigen-encoding nucleic acids; a further immunotherapeutic agent; one or more auxiliary substances (cytokines, such as monokines, lymphokines, interleukins or chemokines); or any further compound, which is known to be immune stimulating due to its binding affinity (as ligands) to human Toll-like receptors; and/or an adjuvant nucleic acid, preferably an immunostimulatory RNA (isRNA), e.g. CpG-RNA etc.

Rotavirus Vaccine:

In a third aspect, the present invention provides a Rotavirus vaccine.

In preferred embodiments of the third aspect, the vaccine comprises at least coding RNA of the first aspect, or the composition of the second aspect.

Notably, embodiments relating to the composition of the second aspect may likewise be read on and be understood as suitable embodiments of the vaccine of the third aspect. Also, embodiments relating to the vaccine of the third aspect may likewise be read on and be understood as suitable embodiments of the composition of the second aspect (comprising the RNA of the first aspect).

The term “vaccine” will be recognized and understood by the person of ordinary skill in the art, and is for example intended to be a prophylactic or therapeutic material providing at least one epitope or antigen, preferably an immunogen. In the context of the invention the antigen or antigenic function is provided by the inventive coding RNA of the first aspect (said RNA comprising a coding sequence encoding a antigenic peptide or protein derived from Rotavirus, e.g. VP8*) or the composition of the second aspect (comprising at least one RNA of the first aspect).

In preferred embodiments of the third aspect, the vaccine of the third aspect, or the composition of the second aspect, elicits an adaptive immune response, preferably an adaptive immune response against a Rotavirus.

In preferred embodiments of the third aspect, the vaccine of the third aspect, or the composition of the second aspect, induces specific and functional humoral immune response against Rotavirus; and/or broad, functional cellular T-cell responses against Rotavirus.

In preferred embodiments of the third aspect, the vaccine of the third aspect, or the composition of the second aspect, induces high levels of virus neutralizing antibodies to prevent a Rotavirus infection, preferably high levels of virus neutralizing antibodies against homologous and heterologous Rotavirus strains.

According to a preferred embodiment of the third aspect, the vaccine as defined herein may further comprise a pharmaceutically acceptable carrier and optionally at least one adjuvant as specified in the context of the second aspect.

Suitable adjuvants in that context may be selected from adjuvants disclosed in claim 17 of WO2016/203025.

In a preferred embodiment, the vaccine is a monovalent vaccine.

In embodiments, the vaccine is a polyvalent vaccine comprising a plurality or at least more than one of the coding RNA species. Embodiments relating to a polyvalent composition as disclosed in the context of the second aspect may likewise be read on and be understood as suitable embodiments of the polyvalent vaccine of the third aspect. In a preferred embodiment, the vaccine is a trivalent vaccine.

Said trivalent vaccine may suitably comprise one coding RNA species encoding a VP8* antigen construct, wherein the VP8* is or is derived from a Rotavirus A [P4] serotype; one coding RNA species encoding a VP8* antigen construct, wherein the VP8* is or is derived from a Rotavirus A [P6] serotype; one coding RNA species encoding a VP8* antigen construct, wherein the VP8* is or is derived from a Rotavirus A [P8] serotype. Embodiments relating to a trivalent composition as described in the context of the second aspect may likewise be read on and be understood as suitable embodiments of the trivalent vaccine.

The Rotavirus vaccine typically comprises a safe and effective amount of coding RNA of the first aspect or composition of the second aspect. As used herein, “safe and effective amount” means an amount of coding RNA or composition sufficient to significantly induce a positive modification of a disease or disorder related to an infection with Rotavirus. At the same time, a “safe and effective amount” is small enough to avoid serious side-effects. In relation to coding RNA, composition, or vaccine of the present invention, the expression “safe and effective amount” preferably means an amount of coding RNA, composition, or vaccine that is suitable for stimulating the adaptive immune system against Rotavirus in such a manner that no excessive or damaging immune reactions (e.g. innate immune responses) are achieved.

A “safe and effective amount” of coding RNA, composition, or vaccine as defined above will vary in connection with the particular condition to be treated and also with the age and physical condition of the patient to be treated, the severity of the condition, the duration of the treatment, the nature of the accompanying therapy, of the particular pharmaceutically acceptable carrier used, and similar factors, within the knowledge and experience of the skilled person. Moreover, the “safe and effective amount” of coding RNA, composition, or vaccine may depend from application/delivery route (intradermal, intramuscular), application device (jet injection, needle injection, microneedle patch) and/or complexation/formulation (protamine complexation or LNP encapsulation). Moreover, the “safe and effective amount” of coding RNA, composition, or vaccine may depend from the physical condition of the treated subject (infant, pregnant women, immunocompromised human subject etc.).

In some embodiments, the “safe and effective amount” is a dose equivalent to an at least 2-fold, at least 4-fold, at least 10-fold, at least 100-fold, at least 1000-fold reduction in the standard of care dose of a Rotavirus vaccine, wherein the anti-antigenic antibody titer produced in the subject is at least equivalent to an anti-antigenic antibody titer produced in a control subject administered the standard of care dose of a Rotavirus vaccine based on live attenuated Rotavirus vaccine.

The Rotavirus vaccine can be used according to the invention for human medical purposes and also for veterinary medical purposes (mammals, vertebrates, or avian species).

The pharmaceutically acceptable carrier as used herein preferably includes the liquid or non-liquid basis of the inventive Rotavirus vaccine. If the inventive vaccine is provided in liquid form, the carrier will be water, typically pyrogen-free water; isotonic saline or buffered (aqueous) solutions, e.g. phosphate, citrate etc. buffered solutions. Preferably, Ringer-Lactate solution is used as a liquid basis for the vaccine or the composition according to the invention as described in WO2006/122828, the disclosure relating to suitable buffered solutions incorporated herewith by reference. Other preferred solutions used as a liquid basis for the vaccine or the composition, in particular for compositions/vaccines comprising LNPs, comprise Sucrose.

The choice of a pharmaceutically acceptable carrier as defined herein is determined, in principle, by the manner, in which the pharmaceutical composition(s) or vaccine according to the invention is administered. The Rotavirus vaccine is preferably administered locally. Routes for local administration in general include, for example, topical administration routes but also intradermal, transdermal, subcutaneous, or intramuscular injections or intralesional, intracranial, intrapulmonal, intracardial, intraarticular and sublingual injections. More preferably, composition or vaccines according to the present invention may be administered by an intradermal, subcutaneous, or intramuscular route, preferably by injection, which may be needle-free and/or needle injection. Preferred in the context of the invention is intramuscular injection. Compositions/vaccines are therefore preferably formulated in liquid or solid form. The suitable amount of the vaccine or composition according to the invention to be administered can be determined by routine experiments, e.g. by using animal models. Such models include, without implying any limitation, rabbit, sheep, mouse, rat, dog and non-human primate models. Preferred unit dose forms for injection include sterile solutions of water, physiological saline or mixtures thereof. The pH of such solutions should be adjusted to about 7.4.

The inventive Rotavirus vaccine or composition as defined herein may comprise one or more auxiliary substances or adjuvants as defined above in order to further increase the immunogenicity. A synergistic action of the coding RNA contained in the inventive composition/vaccine and of an auxiliary substance, which may be optionally be co-formulated (or separately formulated) with the inventive vaccine or composition as described above, is preferably achieved thereby. Such immunogenicity increasing agents or compounds may be provided separately (not co-formulated with the inventive vaccine or composition) and administered individually.

The Rotavirus vaccine is preferably provided in lyophilized or spray-dried form (as described in the context of the second aspect).

Kit or Kit of Parts, Application, Medical Uses, Method of Treatment:

In a fourth aspect, the present invention provides a kit or kit of parts suitable for treating or preventing a Rotavirus infection.

In preferred embodiments, the kit or kit of parts comprises at least one coding RNA of the first aspect, at least one composition of the second aspect (comprising coding RNA), and/or at least one vaccine of the third aspect. In addition, the kit or kit of parts of the fourth aspect may comprise a liquid vehicle for solubilising, and/or technical instructions providing information on administration and dosage of the components.

The kit may further comprise additional components as described in the context of the composition of the second aspect, and/or the vaccine of the third aspect.

The technical instructions of said kit may contain information about administration and dosage and patient groups. Such kits, preferably kits of parts, may be applied e.g. for any of the applications or uses mentioned herein, preferably for the use of the coding RNA of the first aspect, the composition of the second aspect, or the vaccine of the third aspect, for the treatment or prophylaxis of an infection or diseases caused by a Rotavirus or disorders related thereto. Preferably, the coding RNA of the first aspect, the composition of the second aspect, or the vaccine of the third aspect is provided in a separate part of the kit, wherein the coding RNA of the first aspect, the composition of the second aspect, or the vaccine of the third aspect is preferably lyophilised. The kit may further contain as a part a vehicle (e.g. buffer solution) for solubilising the coding RNA of the first aspect, the composition of the second aspect, or the vaccine of the third aspect.

In preferred embodiments, the kit or kit of parts as defined herein comprises Ringer lactate solution.

Any of the above kits may be used in a treatment or prophylaxis as defined herein. More preferably, any of the above kits may be used as a vaccine, preferably a vaccine against infections caused by a Rotavirus.

First and Second Medical Use:

A further aspect relates to the first medical use of the provided coding RNA, composition, vaccine, or kit.

Accordingly, the invention provides at least one coding RNA as defined in the first aspect for use as a medicament, the composition as defined in the second aspect for use as a medicament, the Rotavirus vaccine as defined in the third aspect for use as a medicament, and the kit or kit of parts as defined in the third aspect for use as a medicament.

The present invention furthermore provides several applications and uses of the coding RNA, the composition, the vaccine, or the kit or kit of parts.

In particular, said coding RNA, composition, vaccine, or the kit or kit of parts may be used for human medical purposes and also for veterinary medical purposes, preferably for human medical purposes.

In particular, said coding RNA, composition, vaccine, or the kit or kit of parts is for use as a medicament for human medical purposes, wherein said RNA, composition, vaccine, or the kit or kit of parts may be particularly suitable for young infants, newborns, immunocompromised recipients, as well as pregnant and breast-feeding women and elderly people. Said coding RNA, composition, vaccine, or the kit or kit of parts is for use as a medicament for human medical purposes, wherein said RNA, composition, vaccine, or the kit or kit of parts may be particularly suitable for intramuscular injection.

In yet another aspect, the invention relates to the second medical use of the provided coding RNA, composition, vaccine, or kit.

Accordingly, the invention provides at least one coding RNA as defined in the first aspect, for use in the treatment or prophylaxis of an infection with a Rotavirus, or a disorder related to such an infection, the composition as defined in the second aspect, for use in the treatment or prophylaxis of an infection with a Rotavirus, or a disorder related to such an infection, the Rotavirus vaccine as defined in the third aspect, for use in the treatment or prophylaxis of an infection with a Rotavirus or a disorder related to such an infection, and the kit or kit of parts as defined in the third aspect, for use in the treatment or prophylaxis of an infection with a Rotavirus, or a disorder related to such an infection.

In embodiments, the coding RNA of the first aspect, the composition of the second aspect, the vaccine of the third aspect, or the kit or kit of parts of the fourth aspect is for use in the treatment or prophylaxis of an infection with a Rotavirus, preferably with Rotavirus A, in particular Rotavirus A of serotypes [P4], [P6], and/or [P8], preferably derived from RVA/BE1058/P[4], RVA/F01322/P[6], RVA/BE1128/P[8] and/or RVA/Wa-VirWa/P[8].

In preferred embodiments, the composition of the second aspect, the vaccine of the third aspect, or the kit or kit of parts of the fourth aspect is for use in the treatment or prophylaxis of an infection with Rotavirus, wherein administration of said composition, vaccine, or kit provides protection against three different Rotavirus A serotypes [P4], [P6], [P8] (e.g. when administered as a trivalent composition or vaccine as defined herein).

As used herein, “a disorder related to a Rotavirus infection” may preferably comprise a typical symptom or a complication of a Rotavirus infection, including gastrointestinal complications/symptoms or fewer etc.

Particularly, the coding RNA of the first aspect, the composition of the second aspect, the vaccine of the third aspect, or the kit or kit of parts of the fourth aspect may be used in a method of prophylactic (pre-exposure prophylaxis or post-exposure prophylaxis) and/or therapeutic treatment of infections caused by a Rotavirus.

The coding RNA, the composition or the vaccine may preferably be administered locally. In particular, composition or vaccines may be administered by an intradermal, subcutaneous, intranasal, or intramuscular route. In embodiments, the inventive coding RNA, composition, vaccine may be administered by conventional needle injection or needle-free jet injection. Preferred in that context is intramuscular injection.

In embodiments, the coding RNA as comprised in a composition or vaccine as defined herein is provided in an amount of about 100 ng to about 500 ug, in an amount of about 1 ug to about 200 ug, in an amount of about 1 ug to about 100 ug, in an amount of about 5 ug to about 100 ug, preferably in an amount of about 10 ug to about 50 ug, specifically, in an amount of about 5 ug, 10 ug, 15 ug, 20 ug, 25 ug, 30 ug, 35 ug, 40 ug, 45 ug, 50 ug, 55 ug, 60 ug, 65 ug, 70 ug, 75 ug, 80 ug, 85 ug, 90 ug, 95 ug or 100 ug.

In some embodiments, the vaccine comprising the coding RNA, or the composition comprising the coding RNA is formulated in an effective amount to produce an antigen specific immune response in a subject. In some embodiments, the effective amount is a total dose of 1 ug to 200 ug, 1 ug to 100 ug, or 5 ug to 100 ug.

In some embodiments, the subject is about 5 years old or younger. For example, the subject may be between the ages of about 1 year and about 5 years (e.g., about 1, 2, 3, 4 or 5 years), or between the ages of about 6 months and about 1 year (e.g., about 6, 7, 8, 9, 10, 11 or 12 months). In some embodiments, the subject is about 12 months or younger (e.g., 12, 11, 10, 9, 8, 7, 6, 5, 4, 3, 2 months or 1 month). In some embodiments, the subject is about 6 months or younger.

In one embodiment, the immunization protocol for the treatment or prophylaxis of a subject against Rotavirus comprises one single doses of the composition or the vaccine.

In some embodiments, the effective amount is a dose of 5 ug administered to the subject in one vaccination. In some embodiments, the effective amount is a dose of 10 ug administered to the subject in one vaccination. In some embodiments, the effective amount is a dose of 20 ug administered to the subject in one vaccination. In some embodiments, the effective amount is a dose of 30 ug administered to the subject in one vaccination. In some embodiments, the effective amount is a dose of 40 ug administered to the subject in one vaccination. In some embodiments, the effective amount is a dose of 50 ug administered to the subject in one vaccination. In some embodiments, the effective amount is a dose of 100 ug administered to the subject in one vaccination. In some embodiments, the effective amount is a dose of 200 ug administered to the subject in one vaccination.

In preferred embodiments, the immunization protocol for the treatment or prophylaxis of a Rotavirus infection comprises a series of single doses or dosages of the composition or the vaccine. A single dosage, as used herein, refers to the initial/first dose, a second dose or any further doses, respectively, which are preferably administered in order to “boost” the immune reaction.

In some embodiments, the effective amount is a dose of 5 ug administered to the subject a total of two times. In some embodiments, the effective amount is a dose of 10 ug administered to the subject a total of two times. In some embodiments, the effective amount is a dose of 20 ug administered to the subject a total of two times. In some embodiments, the effective amount is a dose of 30 ug administered to the subject a total of two times. In some embodiments, the effective amount is a dose of 40 ug administered to the subject a total of two times. In some embodiments, the effective amount is a dose of 50 ug administered to the subject a total of two times. In some embodiments, the effective amount is a dose of 100 ug administered to the subject a total of two times. In some embodiments, the effective amount is a dose of 200 ug administered to the subject a total of two times.

In preferred embodiments, the vaccine/composition immunizes the subject against a Rotavirus infection (upon administration as defined herein) for at least 1 year, preferably at least 2 years. In preferred embodiments, the vaccine/composition immunizes the subject against a Rotavirus infection for more than 2 years, more preferably for more than 3 years, even more preferably for more than 4 years, even more preferably for more than 5-10 years.

Method of Treatment and Use, Diagnostic Method and Use:

In another aspect, the present invention relates to a method of treating or preventing a disorder.

In preferred embodiments, the present invention relates to a method of treating or preventing a disorder, wherein the method comprises applying or administering to a subject in need thereof at least one coding RNA of the first aspect, the composition of the second aspect, the vaccine of the third aspect, or the kit or kit of parts of the fourth aspect.

In preferred embodiments, the disorder is an infection with a Rotavirus, or a disorder related to such infections, in particular an infection with Rotavirus A, or a disorder related to such infections. In particular, the disorder is an infection with Rotavirus A serotypes [P4], [P6], and/or [P8].

In preferred embodiments, the present invention relates to a method of treating or preventing a disorder as defined above, wherein the method comprises applying or administering to a subject in need thereof at least one coding RNA of the first aspect, the composition of the second aspect, the vaccine of the third aspect, or the kit or kit of parts of the fourth aspect, wherein the subject in need is preferably a mammalian subject.

In particularly preferred embodiments, the subject in need is a mammalian subject, preferably a human subject. Suitably, the human subject may is an infant, a newborn, a pregnant women, a breast-feeding woman, an elderly, or an immunocompromised human subject. Most preferably, the human subject is an infant or a newborn. For example, the infant human subject may be between the ages of about 1 year and about 5 years (e.g., about 1, 2, 3, 4 or 5 years), or the newborn human subject may be between the ages of about 6 months and about 1 year (e.g., about 6, 7, 8, 9, 10, 11 or 12 months). In some embodiments, the newborn human subject is younger than about 6 months,

In particular, such the method of treatment may comprise the steps of:
a) providing at least one coding RNA of the first aspect, at least one composition of the second aspect, at least one vaccine of the third aspect, or the kit or kit of parts of the fourth aspect;
b) applying or administering said RNA, composition, vaccine, or kit or kit of parts to a subject as a first dose
c) optionally, applying or administering said RNA, composition, vaccine, or kit or kit of parts to a subject as a second dose or a further dose, preferably at least 3, 4, 5, 6, 7, 8, 9, 10, 11, 12, months after the first dose.

The first dosage, as used herein, refers to the initial/first dose, a second dose or any further doses, respectively, which are preferably administered in order to “boost” the immune reaction.

According to a further aspect, the present invention also provides a method for expression of at least one polypeptide comprising at least one peptide or protein derived from a Rotavirus, or a fragment or variant thereof, wherein the method preferably comprises the following steps:

a) providing at least one coding RNA of the first aspect or at least one composition of the second aspect; and

b) applying or administering said RNA or composition to an expression system (cells), a tissue, an organism.

The method for expression may be applied for laboratory, for research, for diagnostic, for commercial production of peptides or proteins and/or for therapeutic purposes. The method may furthermore be carried out in the context of the treatment of a specific disease, particularly in the treatment of infectious diseases, particularly Rotavirus infections.

Likewise, according to another aspect, the present invention also provides the use of the coding RNA of the first aspect, the composition of the second aspect, the vaccine of the third aspect, or the kit or kit of parts of the fourth aspect preferably for diagnostic or therapeutic purposes, e.g. for expression of an encoded Rotavirus antigenic peptide or protein, e.g. by applying or administering said coding RNA, composition comprising said coding RNA, vaccine comprising said coding RNA, e.g. to a cell-free expression system, a cell (e.g. an expression host cell or a somatic cell), a tissue or an organism. In specific embodiments, applying or administering said coding RNA, composition comprising said coding RNA, vaccine comprising said coding RNA to a tissue or an organism may be followed by e.g. a step of obtaining induced Rotavirus antibodies e.g. Rotavirus specific (monoclonal) antibodies or a step of obtaining generated Rotavirus VP8* protein constructs.

The use may be applied for a (diagnostic) laboratory, for research, for diagnostics, for commercial production of peptides, proteins, or Rotavirus antibodies and/or for therapeutic purposes. The use may be carried out in vitro, in vivo or ex vivo. The use may furthermore be carried out in the context of the treatment of a specific disease, particularly in the treatment of a Rotavirus infection or a related disorder.

According to a further aspect, the present invention also provides a method of manufacturing a composition or a Rotavirus vaccine, comprising the steps of:
a) RNA in vitro transcription step using a DNA template in the presence of a trinucleotide cap analogue to obtain cap1 comprising coding RNA, preferably as provided in Table 4;
b) Purifying the obtained cap1 comprising coding RNA of step a) using RP-HPLC, and/or TFF, and/or Oligo(dT) purification and/or AEX, preferably using RP-HPLC;
c) Providing a first liquid composition comprising the purified cap1 comprising coding RNA of step b);
d) Providing a second liquid composition comprising at least one cationic lipid as defined herein, a neutral lipid as defined herein, a steroid or steroid analogue as defined herein, and a PEG-lipid as defined herein;
e) Introducing the first liquid composition and the second liquid composition into at least one mixing means to allow the formation of LNPs comprising cap1 comprising coding RNA;
f) Purifying the obtained LNPs comprising cap1 comprising coding RNA;
g) optionally, lyophilizing the purified LNPs comprising cap1 comprising coding RNA.

Preferably, the mixing means of step e) is a T-piece connector or a microfluidic mixing device. Preferably, the purifying step f) comprises at least one step selected from precipitation step, dialysis step, filtration step, TFF step. Optionally, an enzymatic polyadenylation step may be performed after step a) or b). Optionally, further purification steps may be implemented to e.g. remove residual DNA, buffers, small RNA by-products etc. Optionally, RNA in vitro transcription is performed in the absence of a cap analog, and an enzymatic capping step is performed after RNA vitro transcription. Optionally, RNA in vitro transcription is performed in the presence of at least one modified nucleotide as defined herein.

### LIST OF PREFERRED EMBODIMENTS/ITEMS

In the following, particularly preferred embodiments (items 1-64) of the invention are provided.

Item 1:

A coding RNA for a Rotavirus vaccine comprising
a) at least one heterologous 5′ untranslated region (5′-UTR) and/or at least one heterologous 3′ untranslated region (3′-UTR); and
b) at least one coding sequence operably linked to said 3′-UTR and/or 5′-UTR encoding at least one antigenic protein of a Rotavirus, wherein said antigenic protein is or is derived from VP8* or an immunogenic fragment or immunogenic variant thereof.

Item 2:

Coding RNA of item 1, wherein the Rotavirus is selected from species A, B or C, preferably wherein the Rotavirus is Rotavirus A.

Item 3:

Coding RNA of claim 1 or 2, wherein the Rotavirus is selected from the G-serotypes or P-serotypes G1, G2, G3, G4, G9, G12, P[4], P[6] or P[8].

Item 4:

Coding RNA of any one of the preceding items, wherein the Rotavirus is a Rotavirus A selected from the P serotypes P[4], P[6] or P[8]

Item 5

Coding RNA of any one of the preceding items, wherein the Rotavirus is a Rotavirus A selected from Human rotavirus A BE1058 (RVA/Human-wt/BEL/BE1058/2008/G2P[4], G2P[4], JN849123.1, GI:371455744, AEX30665.1, acronym: RVA/BE1058/P[4]), Human rotavirus A F01322 (Hu/BEL/F01322/2009/G3P[6], G3P[6], JF460826.1. GI: 37531451, AFA51886.1, acronym: RVA/F01322/P[6]), Human rotavirus A BE1128 (RVA/Human-wt/BEL/BE1128/2009/G1P[8], G1P[8], JN849135.1. GI: 371455756, AEX30671, acronym: RVA/BE1128/P[8]), or Human rotavirus A WA-VirWa (Wa variant VirWa, G1P[8], ACR22783.1, GI: 237846292, FJ423116, acronym: RVA/Wa-VirWa/P[8]).

Item 6:

Coding RNA of any one of the preceding items, wherein the VP8* is a full length VP8* protein having an amino acid sequence comprising or consisting of amino acid 1 to amino acid 240, or a fragment of a VP8* protein.

Item 7:

Coding RNA of any one of items 6, wherein the fragment of a VP8* comprises the lectin domain and lacks the N-terminal alpha helix-domain, wherein the fragment has preferably an amino acid sequence comprising or consisting of amino acid 41 to amino acid 223, or amino acid 65 to amino acid 223 (of a corresponding full length VP8*).

Item 8:

Coding RNA of any one of the preceding items, wherein the amino acid sequences of the at least one antigenic protein derived from VP8* is mutated to delete at least one predicted or potential glycosylation site.

Item 9:

Coding RNA of any one of the preceding items, wherein the amino acid sequences of the at least one antigenic protein derived from VP8* is mutated to delete all predicted or potential glycosylation sites.

Item 10:

Coding RNA of any one of the preceding items, wherein the at least one coding sequence encodes at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 19-45, or an immunogenic fragment or immunogenic variant of any of these.

Item 11:

Coding RNA of any one of the preceding items, wherein the at least one coding sequence additionally encodes one or more heterologous peptide or protein elements selected from a signal peptide, a linker, a helper epitope, an antigen clustering domain, or a transmembrane domain.

Item 12:

Coding RNA of item 11, wherein the signal peptide is or is derived from HsPLAT, HsALB, IgE, wherein the amino acid sequences of said heterologous signal peptides is identical or at least 70%, 80%, 85%, 86%, 87%, 88%, 89%, 90%, 91%, 92%, 93%, 94%, 95%, 96%, 97%, 98%, or 99% identical to any one of amino acid sequences SEQ ID NOs: 1738-1740, or fragment or variant of any of these.

Item 13:

Coding RNA of item 11, wherein the helper epitope is or is derived from P2, wherein the amino acid sequences of said helper epitopes is identical or at least 70%, 80%, 85%, 86%, 87%, 88%, 89%, 90%, 91%, 92%, 93%, 94%, 95%, 96%, 97%, 98%, or 99% identical to amino acid sequence SEQ ID NOs: 1750 or fragment or variant of any of these.

Item 14:

Coding RNA of item 11, wherein the antigen clustering domain is or is derived from ferritin or lumazine-synthase, wherein the amino acid sequences of said antigen clustering domain is identical or at least 70%, 80%, 85%, 86%, 87%, 88%, 89%, 90%, 91%, 92%, 93%, 94%, 95%, 96%, 97%, 98%, or 99% identical to any one of amino acid sequences SEQ ID NOs: 1759, 1764, or fragment or variant of any of these.

Item 15:

Coding RNA of item 11, wherein the transmembrane domain is or is derived from an influenza HA transmembrane domain, preferably derived from an influenza A HA H1N1, more preferably from H1N1/A/Netherlands/602/2009, TM domain_HA, aa521-566, NCBI Acc. No.: ACQ45338.1, CY039527.1) or fragment or variant thereof.

Item 16:

Coding RNA of any one of the preceding items, wherein the at least one coding sequence encodes the following elements preferably in N-terminal to C-terminal direction:

a) helper epitope, VP8*protein or VP8*fragment; or

b) helper epitope, VP8*protein or VP8*fragment; antigen clustering domain; or

c) Signal peptide, helper epitope, VP8*protein or fragment thereof; or

d) Signal peptide, helper epitope, VP8*protein or VP8*fragment, antigen clustering domain; or

e) Signal peptide, helper epitope, VP8*protein or VP8*fragment, transmembrane domain; or

f) antigen clustering domain, helper epitope; VP8*protein or VP8*fragment.

Item 17:

Coding RNA of any one of the preceding items, wherein the at least one coding sequence encodes at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 1-6, 46-117, 1899, 1900, or an immunogenic fragment or immunogenic variant of any of these.

Item 18:

Coding RNA of item 17, wherein the at least one coding sequence encodes at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 1-3, 4-6, 46-54, 64-72, 91-99, 109-117, or an immunogenic fragment or immunogenic variant of any of these.

Item 19:

Coding RNA of any one of the preceding items, wherein the at least one coding sequence comprises a codon modified coding sequence comprising or consisting of a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 190-261, 298-369, 406-477, 514-585, 1901-1906, or a fragment or variant of any of these sequences.

Item 20:

Coding RNA of any one of the preceding items, wherein the at least one coding sequence comprises at least one modified nucleotide selected from pseudouridine (ψ) and N1-methylpseudouridine (m1ψ), preferably wherein all uracil nucleotides are replaced by pseudouridine (ψ) nucleotides and/or N1-methylpseudouridine (m1ψ) nucleotides.

Item 21:

Coding RNA of any one of the preceding items, wherein the at least one coding sequence is a codon modified coding sequence, wherein the amino acid sequence encoded by the at least one codon modified coding sequence is preferably not being modified compared to the amino acid sequence encoded by the corresponding wild type coding sequence.

Item 22:

Coding RNA according to item 21, wherein the at least one codon modified coding sequence is selected from C maximized coding sequence, CAI maximized coding sequence, human codon usage adapted coding sequence, G/C content modified coding sequence, and G/C optimized coding sequence, or any combination thereof.

Item 23:

Coding RNA of item 21 or 22, wherein the at least one coding sequence comprises or consists of a codon modified coding sequence comprising or consisting of a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 154-585, 1901-1906 or a fragment or variant of any of these sequences.

Item 24:

Coding RNA of any one of items 21 to 23, wherein the at least one coding sequence comprises or consists of a codon modified coding sequence comprising or consisting of a nucleic acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 190-198, 208-216, 235-243, 253-261, 298-306, 316-324, 343-351, 361-369, 1901-1906 or a fragment or variant of any of these sequences.

Item 25:

Coding RNA of any one of the preceding items, wherein the coding RNA is an mRNA, a self-replicating RNA, a circular RNA, a viral RNA, or a replicon RNA.

Item 26:

Coding RNA of any one of the preceding items, wherein the coding RNA is an mRNA.

Item 27:

Coding RNA of any one of the preceding items, wherein the coding RNA comprises a 5′-cap structure, preferably cap0, cap1, cap2, a modified cap0 or a modified cap1 structure.

Item 28:

Coding RNA of item 27, wherein the 5′-cap structure is a cap1 structure.

Item 29:

Coding RNA of any one of the preceding items, wherein the coding RNA comprises a cap1 structure, wherein said cap1 structure is obtainable by co-transcriptional capping preferably using a trinucleotide cap1 analogue.

Item 30:

Coding RNA of any one of item 27 to 29, wherein about 70%, 75%, 80%, 85%, 90%, 95% of the coding RNA (species) comprises a cap1 structure as determined using a capping assay.

Item 31:

Coding RNA of any one of the preceding items, wherein the coding RNA comprises at least one poly(A) sequence comprising about 30 to about 200 adenosine nucleotides, preferably comprising about 100 adenosine nucleotides.

Item 32:

Coding RNA of item 31, wherein the at least one poly(A) sequence is located at the 3′ terminus, preferably wherein the 3′-terminal nucleotide of the coding RNA is the 3-terminal A nucleotide of the poly(A) sequence.

Item 33:

Coding RNA of any one of the preceding items, wherein the coding RNA comprises a cap1 structure as defined in items 27 to 30 and at least one poly(A) sequence as defined in items 31 to 32.

Item 34:

Coding RNA of any one of the preceding items, wherein the RNA comprises at least one histone stem-loop, wherein the histone stem-loop preferably comprises or consists of a nucleic acid sequence identical or at least 70%, 80%, 90%, 91%, 92%, 93%, 94%, 95%, 96%, 97%, 98%, or 99% identical to SEQ ID NOs: 1819 or 1820, or fragments or variants thereof.

Item 35:

Coding RNA of any one of the preceding items, wherein the RNA comprises at least one 3′-terminal sequence element comprising or consisting of a nucleic acid sequence being identical or at least 70%, 80%, 90%, 91%, 92%, 93%, 94%, 95%, 96%, 97%, 98%, or 99% identical to SEQ ID NOs: 1825-1856, or a fragment or variant thereof.

Item 36:

Coding RNA of any one of the preceding items, wherein the at least one heterologous 3′-UTR comprises or consisting of a nucleic acid sequence derived from a 3′-UTR of a gene selected from PSMB3, alpha-globin, ALB7, CASP1, COX6B1, GNAS, NDUFA1 and RPS9, or from a homolog, a fragment or a variant of any one of these genes.

Item 37:

Coding RNA of any one of the preceding items, wherein the at least one heterologous 5′-UTR comprises or consisting of a nucleic acid sequence derived from a 5′-UTR of a gene selected from HSD17B4, RPL32, ASAH1, ATP5A1, MP68, NDUFA4, NOSIP, RPL31, SLC7A3, TUBB4B and UBQLN2, or from a homolog, a fragment or variant of any one of these genes.

Item 38:

Coding RNA of any one of the preceding items, wherein
the at least one heterologous 5′-UTR is derived from a 5′-UTR of a HSD17B4 gene, or from a corresponding RNA sequence, homolog, fragment or variant thereof and the at least one 3′-UTR is derived from a 3′-UTR of a PSMB3 gene, or from a corresponding RNA sequence, homolog, fragment or variant thereof, preferably wherein said 5′-UTR comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 1781 or 1782 or a fragment or a variant thereof, and wherein said 3′-UTR comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 1803 or 1804 or a fragment or a variant thereof; or
the at least one heterologous 3′-UTR is derived from a 3′-UTR of an alpha-globin gene, or from a corresponding RNA sequence, homolog, fragment or variant thereof, preferably wherein said 3-UTR comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 1817 or 1818 or a fragment or a variant thereof.

Item 39:

Coding RNA of any one of the preceding items, wherein the coding RNA comprises or consists of an RNA sequence which is identical or at least 70%0, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 586-1737, 1862-1882, 1885-1898, 1907-1930 or a fragment or variant of any of these sequences.

Item 40:

Coding RNA of item 39, wherein the coding RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 586-594, 604-612, 631-639, 649-666, 676-684, 703-711, 721-738, 748-756, 775-783, 793-810, 820-828, 847-855, 865-882, 892-900, 919-927, 937-954, 964-972, 991-999, 1009-1026, 1036-1044, 1063-1071, 1081-1098, 1108-1116, 1135-1143, 1153-1170, 1180-1188, 1207-1215, 1225-1242, 1252-1260, 1279-1287, 1297-1314, 1324-1332, 1351-1359, 1369-1386, 1396-1404, 1423-1431, 1441-1458, 1468-1476, 1495-1503, 1513-1530, 1540-1548, 1567-1575, 1585-1602, 1612-1620, 1639-1647, 1657-1674, 1684-1692, 1711-1719, 1729-1737, 1862-1870, 1872-1877, 1885, 1898, 1907-1930, or a fragment or variant of any of these sequences.

Item 41:

A composition comprising at least one coding RNA as defined in any one of items 1 to 40, wherein the composition optionally comprises at least one pharmaceutically acceptable carrier or excipient.

Item 42:

Composition of item 41, wherein the composition comprises more than one or a plurality, preferably 2, 3, 4, 5, 6, 7, 8, 9, or 10 different coding RNAs each defined in any one of items 1 to 40.

Item 43:

Composition of item 42, wherein the composition comprises
(i) at least one coding RNA encoding at least one antigenic protein that is or is derived from VP8* of a Rotavirus A from a P[4] serotype, preferably according to SEQ ID NOs: 586-588, 595-597, 604-606, 613-615, 622-624, 631-633, 640-642, 649-651, 658-660, 667-669, 676-678, 685-687, 694-696, 703-705, 712-714, 721-723, 730-732, 739-741, 748-750, 757-759, 766-768, 775-777, 784-786, 793-795, 802-804, 811-813, 820-822, 829-831, 838-840, 847-849, 856-858, 865-867, 874-876, 883-885, 892-894, 901-903, 910-912, 919-921, 928-930, 937-939, 946-948, 955-957, 964-966, 973-975, 982-984, 991-993, 1000-1002, 1009-1011, 1018-1020, 1027-1029, 1036-1038, 1045-1047, 1054-1056, 1063-1065, 1072-1074, 1081-1083, 1090-1092, 1099-1101, 1108-1110, 1117-1119, 1126-1128, 1135-1137, 1144-1146, 1153-1155, 1162-1164, 1171-1173, 1180-1182, 1189-1191, 1198-1200, 1207-1209, 1216-1218, 1225-1227, 1234-1236, 1243-1245, 1252-1254, 1261-1263, 1270-1272, 1279-1281, 1288-1290, 1297-1299, 1306-1308, 1315-1317, 1324-1326, 1333-1335, 1342-1344, 1351-1353, 1360-1362, 1369-1371, 1378-1380, 1387-1389, 1396-1398, 1405-1407, 1414-1416, 1423-1425, 1432-1434, 1441-1443, 1450-1452, 1459-1461, 1468-1470, 1477-1479, 1486-1488, 1495-1497, 1504-1506, 1513-1515, 1522-1524, 1531-1533, 1540-1542, 1549-1551, 1558-1560, 1567-1569, 1576-1578, 1585-1587, 1594-1596, 1603-1605, 1612-1614, 1621-1623, 1630-1632, 1639-1641, 1648-1650, 1657-1659, 1666-1668, 1675-1677, 1684-1686, 1693-1695, 1702-1704, 1711-1713, 1720-1722, 1729-1731, 1886, 1907, 1909, 1911, 1913, 1915, 1917, 1919, 1921, 1923, 1925, 1927, 1929 or fragments or variants thereof; and
(ii) at least one coding RNA encoding at least one antigenic protein that is or is derived from VP8* of a Rotavirus A from a P[6] serotype, preferably according to SEQ ID NOs: 589, 590, 598, 599, 607, 608, 616, 617, 625, 626, 634, 635, 643, 644, 652, 653, 661, 662, 670, 671, 679, 680, 688, 689, 697, 698, 706, 707, 715, 716, 724, 725, 733, 734, 742, 743, 751, 752, 760, 761, 769, 770, 778, 779, 787, 788, 796, 797, 805, 806, 814, 815, 823, 824, 832, 833, 841, 842, 850, 851, 859, 860, 868, 869, 877, 878, 886, 887, 895, 896, 904, 905, 913, 914, 922, 923, 931, 932, 940, 941, 949, 950, 958, 959, 967, 968, 976, 977, 985, 986, 994, 995, 1003, 1004, 1012, 1013, 1021, 1022, 1030, 1031, 1039, 1040, 1048, 1049, 1057, 1058, 1066, 1067, 1075, 1076, 1084, 1085, 1093, 1094, 1102, 1103, 1111, 1112, 1120, 1121, 1129, 1130, 1138, 1139, 1147, 1148, 1156, 1157, 1165, 1166, 1174, 1175, 1183, 1184, 1192, 1193, 1201, 1202, 1210, 1211, 1219, 1220, 1228, 1229, 1237, 1238, 1246, 1247, 1255, 1256, 1264, 1265, 1273, 1274, 1282, 1283, 1291, 1292, 1300, 1301, 1309, 1310, 1318, 1319, 1327, 1328, 1336, 1337, 1345, 1346, 1354, 1355, 1363, 1364, 1372, 1373, 1381, 1382, 1390, 1391, 1399, 1400, 1408, 1409, 1417, 1418, 1426, 1427, 1435, 1436, 1444, 1445, 1453, 1454, 1462, 1463, 1471, 1472, 1480, 1481, 1489, 1490, 1498, 1499, 1507, 1508, 1516, 1517, 1525, 1526, 1534, 1535, 1543, 1544, 1552, 1553, 1561, 1562, 1570, 1571, 1579, 1580, 1588, 1589, 1597, 1598, 1606, 1607, 1615, 1616, 1624, 1625, 1633, 1634, 1642, 1643, 1651, 1652, 1660, 1661, 1669, 1670, 1678, 1679, 1687, 1688, 1696, 1697, 1705, 1706, 1714, 1715, 1723, 1724, 1732, 1733, 1887, 1890, 1895-1897, 1908, 1910, 1912, 1914, 1916, 1918, 1920, 1922, 1924, 1926, 1928, 1930, or fragments or variants thereof; and
(iii) at least one coding RNA encoding at least one antigenic protein that is or is derived from VP8* of a Rotavirus A from a P[8] serotype, preferably according to SEQ ID NOs: 591-594, 600-603, 609-612, 618-621, 627-630, 636-639, 645-648, 654-657, 663-666, 672-675, 681-684, 690-693, 699-702, 708-711, 717-720, 726-729, 735-738, 744-747, 753-756, 762-765, 771-774, 780-783, 789-792, 798-801, 807-810, 816-819, 825-828, 834-837, 843-846, 852-855, 861-864, 870-873, 879-882, 888-891, 897-900, 906-909, 915-918, 924-927, 933-936, 942-945, 951-954, 960-963, 969-972, 978-981, 987-990, 996-999, 1005-1008, 1014-1017, 1023-1026, 1032-1035, 1041-1044, 1050-1053, 1059-1062, 1068-1071, 1077-1080, 1086-1089, 1095-1098, 1104-1107, 1113-1116, 1122-1125, 1131-1134, 1140-1143, 1149-1152, 1158-1161, 1167-1170, 1176-1179, 1185-1188, 1194-1197, 1203-1206, 1212-1215, 1221-1224, 1230-1233, 1239-1242, 1248-1251, 1257-1260, 1266-1269, 1275-1278, 1284-1287, 1293-1296, 1302-1305, 1311-1314, 1320-1323, 1329-1332, 1338-1341, 1347-1350, 1356-1359, 1365-1368, 1374-1377, 1383-1386, 1392-1395, 1401-1404, 1410-1413, 1419-1422, 1428-1431, 1437-1440, 1446-1449, 1455-1458, 1464-1467, 1473-1476, 1482-1485, 1491-1494, 1500-1503, 1509-1512, 1518-1521, 1527-1530, 1536-1539, 1545-1548, 1554-1557, 1563-1566, 1572-1575, 1581-1584, 1590-1593, 1599-1602, 1608-1611, 1617-1620, 1626-1629, 1635-1638, 1644-1647, 1653-1656, 1662-1665, 1671-1674, 1680-1683, 1689-1692, 1698-1701, 1707-1710, 1716-1719, 1725-1728, 1734-1737, 1862-1882, 1885, 1888, 1889, 1891-1894, 1898, or fragments or variants thereof,

wherein preferably the at least one antigenic protein comprises a heterologous element selected from a signal peptide, a linker, a helper epitope, an antigen clustering domain, or a transmembrane domain.

Item 44:

Composition of any one of items 41 to 43, wherein the at least one coding RNA or the plurality of coding RNAs is complexed or associated with or at least partially complexed or partially associated with one or more cationic or polycationic compound, preferably cationic or polycationic polymer, cationic or polycationic polysaccharide, cationic or polycationic lipid, cationic or polycationic protein, cationic or polycationic peptide, or any combinations thereof.

Item 45:

Composition of item 44, wherein the at least one coding RNA or the plurality of coding RNAs is complexed, encapsulated, partially encapsulated, or associated with one or more lipids, thereby forming liposomes, lipid nanoparticles, lipoplexes, and/or nanoliposomes.

Item 46:

Composition of item 45, wherein the at least one coding RNA or the plurality of coding RNAs is complexed with one or more lipids thereby forming lipid nanoparticles (LNP).

Item 47:

Composition of item 46, wherein the LNP comprises a cationic lipid according to formula III-3:

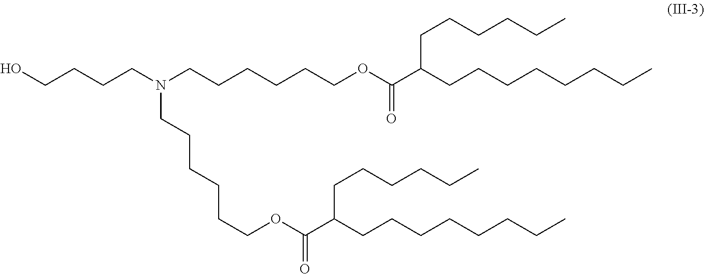 [SMILES: CCCCCCCCC(CCCCCC)C(=O)OCCCCCCN(CCCCO)CCCCCCOC(=O)C(CCCCCC)CCCCCCCC]

Item 48:

Composition of any one of items 46 to 47, wherein the LNP comprises a PEG lipid, wherein the PEG-lipid is of formula (IVa)

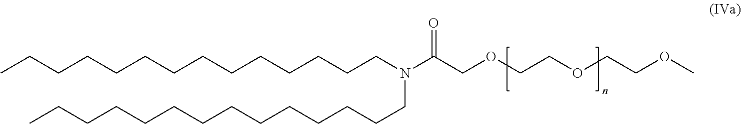

wherein n has a mean value ranging from 30 to 60, preferably wherein n has a mean value of about 45, 46, 47, 48, 49, 50, 51, 52, 53, 54, most preferably wherein n has a mean value of 49.

Item 49:

Composition of any one of items 46 to 48, wherein the LNP comprises one or more neutral lipids and/or one or more steroid or steroid analogues.

Item 50:

Composition of item 49, wherein the neutral lipid is 1,2-distearoyl-sn-glycero-3-phosphocholine (DSPC), preferably wherein the molar ratio of the cationic lipid to DSPC is in the range from about 2:1 to about 8:1.

Item 51:

Composition of item 49, wherein the steroid is cholesterol, preferably wherein the molar ratio of the cationic lipid to cholesterol is in the range from about 2:1 to about 1:1.

Item 52:

Composition of any one of items 44 to 49, wherein the LNP comprises or consisting of

(i) at least one cationic lipid, preferably as defined in item 47;

(ii) a neutral lipid, preferably as defined in item 50;

(iii) a steroid or steroid analogue, preferably as defined in item 51; and

(iv) a PEG-lipid, e.g. PEG-DMG or PEG-cDMA, preferably as defined in item 48.

Item 53:

Composition according to any one of items 52, wherein (i) to (iv) are in a molar ratio of about 20-60% cationic lipid, 5-25% neutral lipid, 25-55% sterol, and 0.5-15% PEG-lipid.

Item 54:

Composition of item 46, wherein the LNP comprises COATSOME® SS-EC.

Item 55:

Composition of any one of items 46 and 54, wherein the LNP comprises a PEG lipid, wherein the PEG-lipid is DMG-PEG 2000.

Item 56:

Composition of any one of items 46 and 54 to 55, wherein the LNP further comprises 1,2-diphytanoyl-sn-glycero-3-phosphoethanolamine (DPhyPE) and cholesterol.

Item 57:

Composition of items 46 to 56, wherein the LNPs are preferably selected from GN01-LNP or LNP-III-3.

Item 58:

A vaccine comprising at least one coding RNA as defined in any one of items 1 to 40, or the composition as defined in any one of items 41 to 58.

Item 59:

Vaccine of item 58, wherein the vaccine elicits an adaptive immune response.

Item 60:

Vaccine of item 58 to 59, wherein the vaccine elicits an adaptive immune response

Item 61:

Vaccine of item 58 to 60, wherein the vaccine induces specific and functional humoral immune responses against Rotavirus; and/or broad, functional cellular T-cell responses against Rotavirus.

Item 62:

Vaccine of item 58 to 61, wherein the vaccine induces high levels of virus neutralizing antibodies to prevent a Rotavirus infection, preferably high levels of virus neutralizing antibodies against homologous and heterologous Rotavirus strains.

Item 63:

Vaccine of items 58 to 62, wherein the vaccine is a polyvalent vaccine, preferably a trivalent vaccine.

Item 64:

A Kit or kit of parts, comprising at least one coding RNA as defined in any one of items 1 to 40, at least one composition as defined in any one of items 41 to 57, and/or at least one vaccine as defined in any one of items 58 to 62, optionally comprising a liquid vehicle for solubilising, and, optionally, technical instructions providing information on administration and dosage of the components.

Item 65:

Coding RNA as defined in any one of items 1 to 40, the composition as defined in any one of items 41 to 57, the vaccine as defined in any one of items 58 to 62, or the kit or kit of parts as defined in item 65, for use as a medicament.

Item 66:

Coding RNA as defined in any one of items 1 to 40, the composition as defined in any one of items 41 to 57, the vaccine as defined in any one of items 58 to 62, or the kit or kit of parts as defined in item 65, for use in the treatment or prophylaxis of a Rotavirus infection, or of a disorder related to such an infection.

Item 67:

Use according to item 66, wherein the Rotavirus infection is a Rotavirus A infection, in particular a Rotavirus A infection of serotypes [P4], [P6], and/or [P8].

Item 68:

A method of treating or preventing a disorder, wherein the method comprises applying or administering to a subject in need thereof at least one coding RNA as defined in any one of items 1 to 40, at least one composition as defined in any one of items 41 to 57, at least one vaccine as defined in any one of items 58 to 62, or at least one kit or kit of parts as defined in item 65.

Item 69:

Method of item 68, wherein the disorder is an infection with a Rotavirus, or a disorder related to such an infection, preferably a Rotavirus A, or a disorder related to such an infection.

Item 70:

Method of items 68 to 69, wherein the subject in need is a mammalian subject, preferably a human subject.

Item 71:

Method of any one of items 68 to 70, wherein applying or administering to a subject is performed using intramuscular administration, preferably intramuscular injection.

### BRIEF DESCRIPTION OF TABLES

Table 1: Preferred VP8* antigen constructs

Table X1: Suitable heterologous elements for VP8* antigen constructs

Table 2: Human codon usage table with frequencies indicated for each amino acid

Table 3: Preferred coding sequences encoding Rotavirus VP8* antigen constructs:

Table 4: Preferred coding RNA, e.g. mRNA, encoding Rotavirus VP8* antigen constructs

Table X4: Coding RNA, e.g. mRNA, encoding Rotavirus VP8* antigen constructs and others

Table 5: RNA constructs encoding different Rotavirus antigen design used in the present examples

Table 6: RNA constructs used for Western blot analysis (Example 2)

Table 7: Vaccination scheme of Example 2

Table 8: Vaccination scheme of Example 3.1

Table 9 Rotavirus VP8* peptide mix for ICS

Table 10: Vaccination scheme of Example 3.2

Table 11: RNA constructs used for Western blot analysis (Example 4)

Table 12: Vaccination scheme of Example 5

Table 13: RNA constructs used for Western blot analysis (Example 6)

Table 14: Vaccination scheme of Example 6.2

Table 15: Vaccination scheme of Example 8

Table 16: Vaccination scheme of Example 9

Table 17: Vaccination scheme of Example 10

Table 18: Vaccination scheme of Example 11

Table 19: Vaccination scheme of Example 12

Table 20: Vaccination scheme of Example 13

### BRIEF DESCRIPTION OF THE DRAWINGS

FIG. 1 shows schematic drawings of preferred VP8* constructs. P2: T cell helper epitope from tetanus toxin; VP8*: Virus protein 8*, cleavage product of rotavirus VP4 protein (preferably having a length of 65-223, 41-223, 1-223, 20-240, 1-230, 2-230, 10-223 or 11-223, preferably 1-223 or 65-223); SP: Signal peptide; L: Linker; Ferritin: Iron storage protein ferritin; Lum. synt.: Lumazine synthase (LumSynt, LS).

FIG. 2 shows that mRNA constructs encoding different Rotavirus antigen designs were expressed and partially secreted in mammalian cells using Western blot analysis. The experiment was performed as described in Example 2.1. Further details are provided in Table 6.

FIG. 3 shows that formulated mRNA constructs encoding different Rotavirus antigen designs induced humoral immune responses in mice. IgG1 and IgG2a antibody titers assessed by ELISA using recombinant Rotavirus protein P2-VP8*P[8] protein as a coating reagent. The experiment was performed as described in Example 2.2. Further construct details are provided in Table 7. Significant IgG1 and IgG2a responses were detectable for all groups vaccinated with the mRNA vaccine encoding different Rotavirus antigen designs.

FIG. 4 shows the reactivity of Rotavirus serotype P[6] antigen designs.

FIG. 4-A shows that formulated mRNA constructs encoding different Rotavirus antigen designs induced humoral immune responses in mice. IgG1 and IgG2a antibody titers assessed by ELISA using P2-VP8*P[6] protein as a coating reagent. The experiment was performed as described in Example 3.1.1. Further construct details are provided in Table 8. Significant IgG1 and IgG2a responses were detectable for all groups vaccinated with the mRNA vaccine encoding different Rotavirus antigen designs.

FIG. 4-B shows cross reactive responses in mice vaccinated with P[6] designs with P[8] serotype protein as a coating reagent. mRNA constructs encoding different Rotavirus antigen designs induced cross reactive humoral immune responses in mice. The experiment was performed as described in Example 3.1.1. Further construct details are provided in Table 8.

FIG. 4-C shows that formulated mRNA constructs encoding different Rotavirus antigen designs induced cellular immune responses of CD4 and CD8 positive T-cells in mice, using an intracellular cytokine staining assay. The experiment was performed as described in Example 3.1.2. Further construct details are provided in Table 8.

FIG. 5 shows the reactivity of Rotavirus serotype P[8] antigen designs.

FIG. 5-A shows that formulated mRNA constructs encoding different Rotavirus antigen designs induced humoral immune responses in mice. IgG1 and IgG2a antibody titers assessed by ELISA using recombinant Rotavirus protein VP8*P[8] as a coating reagent. The experiment was performed as described in Example 3.2.1. Further construct details are provided in Table 10. Significant IgG1 and IgG2a responses were detectable for all groups vaccinated with the mRNA vaccine encoding different Rotavirus antigen designs.

FIG. 5-B shows cross reactive responses in mice vaccinated with P[8] designs with recombinant Rotavirus protein P2-VP8*P[6] as a coating reagent. mRNA constructs encoding different Rotavirus antigen designs induced cross reactive humoral immune responses in mice. The experiment was performed as described in Example 3.1.2. Further construct details are provided in Table 10.

FIG. 5-C shows that formulated mRNA constructs encoding different Rotavirus antigen designs induced cellular immune responses of CD4 positive T-cells in mice, using an intracellular cytokine staining assay. In addition Group 8 shows cellular immune responses of CD8 positive T cells. The experiment was performed as described in Example 3.2.2. Further construct details are provided in Table 10.

FIG. 6 shows that different mRNA designs encoding Rotavirus antigens were expressed in mammalian cells using Western blot analysis. The experiment was performed as described in Example 4. Further details are provided in Table 11.

FIG. 7 shows significant IgG1 and IgG2a responses for all groups vaccinated with the cap1 mRNA design (Group 4, 5, 6 and 7) encoding the same Rotavirus antigen construct. mRNA designs with a poly(A) sequence, located at 3′ terminus (Group 6 and 7) shown higher IgG responses compared to mRNA designs without a poly(A) sequence, located at 3′ terminus (Group 4 and 5). In addition FIG. 7 shows comparable IgG1 and IgG2a responses for all mRNA designs vaccinated with modified (grey bars) or unmodified (black bars) nucleotides. IgG1 and IgG2a antibody titers assessed by ELISA using Rotavirus protein P2-VP8*P[8] as a coating reagent. The experiment was performed as described in Example 5. Further construct details are provided in Table 12.

FIG. 8 shows that all mRNA designs with cap1 and a poly(A) sequence, located at 3′ terminus (Group 6 and 7) induced the formation of Rotavirus specific functional antibodies in mice as shown by robust virus neutralizing antibody titers. cap1 mRNA designs without a poly(A) sequence, located at 3′ terminus (Group 4 and 5) shown an effect compared to the negative control (Group 1). The experiment was performed as described in Example 5.2. Further details are provided in Table 12.

FIG. 9 shows that different mRNA designs encoding Rotavirus antigens were expressed in mammalian cells using Western blot analysis. mRNA designs with co-transcriptional capping and a beneficial UTR combination (Group 1, 2 and 3) showed higher expression compared to the corresponding constructs with enzymatical capping and another UTR combination (Group 4, 5 and 6). The experiment was performed as described in Example 6.1. Further details are provided in Table 13.

FIG. 10 shows the in vivo analysis of immunogenicity of different mRNA constructs encoding a Rotavirus antigen.

FIG. 10-A shows early (day 21) IgG1 and IgG2a responses for all groups with a poly(A) sequence, located at 3′ terminus (Group 4, 5, 7, 8, 10, 11 and 13), independent of UTR combination (black bars, striped bars or dotted bars) and modification of nucleotides. The experiment was performed as described in Example 6.2. Further construct details are provided in Table 14.

FIG. 10-B shows high IgG1 and IgG2a responses after day 56 for all groups vaccinated with different mRNA designs. The experiment was performed as described in Example 6.2. Further construct details are provided in Table 14.

FIG. 10-C shows early (day 21) IgG1 and IgG2a responses for all groups vaccinated with mRNA designs that are co-transcriptional capped and have a poly(A) sequence, located at 3′ terminus and a UTR combination of HSD17B4/PSMB3 (black bars) compared to mRNA designs with an enzymatical cap, a poly(A) sequence, located at 3 terminus and other UTR combinations (striped bars). The experiment was performed as described in Example 6.2. Further construct details are provided in Table 14.

FIG. 10-D shows high IgG1 and IgG2a responses after day 56 for all groups vaccinated with different mRNA designs. The experiment was performed as described in Example 6.2. Further construct details are provided in Table 14.

FIG. 11 shows that all mRNA designs with a poly(A) sequence, located at 3′ terminus (Group 4, 5, 7, 8, 10, 11 and 13) induced the formation of Rotavirus specific functional antibodies in mice as shown by robust virus neutralizing antibody titers. mRNA designs that are co-transcriptional capped and have a poly(A) sequence, located at 3′ terminus and a UTR combination of HSD17B4/PSMB3 (Group 4 and 10) induced higher VNT titers compared to mRNA designs with enzymatical cap (Group 7 and 3). This effect is independent of modified nucleotides. The experiment was performed as described in Example 6.3. Further details are provided in Table 14.

FIG. 12 shows INFalpha levels in the sera 14 hours after prime immunization. Group 2, the recombinant Rotavirus protein P2-VP8*P[8]+Alum, and Groups with modified nucleotides (Group 9, 10, 11, 12 and 13) induced no increasing of INFalpha levels. Group 6, 7 and 8 induced high INFalpha levels compared to group Group 3, 4 and 5. Group 3, 4 and 5 showed only a moderate increasing of INFalpha levels in the sera. The experiment was performed as described in Example 6.2 and 6.4. Further details are provided in Table 14.

FIG. 13 shows cellular immune responses of CD8 and CD4 positive T-cells in mice, using an intracellular cytokine staining assay. Group 4, 5, 10 and 11 induced the highest cellular immune responses of CD8 positive T-cells. All groups of Rotavirus vaccines with a poly(A) sequence, located at 3′ terminus showed higher cellular immune responses of CD4 positive T-cells compared to the recombinant Rotavirus protein P2-VP8*P[8]+Alum (Group 2). The experiment was performed as described in Example 6.5. Further construct details are provided in Table 14.

FIG. 14 shows that formulated Rotavirus VP8* mRNA vaccines encoding different antigen designs induce humoral immune responses in guinea pigs, using an ELISA and VNT assay. FIG. 14A: coating: P2-VP8*P[8]; IgG endpoint titers at day 21 post prime vaccination; FIG. 14B: coating: P2-VP8*P[8]; IgG endpoint titers at day 42 post prime vaccination; FIG. 14C: coating: P2-VP8*P[8]; IgG endpoint titers at day 56 post prime vaccination; FIG. 14D: VNTs against Rotavirus virus strain Wa (G1P[8]) at day 56 post prime vaccination. Groups 3 to 5 received 6 μg Rotavirus VP8* mRNA vaccine, Groups 6 to 10 received 25 μg Rotavirus VP8* mRNA vaccine, Groups 11 to 13 received 100 μg Rotavirus VP8* mRNA vaccine, Group 1 is the negative control sham-immunized with NaCl and Group 2 is the positive control immunized with 20 μg recombinant Rotavirus VP8* P[8] protein. For vaccination scheme, see Table 15. Further details provided in Example 8.

FIG. 15 shows that formulated Rotavirus VP8* mRNA vaccines encoding different antigen designs induce longevity humoral immune responses in guinea pigs, using an ELISA assay. Coating: P2-VP8*P[8]; IgG endpoint titers at day 21, 42, 56, 84, 112 and 140 post prime vaccination; for longevity ELISA only Groups 1, 2, 6, 8 and 10 were selected. Groups 6 to 10 received 25 μg Rotavirus VP8* mRNA vaccine, Group 1 is the negative control sham-immunized with NaCl and Group 2 is the positive control immunized with 20 μg recombinant Rotavirus VP8* P[8] protein. For vaccination scheme, see Table 15. Further details provided in Example 8.

FIG. 16 shows that formulated Rotavirus VP8* mRNA vaccines encoding different antigen designs induce humoral immune responses in guinea pigs, using an ELISA assay. FIG. 16A: coating: P2-VP8*P[8]; IgG endpoint titers at day 21 post prime vaccination; FIG. 16B: coating: P2-VP8*P[8]; IgG endpoint titers at day 42 post prime vaccination; FIG. 16C: coating: P2-VP8*P[8]; IgG endpoint titers at day 56 post prime vaccination; FIG. 16D: coating: P2-VP8*P[8]; IgA endpoint titers at day 56 post prime vaccination; Groups 3 to 6 received 6 μg Rotavirus VP8* mRNA vaccine, Groups 7 to 10 received 25 μg Rotavirus VP8* mRNA vaccine, Group 1 is the negative control sham-immunized with NaCl and Group 2 is the positive control immunized with 20 μg recombinant Rotavirus VP8* P[8] protein. For vaccination scheme, see Table 16. Further details provided in Example 9.

FIG. 17 shows that formulated Rotavirus VP8* mRNA vaccines encoding different antigen designs induce longevity humoral immune responses in guinea pigs, using an ELISA assay. Coating: P2-VP8*P[8]; IgG endpoint titers at day 21, 42, 56, 84, 112, 140, 168 and 196 post prime vaccination; for longevity ELISA only Groups 1, 2, 7, 8, 9 and 10 were selected. Groups 7 to 10 received 25 μg Rotavirus VP8* mRNA vaccine, Group 1 is the negative control sham-immunized with NaCl and Group 2 is the positive control immunized with 20 μg recombinant Rotavirus VP8* P[8] protein. For vaccination scheme, see Table 16. Further details provided in Example 9.

FIG. 18 shows that formulated Rotavirus VP8* mRNA vaccines encoding different antigen designs induce humoral immune responses in guinea pigs, using a VNT assay. FIG. 18A: serum incubation with Wa (G1P[8]) at day 56 post prime vaccination; FIG. 18B: serum incubation with rotavirus 1076 (G2P[6]) at day 56 post prime vaccination; FIG. 18C: serum incubation with rotavirus DS-1 (G2P[4]) at day 56 post prime vaccination; Groups 3 to 6 received 6 μg Rotavirus VP8* mRNA vaccine, Groups 7 to 10 received 25 μg Rotavirus VP8* mRNA vaccine, Group 1 is the negative control sham-immunized with NaCl and Group 2 is the positive control immunized with 20 μg recombinant Rotavirus VP8* P[8] protein. For vaccination scheme, see Table 16. Further details provided in Example 9.

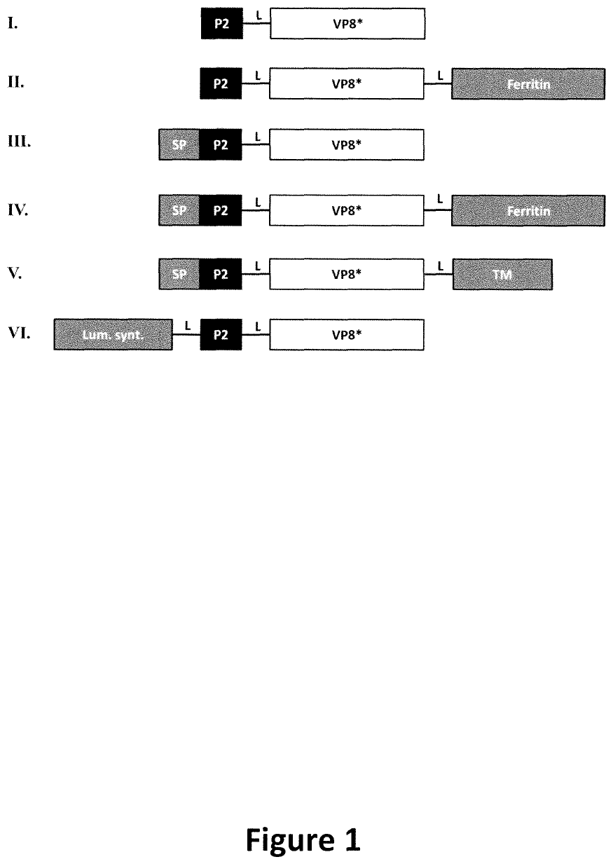

### EXAMPLES

In the following, particular examples illustrating various embodiments and aspects of the invention are presented. However, the present invention shall not to be limited in scope by the specific embodiments described herein. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below. All such modifications fall within the scope of the appended claims.

### Example 1: Preparation of DNA and RNA Constructs, Compositions, and Vaccines

The present Example provides methods of obtaining the RNA of the invention as well as methods of generating a composition or a vaccine of the invention.

1.1. Preparation of DNA and RNA Constructs:

DNA sequences encoding different Rotavirus antigenic proteins were prepared and used for subsequent RNA in vitro transcription. Said DNA sequences were prepared by modifying the wild type CDS sequences by introducing an optimized CDS. Sequences were introduced into a plasmid vector to comprise optionally (i) advantageous 3′-UTR sequences derived from PSMB3, ALB7 or alpha-globin (“muag”) and (ii) advantageous 5′-UTR sequences selected from HSD17B4 or RPL32, additionally comprising (iii) a stretch of adenosines, and optionally a histone-stem-loop structure, and optionally a stretch of 30 cytosines (Table 5).

Obtained plasmid DNA was transformed and propagated in bacteria using common protocols and plasmid DNA was extracted, purified, and used for subsequent RNA in vitro transcription (see section 1.2.). Alternatively, DNA plasmids were used as DNA template for PCR-based amplification. The generated PCR products were purified and used for subsequent RNA in vitro transcription (see section 1.3.).

1.2. RNA In Vitro Transcription from Plasmid DNA Templates:

DNA plasmids prepared according to section 1.1 were enzymatically linearized using a restriction enzyme and used for DNA dependent RNA in vitro transcription using T7 RNA polymerase in the presence of a nucleotide mixture (ATP/GTP/CTP/UTP) and cap analogue (e.g., m7GpppG or m7G(5′)ppp(5′)(2′OMeA)pG or m7G(5′)ppp(5′)(2′OMeG)pG)) under suitable buffer conditions. m7G(5′)ppp(5′)(2′OMeA)pG cap analog was used for preparation of some RNA constructs to generate co-transcriptionally a cap1 structure. The obtained RNA constructs were purified using RP-HPLC (PureMessenger®, CureVac AG, Tubingen, Germany; WO2008/077592) and used for in vitro and in vivo experiments. DNA templates may also be generated using PCR. Such PCR templates were used for DNA dependent RNA in vitro transcription using an RNA polymerase as outlined herein.

To obtain modified mRNA RNA in vitro transcription was performed in the presence of a modified nucleotide mixture (ATP, GTP, CTP, pseudouridine (Ψ) and cap analogue (m7GpppG or m7G(5′)ppp(5′)(2′OMeA)pG) under suitable buffer conditions. The obtained Ψ-modified mRNAs were purified using RP-HPLC (PureMessenger®, CureVac AG, Tübingen, Germany; WO2008/077592) and used for further experiments. Some RNA constructs were in vitro transcribed in the absence of a cap analog. The cap-structure (cap0 or cap1) was then added enzymatically using capping enzymes as commonly known in the art. In short, in vitro transcribed RNA was capped using a capping kit to obtain cap0-RNA. cap0-RNA was additionally modified using cap specific 2′-O-methyltransferase to obtain cap1-RNA. cap1-RNA was purified e.g. as explained above and used for further experiments.

RNA for clinical development is produced under current good manufacturing practice e.g. according to WO2016/180430, implementing various quality control steps on DNA and RNA level.

The generated RNA sequences/constructs are provided in Table 5 with the encoded antigenic protein and the respective UTR elements indicated therein.

1.3. RNA In Vitro Transcription from PCR Amplified DNA Templates:

Purified PCR amplified DNA templates prepared according to paragraph 1.1 are transcribed in vitro using DNA dependent T7 RNA polymerase in the presence of a nucleotide mixture (ATP/GTP/CTP/UTP) and cap analogue (m7GpppG or 3′-O-Me-m7G(5′)ppp(5′)G)) under suitable buffer conditions. Alternatively, PCR amplified DNA is transcribed in vitro using DNA dependent T7 RNA polymerase in the presence of a modified nucleotide mixture (ATP, GTP, CTP, N1-methylpseudouridine (m1Ψ) or pseudouridine (Ψ) and cap analogue (m7GpppG, m7G(5′)ppp(5′)(2′OMeA)pG or m7G(5′)ppp(5′)(2′OMeG)pG) under suitable buffer conditions. Some RNA constructs are in vitro transcribed in the absence of a cap analogue and the cap-structure (cap0 or cap1) is added enzymatically using capping enzymes as commonly known in the art. The obtained RNA is purified e.g. as explained above and used for further experiments.

TABLE 5
RNA constructs encoding different Rotavirus antigen design used in the present examples

| | | | mRNA Design | | | | | |
|---|---|---|---|---|---|---|---|---|
| | | | | | Poly(A) | | | |
| | | | | UTR | sequence, | | | |
| | | | 5′-cap | design | located at 3′ | | | |
| RNA | Construct | Rotavirus | structure | 5′-UTR/ | terminus | Modified | SEQ ID | SEQ ID |
| ID | design | Serotype | (Description) | 3′-UTR | (Description) | nucleotides | NO: RNA | NO: PRT |
| R5470 | P2-Linker- | P[8] | cap0 | RPL32/ | — | — | 1865 | 4, 51 |
| | VP8*(65-223) | | | ALB7 | | | | |
| R6326 | P2-Linker- | P[8] | cap0 | RPL32/ | — | — | 1874 | 69 |
| | VP8*(65- | | | ALB7 | | | | |
| | 223)-Linker- | | | | | | | |
| | Ferritin | | | | | | | |
| R5488 | SP(IgE)-P2- | P[8] | cap0 | RPL32/ | — | — | 1880 | 114 |
| | Linker- | | | ALB7 | | | | |
| | VP8*(41-223) | | | | | | | |
| R6322 | SP(IgE)-P2- | P[8] | cap0 | RPL32/ | — | — | 1881 | — |
| | Linker- | | | ALB7 | | | | |
| | VP8*(41- | | | | | | | |
| | 223)-Linker- | | | | | | | |
| | Ferritin | | | | | | | |
| R6324 | SP(IgE)-P2- | P[8] | cap0 | RPL32/ | — | — | 1882 | — |
| | Linker- | | | ALB7 | | | | |
| | VP8*(41- | | | | | | | |
| | 223)-Linker- | | | | | | | |
| | TM(HA) | | | | | | | |
| R6328 | LumSynt.- | P[8] | cap0 | RPL32/ | — | — | 1877 | 1, 96 |
| | Linker-P2- | | | ALB7 | | | | |
| | Linker- | | | | | | | |
| | VP8*(41-223) | | | | | | | |
| R5433 | SP(HsPLAT)- | P[6] | cap0 | RPL32/ | — | — | 1895 | — |
| | VP8*(41-223) | | | ALB7 | | | | |
| R5434 | SP(HsPLAT)- | P[6] | cap0 | RPL32/ | — | — | 1896 | — |
| | P2-Linker- | | | ALB7 | | | | |
| | VP8*(41-223) | | | | | | | |
| R5435 | SP(HsPLAT)- | P[6] | cap0 | RPL32/ | — | — | 1897 | — |
| | VP8*(2-230) | | | ALB7 | | | | |
| R5436 | SP(HsPLAT)- | P[6] | cap0 | RPL32/ | — | — | 1890 | — |
| | VP8*(21-240) | | | ALB7 | | | | |
| | (N => Q | | | | | | | |
| | mutation) | | | | | | | |
| R5480 | SP(HSA)- | P[8] | cap0 | RPL32/ | — | — | 1891 | — |
| | VP8*(2-230) | | | ALB7 | | | | |
| R5482 | SP(HSA)- | P[8] | cap0 | RPL32/ | — | — | 1892 | — |
| | VP8*(11-223) | | | ALB7 | | | | |
| R5484 | SP(HSA)- | P[8] | cap0 | RPL32/ | — | — | 1893 | — |
| | VP8*(41-223) | | | ALB7 | | | | |
| R5486 | SP(HSA)-P2- | P[8] | cap0 | RPL32/ | — | — | 1894 | — |
| | Linker- | | | ALB7 | | | | |
| | VP8*(41-223) | | | | | | | |
| R8044 | P2-Linker- | P[8] | cap1 (co- | HSD17B4/ | — | — | 1864 | 4, 51 |
| | VP8*(65-223) | | trans. cap) | PSMB3 | | | | |
| R8046 | P2-Linker- | P[8] | cap1 (enzym. | RPL32/ | + (enzym. | — | 1865 | 4, 51 |
| | VP8*(65-223) | | cap) | ALB7 | poly(A)) | | | |
| R8047 | P2-Linker- | P[8] | cap0 | RPL32/ | — | m1Ψ | 1870 | 4, 51 |
| | VP8*(65-223) | | | ALB7 | | | | |
| R8049 | P2-Linker- | P[8] | cap1 (co- | HSD17B4/ | — | m1Ψ | 1868 | 4, 51 |
| | VP8*(65-223) | | trans. cap) | PSMB3 | | | | |
| R7411 | P2-Linker- | P[8] | cap1 (enzym. | —/muag | + (enzym. | m1Ψ | 1869 | 4, 51 |
| | VP8*(65-223) | | cap) | | poly(A)) | | | |
| R8134 | P2-Linker- | P[8] | cap1 (co- | HSD17B4/ | + (enzym. | — | 1864 | 4, 51 |
| | VP8*(65-223) | | trans. cap) | PSMB3 | poly(A)) | | | |
| R8131 | P2-Linker- | P{8] | cap1 (co- | HSD17B4/ | + (A100) | — | 1862, | 4, 51 |
| | VP8*(65-223) | | trans. cap) | PSMB3 | | | 591 | |
| R8135 | P2-Linker- | P[8] | cap1 (enzym. | RPL32/ | — | — | 1865 | 4, 51 |
| | VP8*(65-223) | | cap) | ALB7 | | | | |
| R8138 | P2-Linker- | P[8] | cap1 (enzym. | RPL32/ | + (A100) | — | 1898 | 4, 51 |
| | VP8*(65-223) | | cap) | ALB7 | | | | |
| R8136 | P2-Linker- | P[8] | cap1 (co- | HSD17B4/ | + (enzym. | m1Ψ | 1868 | 4, 51 |
| | VP8*(65-223) | | trans. cap) | PSMB3 | poly(A)) | | | |
| R8133 | P2-Linker- | P[8] | cap1 (co- | HSD17B4/ | + (A100) | m1Ψ | 1866 | 4, 51 |
| | VP8*(65-223) | | trans. cap) | PSMB3 | | | | |
| R8137 | P2-Linker- | P[8] | cap1 (enzym. | —/muag | — | m1Ψ | 1869 | 4, 51 |
| | VP8*(65-223) | | cap) | | | | | |
| R8628 | P2-Linker- | P[8] | cap1 (co- | HSD17B4/ | + (A100) | — | 1863, | 4. 51 |
| | VP8*(65-223) | | trans. cap) | PSMB3 | | | 1167 | |
| R8575 | P2-Linker- | P[8] | cap1 (co- | HSD17B4/ | + (A100) | m1Ψ | 1863, | 4, 51 |
| | VP8*(65-223) | | trans. cap) | PSMB3 | | | 1167 | |
| R8576 | P2-Linker- | P[8] | cap1 (co- | HSD17B4/ | + (A100) | Ψ | 1863, | 4, 51 |
| | VP8*(65-223) | | trans. cap) | PSMB3 | | | 1167 | |
| R8629 | P2-Linker- | P[8] | cap1 (co- | HSD17B4/ | + (A100) | Ψ | 1867 | 4, 51 |
| | VP8*(65-223) | | trans. cap) | PSMB3 | | | | |
| R8577 | P2-Linker- | P[8] | cap1 (co- | HSD17B4/ | + (A100) | — | 1873, | 69 |
| | VP8*(65- | | trans. cap) | PSMB3 | | | 1185 | |
| | 223)-Linker- | | | | | | | |
| | Ferritin | | | | | | | |
| R8578 | LumSynt.- | P[8] | cap1 (co- | HSD17B4/ | + (A100) | — | 1876, | 1, 96 |
| | Linker-P2- | | trans. cap) | PSMB3 | | | 1212 | |
| | Linker- | | | | | | | |
| | VP8*(41-223) | | | | | | | |
| R8579 | SP(IgE)-P2- | P[8] | cap1 (co- | HSD17B4/ | + (A100) | — | 1879, | 114 |
| | Linker- | | trans. cap) | PSMB3 | | | 1230 | |
| | VP8*(41-223) | | | | | | | |
| R9247 | P2-Linker- | P[4] | cap1 (co- | HSD17B4/ | + (A100) | — | 1919 | 1899 |
| | VP8*(64-223) | | trans. cap) | PSMB3 | | | | |
| R9246 | P2-Linker- | P[6] | cap1 (co- | HSD17B4/ | + (A100) | — | 1920 | 1900 |
| | VP8*(64-223) | | trans. cap) | PSMB3 | | | | |
| R9078 | P2-Linker- | P[4] | cap1 (co- | HSD17B4/ | + (A100) | — | 1921 | 6, 46 |
| | VP8*(65-223) | | trans. cap) | PSMB3 | | | | |
| R9077 | P2-Linker- | P[6] | cap1 (co- | HSD17B4/ | + (A100) | — | 1922 | 5, 49 |
| | VP8*(65-223) | | trans. cap) | PSMB3 | | | | |
| R9092 | LumSynt.- | P[4] | cap1 (co- | HSD17B4/ | + (A100) | — | 1923 | 3, 91 |
| | Linker-P2- | | trans. cap) | PSMB3 | | | | |
| | Linker- | | | | | | | |
| | VP8*(41-223) | | | | | | | |
| R9091 | LumSynt.- | P[6] | cap1 (co- | HSD17B4/ | + (A100) | — | 1924 | 2, 94 |
| | Linker-P2- | | trans. cap) | PSMB3 | | | | |
| | Linker- | | | | | | | |
| | VP8*(41-223) | | | | | | | |

Brief Description of the Table 5:

P2: T cell helper epitope from tetanus toxin; VP8*: Virus protein 8*, cleavage product of rotavirus VP4 protein (preferably having a length of 65-223, 41-223, 1-223, 20-240, 1-230, 2-230, 10-223 or 11-223); SP: signal peptide (preferably derived from secretory signal peptides, preferably human serum albumin (HSA), tissue plasminogen activator (HsPLAT) or immunoglobulin IgE (IgE)); Ferritin: iron storage protein ferritin, heterologous antigen-clustering element; LumSynt: Lumazine synthase, heterologous antigen-clustering element; TM: Transmembrane domain (preferably derived from an influenza HA); P[X]: Rotavirus P serotypes; m1Ψ: modified nucleotide (N1-methylpseudouridine); Ψ: modified nucleotide (pseudouridine); poly(A) sequence, located at 3′ terminus: poly(A) sequence obtained by enzymatic polyadenylation (enzym. poly(A)) or a DNA template (A100); cap0: methylation of the first nucleobase, e.g. m7GpppN; cap1: additional methylation of the ribose of the adjacent nucleotide of m7GpppN; co trans. cap: co-transcriptional capping (preferably CleanCap); enzym. cap: enzymatically capping (preferably ScriptCap); N=>Q mutation: mutation in position N32Q, N56Q, N97Q, N111Q, N114Q, N132Q, N171Q and/or N182Q.

1.4. Preparation of Vaccine:

1.4.1 LNP Formulation

Lipid nanoparticles (LNP), cationic lipids, and polymer conjugated lipids (PEG-lipid) were prepared and tested essentially according to the general procedures described in WO2015/199952, WO2017/004143 and WO2017/075531, the full disclosures of which are incorporated herein by reference. LNP formulated RNA was prepared using an ionizable amino lipid (cationic lipid), phospholipid, cholesterol and a PEGylated lipid. Briefly, cationic lipid compound of formula III-3, DSPC, cholesterol, and PEG-lipid of formula IVa were solubilized in ethanol at a molar ratio (%) of approximately 50:10:38.5:1.5 or 47.4:10:40.9:1.7. LNPs comprising cationic lipid compound of formula III-3 and PEG-lipid compound of formula IVa were prepared at a ratio of RNA to total Lipid of 0.03-0.04 w/w. The RNA was diluted to 0.05 mg/mL to 0.2 mg/mL in 10 mM to 50 mM citrate buffer, pH4. Syringe pumps were used to mix the ethanolic lipid solution with the RNA aqueous solution at a ratio of about 1:5 to 1:3 (vol/vol) with total flow rates above 15 ml/min. The ethanol was then removed and the external buffer replaced with a PBS buffer comprising Sucrose by dialysis. Finally, the lipid nanoparticles were filtered through a 0.2 um pore sterile filter and the LNP-formulated RNA composition was adjusted to about 1 mg/ml total RNA. Lipid nanoparticle particle diameter size was 60-90 nm as determined by quasi-elastic light scattering using a Malvern Zetasizer Nano (Malvern, UK). For other cationic lipid compounds mentioned in the present specification, the formulation process is essentially similar.

In general, LNPs were prepared using cationic lipids, structural lipids, a PEG-lipids, and cholesterol. Lipid solution (in ethanol) was mixed with RNA solution (aqueous buffer) using a microfluidic mixing device or using T-piece formulation. Obtained LNPs were re-buffered in a carbohydrate buffer via dialysis, and up-concentrated to a target concentration using ultracentrifugation tubes. LNP-formulated mRNA can be stored at −80° C. prior to use in in vitro or in vivo experiments.

The obtained LNP-formulated RNA composition (1 mg/ml total RNA) was diluted to the desired target concentration using Saline before in vivo application.

1.4.2 Protamine Complexation:

The mRNA vaccine consisted of a mixture of 50% free mRNA and 50% mRNA complexed with protamine at a weight ratio of 2:1. First, mRNA was complexed with protamine by addition of protamine-Ringer's lactate solution to mRNA. After incubation for 10 minutes, when the complexes were stably generated, free mRNA was added, and the final concentration of the vaccine was adjusted with Ringer's lactate solution.

### Example 2: Analysis of Rotavirus Antigen Designs

2.1 In Vitro Analysis of Expression and Secretion of Rotavirus Antigen Designs

The present example shows that RNA constructs encoding different Rotavirus antigen designs were expressed and secreted in mammalian cells.

To determine in vitro protein expression of some of the RNA constructs, HEK 293T cells were transfected with unformulated mRNA encoding different Rotavirus antigen designs using Lipofectamine 2000. 24 h-48 h after transfection, cell lysates and cell culture supernatants were subjected to SDS-PAGE and Western blot analysis using rabbit anti-VP8* P[4 and 8] antibody (1:1000; Aldevron) or mouse anti-alpha-tubulin antibody (1:1000; Abcam) as primary antibodies as well as goat anti-rabbit IgG IRDye®800CW or 680RD antibody (1:10000; Li-Cor) or goat anti-mouse IgG IRDye® 680RD or 800CW antibody (1:10000; Li-Cor) as secondary antibodies. Detection and quantification was performed using a Li-Cor detection system (Odyssey CLx image system) in combination with Image Studio Lite software. Table 6 contains mRNA constructs that were used in the experiment:

TABLE 6
RNA constructs used for Western blot analysis (Example 2)

| | RNA | | Size | SEQ ID |
|---|---|---|---|---|
| Group | ID | Construct design | kDa | NO: RNA |
| 1 | R5470 | P2-Linker-VP8*(65-223) | 19 | 1865 |
| 2 | R6326 | P2-Linker-VP8*(65-223)-Linker-Ferritin | 38 | 1874 |
| 3 | R5488 | SP(IgE)-P2-Linker-VP8*(41-223) | 24 | 1880 |
| 4 | R6322 | SP(IgE)-P2-Linker-VP8*(41-223)-Linker-Ferritin | 41 | 1881 |
| 5 | R6324 | SP(IgE)-P2-Linker-VP8*(41-223)-Linker-TM | 30 | 1882 |
| 6 | R6328 | LumSynt-Linker-P2-Linker-VP8*(41-223) | 43 | 1877 |
| 7 | — | Negative control (WFI = water for infusion) | — | 1883 |

Results:

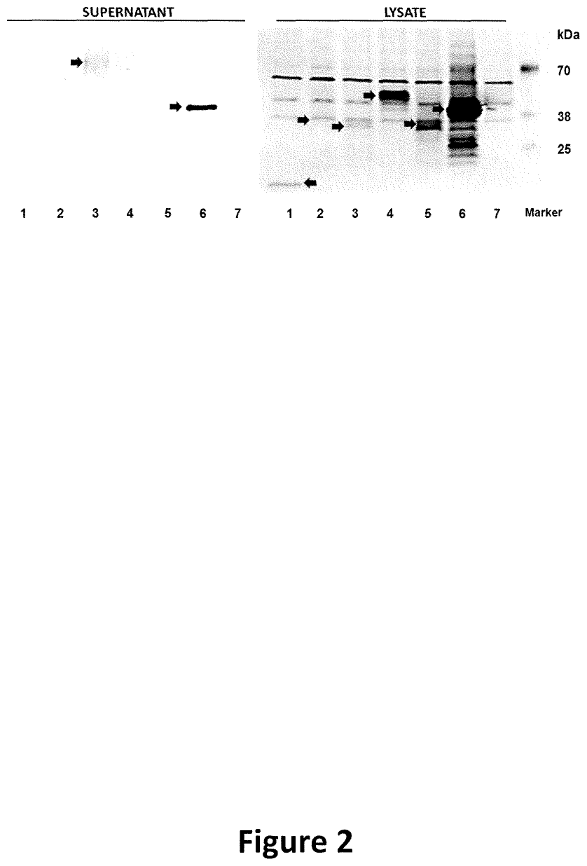

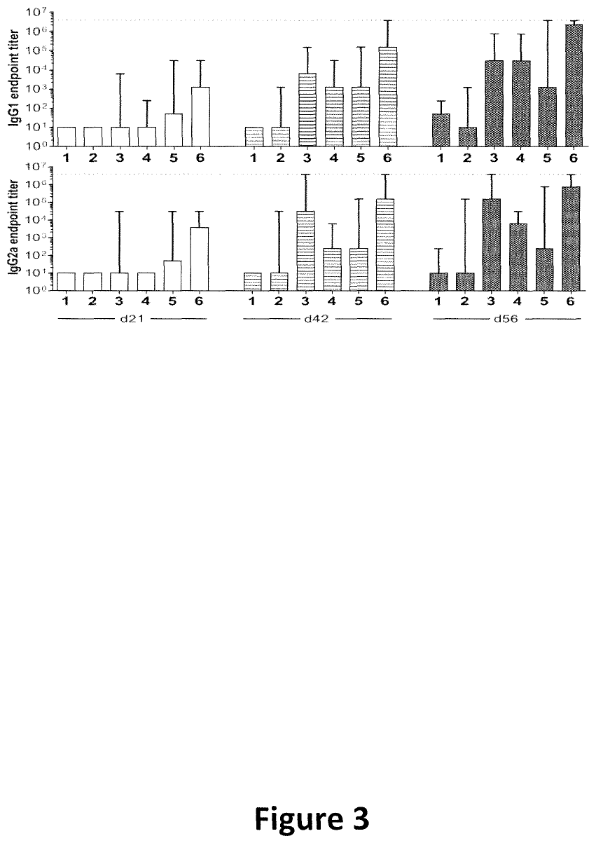

Expression of all six RNA constructs was demonstrated in the corresponding cell lysates (see FIG. 2). The tested Rotavirus antigen design of Group 6 (corresponding Table 6) was detectable in the supernatant of transfected 293T cells (see FIG. 2). Indistinct (e.g. Group 3, corresponding Table 6, see FIG. 2) gel bands or shifts in their positions likely due to the glycosylation of the protein.

2.2: In Vivo Analysis of Immunogenicity of Rotavirus Antigen Designs

The present example shows that Rotavirus mRNA vaccines encoding different antigen designs induce humoral immune responses in mice (Balb/c).

mRNA constructs encoding different Rotavirus antigen designs (see Table 7) were prepared according to Example 1. The mRNA was formulated with protamine (see Example 1.4.2 Protamine complexation). The different mRNA vaccine candidates were applied on day 0, 21, and 42 and administered intradermal (i.d.) with 80 ug of RNA as shown in Table 7. One negative control group (1) received an irrelevant mRNA. Blood samples were taken at day 21, 42, and 56 for determination of humoral immune responses.

ELISA was performed using recombinant Rotavirus protein P2-VP8*P[8] for coating. Coated plates were incubated using respective serum dilutions, and binding of specific antibodies to the respective recombinant Rotavirus protein P2-VP8*P[8] was detected using biotinylated isotype specific anti-mouse antibodies followed by streptavidin-HRP (horse radish peroxidase) with Amplex as substrate. Endpoint titers of antibodies (IgG1, IgG2a) directed against the recombinant Rotavirus protein P2-VP8*P[8] were measured by ELISA on day 21, 42, and 56 post vaccinations.

TABLE 7
Vaccination scheme of Example 2

| | No. of | | | RNA | | SEQ ID |
|---|---|---|---|---|---|---|
| Group | mice | Dose | Volume | ID | Construct design | NO: RNA |
| 1 | 7 | 80 μg | 2 × 50 μl | — | Irrelevant RNA | 1883 |
| 2 | 7 | 80 μg | 2 × 50 μl | R5470 | P2-Linker-VP8*(65-223) | 1865 |
| 3 | 7 | 80 μg | 2 × 50 μl | R6326 | P2-Linker-VP8*(65-223)-Linker-Ferritin | 1874 |
| 4 | 7 | 80 μg | 2 × 50 μl | R5488 | SP(IgE)-P2-Linker-VP8*(41-223) | 1880 |
| 5 | 7 | 80 μg | 2 × 50 μl | R6324 | SP(IgE)-P2-Linker-VP8*(41-223)-Linker-TM | 1882 |
| 6 | 7 | 80 μg | 2 × 50 μl | R6328 | LumSynt-Linker-P2-Linker-VP8*(41-223) | 1877 |

### Example 3: (Cross) Reactivity of Rotavirus Antigen Designs

3.1 Cross Reactivity of Rotavirus Serotype P[6] Antigen Designs

3.1.1 Determination of Specific and Cross Reactive Humoral Immune Responses by ELISA

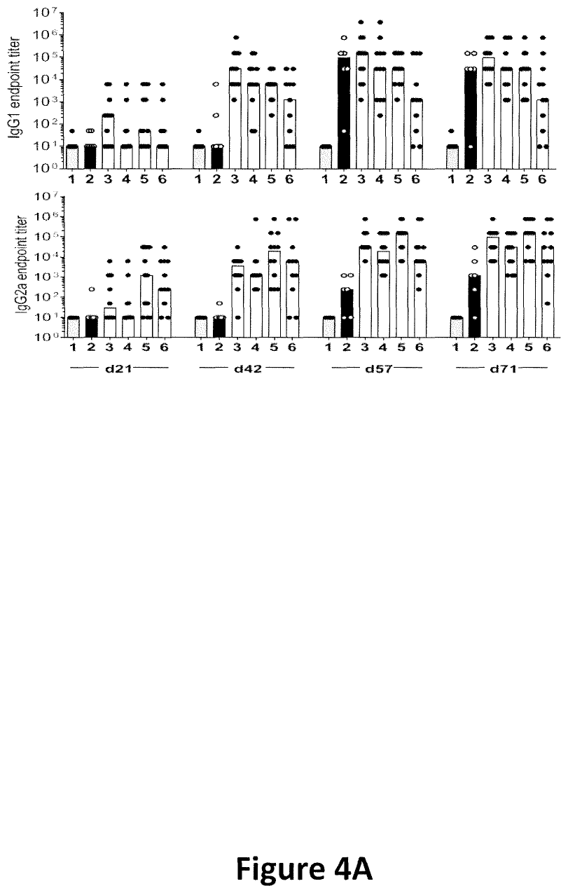

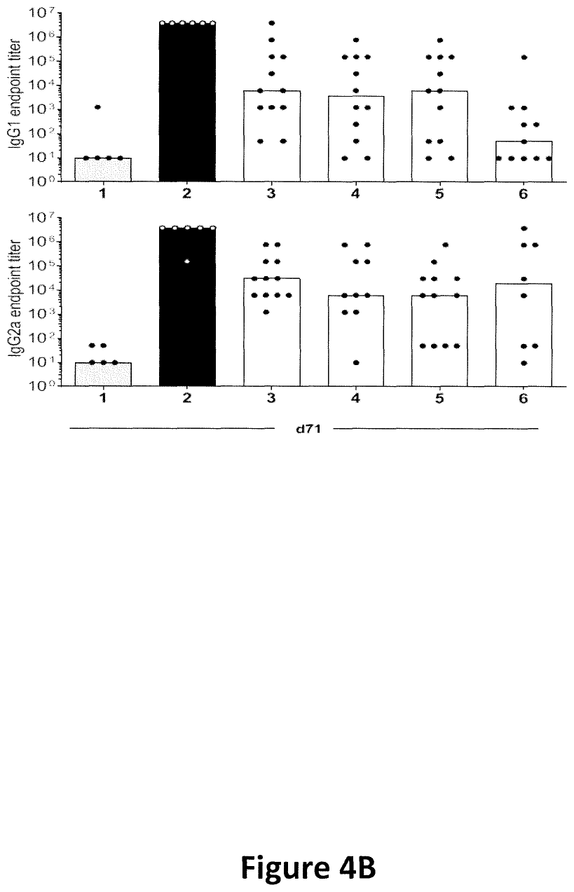

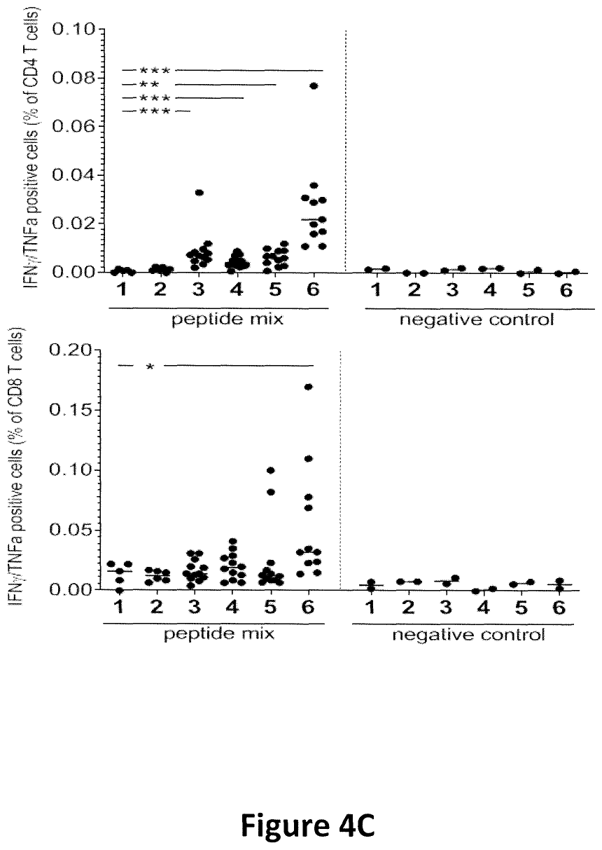

Balb/c mice were immunized with RNA vaccines (as prepared in Example 1) encoding different VP8* antigen designs from serotype P[6], recombinant Rotavirus protein P2-VP8*P[8] as a positive control or RiLa (Ringer lactate) as a negative control as indicated in Table X4 below. The mRNA was formulated with protamine (see Example 1.4.2 Protamine complexation). Vaccinations were performed on day 0, 21 and 42. Blood samples taken on day 21, 42, 57 and 71 were analyzed for the presence of VP8* specific IgG1 and IgG2a antibodies by ELISA using recombinant Rotavirus protein P2-VP8*P[6] (FIG. 4-A), or recombinant Rotavirus protein P2-VP8*P[8] (FIG. 4-B) as a coating reagent.

3.1.2 Intracellular Cytokine Staining (ICS):

Splenocytes from vaccinated mice were isolated on day 71 according to a standard protocol known in the art. Briefly, isolated spleens were grinded through a cell strainer and washed in PBS/1% FBS followed by red blood cell lysis. After an extensive washing step with PBS/1% FBS, splenocytes were seeded into 96-well plates (2×10⁶ cells per well). Cells were stimulated with a mixture of Rotavirus VP8* peptides (see Table 9) (5 ug/ml each) in the presence of 2.5 ug/ml each of an anti-CD28 antibody (BD Biosciences) and a protein transport inhibitor for 6 h at 37° C. After stimulation, cells were washed and stained for intracellular cytokines using the Cytofix/Cytoperm reagent (BD Biosciences) according to the manufacturer's instructions. The following antibodies were used for staining: Thy1.2-FITC (1:200), CD8-APC-H7 (1:100), TNF-PE (1:100), IFNγ-APC (1:100) (eBioscience), CD4-BD Horizon V450 (1:200) (BD Biosciences) and incubated with Fcγ-block diluted 1:100. Aqua Dye was used to distinguish live/dead cells (Invitrogen). Cells were acquired using a BD FACS Canto II flow cytometer (Beckton Dickinson). Flow cytometry data was analyzed using FlowJo software (Tree Star, Inc.). Results are shown in FIG. 4-C.

TABLE 8
Vaccination scheme of Example 3.1

| | No. of | | | RNA | | SEQ ID |
|---|---|---|---|---|---|---|
| Group | mice | Dose | Volume | ID | Construct design | NO: RNA |
| 1 | 6 | — | 2 × 50 μl | — | RiLa - negative control | — |
| 2 | 6 | 6 μg | 4 × 25 μl | — | Recombinant Rotavirus protein | — |
| | | | | | VP8*P[8] + Alum | |
| 3 | 12 | 80 μg | 2 × 50 μl | R5433 | SP(HsPLAT)-VP8*[P6](41-223) | 1895 |
| 4 | 12 | 80 μg | 2 × 50 μl | R5434 | SP(HsPLAT)-P2-VP8*[P6](41-223) | 1896 |
| 5 | 12 | 80 μg | 2 × 50 μl | R5435 | SP(HsPLAT)-VP8*[P6](1-223) | 1897 |
| 6 | 12 | 80 μg | 2 × 50 μl | R5436 | SP(HsPLAT)-VP8*[P6](20-240) (N→Q mutation) | 1890 |

TABLE 9
Rotavirus VP8* peptide mix for ICS

| MHC class | Serotype | Peptide sequence |
|---|---|---|
| MHCI | P[4]/P[6]/P[8] | FYIIPRSQE |
| MHCI | P[4]/P[8] | KYGGRVWTF |
| MHCI | P[4]/[P8] | VYESTNNSD |
| MHCI | P[6] | FYNSVWTFH |
| MHCI | P[6] | GFMKFYNSV |
| MHCII | P[4]/P[8] | SDFWTAVIAVEPHVN |
| MHCII | P[6] | TNKTDIWVALLLVEP |
| MHCII | P[6] | HKRTLTSDTKLAGFM |

Results:

As shown in FIG. 4-A, statistically significant IgG1 and IgG2a responses compared to the negative control were detectable for all groups vaccinated with the mRNA vaccine of different VP8*P[6] antigen designs when recombinant Rotavirus protein P2-VP8*P[6] was used as a coating reagent.

FIG. 4-B shows cross-reactive responses in mice vaccinated with VP8*P[6] designs with the recombinant Rotavirus protein P2-VP8*P[8] used as a coating reagent.

CD4 positive T cells play an important role in the immune system, particularly in the adaptive immune system. They help the activity of other immune cells by releasing T cell cytokines and are essential in B cell antibody class switching, in the activation and growth of cytotoxic T cells. An effective Rotavirus vaccine should induce CD4+ T cell responses. CD8+ T cells are a major protective immune mechanism against intracellular infections, like Rotavirus virus infections. An effective Rotavirus vaccine should also induce CD8+ T cells responses.

As shown in FIG. 4-C, statistically significant CD4 positive T cell responses were detectable for all groups vaccinated with the mRNA vaccine. Additionally, Group 6 (see Table 8) shows CD8 positive T cell responses.

Additional improvements to the mRNA design or formulation could lead to an enhanced immune response after vaccination (see Example 5).

Accordingly, these findings highlight one of the advantageous features of the inventive mRNA-based Rotavirus vaccine designs.

3.2: Cross Reactivity of Rotavirus Serotype P[8] Antigen Designs

3.2.1 Determination of Specific and Cross Reactive Humoral Immune Responses by ELISA

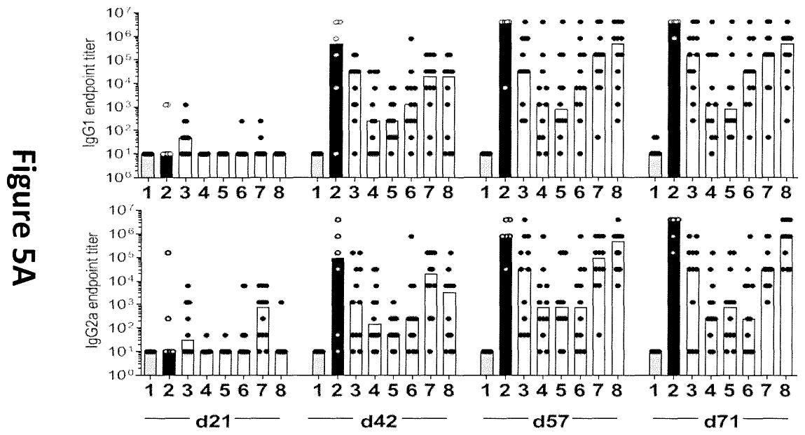

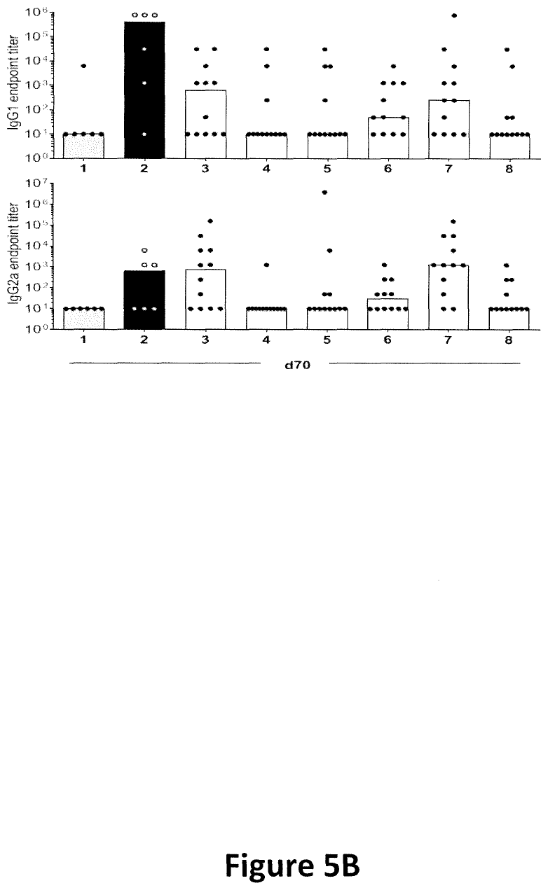

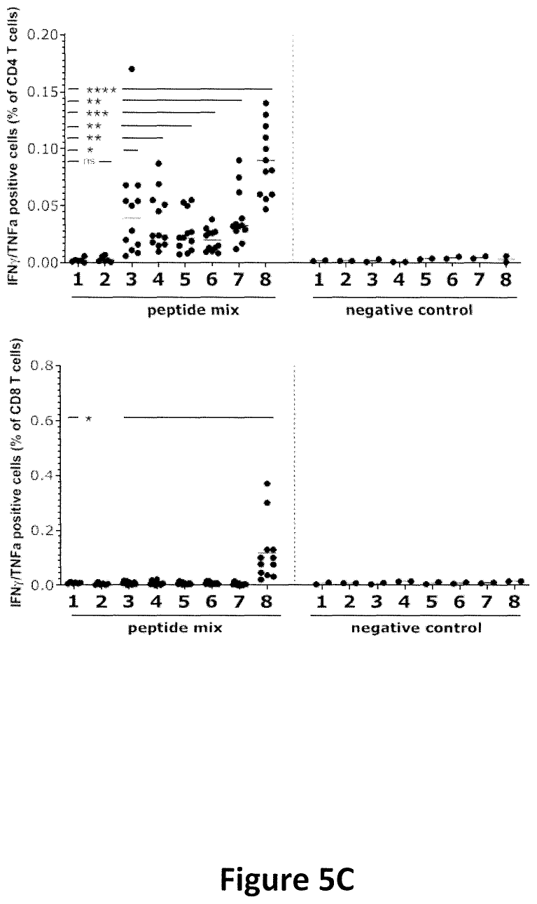

Balb/c mice were immunized with RNA vaccines (as prepared in Example 1) encoding different VP8* antigen designs from serotype P[8], recombinant protein VP8*P[8] as a positive control or RiLa (Ringer lactate) as a negative control as indicated in Table 10 below. The mRNA was formulated with protamine (see Example 1.4.2 Protamine complexation). Vaccinations were performed on day 0, 21 and 42. Blood samples taken on day 21, 42, 56 and 71 were analyzed for the presence of VP8* specific IgG1 and IgG2a antibodies by ELISA using VP8*P[8] protein (FIG. 5-A), or VP8* P[6] protein (FIG. 5-B) as a coating reagent.

3.2.2: Intracellular Cytokine Staining

Splenocytes from vaccinated mice were isolated on day 71 according to a standard protocol known in the art. Briefly, isolated spleens were grinded through a cell strainer and washed in PBS/1% FBS followed by red blood cell lysis. After an extensive washing step with PBS/1% FBS, splenocytes were seeded into 96-well plates (2×10⁶ cells per well). Cells were stimulated with a mixture of Rotavirus VP8* peptides (see Table 9) (5 ug/ml each) in the presence of 2.5 ug/ml each of an anti-CD28 antibody (BD Biosciences) and a protein transport inhibitor for 6 h at 37° C. After stimulation, cells were washed and stained for intracellular cytokines using the Cytofix/Cytoperm reagent (BD Biosciences) according to the manufacturer's instructions. The following antibodies were used for staining: Thy1.2-FITC (1:200), CD8-APC-H7 (1:100), TNF-PE (1:100), IFNγ-APC (1:100) (eBioscience), CD4-BD Horizon V450 (1:200) (BD Biosciences) and incubated with Fcγ-block diluted 1:100. Aqua Dye was used to distinguish live/dead cells (Invitrogen). Cells were acquired using a BD FACS Canto II flow cytometer (Beckton Dickinson). Flow cytometry data was analyzed using FlowJo software (Tree Star, Inc.). Results are shown in FIG. 5-C.

TABLE 10
Vaccination scheme of Example 3.2

| | No. of | | | | RNA | | SEQ ID |
|---|---|---|---|---|---|---|---|
| Group | mice | Route | Dose | Volume | ID | Construct Design | NO: RNA |
| 1 | 6 | i.d. | — | 2 × 50 μl | — | RiLa (negative control) | |
| 2 | 6 | i.m. | 6 μg | 4 × 25 μl | — | Recombinant Rotavirus protein | |
| | | | | | | VP8*P[8] + Alum | |
| 3 | 12 | i.d. | 80 μg | 2 × 50 μl | R5480 | SP(HSA)-VP8*P[8](2-230) | 1891 |
| 4 | 12 | i.d. | 80 μg | 2 × 50 μl | R5482 | SP(HSA)-VP8*P[8](11-223) | 1892 |
| 5 | 12 | i.d. | 80 μg | 2 × 50 μl | R5484 | SP(HSA)-VP8*P[8](41-223) | 1893 |
| 6 | 12 | i.d. | 80 μg | 2 × 50 μl | R5486 | SP(HSA)-P2-VP8*P[8](41-223) | 1894 |
| 7 | 12 | i.d. | 80 μg | 2 × 50 μl | R5488 | SP(IgE)-P2-VP8*P[8](41-223) | 1880 |
| 8 | 12 | i.d. | 80 μg | 2 × 50 μl | R5470 | P2-VP8*[P8](65-223) | 1865 |

Results:

As shown in FIG. 5-A, statistically significant IgG1 and IgG2a responses compared to the negative control were detectable for all groups vaccinated with the mRNA vaccine encoding different VP8* antigen designs when recombinant Rotavirus protein P2-VP8*P[8] was used as a coating reagent.

FIG. 5-B shows cross-reactive responses in mice vaccinated with P[8] designs of group 3, 6 and 7 (see Table 10) with the recombinant Rotavirus protein P2-VP8*P[6] used as a coating reagent.

CD4 positive T cells play an important role in the immune system, particularly in the adaptive immune system. They help the activity of other immune cells by releasing T cell cytokines and are essential in B cell antibody class switching, in the activation and growth of cytotoxic T cells. An effective Rotavirus vaccine should induce CD4+ T cell responses. CD8+ T cells are a major protective immune mechanism against intracellular infections, like Rotavirus virus infections. An effective Rotavirus vaccine should also induce CD8+ T cells responses.

As shown in FIG. 5-C, statistically significant CD4 positive T cell responses were detectable for all groups vaccinated with the mRNA vaccine. Additionally Group 8 (see Table 10) shows CD8 positive T cell responses. Accordingly, these findings highlight one of the advantageous features of the inventive mRNA-based Rotavirus vaccine designs.

### Example 4: In Vitro Analysis of Expression of Different mRNA Design Encoding a Rotavirus Antigen

The present example shows that different mRNA constructs encoding a Rotavirus antigen were expressed in mammalian cells.

To determine in vitro protein expression of the RNA constructs, HEK 293T cells were transfected with unformulated mRNA encoding a Rotavirus antigen using Lipofectamine 2000. 24 h-48 h after transfection, cell lysates and cell culture supernatants were subjected to SDS-PAGE and Western blot analysis using rabbit anti-VP8*P[4] or anti-VP8*P[8] or antibody (1:1000; Aldevron) or mouse anti-alpha-tubulin antibody (1:1000; Abcam) as primary antibodies as well as goat anti-rabbit IgG IRDye®800CW or 680RD antibody (1:10000; Li-Cor) or goat anti-mouse IgG IRDye® 680RD or 800CW antibody (1:10000; Li-Cor) as secondary antibodies. Detection and quantification was performed using a Li-Cor detection system (Odyssey CLx image system) in combination with Image Studio Lite software. Table 11 contains mRNA constructs that were used in the experiment:

TABLE 11
RNA constructs used for Western blot analysis (Example 4)

| | RNA | | | | SEQ ID |
|---|---|---|---|---|---|
| Group | ID | mRNA design | Serotype | Construct design | NO: |
| 1 | R5470 | cap0 | P[8] | P2-Linker-VP8*(65-223) | 1865 |
| 2 | R8044 | cap1 | P[8] | P2-Linker-VP8*(65-223) | 1864 |
| 3 | R8046 | cap1 + poly(A) sequence, | P[8] | P2-Linker-VP8*(65-223) | 1865 |
| | | located at the 3′ terminus | | | |

Results:

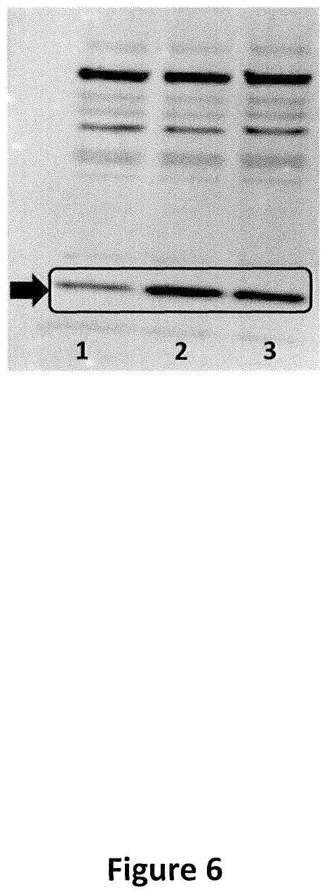

Expression of all three mRNA constructs was demonstrated in the corresponding cell lysates (see FIG. 6). Best expression was detectable for cap1 mRNA designs (Group 2 and 3, Table 11).

### Example 5: Analysis of Different mRNA Designs Encoding a Rotavirus Antigen

Different mRNA constructs encoding a Rotavirus antigen (see Table X8) were prepared according to Example 1. The mRNA was formulated with LNPs (see Example 1.4.1 LNP formulation). The different mRNA vaccine candidates were applied on days 0 and 21 and administered intramuscular (i.m.) with 4 ug of RNA in mice as shown in Table 12. One negative control group (1) received an irrelevant mRNA. Blood samples were taken at day 21, 42, and 56 for determination of humoral immune responses.

ELISA was performed using recombinant Rotavirus protein P2-VP8*P[8] for coating. Coated plates were incubated using respective serum dilutions, and binding of specific antibodies to the recombinant Rotavirus protein P2-VP8*P[8] was detected using biotinylated isotype specific anti-mouse antibodies followed by streptavidin-HRP (horse radish peroxidase) with Amplex as substrate. Endpoint titers of antibodies (IgG1, IgG2a) directed against the recombinant Rotavirus protein P2-VP8*P[8] were measured by ELISA on day 21, 42, and 56 post vaccinations.

5.1: In Vivo Analysis of Immunogenicity

Experimental setting described before in Example 5. Table 12 contains mRNA constructs that were used in the experiment.

TABLE 12
Vaccination scheme of Example 5

| | No. of | | | RNA | mRNA | Modified | | SEQ ID |
|---|---|---|---|---|---|---|---|---|
| Group | mice | Dose | Volume | ID | design | nucleotides | Construct design | NO: |
| 1 | 7 | 4 μg | 25 μl | — | — | — | Irrelevant RNA | 1884 |
| 2 | 7 | 4 μg | 25 μl | R5470 | cap0 | — | P2-Linker-VP8*(65-223) | 1865 |
| 3 | 7 | 4 μg | 25 μl | R8047 | cap0 | m1Ψ | P2-Linker-VP8*(65-223) | 1870 |
| 4 | 7 | 4 μg | 25 μl | R8044 | cap1(co- | — | P2-Linker-VP8*(65-223) | 1864 |
| | | | | | trans . . . cap) | | | |
| 5 | 7 | 4 μg | 25 μl | R8049 | cap1(co-trans. | m1Ψ | P2-Linker-VP8*(65-223) | 1868 |
| | | | | | cap) | | | |
| 6 | 7 | 4 μg | 25 μl | R8046 | cap1(enzym. | — | P2-Linker-VP8*(65-223) | 1865 |
| | | | | | cap) + poly(A) | | | |
| | | | | | sequence, | | | |
| | | | | | located at the | | | |
| | | | | | 3′ terminus | | | |
| 7 | 7 | 4 μg | 25 μl | R7411 | cap1 (enzym. | m1Ψ | P2-Linker-VP8*(65-223) | 1869 |
| | | | | | cap) + poly(A) | | | |
| | | | | | sequence, | | | |
| | | | | | located at the | | | |
| | | | | | 3′ terminus | | | |

Results:

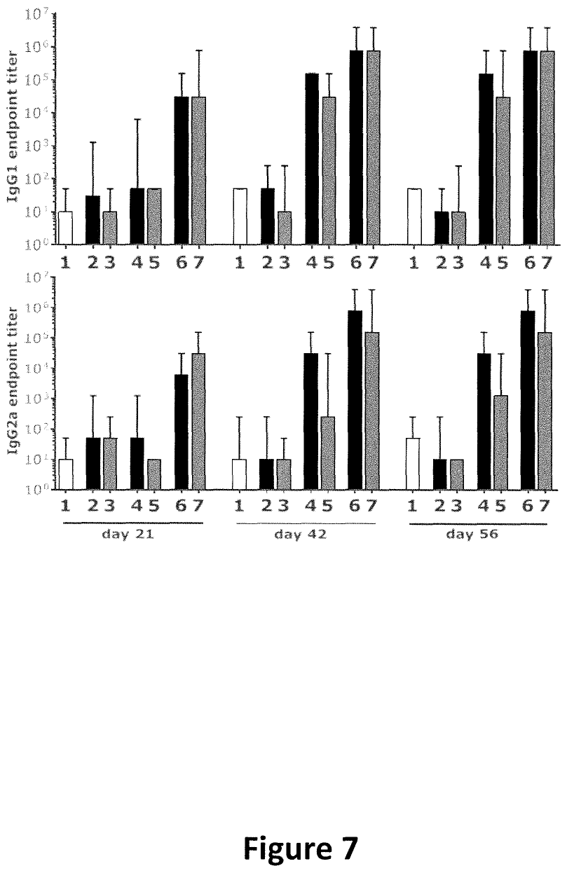

As shown in FIG. 7, the cap1 mRNA designs (Group 4, 5, 6 and 7, Table 12) induced strong, humoral immune responses in mice (shown as IgG1 and IgG2a endpoint titers) compared to mRNA designs with cap0 (Group 2 and 3, Table 12). The mRNA designs with poly(A) sequences, located at the 3′ terminus (Group 6 and 7, FIG. 7) showed higher IgG responses compared to mRNA designs without an poly(A) sequence, located at the 3′ terminus (black bars, FIG. 7).

In addition mRNA designs comprising modified nucleotides (grey bars, FIG. 7) induced comparable humoral immune responses in mice compared to constructs comprising only natural nucleotides (black bars, FIG. 7).

Improvements of the mRNA design enhanced the immune responses to mRNA vaccine.

5.2 Determination of Rotavirus Virus Neutralization Titers (VNTs)

Serum was collected on day 56 after vaccination described in Example 5 and Rotavirus neutralization titers were measured as described below. Table 12 contains mRNA constructs that were used in the experiment.

Diluted serum samples were incubated with a constant amount of Rotavirus virus strain Wa (G1P[8]) for 1 hour at 37° C., before transfer to a 96 well plate containing confluent MA104 cells. After 1 hour the monolayers were washed and incubated with the serum-virus mixture for 14-16 hours, frozen and thawed. VNTs were determined by measuring Rotavirus virus antigen in the lysed monolayers in wells receiving serum compared to viral control wells, using a standard enzyme-linked immunosorbent format. The neutralization titer represents a 60% reduction in the amount of virus.

Results:

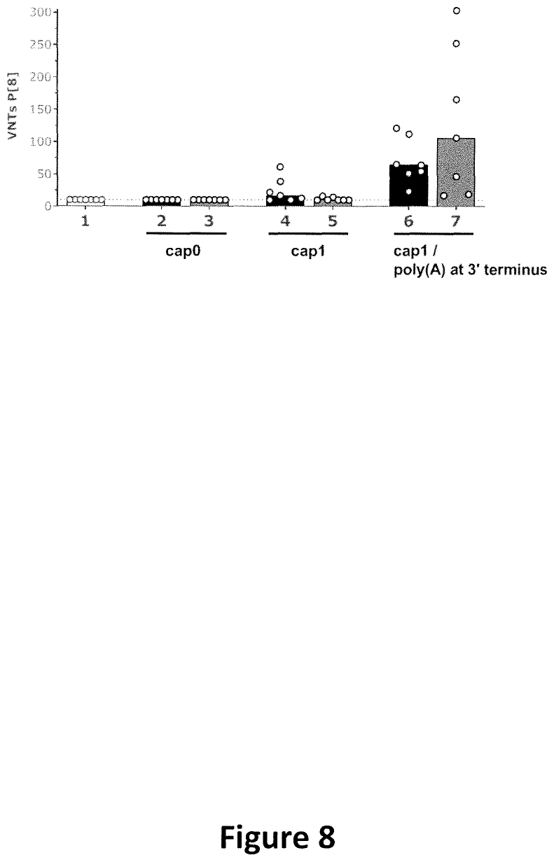

As can be seen from FIG. 8 the mRNA designs with cap1 and poly(A) sequence, located at the 3′ terminus (Group 6 and 7, FIG. 8) induced robust virus neutralization antibody titers compared to the negative control. Without a poly(A) sequence, located at the 3′ terminus (Group 4 and 5, FIG. 8) a slight effect was detectable compared to the negative control (Group 1, FIG. 8).

### Example 6: Analysis of Different mRNA Designs Encoding a Rotavirus Antigen

6.1: In Vitro Analysis of Expression

The present example shows that different mRNA constructs encoding a Rotavirus antigen were expressed in mammalian cells.

To determine in vitro protein expression of the RNA constructs, HEK 293T cells were transfected with unformulated mRNA encoding a Rotavirus antigen using Lipofectamine 2000. 24 h-48 h after transfection, cell lysates and cell culture supernatants were subjected to SDS-PAGE and Western blot analysis using rabbit anti-VP8* P[4 and 8] antibody (1:1000; Aldevron) or mouse anti-alpha-tubulin antibody (1:1000; Abcam) as primary antibodies as well as goat anti-rabbit IgG IRDye®800CW or 680RD antibody (1:10000; Li-Cor) or goat anti-mouse IgG IRDye® 680RD or 8000W antibody (1:10000; Li-Cor) as secondary antibodies. Detection and quantification was performed using a Li-Cor detection system (Odyssey CLx image system) in combination with Image Studio Lite software. Table 13 contains mRNA constructs that were used in the experiment.

TABLE 13
RNA constructs used for Western blot analysis (Example 6)

| | | | | 5′-cap structure/ | | |
|---|---|---|---|---|---|---|
| | | | | poly(A) sequence, | UTRs | |
| | No. of | RNA | | located at 3′ | 5′-UTR/ | SEQ ID |
| Group | mice | ID | Construct design | terminus | 3′-UTR | NO: |
| 1 | 7 | R8044 | P2-Linker-VP8*(65-223) | co-trans. cap/− | HSD17B4/PSMB3 | 1864 |
| 2 | 7 | R8134 | P2-Linker-VP8*(65-223) | co-trans. cap/+ | HSD17B4/PSMB3 | 1864 |
| | | | | (enzym. Poly(A)) | | |
| 3 | 7 | R8131 | P2-Linker-VP8*(65-223) | co-trans. cap/+ | HSD17B4/PSMB3 | 1862 or |
| | | | | (A100) | | 591 |
| 4 | 7 | R8135 | P2-Linker-VP8*(65-223) | enzym. cap/− | RPL32/ALB7 | 1865 |
| 5 | 7 | R8046 | P2-Linker-VP8*(65-223) | enzym. cap/+ | RPL32/ALB7 | 1865 |
| | | | | (enzym. Poly(A)) | | |
| 6 | 7 | R8138 | P2-Linker-VP8*(65-223) | enzym. cap/+ (A100) | RPL32/ALB7 | 1865 |

Results:

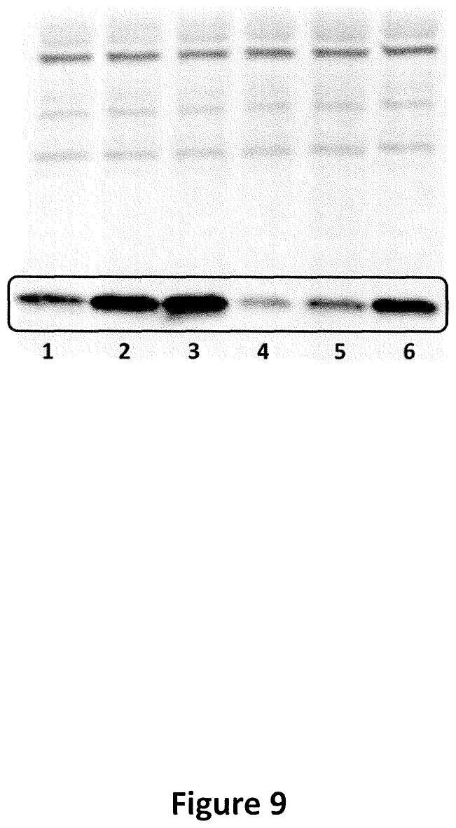

Expression of all six mRNA constructs was demonstrated in the corresponding cell lysates (see FIG. 9). Best expression was detectable for mRNA designs with a cap1 (co-trans. cap), UTR combination HSD17B4/PSMB3 and a poly(A) sequence, located at the 3′ terminus (Group 2 and 3, FIG. 9). mRNA designs with cap1 (enzym. cap), UTR combination RPL32/ALB7 without poly(A) sequence, located at the 3′ terminus (Group 4, FIG. 9) showed lower expression compared to the other mRNA designs.

6.2: In Vivo Analysis of Immunogenicity

Different mRNA constructs encoding a Rotavirus antigen (see Table 14) were prepared according to Example 1. The mRNA was formulated with LNPs (see Example 1.4.1 LNP formulation). The different mRNA vaccine candidates were applied on days 0 and 21 and administered intramuscular (i.m.) with 4 ug of RNA as shown in Table 14. One negative control group (1) received Buffer and one positive control group (2) received recombinant Rotavirus protein P2-VP8*P[8]. Blood samples were taken at day 21, 42, and 56 for determination of humoral immune responses.

ELISA was performed using recombinant Rotavirus protein P2-VP8*P[8] for coating. Coated plates were incubated using respective serum dilutions, and binding of specific antibodies to the recombinant Rotavirus protein P2-VP8*P[8] was detected using biotinylated isotype specific anti-mouse antibodies followed by streptavidin-HRP (horse radish peroxidase) with Amplex as substrate. Endpoint titers of antibodies (IgG1, IgG2a) directed against the recombinant Rotavirus protein P2-VP8*P[8] were measured by ELISA on day 21, 42, and 56 post vaccinations.

TABLE 14
Vaccination scheme of Example 6.2

| | | | | | | 5′-cap | | | |
|---|---|---|---|---|---|---|---|---|---|
| | | | | | | structure/ | | | |
| | | | | | | poly(A) | | | |
| | | | | | | sequence, | UTRs | | |
| | No. of | | | RNA | Construct | located at 3′ | 5′-UTR/ | Modified | SEQ ID |
| Group | mice | Dose | Volume | ID | design | terminus | 3′-UTR | nucleotides | NO: |
| 1 | 6 | — | 25 μl | — | 0.9% NaCl | — | — | − | |
| | | | | | buffer, negative | | | | |
| | | | | | control | | | | |
| 2 | 6 | 6 μg | 4 × | — | Recombinant | — | — | − | |
| | | | 25 μl | | Rotavirus | | | | |
| | | | | | protein P2- | | | | |
| | | | | | VP8*P[8] + | | | | |
| | | | | | Alum | | | | |
| 3 | 7 | 4 μg | 25 μl | R8044 | P2-Linker- | co-trans. cap/− | HSD17B4/ | − | 1864 |
| | | | | | VP8*(65-223) | | PSMB3 | | |
| 4 | 7 | 4 μg | 25 μl | R8134 | P2-Linker- | co-trans. cap/+ | HSD17B4/ | − | 1864 |
| | | | | | VP8*(65-223) | (enzym. Poly(A)) | PSMB3 | | |
| 5 | 7 | 4 μg | 25 μl | R8131 | P2-Linker- | co-trans. cap/+ | HSD17B4/ | − | 1862 or |
| | | | | | VP8*(65-223) | (A100) | PSMB3 | | 591 |
| 6 | 7 | 4 μg | 25 μl | R8135 | P2-Linker- | enzym. cap/− | RPL32/ | − | 1865 |
| | | | | | VP8*(65-223) | | ALB7 | | |
| 7 | 7 | 4 μg | 25 μl | R8046 | P2-Linker- | enzym. cap/+ | RPL32/ | − | 1865 |
| | | | | | VP8*(65-223) | (enzym. Poly(A)) | ALB7 | | |
| 8 | 7 | 4 μg | 25 μl | R8138 | P2-Linker- | enzym. cap/+ | RPL32/ | − | 1865 |
| | | | | | VP8*(65-223) | (A100) | ALB7 | | |
| 9 | 7 | 4 μg | 25 μl | R8049 | P2-Linker- | co-trans. cap/− | HSD17B4/ | + | 1868 |
| | | | | | VP8*(65-223) | | PSMB3 | | |
| 10 | 7 | 4 μg | 25 μl | R8136 | P2-Linker- | co-trans. cap/+ | HSD17B4/ | + | 1868 |
| | | | | | VP8*(65-223) | (enzym. Poly(A)) | PSMB3 | | |
| 11 | 7 | 4 μg | 25 μl | R8133 | P2-Linker- | co-trans. cap/+ | HSD17B4/ | + | 1866 |
| | | | | | VP8*(65-223) | (A100) | PSMB3 | | |
| 12 | 7 | 4 μg | 25 μl | R8137 | P2-Linker- | enzym. cap/− | —/muag | + | 1869 |
| | | | | | VP8*(65-223) | | | | |
| 13 | 7 | 4 μg | 25 μl | R7411 | P2-Linker- | enzym. cap/+ | —/muag | + | 1869 |
| | | | | | VP8*(65-223) | (enzym. Poly(A)) | | | |

Results:

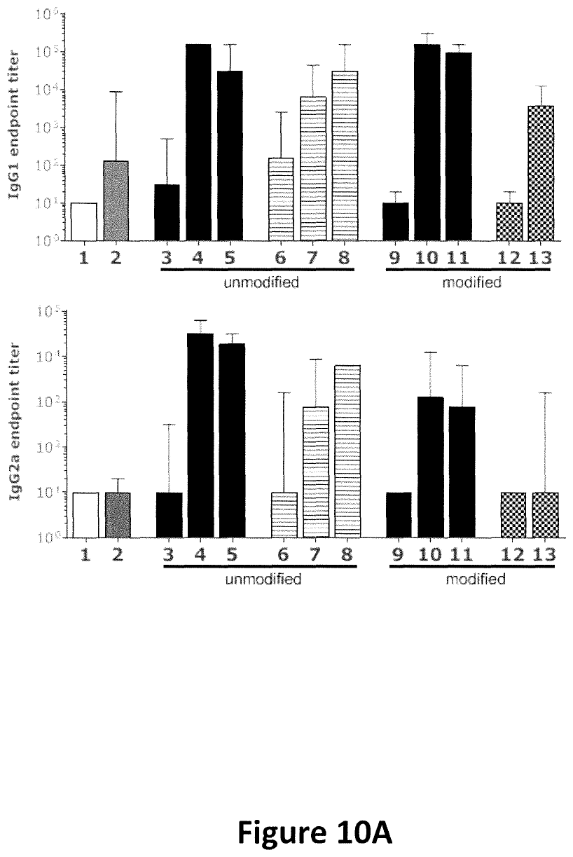

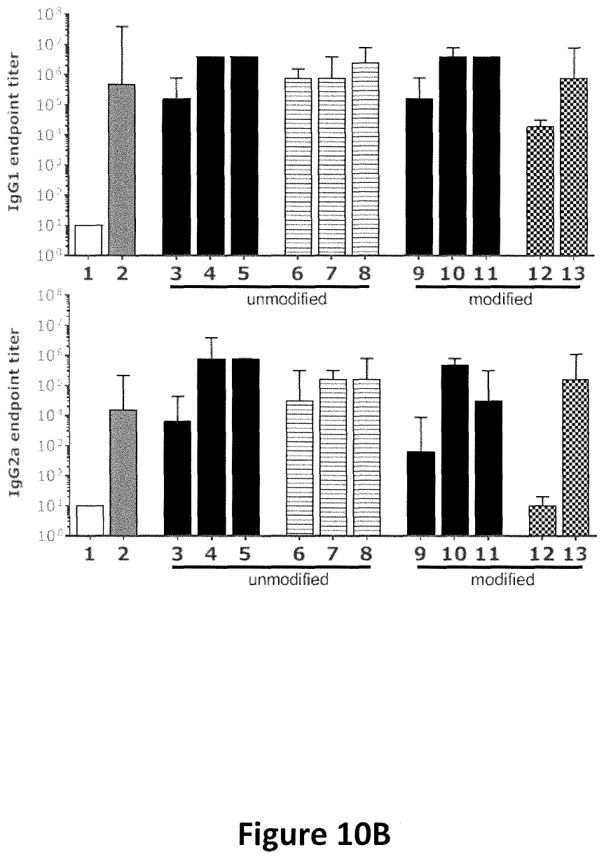

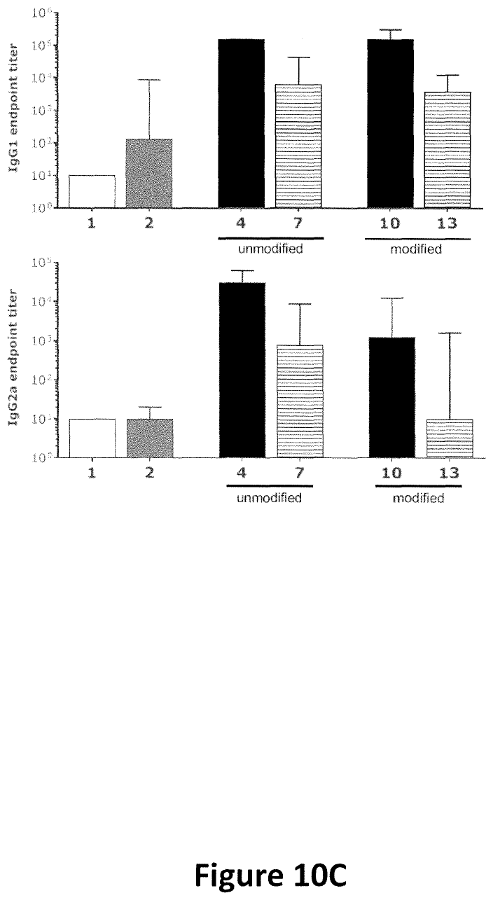

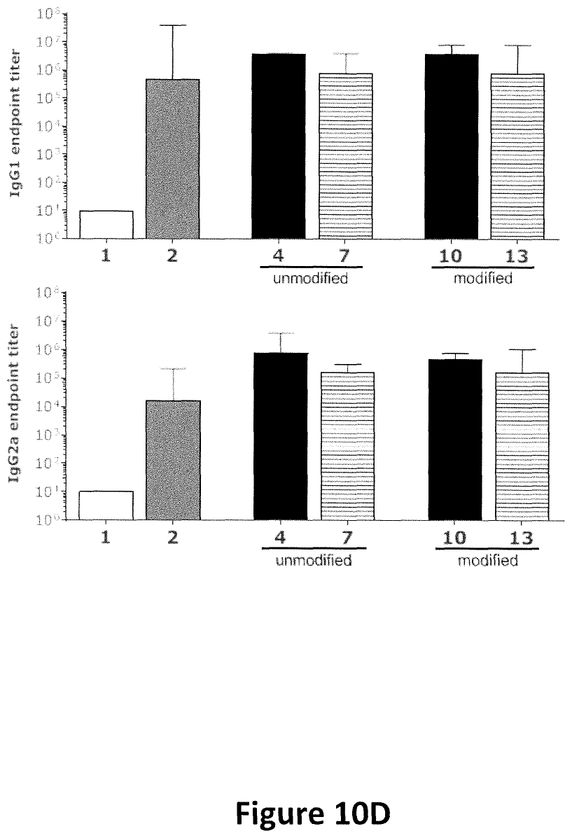

As shown in FIG. 10A, early humoral immune responses (day 21) were detectable for all groups with a poly(A) sequence, located at the 3′ terminus (Group 4, 5, 7, 8, 10, 11 and 13, FIG. 10A), independent of UTR combination (black bars, striped bars or dotted bars, FIG. 10A) and modification of nucleotides. At late time point (day 56), shown in FIG. 10B, nearly all mRNA designs showed high IgG1 and IgG2a endpoint titers. FIG. 10C shows that mRNA designs with co-transcriptional capping, a poly(A) sequence, located at 3′ terminus and UTR combination HSD17B4/PSMB3 (black bars, FIG. 10C) induced to an early time point (day 21) higher IgG1 and IgG2a titers compared to enzymatically capped mRNA designs with, a poly(A) sequence, located at 3′ terminus and other UTR combinations (striped bars, FIG. 10C). This effect was independently of modified nucleotides. At day 56 shown in FIG. 10D all mRNA designs induced high humoral immune responses with a slight trend of higher responses for mRNA designs with co-transcriptional capping, a poly(A) sequence, located at 3′ terminus and UTR combination HSD17B4/PSMB3.

Early immune responses are very important for a fast and robust protection against Rotavirus virus infections. The high titers in later time points show that this immune response resulting from mRNA vaccination can be boosted.

6.3 Determination of Rotavirus Virus Neutralization Titers (VNTs)

Serum was collected on day 56 after vaccination described in Example 6.2 and Rotavirus neutralization titers were measured as described below. Table 14 contains mRNA constructs that were used in the experiment.

Diluted serum samples were incubated with a constant amount of Rotavirus virus strain Wa (G1P[8]) for 1 hour at 37° C., before transfer to a 96 well plate containing confluent MA104 cells. After 1 hour the monolayers were washed and incubated with the serum-virus mixture for 14-16 hours, frozen and thawed. VNTs were determined by measuring Rotavirus virus antigen in the lysed monolayers in wells receiving serum compared to viral control wells, using a standard enzyme-linked immunosorbent format. The neutralization titer represents a 60% reduction in the amount of virus.

Results:

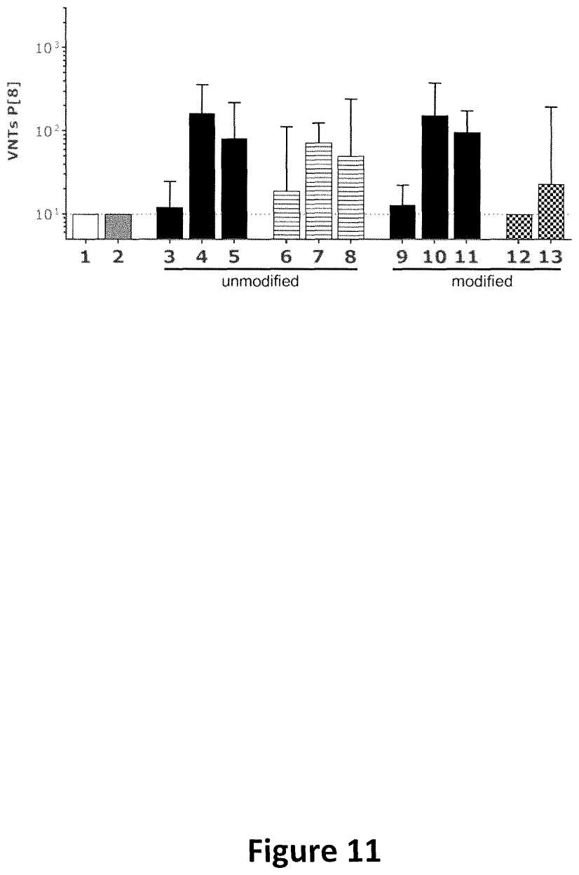

FIG. 11 shows that all mRNA designs with a poly(A) sequence, located at the 3′ terminus (Group 4, 5, 7, 8, 10, 11 and 13, FIG. 11) induced the formation of Rotavirus specific functional antibodies in mice as shown by high virus neutralizing antibody titers. For the Alum adjuvanted recombinant Rotavirus protein VP8*P[8] no VNT titer could be detected. mRNA designs with co-transcriptional capping, a poly(A) sequence, located at 3′ terminus and UTR combination HSD17B4/PSMB3 (Group 4 and 10, FIG. 11) induced higher VNT titers compared to enzymatically capped mRNA designs (Group 7 and 13, FIG. 11). This effect was independent of modified nucleotides.

6.4: In Vivo Analysis of Cytokines

Appropriate dilutions of sera collected 14 hours after prime immunization (see Example 6.2) were analyzed by a mouse IFN-alpha ELISA kit according to the manufacturer's protocol (PBL, cat.: 42115-1). Table 14 contains mRNA constructs that were used in the experiment.

Results:

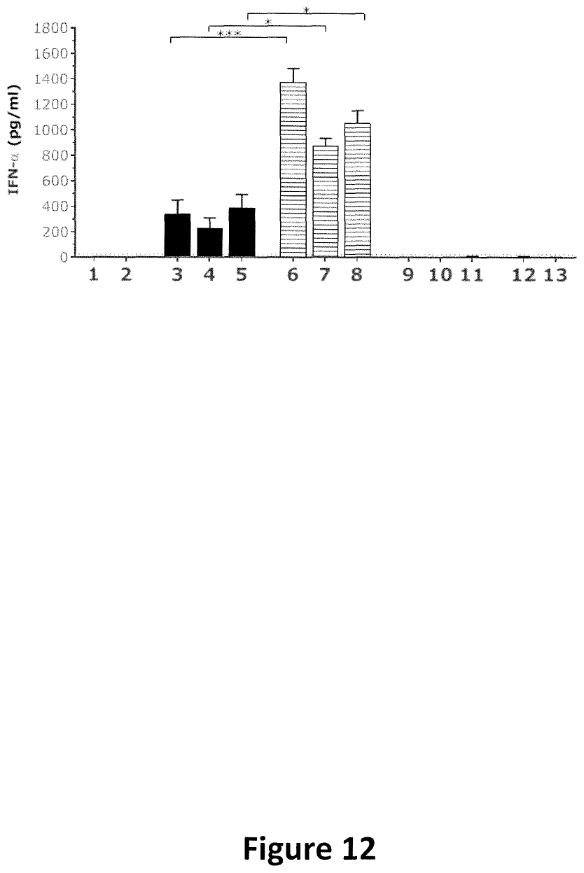

The recombinant Rotavirus protein P2-VP8*P[8] (Group 2, see FIG. 12) and Groups with modified nucleotides (Group 9, 10, 11, 12 and 13, see Table 14) showed no increased INF alpha levels. Group 6, 7 and 8 induced strong INFalpha levels compared to group Group 3, 4 and 5, which showed only a moderate increasing of INFalpha levels in the sera.

INFalpha has a main role in the immune response against viruses. It activates immune cells, such as natural killer cells and macrophages, increases host defenses by up-regulating antigen presentation by increasing the expression of major histocompatibility complex (MHC) antigens. This activation of the innate immune system can be seen as supportive for the subsequent development of a strong adaptive immune response. However, high levels of INFalpha can lead to fever, muscle pain and flu like symptoms. Therefore a moderate increasing of INFalpha could be marker for an optimal immune response to a vaccine against a Rotavirus virus infection.

6.5 Intracellular Cytokine Staining (ICS):

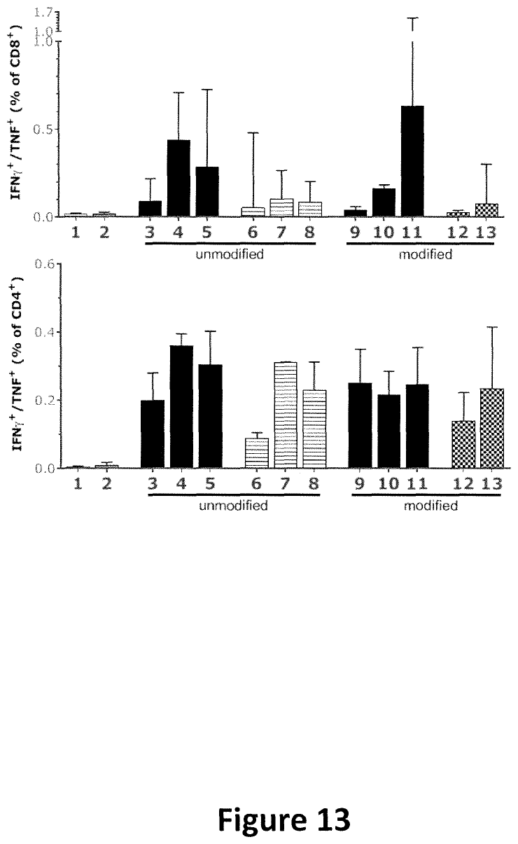

Splenocytes from vaccinated mice (see Example 6.2) were isolated on day 71 according to a standard protocol known in the art. Briefly, isolated spleens were grinded through a cell strainer and washed in PBS/1% FBS followed by red blood cell lysis. After an extensive washing step with PBS/1% FBS, splenocytes were seeded into 96-well plates (2×10⁶ cells per well). Cells were stimulated with a mixture of Rotavirus VP8* peptides (see Table 9) (5 ug/ml each) in the presence of 2.5 ug/ml each of an anti-CD28 antibody (BD Biosciences) and a protein transport inhibitor for 6 h at 37° C. After stimulation, cells were washed and stained for intracellular cytokines using the Cytofix/Cytoperm reagent (BD Biosciences) according to the manufacturer's instructions. The following antibodies were used for staining: Thy1.2-FITC (1:200), CD8-APC-H7 (1:100), TNF-PE (1:100), IFNγ-APC (1:100) (eBioscience), CD4-BD Horizon V450 (1:200) (BD Biosciences) and incubated with Fcγ-block diluted 1:100. Aqua Dye was used to distinguish live/dead cells (Invitrogen). Cells were acquired using a BD FACS Canto II flow cytometer (Beckton Dickinson). Flow cytometry data was analyzed using FlowJo software (Tree Star, Inc.). Results are shown in FIG. 13.

Results:

Groups contained mRNA designs with co-transcriptional capping, a poly(A) sequence, located at 3′ terminus and a UTR combination HSD17B4/PSMB3 (Group 4, 5, 10 and 11, see FIG. 13) induced higher cellular immune responses of CD8 positive T-cells compared to the other groups. All groups of Rotavirus vaccines with a poly(A) sequence, located at 3′ terminus showed higher cellular immune responses of CD4 positive T-cells compared to the recombinant Rotavirus protein P2-VP8*P[8]+Alum (Group 2, see FIG. 13). In addition nearly all groups with a poly(A) sequence, located at 3′ terminus, induced slightly higher immune responses compared to the groups without a poly(A) sequence, located at 3′ terminus (Group 3, 6 and 12, see FIG. 13).

### Example 7: Clinical Development of a Rotavirus mRNA Vaccine

To demonstrate safety and efficacy of the Rotavirus mRNA vaccine, a double blind, randomised, placebo controlled, dose escalation clinical trial will be initiated.

For clinical development, mRNA produced under GMP conditions (e.g. using a procedure as described in WO2016/180430) will be used.

First in Human exposure would most likely be performed in healthy young adults, to establish safety/tolerability and immunogenicity of the candidate vaccine. In an example of a subsequent clinical trial a cohort of healthy HIV uninfected toddlers (aged 1 to <3 years) and infants (aged 6 to <8 weeks) without previous rotavirus vaccination, is intramuscularly injected with the Rotavirus mRNA vaccine. Exclusion criteria can include acute illness at time of enrolment, presence of malnutrition or any systemic disorder, congenital defects, known or suspected impaired immunological function and immunoglobulin therapy or chronic immunosuppressant medications.

The dose escalation phase can be designed to test 2 ug to 200 ug dose levels of vaccine, depending on non-clinical data and clinical data with related vaccines at the time, first in toddlers and then in infants. Toddlers and infants are randomly assigned to receive vaccine or placebo, beginning with the lowest dose. Toddlers in the dose escalation phase of the trial receive up to three intramuscular injections of vaccine or placebo in the anterolateral thigh in a 2 to 8 weeks interval. Infants in the dose escalation phase and the expanded cohort receive three intramuscular injections at 2 to 8-week intervals of vaccine or placebo.

Infants also receive three doses of the oral Rotarix rotavirus vaccine (GlaxoSmithKline, Rixensart, Belgium) as part of this study, at 4, 8, and 12 weeks after the third study injection. Participants are observed for at least 30 min after administration of each injection.

The primary objectives are to assess the safety and reactogenicity of the Rotavirus mRNA vaccine at escalating doses in toddlers and infants, and to investigate the immunogenicity at different doses.

Primary safety endpoints are local and systemic reactions within 7 days after each injection, adverse events within 28 days after each injection, and all serious adverse events, assessed in toddlers and infants who receive at least one dose. Primary immunogenicity endpoints are IgA and IgG titers against P2-VP8 and neutralising antibody sera responses and geometric mean titers 4 weeks after third injection. Therefore serum is collected at baseline, after each vaccination and after the final study injection.

The secondary objective is to assess the effect of Rotavirus mRNA vaccination on shedding of Rotarix vaccine virus subsequently administered in infants, with the endpoint being the proportion of infants shedding rotavirus (determined by ELISA) at 5, 7, or 9 days after administration of the first dose of Rotarix (4 weeks after the third Rotavirus mRNA or placebo injection). Therefore stool samples are collected from infants at 5, 7, and 9 days after the first dose of Rotarix and tested for the presence of rotavirus using e.g. the commercially available ProsPecT Rotavirus Microplate Assay (Oxoid Ltd, Ely, UK), according to the manufacturer's instructions.

Subsequent phase 2b/3 trials would follow to assess efficacy in larger populations, also including specific at-risk populations such as, e.g., HIV positive and malnourished infants.

### Example 8: Analysis of Different mRNA Designs Encoding a Rotavirus Antigen

The present example shows that Rotavirus VP8* mRNA vaccines encoding different antigen designs with modified or unmodified nucleotides induce humoral immune responses in guinea pigs. Binding antibodies were measured using ELISA and virus-neutralizing antibodies to a homologous strain (Wa(G1P[8]), the vaccine strain were analyzed.

mRNA constructs encoding a P2-linker-VP8* (65-223) construct containing either natural nucleotides or modified nucleotides (m1Ψ or Ψ). The constructs were prepared according to Example 1. The mRNAs were formulated with LNPs (see Example 1.4.1 LNP formulation). The different mRNA vaccine candidates (see Table 15) were applied on day 0 and 21 and administered intramuscular (i.m.) with different doses of RNA. Blood samples were taken at day 1, 21, 42, and 56 for determination of humoral immune responses.

Groups 1, 2, 6, 8 and 10 were selected for longevity testing with additional serum sampling time points at day 84, 112 and 140.

TABLE 15
Vaccination scheme of Example 8

| | | | | | | | 5′-cap | | | |
|---|---|---|---|---|---|---|---|---|---|---|
| | | | | | | | structure/ | | | |
| | | | | | | | Poly(A) | | | |
| | No. of | | | | | | sequence, | UTRs | | |
| | guinea | | | | RNA | Construct | located at 3′ | 5′-UTR/ | Modified | SEQ ID |
| Group | pigs | Dose | | Volume | ID | design | terminus | 3′-UTR | nucleotides | NO: |
| 1 | 7 | — | | 100 μl | — | 0.9% NaCl | — | — | — | — |
| | | | | | | buffer, negative | | | | |
| | | | | | | control | | | | |
| 2 | 7 | 20 | μg | 2 × | — | Recombinant | — | — | — | — |
| | | | | 167 μl | | Rotavirus | | | | |
| | | | | | | protein P2-VP8* | | | | |
| | | | | | | P[8] + Alum | | | | |
| 3 | 7 | 8 | μg | 100 μl | R8628 | P2-Linker- | co-trans. | HSD17B4/ | — | 1863 or |
| | | | | | | VP8*(65-223) | cap/A100 | PSMB3 | | 1167 |
| 4 | 7 | 6 | μg | 100 μl | R8575 | P2-Linker- | co-trans. | HSD17B4/ | m1Ψ | 1863 or |
| | | | | | | VP8*(65-223) | cap/A100 | PSMB3 | | 1167 |
| 5 | 7 | 6 | μg | 100 μl | R8576 | P2-Linker- | co-trans. | HSD17B4/ | Ψ | 1863 or |
| | | | | | | VP8*(65-223) | cap/A100 | PSMB3 | | 1167 |
| 6 | 7 | 25 | μg | 100 μl | R8628 | P2-Linker- | co-trans. | HSD17B4/ | — | 1863 or |
| | | | | | | VP8*(65-223) | cap/A100 | PSMB3 | | 1167 |
| 7 | 7 | 25 | μg | 100 μl | R8131 | P2-Linker- | co-trans. | HSD17B4/ | — | 1862 or |
| | | | | | | VP8*(65-223) | cap/A100 | PSMB3 | | 591 |
| 8 | 7 | 25 | μg | 100 μl | R8575 | P2-Linker- | co-trans. | HSD17B4/ | m1Ψ | 1863 or |
| | | | | | | VP8*(65-223) | cap/A100 | PSMB3 | | 1167 |
| 9 | 7 | 25 | μg | 100 μl | R8629 | P2-Linker- | co-trans. | HSD17B4/ | m1Ψ | 1867 |
| | | | | | | VP8*(65-223) | cap/A100 | PSMB3 | | |
| 10 | 7 | 25 | μg | 100 μl | R8576 | P2-Linker- | co-trans. | HSD17B4/ | Ψ | 1863 or |
| | | | | | | VP8*(65-223) | cap/A100 | PSMB3 | | 1167 |
| 11 | 7 | 100 | μg | 100 μl | R8628 | P2-Linker- | co-trans. | HSD17B4/ | — | 1863 or |
| | | | | | | VP8*(65-223) | cap/A100 | PSMB3 | | 1167 |
| 12 | 7 | 100 | μg | 100 μl | R8575 | P2-Linker- | co-trans. | HSD17B4/ | m1Ψ | 1863 or |
| | | | | | | VP8*(65-223) | cap/A100 | PSMB3 | | 1167 |
| 13 | 7 | 100 | μg | 100 μl | R8576 | P2-Linker- | co-trans. | HSD17B4/ | Ψ | 1863 or |
| | | | | | | VP8*(65-223) | cap/A100 | PSMB3 | | 1167 |

8.1. Determination of Specific Humoral Immune Responses by ELISA:

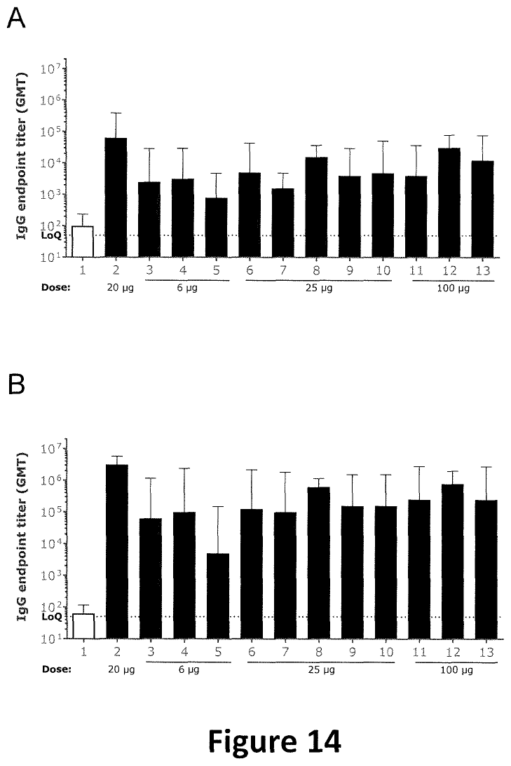

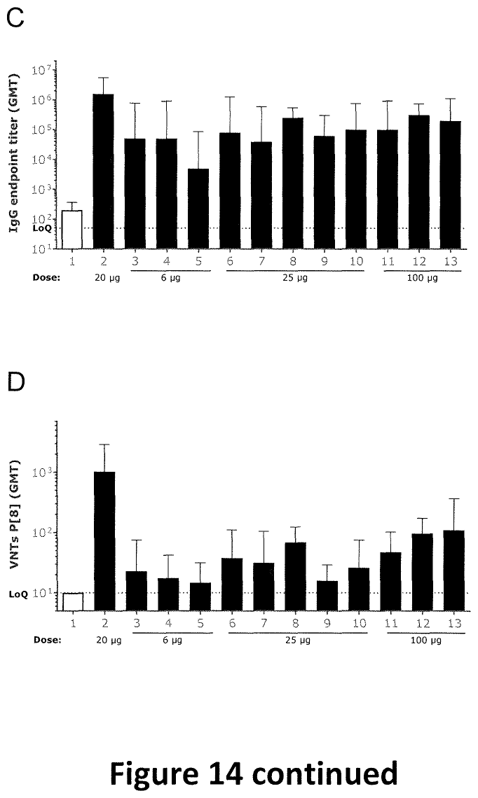

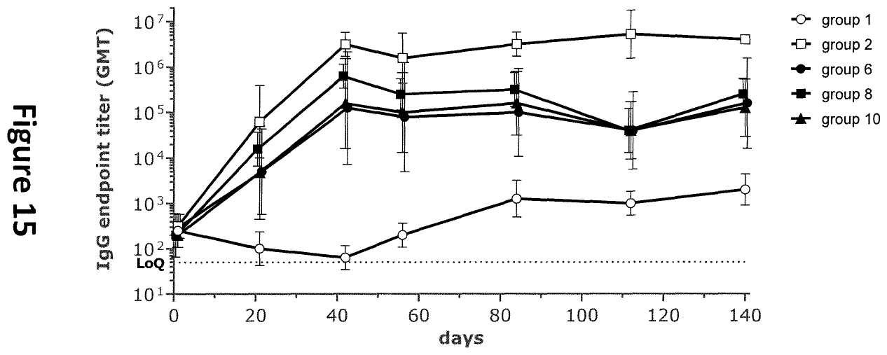

ELISA was performed using recombinant Rotavirus protein P2-VP8*P[8] for coating. Coated plates were incubated using respective serum dilutions, and binding of specific antibodies to the recombinant Rotavirus protein P2-VP8*P[8] was detected using biotinylated isotype-specific anti-guinea pig antibodies followed by streptavidin-HRP (horse radish peroxidase) with Amplex as substrate. Endpoint titers of antibodies (IgG) directed against the recombinant Rotavirus protein P2-VP8*P[8] were measured by ELISA on day 21, 42, and 56 post prime vaccinations for all groups and for groups 1, 2, 6, 8 and 10 also at day 84, 112 and 140. Results are shown in FIGS. 14 A-C (for days 21, 42, 56) and FIG. 15 (also for later time points).

8.2 Determination of Virus-Neutralizing Antibody Titers (VNTs) Against Rotavirus

Serum samples were collected on day 56 after prime vaccination described in Example 5 and virus-neutralizing antibody titers against Rotavirus were measured as described below.

Diluted serum samples were incubated with a constant amount of Rotavirus virus strain Wa (G1P[8]) for 1 hour at 37° C., before transfer to a 96 well plate containing confluent MA104 cells. After 1 hour the monolayers were washed and incubated with the serum-virus mixture for 14-16 hours, frozen and thawed. VNTs were determined by measuring Rotavirus virus antigen in the lysed monolayers in wells receiving serum compared to viral control wells, using a standard enzyme-linked immunosorbent format. The neutralization titer represents a 60% reduction in the amount of virus. Results are shown in FIG. 14 D.

Results

As shown in FIG. 14 A-C, the different Rotavirus antigen constructs induced strong, humoral immune responses in guinea pigs in a dose dependent manner. There were no differences between natural or modified nucleotides detectable.

As shown in FIG. 14 D all groups induced functional antibodies against the Rotavirus strain Wa (G1P[8]).

As shown in FIG. 15, the IgG levels of all tested groups remained stable between day 42 and day 140. There were no differences between natural or modified nucleotides detectable.

### Example 9: Analysis of Different Rotavirus Antigen Designs in Combination with Different mRNA Designs

The present example shows different that Rotavirus mRNA vaccines encoding different antigen designs induce humoral immune responses in guinea pigs. Binding antibodies were measured using ELISA and virus-neutralizing antibodies to homolgous and heterologous strain were analyzed.

mRNA constructs encoding different VP8* constructs were prepared according to Example 1. The mRNAs were formulated with LNPs (see Example 1.4.1 LNP formulation). The different mRNA vaccine candidates (see Table 16) were applied on day 0 and 21 and administered intramuscular (i.m.) with different doses of RNA. Blood samples were taken at day 1, 21, 42, and 56 for determination of humoral immune responses.

Group 1, 2, 7, 8, 9 and 10 were selected for longevity testing with additional serum sampling time points at day 84, 112, 140, 168 and 196.

TABLE 16
Vaccination scheme of Example 9

| | | | | | | | 5′-cap | | |
|---|---|---|---|---|---|---|---|---|---|
| | | | | | | | structure/ | | |
| | | | | | | | poly(A) | | |
| | No. of | | | | | | sequence, | UTRs | |
| | guinea | | | | RNA | | located at 3′ | 5′-UTR/ | SEQ ID |
| Group | pigs | Dose | | Volume | ID | Construct design | terminus | 3′-UTR | NO: |
| 1 | 8 | — | | 100 μl | — | 0.9% NaCl buffer, | — | — | — |
| | | | | | | negative control | | | |
| 2 | 8 | 20 | μg | 2 × | — | Recombinant Rotavirus | — | — | — |
| | | | | 167 μl | | protein P2-VP8*P(8] + | | | |
| | | | | | | Alum | | | |
| 3 | 8 | 6 | μg | 100 μl | R8628 | P2-Linker-VP8*(65- | co-trans. | HSD17B4/ | 1863 or |
| | | | | | | 223) | cap/A100 | PSMB3 | 1167 |
| 4 | 8 | 6 | μg | 100 μl | R8577 | P2-Linker-VP8*(65- | co-trans. | HSD17B4/ | 1873 or |
| | | | | | | 223)-Linker-Ferritin | cap/A100 | PSMB3 | 1185 |
| 5 | 8 | 6 | μg | 100 μl | R8578 | LumSynt-Linker-P2- | co-trans. | HSD17B4/ | 1876 or |
| | | | | | | Linker-VP8*(41-223) | cap/A100 | PSMB3 | 1212 |
| 6 | 8 | 6 | μg | 100 μl | R8579 | SP(IgE)-P2-Linker- | co-trans. | HSD17B4/ | 1879 or |
| | | | | | | VP8*(41-223) | cap/A100 | PSMB3 | 1230 |
| 7 | 8 | 25 | μg | 100 μl | R8628 | P2-Linker-VP8*(65- | co-trans. | HSD17B4/ | 1863 or |
| | | | | | | 223) | cap/A100 | PSMB3 | 1167 |
| 8 | 8 | 25 | μg | 100 μl | R8577 | P2-Linker-VP8*(65- | co-trans. | HSD17B4/ | 1873 or |
| | | | | | | 223)-Linker-Ferritin | cap/A100 | PSMB3 | 1185 |
| 9 | 8 | 25 | μg | 100 μl | R8578 | LumSynt-Linker-P2- | co-trans. | HSD17B4/ | 1876 or |
| | | | | | | Linker-VP8*(41-223) | cap/A100 | PSMB3 | 1212 |
| 10 | 8 | 25 | μg | 100 μl | R8579 | SP(IgE)-P2-Linker- | co-trans. | HSD17B4/ | 1879 or |
| | | | | | | VP8*(41-223) | cap/A100 | PSMB3 | 1230 |

9.1. Determination of Specific Humoral Immune Responses by ELISA:

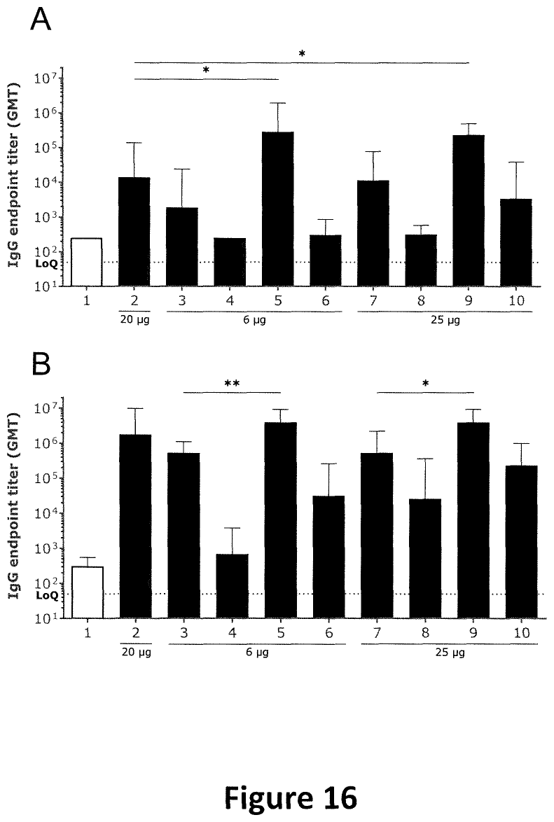

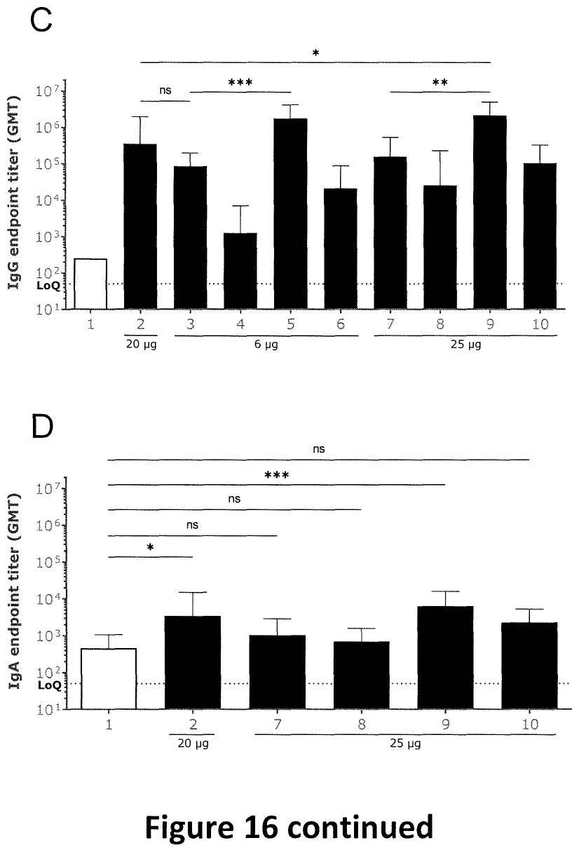

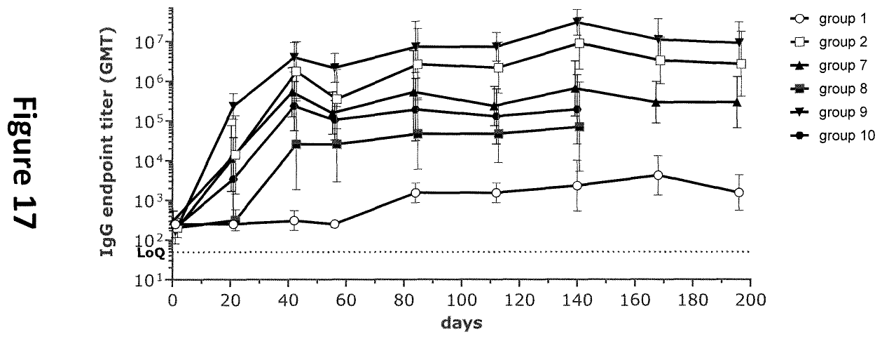

ELISA was performed using recombinant Rotavirus protein P2-VP8*P[8] for coating. Coated plates were incubated using respective serum dilutions, and binding of specific antibodies to the recombinant Rotavirus protein P2-VP8*P[8] was detected using biotinylated isotype specific anti-guinea pig antibodies followed by streptavidin-HRP (horse radish peroxidase) with Amplex as substrate. Endpoint titers of antibodies (IgG) directed against the recombinant Rotavirus protein P2-VP8*P[8] were measured by ELISA on day 21, 42, and 56 post prime vaccinations for all groups and for group 1, 2, 7, 8, 9 and 10 also at day 84, 112, 140, 168 and 196. Results are shown in FIGS. 16 A-C and FIG. 17.

Endpoint titers of antibodies (IgA) directed against the recombinant Rotavirus protein P2-VP8*P[8] were measured by ELISA on day 56 post prime vaccinations for all groups. Results are shown in FIG. 16D.

9.2 Determination of Virus-Neutralizing Antibody Titers (VNTs) Against Rotavirus Serum samples were collected on day 56 after prime vaccination described in Example 5 and virus-neutralizing antibody titers against Rotavirus were measured as described below.

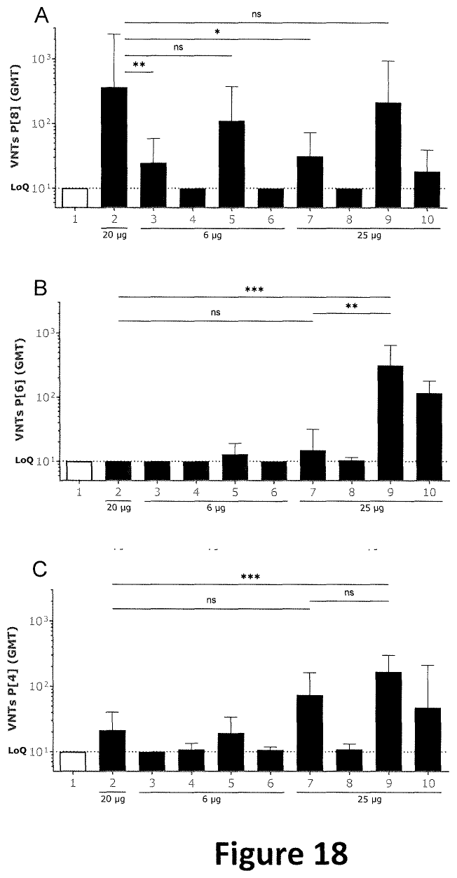

Diluted serum samples were incubated with a constant amount of Rotavirus virus strain Wa (G1P[8]), 1076 (G2P[6]) or DS-1 (G2P[4]) for 1 hour at 37° C., before transfer to a 96 well plate containing confluent MA104 cells. After 1 hour the monolayers were washed and incubated with the serum-virus mixture for 14-16 hours, frozen and thawed. VNTs were determined by measuring Rotavirus virus antigen in the lysed monolayers in wells receiving serum compared to viral control wells, using a standard enzyme-linked immunosorbent format. The neutralization titer represents a 60% reduction in the amount of virus. Results are shown in FIG. 18.

Results

As shown in FIG. 16, the different Rotavirus antigen constructs induced strong, humoral immune responses in guinea pigs. The Lumazine synthase construct (group 5 and 9, FIG. 16A) induced higher IgG immune response at day 21 than the recombinant Rotavirus protein P2-VP8*P[8]+Alum (Group 2, see FIG. 16A) The lumazine synthase P2-VP8*P[8] mRNA vaccine induced significant higher IgG immune response than the P2-VP8*P[8] mRNA vaccine at all days (Group 5 and 9 compared to Groups 3 and 7, FIGS. 16A-C).

As shown in FIG. 17 the IgG levels of the groups remained stable between day 42 and day 196. The anti-VP8* serum IgG level for the lumazine synthase P2-VP8*P[8] mRNA vaccine increased at very early time point and remained the most prominent over time.

As shown in FIG. 16D, as expected for parenteral vaccination, the serum IgA levels were lower than the IgG levels, but for the recombinant Rotavirus protein P2-VP8*P[8]+Alum (Group 2) and the lumazine synthase P2-VP8* P[8] mRNA vaccine (Groups 5 and 9) the levels were significantly increased compared to the negative control group (Group 1).

As shown in FIG. 18A the recombinant Rotavirus protein P2-VP8*P[8]+Alum (Group 2) and the lumazine synthase P2-VP8*P[8] mRNA vaccine (Groups 5 and 9) induced high VNTs against the homologous Rotavirus strain Wa(G1P[8]), but there was no significant difference between these two constructs. The lumazine synthase P2-VP8* P[8] mRNA vaccine was superior compared to the P2-VP8*P[8] mRNA vaccine (Group 3 and 7). As shown in FIGS. 18B-C the recombinant Rotavirus protein P2-VP8*P[8]+Alum (Group 2) had no or only weak heterologous functional antibody responses against the heterologous Rotavirus strain 1076 (G2P[6]) or DS-1 (G2P[4]). In contrast, the lumazine synthase-P2-VP8* P[8] (Group 5 and 9) and the IgE-P2-VP8*P[8](Group 10) mRNA vaccine induced high levels of virus neutralizing antibodies against both heterologous Rotavirus strains (even against the more distantly related P[6] strain).

For the homologous VNT responses (FIG. 18A) only a slight dose response was visible, but for the heterologous VNT responses a dose response was detectable (FIG. 18B-C).

### Example 10: Analysis of Multivalent Rotavirus Antigen Vaccine

The present example shows different mRNA vaccines encoding different Rotavirus serotypes and combinations that induce humoral immune responses in guinea pigs. Binding antibodies will be measured using ELISA and moreover virus neutralizing antibodies to the vaccine strain will be analysed.

mRNA constructs encoding VP8* of different Rotavirus serotypes are prepared according to Example 1. The mRNAs are formulated with LNPs (see Example 1.4.1 LNP formulation). The different mRNA vaccines (see Table 17) are applied on day 0 and 21 and administered intramuscular (i.m.) with different doses of RNA. Blood samples are taken at day 1, 21, 42, and 56 for determination of humoral immune responses.

TABLE 17
Vaccination scheme of Example 10

| | No. of | | Modified |
|---|---|---|---|
| Group | guinea pigs | Rotavirus Serotype | nucleotides |
| 1 | 8 | 0.9% NaCl buffer, negative control | — |
| 2 | 8 | Recombinant Rotavirus protein | — |
| | | P2-VP8*P[8] + Alum | |
| 3 | 8 | VP8* construct P[4] | — |
| 4 | 8 | VP8* construct P[6] | — |
| 5 | 8 | VP8* construct P[8] | — |
| 6 | 8 | VP8* construct combination of P[4], | — |
| | | P[6], P[8] | |
| 7 | 8 | VP8* construct P[4] | m1Ψ |
| 8 | 8 | VP8* construct P[4] | m1Ψ |
| 9 | 8 | VP8* construct P[4] | m1Ψ |
| 10 | 8 | VP8* construct combination of P[4], | m1Ψ |
| | | P[6], P[8] | |
| 11 | 8 | VP8* construct P[4] | Ψ |
| 12 | 8 | VP8* construct P[4] | Ψ |
| 13 | 8 | VP8* construct P[4] | Ψ |
| 14 | 8 | VP8* construct combination of P[4], | Ψ |
| | | P[6], P[8] | |

### Example 11: Analysis of Multivalent Rotavirus Antigen Vaccine with Different Ratios

The present example shows different mRNA vaccine combinations encoding different Rotavirus serotypes with different ratios that induce humoral immune responses in guinea pigs. Binding antibodies will be measured using ELISA and moreover virus neutralizing antibodies to the vaccine strain will be analyzed.

mRNA constructs encoding VP8* of different Rotavirus serotypes are prepared according to Example 1. The mRNAs are formulated with LNPs (see Example 1.4.1 LNP formulation). The different mRNA vaccines (see Table 18) are applied on day 0 and 56 and administered intramuscular (i.m.) with different doses of RNA. Blood samples are taken at day 1, 28, 56 and 84 for determination of humoral immune responses.

TABLE 18
Vaccination scheme of Example 11

| | No. of | | |
|---|---|---|---|
| Group | guinea pigs | Rotavirus Serotype | Ratios |
| 1 | 8 | 0.9% NaCl buffer, negative control | — |
| 2 | 8 | Recombinant Rotavirus protein P2-VP8*P[8] + Alum | — |
| 3 | 8 | VP8* construct combination of P[4], P[6], P[8] | Ratio1 |
| 4 | 8 | VP8* construct combination of P[4], P[6], P[8] | Ratio2 |
| 5 | 8 | VP8* construct combination of P[4], P[6], P[8] | Ratio3 |
| 6 | 8 | VP8* construct combination of P[4], P[6], P[8] | Ratio4 |
| 7 | 8 | VP8* construct combination of P[4], P[6], P[8] | Ratio5 |
| 8 | 8 | VP8* construct combination of P[4], P[6], P[8] | Ratio6 |
| 9 | 8 | VP8* construct combination of P[4], P[6], P[8] | Ratio7 |
| 10 | 8 | VP8* construct combination of P[4], P[6], P[8] | Ratio8 |

### Example 12: Analysis and Challenge of Multivalent Rotavirus Antigen Vaccine

The present example shows different mRNA vaccine combinations encoding different Rotavirus serotypes with two different doses that induce protective immune responses in gnotobiotic pigs. Binding antibodies are measured using ELISA and moreover virus-neutralizing antibodies to the vaccine strain is analysed.

mRNA constructs encoding VP8* constructs of different Rotavirus P-serotypes are prepared according to Example 1. The mRNAs are formulated with LNPs (see Example 1.4.1 LNP formulation). The different mRNA vaccines (see Table 19) are applied on day 0, 14 and 28 and administered intramuscular (i.m.) with two different doses of RNA. Blood samples are taken at day 1, 14, 28, 35, and 42 for determination of humoral immune responses. To test the protective efficacy of the mRNA vaccines, an oral challenge with Rotavirus Wa (G1P[8]) will be performed at day 35. To assess the effect of Rotavirus mRNA vaccination on shedding of virus, stool samples are collected from pigs at day 35 to 42.

TABLE 19
Vaccination scheme of Example 12

| | No. of | | | |
|---|---|---|---|---|
| Group | guinea pigs | Dosis | Rotavirus Serotype | Challenge |
| 1 | 7 | — | 0.9% NaCl buffer, negative control | Rotavirus Wa |
| | | | | (G1P[8] strain) |
| 2 | 7 | 3 × 30 μg | Trivalent recombinant Rotavirus protein P2-VP8* P[8], | Rotavirus Wa |
| | | | P[6], P[4] + Alum | (G1P[8] strain) |
| 3 | 7 | Dose 1 | VP8* mRNA construct 1 combination of P[4], P[6], | Rotavirus Wa |
| | | | P[8] | (G1P[8] strain) |
| 4 | 7 | Dose 1 | VP8* construct 2 combination of P[4], P[6], P[8] | Rotavirus Wa |
| | | | | (G1P[8] strain) |
| 5 | 7 | Dose 2 | VP8* construct 2 combination of P[4], P[6], P[8] | Rotavirus Wa |
| | | | | (G1P[8] strain) |

### Example 13: Analysis of Trivalent Rotavirus Antigen Vaccine with Different Candidates

The present example shows trivalent mRNA vaccine combinations encoding different Rotavirus serotypes always with a 1:1:1 ratio that are tested for their capacity to induce humoral immune responses in guinea pigs. Binding antibodies are measured using ELISA and moreover virus-neutralizing antibodies to the vaccine strain are analyzed.

mRNA constructs encoding VP8* of different Rotavirus serotypes are prepared according to Example 1. The mRNAs are formulated with LNPs (see Example 1.4.1 LNP formulation). The different mRNA vaccines are applied at two or three time points and administered intramuscular (i.m.) to seven guinea pigs per group with different doses of RNA (see Table 20). Blood samples are taken at day 1, 21/22, 42 and 70 for determination of humoral immune responses.

TABLE 20
Vaccination scheme of Example 13

| | | | | | | 5′-cap | | |
|---|---|---|---|---|---|---|---|---|
| | | | | | | structure/ | | |
| | | | | | | poly(A) | | |
| | | | | | | sequence, | UTRs | |
| | | RNA | Construct | | Serum | located at 3′ | 5′-UTR/ | SEQ ID |
| Group | Dose | ID | design | Immunization | samples | terminus | 3′-UTR | NO: |
| 1 | 100 μl | — | 0.9% NaCl | d 0, d 21, | d 1, d 21, | — | — | — |
| | | | buffer, negative | d 42 | d 42, d 70 | | | |
| | | | control | | | | | |
| 2 | 1 × | — | Monovalent rec. | d 0, d 21, | d 1, d 21, | — | — | — |
| | 20 μg | | Rotavirus | d 42 | d 42, d 70 | | | |
| | | | protein P2- | | | | | |
| | | | VP8*P[8] + | | | | | |
| | | | Alum | | | | | |
| 3 | 3 × | — | Trivalent rec. | d 21, d 42 | d 22, d 42, | — | — | — |
| | 6.7 μg | | Rotavirus | | d 70 | | | |
| | | | protein P2- | | | | | |
| | | | VP8*P[8], P[6], | | | | | |
| | | | P[4] + Alum | | | | | |
| 4 | 3 × | — | Trivalent rec. | d 0, d 21, | d 1, d 21, | — | — | — |
| | 6.7 μg | | Rotavirus | d 42 | d 42, d 70 | | | |
| | | | protein P2- | | | | | |
| | | | VP8*P[8], P[6], | | | | | |
| | | | P[4] + Alum | | | | | |
| 5 | 1 × | R8628 | P2-Linker- | d 0, d 21, | d 1, d 21, | co-trans. | HSD17B4/ | 1863 or |
| | 25 μg | | VP8*(65-223) | d 42 | d 42, d 70 | cap/A100 | PSMB3 | 1167 |
| | | | P[8] | | | | | |
| 6 | 1 × | R9077 | P2-Linker- | d 0, d21, | d 1, d 21, | co-trans. | HSD17B4/ | 1922 |
| | 25 μg | | VP8*(65-223) | d42 | d 42, d 70 | cap/A100 | PSMB3 | |
| | | | P[6] | | | | | |
| 7 | 1 × | R9078 | P2-Linker- | d 0, d 21, | d 1, d 21, | co-trans. | HSD17B4/ | 1921 |
| | 25 μg | | VP8*(65-223) | d 42 | d 42, d 70 | cap/A100 | PSMB3 | |
| | | | P[4] | | | | | |
| 8 | 3 × | R8628 + | Trivalent P2- | d 21, d 42 | d 22, d 42, | co-trans. | HSD17B4/ | 1863 or |
| | 8.3 μg | R9077 + | Linker-VP8*(65-223) | | d 70 | cap/A100 | PSMB3 | 1167, |
| | | R9078 | P[8, | | | | | 1922, |
| | | | P[6], P[4]] | | | | | 1921 |
| 9 | 3 × | R8628 + | Trivalent P2- | d 0, d 21, | d 1, d 21, | co-trans. | HSD17B4/ | 1863 or |
| | 8.3 μg | R9077 + | Linker-VP8*(65-223) | d 42 | d 42, d 70 | cap/A100 | PSMB3 | 1167, |
| | | R9078 | P[8, | | | | | 1922, |
| | | | P[6], P[4] | | | | | 1921 |
| 10 | 1 × | R8578 | LumSynt-Linker- | d 0, d 21, | d 1, d 21, | co-trans. | HSD17B4/ | 1876 or |
| | 25 μg | | P2-Linker- | d 42 | d 42, d 70 | cap/A100 | PSMB3 | 1212 |
| | | | VP8*(41-223) | | | | | |
| | | | P[8] | | | | | |
| 11 | 1 × | R9091 | LumSynt-Linker- | d 0, d 21, | d 1, d 21, | co-trans. | HSD17B4/ | 1924 |
| | 25 μg | | P2-Linker- | d 42 | d 42, d 70 | cap/A100 | PSMB3 | |
| | | | VP8*(41-223) | | | | | |
| | | | P[6] | | | | | |
| 12 | 1 × | R9092 | LumSynt-Linker- | d 0, d 21, | d 1, d 21, | co-trans. | HSD17B4/ | 1923 |
| | 25 μg | | P2-Linker- | d 42 | d 42, d 70 | cap/A100 | PSMB3 | |
| | | | VP8*(41-223) | | | | | |
| | | | P[4] | | | | | |
| 13 | 3 × | R8578, | Trivalent | d 21, d 42 | d 22, d 42, | co-trans. | HSD17B4/ | 1876 or |
| | 1 μg | R9091, | LumSynt-Linker- | | d 70 | cap/A100 | PSMB3 | 1212, |
| | | R9092 | P2-Linker- | | | | | 1924, |
| | | | VP8*(41-223) | | | | | 1923 |
| | | | P[8], P[6], | | | | | |
| | | | P[4] | | | | | |
| 14 | 3 × | R8578, | Trivalent | d 0, d 21, | d 1, d 21, | co-trans. | HSD17B4/ | 1876 or |
| | 1 μg | R9091, | LumSynt-Linker- | d 42 | d 42, d 70 | cap/A100 | PSMB3 | 1212, |
| | | R9092 | P2-Linker- | | | | | 1924, |
| | | | VP8*(41-223) | | | | | 1923 |
| | | | P[8], P[6], | | | | | |
| | | | P[4] | | | | | |
| 15 | 3 × | R8578, | Trivalent | d 21, d 42 | d 22, d 42, | co-trans. | HSD17B4/ | 1876 or |
| | 8.3 μg | R9091, | LumSynt-Linker- | | d 70 | cap/A100 | PSMB3 | 1212, |
| | | R9092 | P2-Linker- | | | | | 1924, |
| | | | VP8*(41-223) | | | | | 1923 |
| | | | P[8], P[6], | | | | | |
| | | | P[4] | | | | | |
| 16 | 3 × | R8578, | Trivalent | d 0, d 21, | d 1, d 21, | co-trans. | HSD17B4/ | 1876 or |
| | 8.3 μg | R9091, | LumSynt-Linker- | d 42 | d 42, d 70 | cap/A100 | PSMB3 | 1212, |
| | | R9092 | P2-Linker- | | | | | 1924, |
| | | | VP8*(41-223) | | | | | 1923 |
| | | | P[8], P[6], | | | | | |
| | | | P[4] | | | | | |

9.1. Determination of Specific Humoral Immune Responses by ELISA:

ELISA are performed using recombinant Rotavirus protein P2-VP8* P[8], P[6] or P[4] for coating. Coated plates are incubated using respective serum dilutions, and binding of specific antibodies to the recombinant Rotavirus protein P2-VP8* is detected using biotinylated isotype specific anti-guinea pig antibodies followed by streptavidin-HRP (horse radish peroxidase) with Amplex as substrate. Endpoint titers of antibodies (IgG) directed against the recombinant Rotavirus protein P2-VP8* P[8], P[6] or P[4] are measured by ELISA on day 21, 42, and 70 post prime immunization for groups 1, 2, 4-7, 9-12, 14 and 16 and on day 21 and 49 post prime immunization for groups 3, 8, 13, 15.

9.2 Determination of Virus-Neutralizing Antibody Titers (VNTs) Against Rotavirus

Serum samples are collected on day 70 post prime for groups 1, 2, 4-7, 9-12, 14 and 16 and on day 49 post prime for groups 3, 8, 13, 15 as described in Example 5 and Rotavirus neutralization titers are measured as described below.

Diluted serum samples are incubated with a constant amount of Rotavirus virus strain Wa (G1P[8]), 1076 (G2P[6]), or DS-1 (G2P[4]) for 1 hour at 37° C., before transfer to a 96 well plate containing confluent MA104 cells. After 1 hour the monolayers are washed and incubated with the serum-virus mixture for 15-17 hours, frozen and thawed. VNTs are determined by measuring Rotavirus virus antigen in the lysed monolayers in wells receiving serum compared to viral control wells, using a standard enzyme-linked immunosorbent format. The neutralization titer represents a 60% reduction in the amount of virus.

1. A coding RNA for a Rotavirus vaccine comprising
a) at least one heterologous 5′ untranslated region (5′-UTR) and/or at least one heterologous 3′ untranslated region (3′-UTR); and
b) at least one coding sequence operably linked to said 3′-UTR and/or 5′-UTR encoding at least one antigenic protein of a Rotavirus, wherein said antigenic protein is or is derived from VP8* or an immunogenic fragment or immunogenic variant thereof.

2. Coding RNA of claim 1, wherein the Rotavirus is selected from species A, B or C, preferably wherein the Rotavirus is Rotavirus A.

3. Coding RNA of claim 1 or 2, wherein the Rotavirus is selected from the G-serotypes or P-serotypes G1, G2, G3, G4, G9, G12, P[4], P[6] or P[8].

4. Coding RNA of any one of the preceding claims, wherein the Rotavirus is a Rotavirus A selected from the P-serotypes P[4], P[6] or P[8].

5. Coding RNA of any one of the preceding claims, wherein the Rotavirus is a Rotavirus A selected from Human rotavirus A BE1058 (RVA/Human-wt/BEL/BE1058/2008/G2P[4], G2P[4], JN849123.1, GI:371455744, AEX30665.1, acronym: RVA/BE1058/P[4]), Human rotavirus A F01322 (Hu/BEL/F01322/2009/G3P[6], G3P[6], JF460826.1, GI: 37531451, AFA51886.1, acronym: RVA/F01322/P[6]), Human rotavirus A BE1128 (RVA/Human-wt/BEL/BE1128/2009/G1P[8], G1P[8], JN849135.1. GI: 371455756, AEX30671, acronym: RVA/BE1128/P[8]), or Human rotavirus A WA-VirWa (Wa variant VirWa, G1P[8], ACR22783.1, GI: 237846292, FJ423116, acronym: RVA/Wa-VirWa/P[8]).

6. Coding RNA of any one of the preceding claims, wherein the VP8* is a full length VP8* protein having an amino acid sequence comprising or consisting of amino acid 1 to amino acid 240, or a fragment of a VP8* protein.

7. Coding RNA of any one of claim 6, wherein the fragment of a VP8* comprises the lectin domain and lacks the N-terminal alpha helix-domain.

8. Coding RNA of any one of the preceding claims, wherein the amino acid sequences of the at least one antigenic protein derived from VP8* is mutated to delete at least one predicted or potential glycosylation site.

9. Coding RNA of any one of the preceding claims, wherein the amino acid sequences of the at least one antigenic protein derived from VP8* is mutated to delete all predicted or potential glycosylation sites.

10. Coding RNA of any one of the preceding claims, wherein the at least one coding sequence encodes at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 19-45, or an immunogenic fragment or immunogenic variant of any of these.

11. Coding RNA of any one of the preceding claims, wherein the at least one coding sequence additionally encodes one or more heterologous peptide or protein elements selected from a signal peptide, a linker, a helper epitope, an antigen clustering domain, or a transmembrane domain.

12. Coding RNA of claim 11, wherein the signal peptide is or is derived from HsPLAT, HsALB, IgE, wherein the amino acid sequences of said heterologous signal peptides is identical or at least 70%, 80%, 85%, 86%, 87%, 88%, 89%, 90%, 91%, 92%, 93%, 94%, 95%, 96%, 97%, 98%, or 99% identical to any one of amino acid sequences SEQ ID NOs: 1738-1740, or fragment or variant of any of these.

13. Coding RNA of claim 11, wherein the helper epitope is or is derived from P2, wherein the amino acid sequences of said helper epitopes is identical or at least 70%, 80%, 85%, 86%, 87%, 88%, 89%, 90%, 91%, 92%, 93%, 94%, 95%, 96%, 97%, 98%, or 99% identical to amino acid sequence SEQ ID NOs: 1750, or fragment or variant thereof.

14. Coding RNA of claim 11, wherein the antigen clustering domain is or is derived from ferritin or lumazine-synthase, wherein the amino acid sequences of said antigen clustering domain is identical or at least 70%, 80%, 85%, 86%, 87%, 88%, 89%, 90%, 91%, 92%, 93%, 94%, 95%, 96%, 97%, 98%, or 99% identical to any one of amino acid sequences SEQ ID NOs1759, 1764, or fragment or variant of any of these.

15. Coding RNA of claim 11, wherein the transmembrane domain is or is derived from an influenza HA transmembrane domain, preferably derived from an influenza A HA H1N1, more preferably from H1N1/A/Netherlands/602/2009, TM domain_HA, aa521-566, NCBI Acc. No.: ACQ45338.1, CY039527.1), or fragment or variant thereof.

16. Coding RNA of any one of the preceding claims, wherein the at least one coding sequence encodes the following elements preferably in N-terminal to C-terminal direction:
a) helper epitope, VP8*protein or VP8*fragment; or
b) helper epitope, VP8*protein or VP8*fragment; antigen clustering domain; or
c) Signal peptide, helper epitope, VP8*protein or fragment thereof; or
d) Signal peptide, helper epitope, VP8*protein or VP8*fragment, antigen clustering domain; or
e) Signal peptide, helper epitope, VP8*protein or VP8*fragment, transmembrane domain; or
f) antigen clustering domain, helper epitope; VP8*protein or VP8*fragment.

17. Coding RNA of any one of the preceding claims, wherein the at least one coding sequence encodes at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 1-6, 46-117, 1899, 1900, or an immunogenic fragment or immunogenic variant of any of these.

18. Coding RNA of claim 17, wherein the at least one coding sequence encodes at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 1-3, 4-6, 46-54, 64-72, 91-99, 109-117, or an immunogenic fragment or immunogenic variant of any of these.

19. Coding RNA of any one of the preceding claims, wherein the at least one coding sequence comprises a codon modified coding sequence comprising or consisting of a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 190-261, 298-369, 406-477, 514-585, 1901-1906, or a fragment or variant of any of these sequences.

20. Coding RNA of any one of the preceding claims, wherein the at least one coding sequence comprises at least one modified nucleotide selected from pseudouridine (LP) and N1-methylpseudouridine (ml P), preferably wherein all uracil nucleotides are replaced by pseudouridine (V) nucleotides and/or N1-methylpseudouridine (ml 4) nucleotides.

21. Coding RNA of any one of the preceding claims, wherein the at least one coding sequence is a codon modified coding sequence, wherein the amino acid sequence encoded by the at least one codon modified coding sequence is preferably not being modified compared to the amino acid sequence encoded by the corresponding wild type coding sequence.

22. Coding RNA according to claim 21, wherein the at least one codon modified coding sequence is selected from C maximized coding sequence, CAI maximized coding sequence, human codon usage adapted coding sequence, G/C content modified coding sequence, and G/C optimized coding sequence, or any combination thereof.

23. Coding RNA of claim 21 or 22, wherein the at least one coding sequence comprises or consists of a codon modified coding sequence comprising or consisting of a nucleic acid sequence being identical or at least 70%, 80%, 85%, 86%, 87%, 88%, 89%, 90%, 91%, 92%, 93%, 94%, 95%, 96%, 97%, 98%, or 99% identical to any one SEQ ID NOs: 154-585, 1901-1906 or a fragment or variant of any of these sequences.

24. Coding RNA of any one of claims 21 to 23, wherein the at least one coding sequence comprises or consists of a codon modified coding sequence comprising or consisting of a nucleic acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 190-198, 208-216, 235-243, 253-261, 298-306, 316-324, 343-351, 361-369, 1901-1906 or a fragment or variant of any of these sequences.

25. Coding RNA of any one of the preceding claims, wherein the coding RNA is an mRNA, a self-replicating RNA, a circular RNA, a viral RNA, or a replicon RNA.

26. Coding RNA of any one of the preceding claims, wherein the coding RNA is an mRNA.

27. Coding RNA of any one of the preceding claims, wherein the coding RNA comprises a 5′-cap structure, preferably cap0, cap1, cap2, a modified cap0 or a modified cap1 structure.

28. Coding RNA of claim 27, wherein the a 5′-cap structure is a cap1 structure,

29. Coding RNA of any one of the preceding claims, wherein the coding RNA comprises a cap1 structure, wherein said cap1 structure is obtainable by co-transcriptional capping preferably using a trinucleotide cap1 analogue.

30. Coding RNA of any one of claim 27 to 29, wherein about 70%, 75%, 80%, 85%, 90%, 95% of the coding RNA (species) comprises a cap1 structure as determined using a capping assay.

31. Coding RNA of any one of the preceding claims, wherein the coding RNA comprises at least one poly(A) sequence comprising about 30 to about 200 adenosine nucleotides, preferably comprising about 100 adenosine nucleotides.

32. Coding RNA of claim 31, wherein the at least one poly(A) sequence is located at the 3′ terminus, preferably wherein the 3′ terminal nucleotide of the coding RNA is the 3′ terminal A nucleotide of the poly(A) sequence.

33. Coding RNA of any one of the preceding claims, wherein the coding RNA comprises a cap1 structure as defined in claims 27 to 30 and at least one poly(A) sequence as defined in claims 31 to 32.

34. Coding RNA of any one of the preceding claims, wherein the RNA comprises at least one histone stem-loop, wherein the histone stem-loop preferably comprises or consists of a nucleic acid sequence identical or at least 70%, 80%, 90%, 91%, 92%, 93%, 94%, 95%, 96%, 97%, 98%, or 99% identical to SEQ ID NOs: 1819 or 1820, or fragments or variants thereof.

35. Coding RNA of any one of the preceding claims, wherein the RNA comprises at least one 3′ terminal sequence element comprising or consisting of a nucleic acid sequence being identical or at least 70%, 80%, 90%, 91%, 92%, 93%, 94%, 95%, 96%, 97%, 98%, or 99% identical to SEQ ID NOs: 1825-1856, or a fragment or variant thereof.

36. Coding RNA of any one of the preceding claims, wherein the at least one heterologous 3′-UTR comprises or consisting of a nucleic acid sequence derived from a 3′-UTR of a gene selected from PSMB3, ALB7, alpha-globin, CASP1, COX6B1, GNAS, NDUFA1 and RPS9, or from a homolog, a fragment or a variant of any one of these genes.

37. Coding RNA of any one of the preceding claims, wherein the at least one heterologous 5′-UTR comprises or consisting of a nucleic acid sequence derived from a 5′-UTR of a gene selected from HSD17B4, RPL32, ASAH1, ATP5A1, MP68, NDUFA4, NOSIP, RPL31, SLC7A3, TUBB4B and UBQLN2, or from a homolog, a fragment or variant of any one of these genes.

38. Coding RNA of any one of the preceding claims, wherein
the at least one heterologous 5′-UTR is derived from a 5′-UTR of a HSD17B4 gene, or from a corresponding RNA sequence, homolog, fragment or variant thereof and the at least one 3-UTR is derived from a 3′-UTR of a PSMB3 gene, or from a corresponding RNA sequence, homolog, fragment or variant thereof, preferably wherein said 5′-UTR comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 1781 or 1782 or a fragment or a variant thereof, and wherein said 3′-UTR comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs:
1803 or 1804 or a fragment or a variant thereof; or
the at least one heterologous 3′-UTR is derived from a 3′-UTR of a alpha-globin gene gene, or from a corresponding RNA sequence, homolog, fragment or variant thereof, preferably wherein said 3′-UTR comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 1817 or 1818 or a fragment or a variant thereof.

39. Coding RNA of any one of the preceding claims, wherein the coding RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 586-1737, 1862-1882, 1885-1898, 1907-1930 or a fragment or variant of any of these sequences.

40. Coding RNA of claim 39, wherein the coding RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 586-594, 604-612, 631-639, 649-666, 676-684, 703-711, 721-738, 748-756, 775-783, 793-810, 820-828, 847-855, 865-882, 892-900, 919-927, 937-954, 964-972, 991-999, 1009-1026, 1036-1044, 1063-1071, 1081-1098, 1108-1116, 1135-1143, 1153-1170, 1180-1188, 1207-1215, 1225-1242, 1252-1260, 1279-1287, 1297-1314, 1324-1332, 1351-1359, 1369-1386, 1396-1404, 1423-1431, 1441-1458, 1468-1476, 1495-1503, 1513-1530, 1540-1548, 1567-1575, 1585-1602, 1612-1620, 1639-1647, 1657-1674, 1684-1692, 1711-1719, 1729-1737, 1862-1870, 1872-1877, 1885, 1898, 1907-1930 or a fragment or variant of any of these sequences.

41. A composition comprising at least one coding RNA as defined in any one of claims 1 to 40, wherein the composition optionally comprises at least one pharmaceutically acceptable carrier or excipient.

42. Composition of claim 41, wherein the composition comprises more than one or a plurality, preferably 2, 3, 4, 5, 6, 7, 8, 9, or 10 different coding RNAs each defined in any one of claims 1 to 40.

43. Composition of claim 42, wherein the composition comprises
(i) at least one coding RNA encoding at least one antigenic protein that is or is derived from VP8* of a Rotavirus A from a P[4] serotype, preferably according to SEQ ID Nos: 586-588, 595-597, 604-606, 613-615, 622-624, 631-633, 640-642, 649-651, 658-660, 667-669, 676-678, 685-687, 694-696, 703-705, 712-714, 721-723, 730-732, 739-741, 748-750, 757-759, 766-768, 775-777, 784-786, 793-795, 802-804, 811-813, 820-822, 829-831, 838-840, 847-849, 856-858, 865-867, 874-876, 883-885, 892-894, 901-903, 910-912, 919-921, 928-930, 937-939, 946-948, 955-957, 964-966, 973-975, 982-984, 991-993, 1000-1002, 1009-1011, 1018-1020, 1027-1029, 1036-1038, 1045-1047, 1054-1056, 1063-1065, 1072-1074, 1081-1083, 1090-1092, 1099-1101, 1108-1110, 1117-1119, 1126-1128, 1135-1137, 1144-1146, 1153-1155, 1162-1164, 1171-1173, 1180-1182, 1189-1191, 1198-1200, 1207-1209, 1216-1218, 1225-1227, 1234-1236, 1243-1245, 1252-1254, 1261-1263, 1270-1272, 1279-1281, 1288-1290, 1297-1299, 1306-1308, 1315-1317, 1324-1326, 1333-1335, 1342-1344, 1351-1353, 1360-1362, 1369-1371, 1378-1380, 1387-1389, 1396-1398, 1405-1407, 1414-1416, 1423-1425, 1432-1434, 1441-1443, 1450-1452, 1459-1461, 1468-1470, 1477-1479, 1486-1488, 1495-1497, 1504-1506, 1513-1515, 1522-1524, 1531-1533, 1540-1542, 1549-1551, 1558-1560, 1567-1569, 1576-1578, 1585-1587, 1594-1596, 1603-1605, 1612-1614, 1621-1623, 1630-1632, 1639-1641, 1648-1650, 1657-1659, 1666-1668, 1675-1677, 1684-1686, 1693-1695, 1702-1704, 1711-1713, 1720-1722, 1729-1731, 1886, 1907, 1909, 1911, 1913, 1915, 1917, 1919, 1921, 1923, 1925, 1927, 1929 or fragments or variants thereof; and
(ii) at least one coding RNA encoding at least one antigenic protein that is or is derived from VP8* of a Rotavirus A from a P[6] serotype, preferably according to SEQ ID Nos: 589, 590, 598, 599, 607, 608, 616, 617, 625, 626, 634, 635, 643, 644, 652, 653, 661, 662, 670, 671, 679, 680, 688, 689, 697, 698, 706, 707, 715, 716, 724, 725, 733, 734, 742, 743, 751, 752, 760, 761, 769, 770, 778, 779, 787, 788, 796, 797, 805, 806, 814, 815, 823, 824, 832, 833, 841, 842, 850, 851, 859, 860, 868, 869, 877, 878, 886, 887, 895, 896, 904, 905, 913, 914, 922, 923, 931, 932, 940, 941, 949, 950, 958, 959, 967, 968, 976, 977, 985, 986, 994, 995, 1003, 1004, 1012, 1013, 1021, 1022, 1030, 1031, 1039, 1040, 1048, 1049, 1057, 1058, 1066, 1067, 1075, 1076, 1084, 1085, 1093, 1094, 1102, 1103, 1111, 1112, 1120, 1121, 1129, 1130, 1138, 1139, 1147, 1148, 1156, 1157, 1165, 1166, 1174, 1175, 1183, 1184, 1192, 1193, 1201, 1202, 1210, 1211, 1219, 1220, 1228, 1229, 1237, 1238, 1246, 1247, 1255, 1256, 1264, 1265, 1273, 1274, 1282, 1283, 1291, 1292, 1300, 1301, 1309, 1310, 1318, 1319, 1327, 1328, 1336, 1337, 1345, 1346, 1354, 1355, 1363, 1364, 1372, 1373, 1381, 1382, 1390, 1391, 1399, 1400, 1408, 1409, 1417, 1418, 1426, 1427, 1435, 1436, 1444, 1445, 1453, 1454, 1462, 1463, 1471, 1472, 1480, 1481, 1489, 1490, 1498, 1499, 1507, 1508, 1516, 1517, 1525, 1526, 1534, 1535, 1543, 1544, 1552, 1553, 1561, 1562, 1570, 1571, 1579, 1580, 1588, 1589, 1597, 1598, 1606, 1607, 1615, 1616, 1624, 1625, 1633, 1634, 1642, 1643, 1651, 1652, 1660, 1661, 1669, 1670, 1678, 1679, 1687, 1688, 1696, 1697, 1705, 1706, 1714, 1715, 1723, 1724, 1732, 1733, 1887, 1890, 1895-1897, 1908, 1910, 1912, 1914, 1916, 1918, 1920, 1922, 1924, 1926, 1928, 1930 or fragments or variants thereof; and
(iii) at least one coding RNA encoding at least one antigenic protein that is or is derived from VP8* of a Rotavirus A from a P[8] serotype, preferably according to SEQ ID Nos: 591-594, 600-603, 609-612, 618-621, 627-630, 636-639, 645-648, 654-657, 663-666, 672-675, 681-684, 690-693, 699-702, 708-711, 717-720, 726-729, 735-738, 744-747, 753-756, 762-765, 771-774, 780-783, 789-792, 798-801, 807-810, 816-819, 825-828, 834-837, 843-846, 852-855, 861-864, 870-873, 879-882, 888-891, 897-900, 906-909, 915-918, 924-927, 933-936, 942-945, 951-954, 960-963, 969-972, 978-981, 987-990, 996-999, 1005-1008, 1014-1017, 1023-1026, 1032-1035, 1041-1044, 1050-1053, 1059-1062, 1068-1071, 1077-1080, 1086-1089, 1095-1098, 1104-1107, 1113-1116, 1122-1125, 1131-1134, 1140-1143, 1149-1152, 1158-1161, 1167-1170, 1176-1179, 1185-1188, 1194-1197, 1203-1206, 1212-1215, 1221-1224, 1230-1233, 1239-1242, 1248-1251, 1257-1260, 1266-1269, 1275-1278, 1284-1287, 1293-1296, 1302-1305, 1311-1314, 1320-1323, 1329-1332, 1338-1341, 1347-1350, 1356-1359, 1365-1368, 1374-1377, 1383-1386, 1392-1395, 1401-1404, 1410-1413, 1419-1422, 1428-1431, 1437-1440, 1446-1449, 1455-1458, 1464-1467, 1473-1476, 1482-1485, 1491-1494, 1500-1503, 1509-1512, 1518-1521, 1527-1530, 1536-1539, 1545-1548, 1554-1557, 1563-1566, 1572-1575, 1581-1584, 1590-1593, 1599-1602, 1608-1611, 1617-1620, 1626-1629, 1635-1638, 1644-1647, 1653-1656, 1662-1665, 1671-1674, 1680-1683, 1689-1692, 1698-1701, 1707-1710, 1716-1719, 1725-1728, 1734-1737, 1862-1882, 1885, 1888, 1889, 1891-1894, 1898 or fragments or variants thereof,
wherein preferably the at least one antigenic protein comprises a heterologous element selected from a signal peptide, a linker, a helper epitope, an antigen clustering domain, or a transmembrane domain.

44. Composition of any one of claims 41 to 43, wherein the at least one coding RNA or the plurality of coding RNAs is complexed or associated with or at least partially complexed or partially associated with one or more cationic or polycationic compound, preferably cationic or polycationic polymer, cationic or polycationic polysaccharide, cationic or polycationic lipid, cationic or polycationic protein, cationic or polycationic peptide, or any combinations thereof.

45. Composition of claim 44, wherein the at least one coding RNA or the plurality of coding RNAs is complexed, encapsulated, partially encapsulated, or associated with one or more lipids, thereby forming liposomes, lipid nanoparticles, lipoplexes, and/or nanoliposomes.

46. Composition of claim 45, wherein the at least one coding RNA or the plurality of coding RNAs is complexed with one or more lipids thereby forming lipid nanoparticles (LNP).

47. Composition of claim 46, wherein the LNP comprises a cationic lipid according to formula III-3:

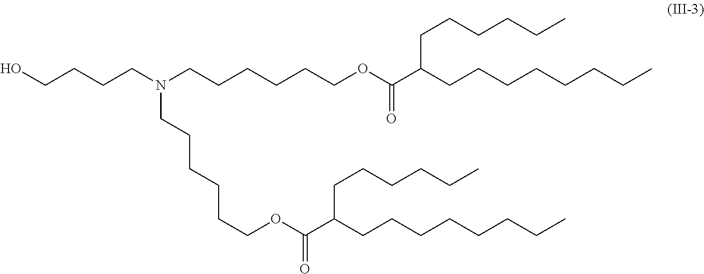 [SMILES: CCCCCCCCC(CCCCCC)C(=O)OCCCCCCN(CCCCO)CCCCCCOC(=O)C(CCCCCC)CCCCCCCC]

48. Composition of any one of claims 46 to 47, wherein the LNP comprises a PEG lipid, wherein the PEG-lipid is of formula (IVa): 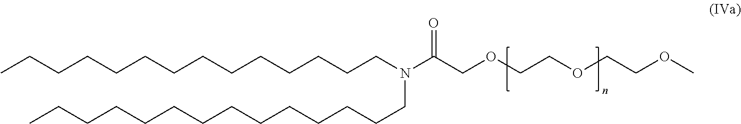
wherein n has a mean value ranging from 30 to 60, preferably wherein n has a mean value of about 45, 46, 47, 48, 49, 50, 51, 52, 53, 54, most preferably wherein n has a mean value of 49.

49. Composition of any one of claims 46 to 48, wherein the LNP comprises one or more neutral lipids and/or one or more steroid or steroid analogues.

50. Composition of claim 49, wherein the neutral lipid is 1,2-distearoyl-sn-glycero-3-phosphocholine (DSPC), preferably wherein the molar ratio of the cationic lipid to DSPC is in the range from about 2:1 to about 8:1.

51. Composition of claim 49, wherein the steroid is cholesterol, preferably wherein the molar ratio of the cationic lipid to cholesterol is in the range from about 2:1 to about 1:1

52. Composition of any one of claims 44 to 49, wherein the LNP comprises or consisting of
(i) at least one cationic lipid, preferably as defined in claim 47;
(ii) a neutral lipid, preferably as defined in claim 50;
(iii) a steroid or steroid analogue, preferably as defined in claim 51; and
(iv) a PEG-lipid, e.g. PEG-DMG or PEG-cDMA, preferably as defined in claim 48.

53. Composition according to any one of claim 52, wherein (i) to (iv) are in a molar ratio of about 20-60% cationic lipid, 5-25% neutral lipid, 25-55% sterol, and 0.5-15% PEG-lipid.

54. Composition of claim 46, wherein the LNP comprises COATSOME® SS-EC.

55. Composition of any one of claims 46 and 54, wherein the LNP comprises a PEG lipid, wherein the PEG-lipid is DMG-PEG 2000.

56. Composition of any one of claims 46 and 54 to 55, wherein the LNP further comprises 1,2-diphytanoyl-sn-glycero-3-phosphoethanolamine (DPhyPE) and cholesterol.

57. Composition of claims 46 to 56, wherein the LNPs are preferably selected from GN01-LNP or LNP-III-3.

58. A vaccine comprising at least one coding RNA as defined in any one of claims 1 to 40, or the composition as defined in any one of claims 41 to 57.

59. Vaccine of claim 58, wherein the vaccine elicits an adaptive immune response.

60. Vaccine of claims 58 to 59, wherein the vaccine is a polyvalent vaccine, preferably a trivalent vaccine.

61. A Kit or kit of parts, comprising at least one coding RNA as defined in any one of claims 1 to 40, at least one composition as defined in any one of claims 41 to 57, and/or at least one vaccine as defined in any one of claims 58 to 60, optionally comprising a liquid vehicle for solubilising, and, optionally, technical instructions providing information on administration and dosage of the components.

62. Coding RNA as defined in any one of claims 1 to 40, the composition as defined in any one of claims 41 to 57, the vaccine as defined in any one of claims 58 to 60, or the kit or kit of parts as defined in claim 57, for use as a medicament.

63. Coding RNA as defined in any one of claims 1 to 40, the composition as defined in any one of claims 41 to 53, the vaccine as defined in any one of claims 54 to 56, or the kit or kit of parts as defined in claim 61, for use in the treatment or prophylaxis of a Rotavirus infection, or of a disorder related to such an infection.

64. Use according to claim 63, wherein the Rotavirus infection is a Rotavirus A infection, in particular a Rotavirus A infection of serotypes [P4], [P6], and/or [P8].

65. A method of treating or preventing a disorder, wherein the method comprises applying or administering to a subject in need thereof at least one coding RNA as defined in any one of claims 1 to 40, at least one composition as defined in any one of claims 41 to 57, at least one vaccine as defined in any one of claims 58 to 60, or at least one kit or kit of parts as defined in claim 61.

66. Method of claim 65 wherein the disorder is an infection with a Rotavirus, or a disorder related to such an infection, preferably a Rotavirus A, or a disorder related to such an infection.

67. Method of claims 65 to 66, wherein the subject in need is a mammalian subject, preferably a human subject.

68. Method of any one of claims 65 to 67, wherein applying or administering to a subject is performed using intramuscular administration, preferably intramuscular injection.
